(19) United States
(12) Patent Application Publication
(10) Pub. No.: US 2014/0193510 A1
(43) Pub. Date: Jul. 10, 2014

(54) Polymer-Agent Conjugates, Particles, Compositions, and Related Methods of Use

(71) Applicant: CERULEAN PHARMA INC., Cambridge, MA (US)

(72) Inventors: Scott Eliasof, Lexington, MA (US); Thomas C Crawford, Essex, CT (US); Geeti Gangal, Cambridge, MA (US); Lawrence Alan Reiter, Mystic, CT (US); Pei-Sze Ng, Cambridge, MA (US)

(73) Assignee: CERULEAN PHARMA INC., Cambridge, MA (US)

(21) Appl. No.: 14/180,101

(22) Filed: Feb. 13, 2014

Related U.S. Application Data
(63) Continuation of application No. 12/748,669, filed on Mar. 29, 2010, now abandoned.
Application No. 12/748,669 is a continuation of application No. PCT/US10/28831, filed on Mar. 26, 2010.
(60) Provisional application No. 61/164,720, filed on Mar. 30, 2009.
(60) Provisional application No. 61/164,722, filed on Mar. 30, 2009.
(60) Provisional application No. 61/164,725, filed on Mar. 30, 2009.
(60) Provisional application No. 61/164,728, filed on Mar. 30, 2009.
(60) Provisional application No. 61/164,731, filed on Mar. 30, 2009.
(60) Provisional application No. 61/164,734, filed on Mar. 30, 2009.
(60) Provisional application No. 61/262,993, filed on Nov. 20, 2009.
(60) Provisional application No. 61/262,994, filed on Nov. 20, 2009.
(60) Provisional application No. 61/263,179, filed on Nov. 20, 2009.
(60) Provisional application No. 61/312,422, filed on Mar. 10, 2010.

(51) Int. Cl. A61K 47/48; A61K 31/69; A61K 31/704; A61K 31/675; A61K 9/14; A61K 31/337

(52) U.S. Cl. CPC A61K 47/482; A61K 9/14; A61K 31/337; A61K 31/704; A61K 31/675; A61K 31/69
USPC 424/501; 424/78.3

(57) ABSTRACT

Described herein are polymer-agent conjugates and particles, which can be used, for example, in the treatment of cancer. Also described herein are mixtures, compositions and dosage forms containing the particles, methods of using the particles (e.g., to treat a disorder), kits including the polymer-agent conjugates and particles, methods of making the polymer-agent conjugates and particles, methods of storing the particles and methods of analyzing the particles.

### RELATED APPLICATIONS

This application is a continuation application of International Application No. PCT/US10/28831, filed on Mar. 26, 2010, which claims the benefit of U.S. Provisional Application No. 61/164,720, filed Mar. 30, 2009, U.S. Provisional Application No. 61/164,722, filed Mar. 30, 2009, U.S. Provisional Application No. 61/164,725, filed Mar. 30, 2009, U.S. Provisional Application No. 61/164,728, filed Mar. 30, 2009, U.S. Provisional Application No. 61/164,731, filed Mar. 30, 2009, U.S. Provisional Application No. 61/164,734, filed Mar. 30, 2009, U.S. Provisional Application No. 61/262,993, filed Nov. 20, 2009, U.S. Provisional Application No. 61/262,994, filed Nov. 20, 2009, U.S. Provisional Application No. 61/263,179, filed Nov. 20, 2009 and U.S. Provisional Application No. 61/312,422, filed Mar. 10, 2010. The entire teachings of all of the foregoing applications are incorporated herein by reference.

### BACKGROUND OF INVENTION

The delivery of a drug with controlled release of the active agent is desirable to provide optimal use and effectiveness. Controlled release polymer systems may increase the efficacy of the drug and minimize problems with patient compliance.

### SUMMARY OF INVENTION

Described herein are polymer-agent conjugates and particles, which can be used, for example, in the treatment of cancer, cardiovascular diseases, inflammatory disorders (e.g., an inflammatory disorder that includes an inflammatory disorder caused by, e.g., an infectious disease) or autoimmune disorders. Also described herein are mixtures, compositions and dosage forms containing the particles, methods of using the particles (e.g., to treat a disorder), kits including the polymer-agent conjugates and particles, methods of making the polymer-agent conjugates and particles, methods of storing the particles and methods of analyzing the particles.

Accordingly, in one aspect, the invention features a polymer-agent conjugate comprising:

a polymer; and

an agent (e.g., a therapeutic or diagnostic agent) attached to the polymer.

In some embodiments, the polymer is a biodegradable polymer (e.g., polylactic acid (PLA), polyglycolic acid (PGA), poly(lactic-co-glycolic acid) (PLGA), polycaprolactone (PCL), polydioxanone (PDO), polyanhydrides, polyorthoesters, or chitosan). In some embodiments, the polymer is a hydrophobic polymer. In some embodiments, the polymer is PLA. In some embodiments, the polymer is PGA.

In some embodiments, the polymer is a copolymer of lactic and glycolic acid (e.g., PLGA). In some embodiments, the polymer is a PLGA-ester. In some embodiments, the polymer is a PLGA-lauryl ester. In some embodiments, the polymer comprises a terminal free acid prior to conjugation to an agent. In some embodiments, the polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the polymer has a glass transition temperature of about 20° C. to about 60° C. In some embodiments, the polymer has a polymer polydispersity index of less than or equal to about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the polymer has a hydrophilic portion and a hydrophobic portion. In some embodiments, the polymer is a block copolymer. In some embodiments, the polymer comprises two regions, the two regions together being at least about 70% by weight of the polymer (e.g., at least about 80%, at least about 90%, at least about 95%). In some embodiments, the polymer is a block copolymer comprising a hydrophobic polymer and a hydrophilic polymer. In some embodiments, the polymer, e.g., a diblock copolymer, comprises a hydrophobic polymer and a hydrophilic polymer. In some embodiments, the polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the polymer is a biodegradable polymer (e.g., PLA, PGA, PLGA, PCL, PDO, polyanhydrides, polyorthoesters, or chitosan). In some embodiments, the hydrophobic portion of the polymer is PLA. In some embodiments, the hydrophobic portion of the polymer is PGA. In some embodiments, the hydrophobic portion of the polymer is a copolymer of lactic and glycolic acid (e.g., PLGA). In some embodiments, the hydrophobic portion of the polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic portion of the polymer is polyethylene glycol (PEG). In some embodiments, the hydrophilic portion of the polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of the weight average molecular weights of the hydrophilic to hydrophobic portions of the polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the polymer has a weight average molecular weight of from about 2 kDa to 3.5 kDa and the ratio of the weight average molecular weight of the hydrophilic to hydrophobic portions of the polymer is from about 1:4 to about 1:6.5 (e.g., 1:4, 1:4.5, 1:5, 1:5.5, 1:6, 1:6.5). In one embodiment, the hydrophilic portion of the polymer has a weight average molecular weight of from about 4 kDa to 6 kDa (e.g., 5 kDa) and the ratio of the weight average molecular weight of the hydrophilic to hydrophobic portions of the polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic portion of the polymer has a terminal hydroxyl moiety prior to conjugation to an agent. In some embodiments, the hydrophilic portion of has a terminal alkoxy moiety. In some embodiments, the hydrophilic portion of the polymer is a methoxy PEG (e.g., a terminal methoxy PEG). In some embodiments, the hydrophilic polymer portion of the polymer does not have a terminal alkoxy moiety. In some embodiments, the terminus of the hydrophilic polymer portion of the polymer is conjugated to a hydrophobic polymer, e.g., to make a triblock copolymer.

In some embodiments, the hydrophilic portion of the polymer is attached to the hydrophobic portion through a covalent bond. In some embodiments, the hydrophilic polymer is attached to the hydrophobic polymer through an amide, ester, ether, amino, carbamate, or carbonate bond (e.g., an ester or an amide).

In some embodiments, a single agent is attached to a single polymer, e.g., to a terminal end of the polymer. In some embodiments, a plurality of agents are attached to a single polymer (e.g., 2, 3, 4, 5, 6, or more). In some embodiments, the agents are the same agent. In some embodiments, the agents are different agents. In some embodiments, the agent is a diagnostic agent.

In some embodiments, the agent is a therapeutic agent. In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine).

In some embodiments, the anti-cancer agent is paclitaxel, attached to the polymer via the hydroxyl group at the 2′ position, the hydroxyl group at the 1 position and/or the hydroxyl group at the 7 position. In some embodiments, the anti-cancer agent is paclitaxel, attached to the polymer via the 2′ position and/or the 7 position.

In some embodiments, the anti-cancer agent is docetaxel, attached to the polymer via the hydroxyl group at the 2′ position, the hydroxyl group at the 7 position, the hydroxyl group at the 10 position and/or the hydroxyl group at the 1 position. In some embodiments, the anti-cancer agent is docetaxel, attached to the polymer via the hydroxyl group at the 2′ position, the hydroxyl group at the 7 position and/or the hydroxyl group at the 10 position.

In some embodiments, the anti-cancer agent is docetaxel-succinate.

In some embodiments, the anti-cancer agent is a taxane that is attached to the polymer via the hydroxyl group at the 7 position and has an acyl group or a hydroxy protecting group on the hydroxyl group at the 2′ position (e.g., wherein the anti-cancer agent is a taxane such as paclitaxel, docetaxel, larotaxel or cabazitaxel). In some embodiments, the anti-cancer agent is larotaxel. In some embodiments, the anti-cancer agent is cabazitaxel.

In some embodiments, the anti-cancer agent is doxorubicin.

In some embodiments, the therapeutic agent is a proteasome inhibitor or a boronic acid containing drug (and particles and compositions including the same), including drugs that are both a proteasome inhibitor and contain a boronic acid group, as described in structural formula A herein. In some embodiments, the therapeutic agent is a bortezomib (Velcade®).

In some embodiments, the therapeutic agent is a boronic acid containing drug described in U.S. Pat. Nos. 5,780,454, 6,083,903, 6,297,217, 6,617,317, 6,713,446, 6,747,150, 6,958,319, 7,119,080, 7,582,621, 7,465,836, 7,393,856, and 7,390,806, and U.S. Published Applications US2009/0239824, US2009/0227541, US2008/0293675, US2007/0286822, US2007/0265226, US2007/0179296, US2007/0155699 and US2006/0234981, the entire teachings of which are incorporated by reference. These patent documents are referred to hereinafter as “PATENTS.”

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of an inflammatory or autoimmune disease, for example as described herein. In some embodiments, the therapeutic agent is an agent for the prevention of an inflammatory or autoimmune disease, for example as described herein.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, a single agent is attached to a polymer. In some embodiments, multiple agents are attached to a polymer (e.g., 2, 3, 4, 5, 6 or more agents). In some embodiments, the agents are the same agent. In some embodiments, the agents are different agents.

In some embodiments, the agent is doxorubicin, and is covalently attached to the polymer through an amide bond.

In some embodiments, the polymer-agent conjugate is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the boronic acid containing drug is as described in the PATENTS. In another alternative, the boronic acid containing drug is bortezomib.

In some embodiments, the polymer-agent conjugate is:

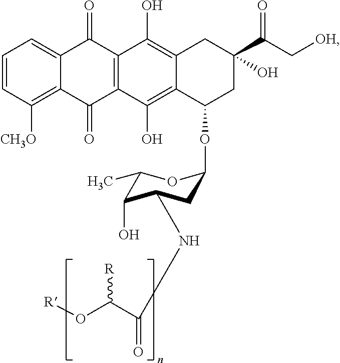

wherein about 30% to about 70%, 35% to about 65%, 40% to about 60%, 45% to about 55% of R substituents are hydrogen (e.g., about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is covalently attached to the polymer through an ester bond. In some embodiments, the agent is paclitaxel, and is attached to the polymer via the hydroxyl group at the 2′ position.

In some embodiments, the polymer-agent conjugate is:

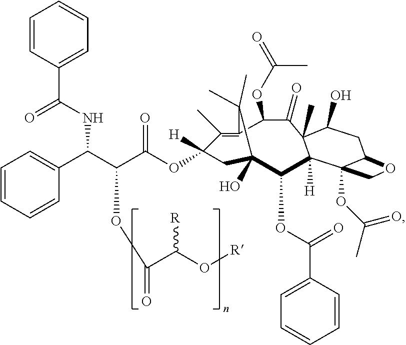

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is attached to the polymer via the hydroxyl group at the 7 position.

In some embodiments, the polymer-agent conjugate is:

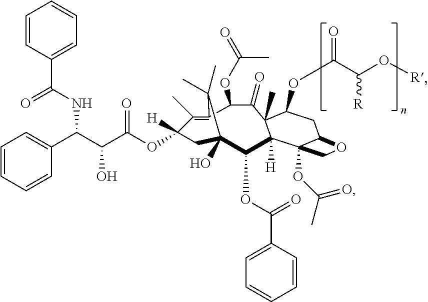

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particle includes a combination of polymer-paclitaxel conjugates described herein, e.g., polymer-paclitaxel conjugates illustrated above.

In some embodiments, the polymer-agent conjugate has the following formula (I):

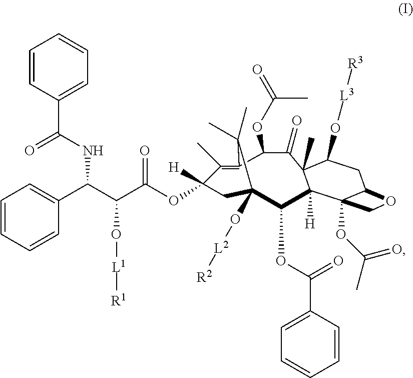

wherein L¹, L² and L³ are each independently a bond or a linker, e.g., a linker described herein;

wherein R¹, R² and R³ are each independently hydrogen, C₁-C₆ alkyl, acyl, or a polymer of formula (II):

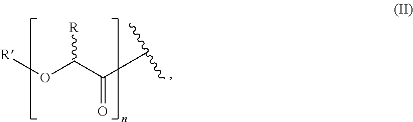

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R² and R³ is a polymer of formula (II).

In some embodiments, L² is a bond and R² is hydrogen.

In some embodiments, the agent is paclitaxel, and is covalently attached to the polymer via a carbonate bond.

In some embodiments, the agent is docetaxel, and is covalently attached to the polymer through an ester bond. In some embodiments, the agent is docetaxel, and is attached to the polymer via the hydroxyl group at the 2′ position.

In some embodiments, the polymer-agent conjugate is:

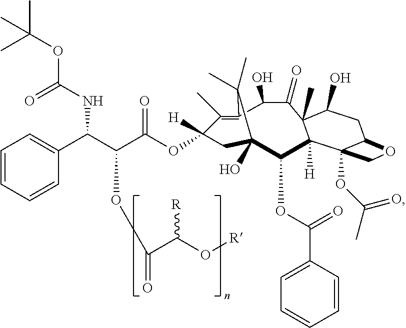

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 7 position.

In some embodiments, the polymer-agent conjugate is:

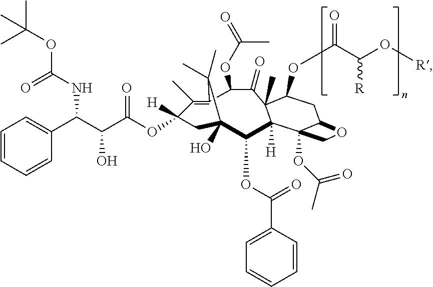

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the polymer-agent conjugate is:

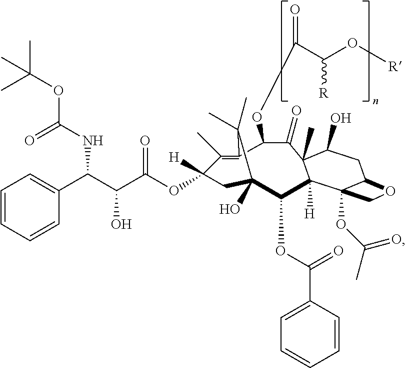

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is covalently attached to the polymer through a carbonate bond.

In some embodiments, the particle includes a combination of polymer-docetaxel conjugates described herein, e.g., polymer-docetaxel conjugates illustrated above.

In some embodiments, the agent is attached to the polymer through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent)

In some embodiments, the polymer-agent conjugate is:

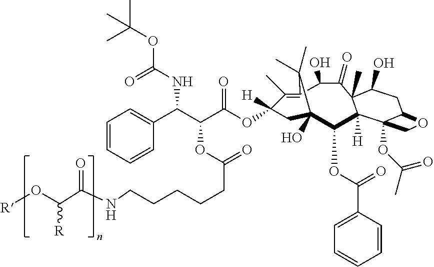

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate is:

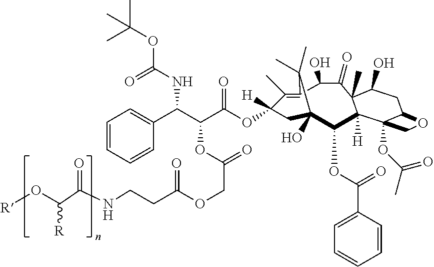

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate has the following formula (III):

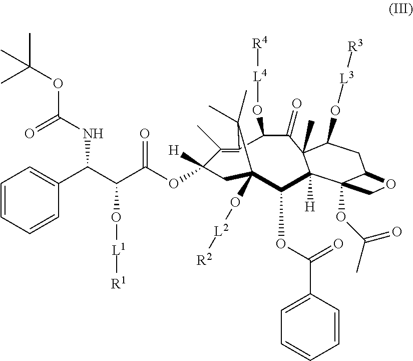

wherein L¹, L², L³ and L⁴ are each independently a bond or a linker, e.g., a linker described herein;

R¹, R², R³ and R⁴ are each independently hydrogen, C₁-C₆ alkyl, acyl, a hydroxy protecting group, or a polymer of formula (IV):

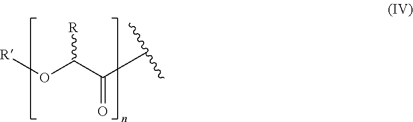

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R², R³ and R⁴ is a polymer of formula (IV).

In some embodiments, L² is a bond and R² is hydrogen.

In some embodiments, two agents are attached to a polymer via a multifunctional linker. In some embodiments, the two agents are the same agent. In some embodiments, the two agents are different agents. In some embodiments, the agent is docetaxel, and is covalently attached to the polymer via a glutamate linker.

In some embodiments, the polymer-agent conjugate is:

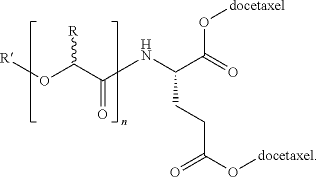

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxy group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., one docetaxel is attached via the hydroxyl group at the 2′ position and the other is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is docetaxel, and is covalently attached to the polymer via a tri(glutamate) linker.

In some embodiments, the polymer-agent conjugate is:

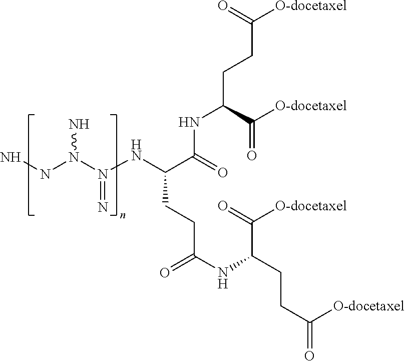

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, docetaxel molecules may be attached via different hydroxyl groups, e.g., three docetaxel molecules are attached via the hydroxyl group at the 2′ position and the other is attached via the hydroxyl group at the 7 position.

In another aspect, the invention features a composition comprising a plurality of polymer-agent conjugates, wherein the polymer-agent conjugate has the following formula:

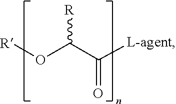

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is a taxane, e.g., docetaxel, paclitaxel, larotaxel or cabazitaxel.

In some embodiments, L is a bond.

In some embodiments, L is a linker, e.g., a linker described herein.

In some embodiments, the composition comprises a plurality of polymer-agent conjugates wherein the polymer-agent conjugates have the same polymer and the same agent, and differ in the nature of the linkage between the agent and the polymer. For example, in some embodiments, the polymer is PLGA, the agent is paclitaxel, and the plurality of polymer-agent conjugates includes PLGA attached to paclitaxel via the hydroxyl group at the 2′ position and PLGA attached to paclitaxel via the hydroxyl group at the 7 position. In some embodiments, the polymer is PLGA, the agent is paclitaxel, and the plurality of polymer-agent conjugates includes PLGA attached to paclitaxel via the hydroxyl group at the 2′ position, PLGA attached to paclitaxel via the hydroxyl group at the 7 position, and/or PLGA attached to paclitaxel via the hydroxyl group at the 1 position.

In some embodiments, the polymer is PLGA, the agent is docetaxel, and the plurality of polymer-agent conjugates includes PLGA attached to docetaxel via the hydroxyl group at the 2′ position and PLGA attached to docetaxel via the hydroxyl group at the 7 position. In some embodiments, the polymer is PLGA, the agent is docetaxel, and the plurality of polymer-agent conjugates includes PLGA attached to docetaxel via the hydroxyl group at the 2′ position, PLGA attached to docetaxel via the hydroxyl group at the 7 position, and/or PLGA attached to docetaxel via the hydroxyl group at the 10 position. In some embodiments, the polymer is PLGA, the agent is docetaxel, and the plurality of polymer-agent conjugates includes PLGA attached to docetaxel via the hydroxyl group at the 2′ position, PLGA attached to docetaxel via the hydroxyl group at the 7 position, PLGA attached to docetaxel via the 10 position and/or PLGA attached to docetaxel via the hydroxyl group at the 1 position.

In another aspect, the invention features a particle. The particle comprises:

a first polymer,

a second polymer having a hydrophilic portion and a hydrophobic portion, an agent (e.g., a therapeutic or diagnostic agent) attached to the first polymer or second polymer, and

optionally, the particle comprises one or more of the following properties:

it further comprises a compound comprising at least one acidic moiety, wherein the compound is a polymer or a small molecule;

it further comprises a surfactant;

the first polymer is a PLGA polymer, wherein the ratio of lactic acid to glycolic acid is from about 25:75 to about 75:25 and, optionally, the agent is attached to the first polymer;

the first polymer is PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or

the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, poly(vinyl alcohol) (PVA), poly(vinylpyrrolidone) (PVP), poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the agent is attached to the first polymer to form a polymer-agent conjugate. In some embodiments, the agent is attached to the second polymer to form a polymer-agent conjugate.

In some embodiments the amount of agent in the particle that is not attached to the first or second polymer is less than about 5% (e.g., less than about 2% or less than about 1%, e.g., in terms of w/w or number/number) of the amount of agent attached to the first polymer or second polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a copolymer, e.g., a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters, or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2:to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an anti-cancer agent or other therapeutic or diagnostic agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single agent is attached to a single polymer (e.g., a single first polymer or a single second polymer), e.g., to a terminal end of the polymer. In some embodiments, a plurality of agents are attached to a single polymer (e.g., a single first polymer or a single second polymer) (e.g., 2, 3, 4, 5, 6, or more). In some embodiments, the agents are the same agent. In some embodiments, the agents are different agents. In some embodiments, the agent is a diagnostic agent.

In some embodiments, the agent is a therapeutic agent. In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine). In other embodiments, therapeutic agent is a boronic acid containing drug.

In some embodiments, the therapeutic agent is a boronic acid containing drug as described in structural formula A herein. In some embodiments, the therapeutic agent is a boronic acid containing drug described in the PATENTS. In some embodiments, the therapeutic agent is a bortezomib.

In some embodiments, the anti-cancer agent is paclitaxel, attached to the polymer via the hydroxyl group at the 2′ position, the hydroxyl group at the 1 position and/or the hydroxyl group at the 7 position. In some embodiments, the anti-cancer agent is paclitaxel, attached to the polymer via the 2′ position and/or the 7 position.

In some embodiments, the anti-cancer agent is docetaxel, attached to the polymer via the hydroxyl group at the 2′ position, the hydroxyl group at the 7 position, the hydroxyl group at the 10 position and/or the hydroxyl group at the 1 position. In some embodiments, the anti-cancer agent is docetaxel, attached to the polymer via the hydroxyl group at the 2′ position, the hydroxyl group at the 7 position and/or the hydroxyl group at the 10 position.

In some embodiments, the anti-cancer agent is docetaxel-succinate.

In some embodiments, the anti-cancer agent is a taxane that is attached to the polymer via the hydroxyl group at the 7 position and has an acyl group or a hydroxy protecting group on the hydroxyl group at the 2′ position (e.g., wherein the anti-cancer agent is a taxane such as paclitaxel, docetaxel, larotaxel or cabazitaxel). In some embodiments, the anti-cancer agent is larotaxel. In some embodiments, the anti-cancer agent is cabazitaxel.

In some embodiments, the anti-cancer agent is doxorubicin.

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, a single agent is attached to a polymer. In some embodiments, multiple agents are attached to a polymer (e.g., 2, 3, 4, 5, 6 or more agents). In some embodiments, the agents are the same agent. In some embodiments, the agents are different agents.

In some embodiments, the agent is doxorubicin, and is covalently attached to the first polymer through an amide bond.

In some embodiments, the polymer-agent conjugate in the particle, e.g., the nanoparticle, is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the polymer-agent conjugate in the particle, e.g., the nanoparticle, is:

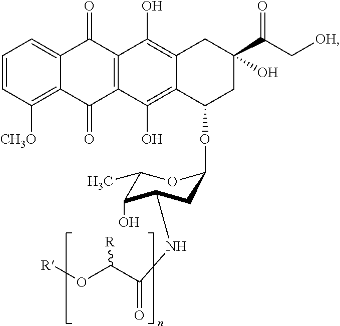

wherein about 30% to about 70%, 35% to about 65%, 40% to about 60%, 45% to about 55% of R substituents are hydrogen (e.g., about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is covalently attached to the polymer through an ester bond. In some embodiments, the agent is paclitaxel, and is attached to the polymer via the hydroxyl group at the 2′ position.

In some embodiments, the polymer-agent conjugate in the particle, e.g., the nanoparticle, is:

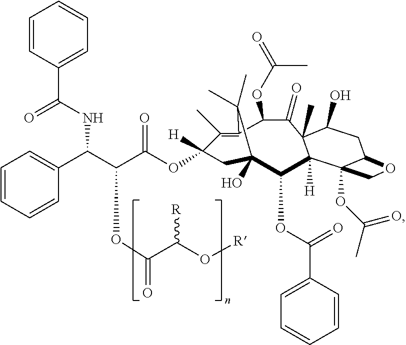

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is attached to the polymer via the hydroxyl group at the 7 position.

In some embodiments, the polymer-agent conjugate in the particle, e.g., the nanoparticle, is:

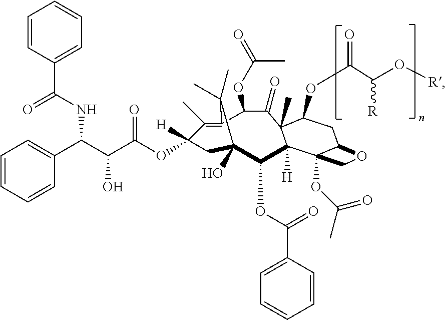

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particle includes a combination of polymer-paclitaxel conjugates described herein, e.g., polymer-paclitaxel conjugates illustrated above.

In some embodiments, the polymer-agent conjugate in the particle, e.g., the nanoparticle, has the following formula (I):

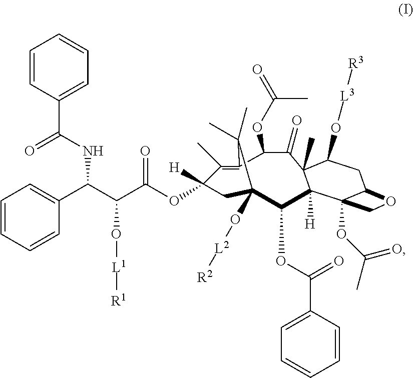

wherein L¹, L² and L³ are each independently a bond or a linker, e.g., a linker described herein;

wherein R¹, R² and R³ are each independently hydrogen, C₁-C₆ alkyl, acyl, or a polymer of formula (II):

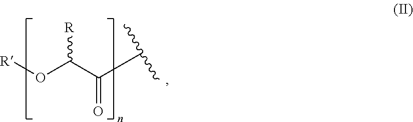

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R² and R³ is a polymer of formula (II).

In some embodiments, L² is a bond and R² is hydrogen.

In some embodiments, the agent is paclitaxel, and is covalently attached to the polymer via a carbonate bond.

In some embodiments, the agent is docetaxel, and is covalently attached to the polymer through an ester bond. In some embodiments, the agent is docetaxel, and is attached to the polymer via the hydroxyl group at the 2′ position.

In some embodiments, the polymer-agent conjugate in the particle, e.g., the nanoparticle, is:

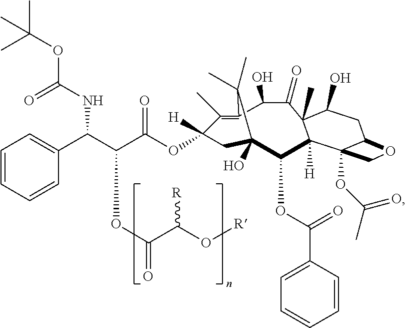

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 7 position.

In some embodiments, the polymer-agent conjugate in the particle, e.g., the nanoparticle, is:

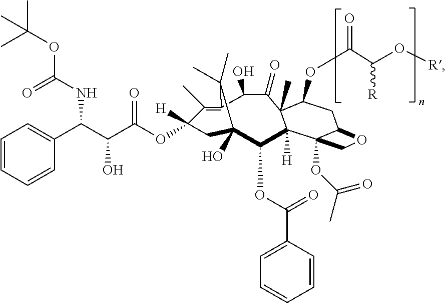

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the polymer-agent conjugate in the particle, e.g., the nanoparticle, is:

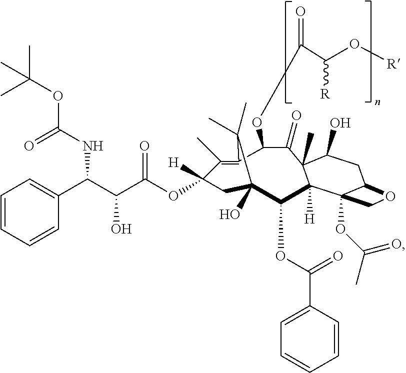

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is covalently attached to the polymer through a carbonate bond.

In some embodiments, the particle includes a combination of polymer-docetaxel conjugates described herein, e.g., polymer-docetaxel conjugates illustrated above.

In some embodiments, the agent is attached to the polymer through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, the polymer-agent conjugate in the particle, e.g., the nanoparticle, is:

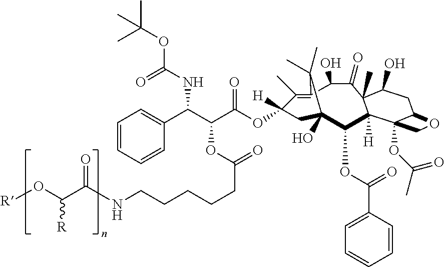

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate is:

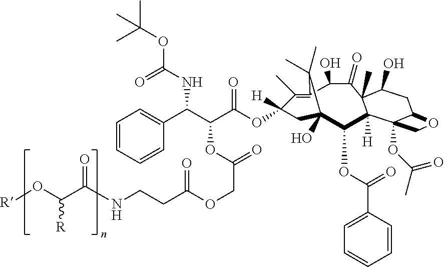

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate in the particle, e.g., the nanoparticle, has the following formula (III):

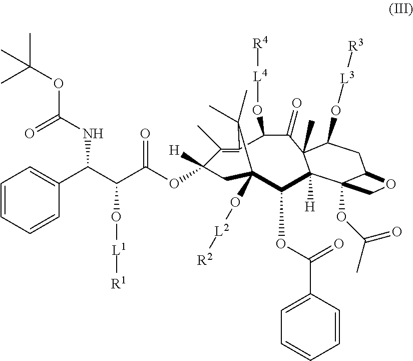

wherein L¹, L², L³ and L⁴ are each independently a bond or a linker, e.g., a linker described herein;

R¹, R², R³ and R⁴ are each independently hydrogen, C₁-C₆ alkyl, acyl, a hydroxy protecting group, or a polymer of formula (IV):

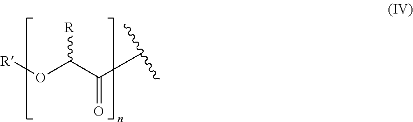

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R², R³ and R⁴ is a polymer of formula (IV).

In some embodiments, L² is a bond and R² is hydrogen.

In some embodiments, two agents are attached to a polymer via a multifunctional linker. In some embodiments, the two agents are the same agent. In some embodiments, the two agents are different agents. In some embodiments, the agent is docetaxel, and is covalently attached to the polymer via a glutamate linker.

In some embodiments, the polymer-agent conjugate in the particle, e.g., the nanoparticle, is:

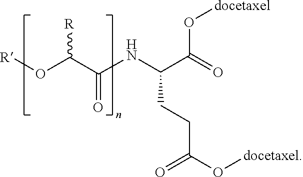

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the 2′ hydroxyl group at the position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., one docetaxel is attached via the hydroxyl group at the 2′ position and the other is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is docetaxel, and is covalently attached to the polymer via a tri(glutamate) linker.

In some embodiments, the polymer-agent conjugate in the particle, e.g., the nanoparticle, is:

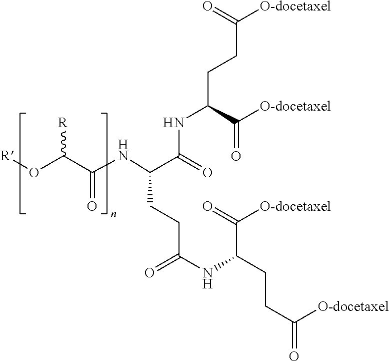

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, docetaxel molecules may be attached via different hydroxyl groups, e.g., three docetaxel molecules are attached via the hydroxyl group at the 2′ position and the other is attached via the hydroxyl group at the 7 position.

In some embodiments, the polymer-agent conjugate has the following formula:

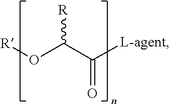

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is a taxane, e.g., docetaxel, paclitaxel, larotaxel or cabazitaxel.

In some embodiments, L is a bond.

In some embodiments, L is a linker, e.g., a linker described herein.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, the agent is paclitaxel, and the plurality of polymer-agent conjugates includes PLGA polymers attached to paclitaxel via the hydroxyl group at the 2′ position, and PLGA polymers attached to paclitaxel via the hydroxyl group at the 7 position. In some embodiments, the polymer is PLGA, the agent is paclitaxel, and the plurality of polymer-agent conjugates includes PLGA polymers attached to paclitaxel via the hydroxyl group at the 2′ position, PLGA polymers attached to paclitaxel via the hydroxyl group at the 7 position, and/or PLGA polymers attached to paclitaxel via the hydroxyl group at the 1 position. In some embodiments, the polymer is PLGA, the agent is paclitaxel, and the plurality of polymer-agent conjugates includes paclitaxel molecules attached to more than one polymer chain, e.g., paclitaxel molecules with PLGA polymers attached to the hydroxyl group at the 2′ position, the hydroxyl group at the 7 position and/or the hydroxyl group at the 1 position.

In some embodiments, the polymer is PLGA, the agent is docetaxel, and the plurality of polymer-agent conjugates includes PLGA attached to docetaxel via the hydroxyl group at the 2′ position and PLGA attached to docetaxel via the hydroxyl group at the 7 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, and/or PLGA polymers attached to docetaxel via the hydroxyl group at the 10 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, PLGA polymers attached to docetaxel via the hydroxyl group at the 10 position and/or PLGA polymers attached to docetaxel via the hydroxyl group at the 1 position. In some embodiments, the polymer is PLGA, the agent is docetaxel, and the plurality of polymer-agent conjugates includes docetaxel molecules attached to more than one polymer chain, e.g., docetaxel molecules with PLGA polymers attached to the hydroxyl group at the 2′ position, the hydroxyl group at the 7 position, the hydroxyl group at the 10 position and/or the hydroxyl group at the 1 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer,

a second polymer having a hydrophilic portion and a hydrophobic portion,

an agent (e.g., a therapeutic or diagnostic agent), wherein the agent is attached to the first polymer to form a polymer-agent conjugate, and

optionally, the particle comprises one or more of the following:

it further comprises a compound comprising at least one acidic moiety, wherein the compound is a polymer or a small molecule;

it further comprises a surfactant;

the first polymer is a PLGA polymer, wherein the ratio of lactic acid to glycolic acid is from about 25:75 to about 75:25 and the agent is attached to the first polymer;

the first polymer is PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or

the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 15%, about 18%, about 20%, about 22%, about 24%, about 26%, about 28%, or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In an embodiment the amount of agent in the particle that is not attached to the first polymer is less than about 5% (e.g., less than about 2% or less than about 1%, e.g., in terms of w/w or number/number) of the amount of agent attached to the first polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters, or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2:to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an anti-cancer agent or other therapeutic or diagnostic agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the therapeutic agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single agent is attached to a single first polymer, e.g., to a terminal end of the polymer. In some embodiments, a plurality of agents are attached to a single first polymer (e.g., 2, 3, 4, 5, 6, or more). In some embodiments, the agents are the same agent. In some embodiments, the agents are different agents. In some embodiments, the agent is a diagnostic agent.

In some embodiments, the agent is a therapeutic agent. In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine). In some embodiments, the therapeutic agent is a boronic acid containing drug.

In some embodiments, the therapeutic agent is a boronic acid containing drug as described in structural formula A herein. In some embodiments, the therapeutic agent is a boronic acid containing drug described in the PATENTS. In some embodiments, the therapeutic agent is a bortezomib.

In some embodiments, the anti-cancer agent is paclitaxel, attached to the polymer via the hydroxyl group at the 2′ position, the hydroxyl group at the 1 position and/or the hydroxyl group at the 7 position. In some embodiments, the anti-cancer agent is paclitaxel, attached to the polymer via the hydroxyl group at the 2′ position and/or the hydroxyl group at the 7 position.

In some embodiments, the anti-cancer agent is docetaxel, attached to the polymer via the hydroxyl group at the 2′ position, the hydroxyl group at the 1 position, the hydroxyl group at the 7 position and/or the hydroxyl group at the 10 position. In some embodiments, the anti-cancer agent is docetaxel, attached to the polymer via the hydroxyl group at the 2′ position, the hydroxyl group at the 7 position and/or the hydroxyl group at the 10 position.

In some embodiments, the anti-cancer agent is docetaxel-succinate.

In some embodiments, the anti-cancer agent is a taxane that is attached to the polymer via the hydroxyl group at the 7 position and has an acyl group or a hydroxy protecting group on the hydroxyl group at the 2′ position (e.g., wherein the anti-cancer agent is a taxane such as paclitaxel, docetaxel, larotaxel or cabazitaxel). In some embodiments, the anti-cancer agent is larotaxel. In some embodiments, the anti-cancer agent is cabazitaxel.

In some embodiments, the anti-cancer agent is doxorubicin.

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, a single agent is attached to the polymer. In some embodiments, multiple agents are attached to the polymer (e.g., 2, 3, 4, 5, 6 or more agents). In some embodiments, the agents are the same agent. In some embodiments, the agents are different agents.

In some embodiments, the agent is doxorubicin, and is covalently attached to the first polymer through an amide bond.

In some embodiments, the polymer-agent conjugate is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the polymer-agent conjugate in the particle, e.g., the nanoparticle, is:

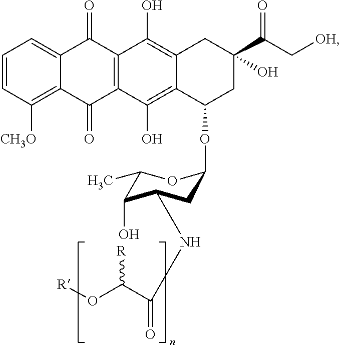

wherein about 30% to about 70%, 35% to about 65%, 40% to about 60%, 45% to about 55% of R substituents are hydrogen (e.g., about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is covalently attached to the polymer through an ester bond. In some embodiments, the agent is paclitaxel, and is attached to the polymer via the hydroxyl group at the 2′ position.

In some embodiments, the polymer-agent conjugate in the particle, e.g., the nanoparticle, is:

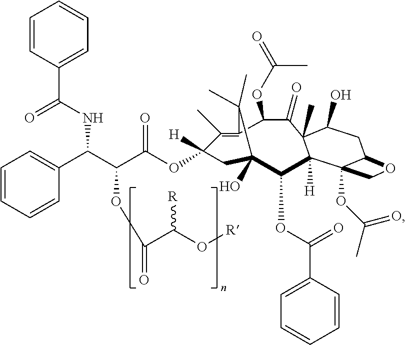

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is attached to the polymer via the hydroxyl group at the 7 position.

In some embodiments, the polymer-agent conjugate in the particle, e.g., the nanoparticle, is:

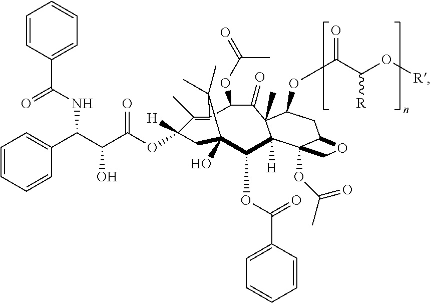

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particle includes a combination of polymer-paclitaxel conjugates described herein, e.g., polymer-paclitaxel conjugates illustrated above.

In some embodiments, the polymer-agent conjugate in the particle, e.g., the nanoparticle, has the following formula (I):

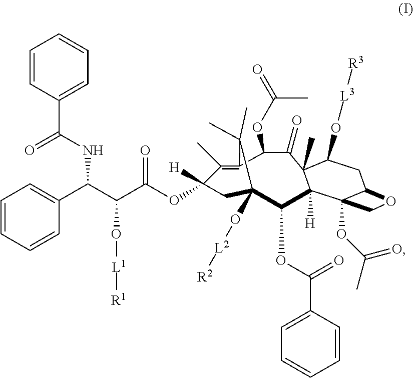

wherein L¹, L² and L³ are each independently a bond or a linker, e.g., a linker described herein;

wherein R¹, R² and R³ are each independently hydrogen, C₁-C₆ alkyl, acyl, or a polymer of formula (II):

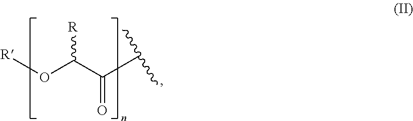

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R² and R³ is a polymer of formula (II).

In some embodiments, L² is a bond and R² is hydrogen.

In some embodiments, the agent is paclitaxel, and is covalently attached to the polymer via a carbonate bond.

In some embodiments, the agent is docetaxel, and is covalently attached to the polymer through an ester bond. In some embodiments, the agent is docetaxel, and is attached to the polymer via the hydroxyl group at the 2′ position.

In some embodiments, the polymer-agent conjugate in the particle, e.g., the nanoparticle, is:

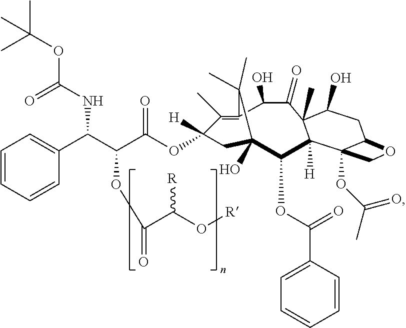

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 7 position.

In some embodiments, the polymer-agent conjugate in the particle, e.g., the nanoparticle, is:

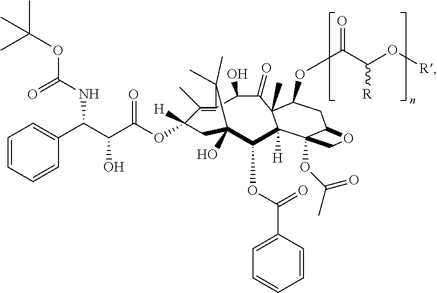

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the polymer-agent conjugate in the particle, e.g., the nanoparticle, is:

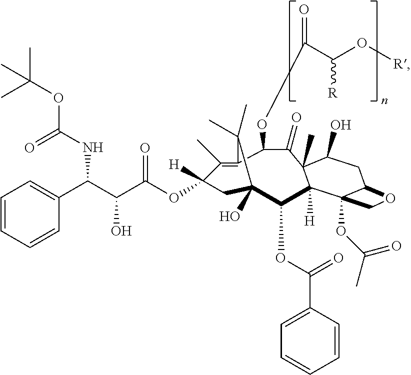

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is covalently attached to the polymer through a carbonate bond.

In some embodiments, the particle includes a combination of polymer-docetaxel conjugates described herein, e.g., polymer-docetaxel conjugates illustrated above.

In some embodiments, the agent is attached to the polymer through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, the polymer-agent conjugate in the particle, e.g., the nanoparticle, is:

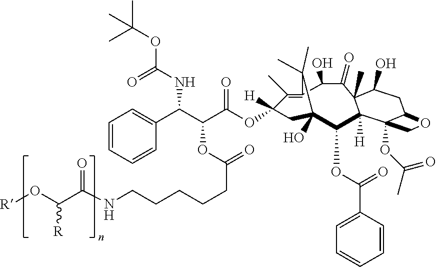

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate is:

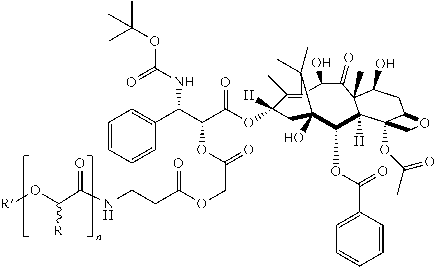

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate in the particle, e.g., the nanoparticle, has the following formula (III):

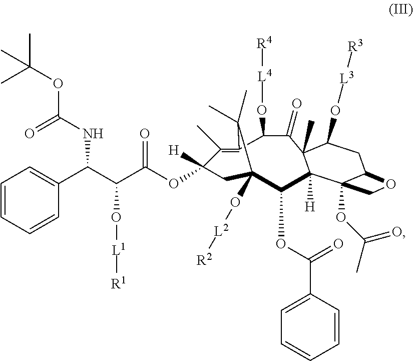

wherein L¹, L², L³ and L⁴ are each independently a bond or a linker, e.g., a linker described herein;

R¹, R², R³ and R⁴ are each independently hydrogen, C₁-C₆ alkyl, acyl, a hydroxy protecting group, or a polymer of formula (IV):

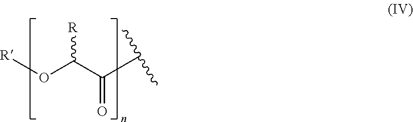

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R², R³ and R⁴ is a polymer of formula (IV).

In some embodiments, L² is a bond and R² is hydrogen.

In some embodiments, two agents are attached to a polymer via a multifunctional linker. In some embodiments, the two agents are the same agent. In some embodiments, the two agents are different agents. In some embodiments, the agent is docetaxel, and is covalently attached to the polymer via a glutamate linker.

In some embodiments, the polymer-agent conjugate in the particle, e.g., the nanoparticle, is:

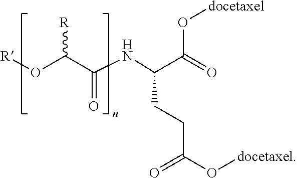

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., one docetaxel is attached via the hydroxyl group at the 2′ position and the other is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is docetaxel, and is covalently attached to the polymer via a tri(glutamate) linker.

In some embodiments, the polymer-agent conjugate in the particle, e.g., the nanoparticle, is:

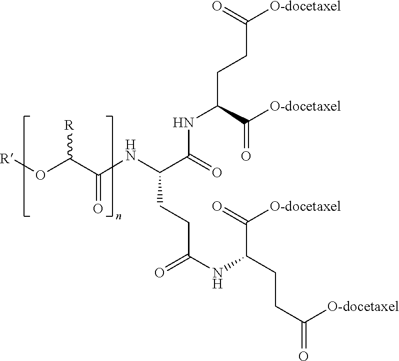

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, docetaxel molecules may be attached via different hydroxyl groups, e.g., three docetaxel molecules are attached via the hydroxyl group at the 2′ position and the other is attached via the hydroxyl group at the 7 position.

In some embodiments, the polymer-agent conjugate has the following formula:

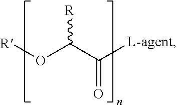

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is a taxane, e.g., docetaxel, paclitaxel, larotaxel or cabazitaxel.

In some embodiments, L is a bond.

In some embodiments, L is a linker, e.g., a linker described herein.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, the agent is paclitaxel, and the plurality of polymer-agent conjugates includes PLGA polymers attached to paclitaxel via the hydroxyl group at the 2′ position, and PLGA polymers attached to paclitaxel via the hydroxyl group at the 7 position. In some embodiments, the polymer is PLGA, the agent is paclitaxel, and the plurality of polymer-agent conjugates includes PLGA polymers attached to paclitaxel via the hydroxyl group at the 2′ position, PLGA polymers attached to paclitaxel via the hydroxyl group at the 7 position, and/or PLGA polymers attached to paclitaxel via the hydroxyl group at the 1 position. In some embodiments, the polymer is PLGA, the agent is paclitaxel, and the plurality of polymer-agent conjugates includes paclitaxel molecules attached to more than one polymer chain, e.g., paclitaxel molecules with PLGA polymers attached to the hydroxyl group at the 2′ position, the hydroxyl group at the 7 position and/or the hydroxyl group at the 1 position.

In some embodiments, the polymer is PLGA, the agent is docetaxel, and the plurality of polymer-agent conjugates includes PLGA attached to docetaxel via the hydroxyl group at the 2′ position and PLGA attached to docetaxel via the hydroxyl group at the 7 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, and/or PLGA polymers attached to docetaxel via the hydroxyl group at the 10 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, PLGA polymers attached to docetaxel via the hydroxyl group at the 10 position and/or PLGA polymers attached to docetaxel via the hydroxyl group at the 1 position. In some embodiments, the polymer is PLGA, the agent is docetaxel, and the plurality of polymer-agent conjugates includes docetaxel molecules attached to more than one polymer chain, e.g., docetaxel molecules with PLGA polymers attached to the hydroxyl group at the 2′ position, the hydroxyl group at the 7 position, the hydroxyl group at the 10 position and/or the hydroxyl group at the 1 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer,

a second polymer having a hydrophilic portion and a hydrophobic portion,

a first agent (e.g., a therapeutic or diagnostic agent) attached to the first polymer or second polymer to form a polymer-agent conjugate, and

a second agent embedded in the particle.

In some embodiments, the second agent embedded in the particle makes up from about 0.1 to about 10% by weight of the particle (e.g., about 0.5% wt., about 1% wt., about 2% wt., about 3% wt., about 4% wt., about 5% wt., about 6% wt., about 7% wt., about 8% wt., about 9% wt., about 10% wt.).

In some embodiments, the second agent embedded in the particle is substantially absent from the surface of the particle. In some embodiments, the second agent embedded in the particle is substantially uniformly distributed throughout the particle. In some embodiments, the second agent embedded in the particle is not uniformly distributed throughout the particle. In some embodiments, the particle includes hydrophobic pockets and the embedded second agent is concentrated in hydrophobic pockets of the particle.

In some embodiments, the second agent embedded in the particle forms one or more non-covalent interactions with a polymer in the particle. In some embodiments, the second agent forms one or more hydrophobic interactions with a hydrophobic polymer in the particle. In some embodiments, the second agent forms one or more hydrogen bonds with a polymer in the particle.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the first agent and the second agent are the same agent (e.g., both the first and second agents are docetaxel). In some embodiments, the first agent and the second agent are different agents (e.g., one agent is docetaxel and the other is doxorubicin).

In some embodiments, the first agent is attached to the first polymer to form a polymer-agent conjugate. In some embodiments, first agent is attached to the second polymer to form a polymer-agent conjugate.

In some embodiments, the second agent is not covalently bound to the first or second polymer.

In an embodiment the amount of the first agent in the particle that is not attached to the first polymer is less than about 5% (e.g., less than about 2% or less than about 1%, e.g., in terms of w/w or number/number) of the amount of the first agent attached to the first polymer.

In some embodiments, the first polymer is a biodegradable polymer (e.g., PLA, PGA, PLGA, PCL, PDO, polyanhydrides, polyorthoesters or chitosan). In some embodiments, the first polymer is a hydrophobic polymer. In some embodiments, the percent by weight of the first polymer within the particle is from about 40% to about 90%, e.g., about 30% to about 70%. In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer is diblock copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2:to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an anti-cancer agent or other therapeutic or diagnostic agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the first agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single first agent is attached to a single first polymer, e.g., to a terminal end of the polymer. In some embodiments, a plurality of first agents are attached to a single first polymer (e.g., 2, 3, 4, 5, 6, or more). In some embodiments, the first agent is a diagnostic agent.

In some embodiments, the first agent is a therapeutic agent. In some embodiment, the therapeutic agent is a boronic acid containing drug. In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent, or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine).

In some embodiments, the therapeutic agent is a boronic acid containing drug as described in structural formula A herein. In some embodiments, the therapeutic agent is a boronic acid containing drug described in the PATENTS. In some embodiments, the therapeutic agent is a bortezomib (Velcade®).

In some embodiments, the anti-cancer agent is paclitaxel, attached to the first polymer via the hydroxyl group at the 2′ position, the hydroxyl group at the 1 position and/or the hydroxyl group at the 7 position. In some embodiments, the anti-cancer agent is paclitaxel, attached to the first polymer via the hydroxyl group at the 2′ position and/or the hydroxyl group at the 7 position.

In some embodiments, the anti-cancer agent is docetaxel, attached to the first polymer via the hydroxyl group at the 2′ position, the hydroxyl group at the 7 position, the hydroxyl group at the 10 position, and/or the hydroxyl group at the 1 position. In some embodiments, the anti-cancer agent is docetaxel, attached to the first polymer via the hydroxyl group at the 2′ position, the hydroxyl group at the 7 position and/or the hydroxyl group at the 10 position.

In some embodiments, the anti-cancer agent is docetaxel-succinate.

In some embodiments, the anti-cancer agent is a taxane that is attached to the polymer via the hydroxyl group at the 7 position and has an acyl group or a hydroxy protecting group on the hydroxyl group at the 2′ position (e.g., wherein the anti-cancer agent is a taxane such as paclitaxel, docetaxel, larotaxel or cabazitaxel). In some embodiments, the anti-cancer agent is larotaxel. In some embodiments, the anti-cancer agent is cabazitaxel.

In some embodiments, the anti-cancer agent is doxorubicin.

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, the first agent is attached to the first polymer to form a polymer-agent conjugate. In some embodiments, a single first agent is attached to the first polymer. In some embodiments, multiple agents are attached to the first polymer (e.g., 2, 3, 4, 5, 6 or more agents). In some embodiments, the agents are the same agent. In some embodiments, the agents are different agents.

In some embodiments, the agent is doxorubicin, and is covalently attached to the first polymer through an amide bond.

In some embodiments, the polymer-agent conjugate in the particle e.g., the nanoparticle, is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the polymer-agent conjugate in the particle, e.g., the nanoparticle, is:

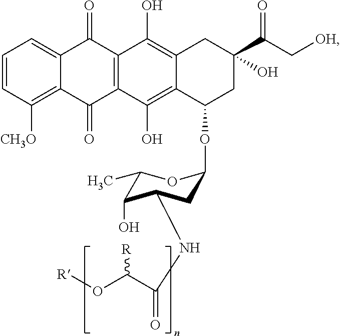

wherein about 30% to about 70%, 35% to about 65%, 40% to about 60%, 45% to about 55% of R substituents are hydrogen (e.g., about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the therapeutic agent is paclitaxel, and is covalently attached to the first polymer through an ester bond. In some embodiments, the agent is paclitaxel, and is attached to the polymer via the hydroxyl group at the 2′ position.

In some embodiments, the polymer-agent conjugate in the particle, e.g., the nanoparticle, is:

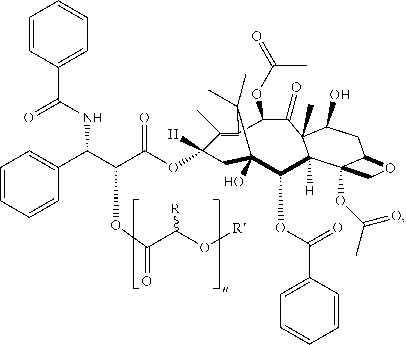

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is attached to the polymer via the hydroxyl group at the 7 position.

In some embodiments, the polymer-agent conjugate in the particle, e.g., the nanoparticle, is:

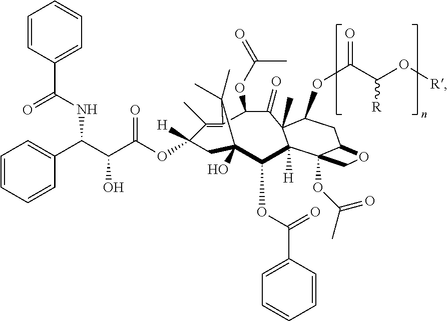

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particle includes a combination of polymer-paclitaxel conjugates described herein, e.g., polymer-paclitaxel conjugates illustrated above.

In some embodiments, the polymer-agent conjugate in the particle, e.g., the nanoparticle, has the following formula (I):

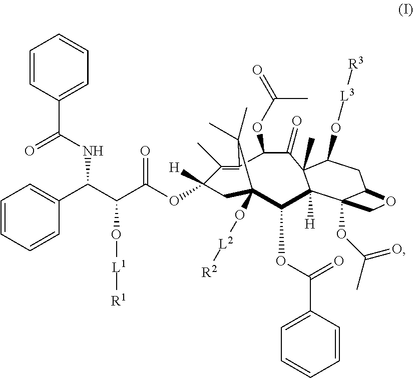

wherein L¹, L² and L³ are each independently a bond or a linker, e.g., a linker described herein;

wherein R¹, R² and R³ are each independently hydrogen, C₁-C₆ alkyl, acyl, or a polymer of formula (II):

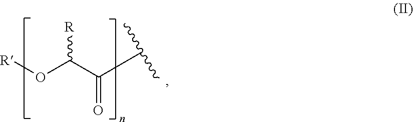

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R² and R³ is a polymer of formula (II).

In some embodiments, L² is a bond and R² is hydrogen.

In some embodiments, the therapeutic agent is paclitaxel, and is covalently attached to the first polymer via a carbonate bond.

In some embodiments, the therapeutic agent is docetaxel, and is covalently attached to the first polymer through an ester bond.

In some embodiments, the agent is docetaxel, and is attached to the polymer via the hydroxyl group at the 2′ position.

In some embodiments, the polymer-agent conjugate in the particle, e.g., the nanoparticle, is:

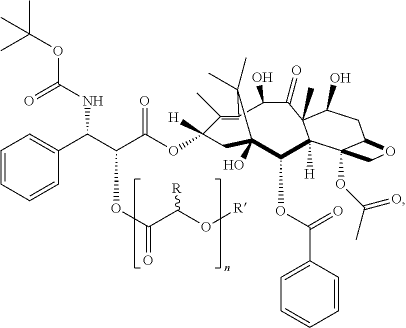

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 7 position.

In some embodiments, the polymer-agent conjugate in the particle, e.g., the nanoparticle, is:

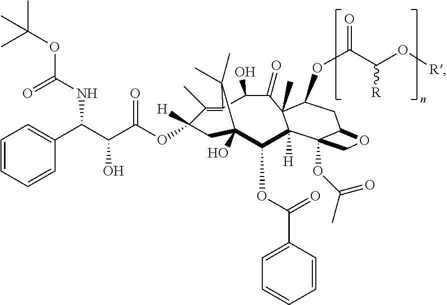

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the polymer-agent conjugate in the particle, e.g., the nanoparticle, is:

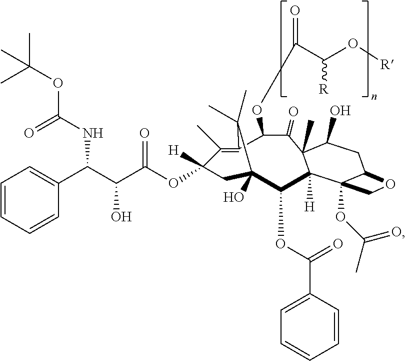

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is covalently attached to the first polymer through a carbonate bond.

In some embodiments, the particle includes a combination of polymer-docetaxel conjugates described herein, e.g., polymer-docetaxel conjugates illustrated above.

In some embodiments, the agent is attached to the polymer through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, the polymer-agent conjugate in the particle, e.g., the nanoparticle, is:

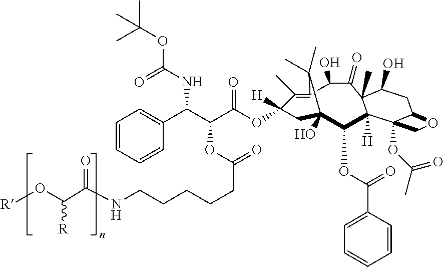

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate is:

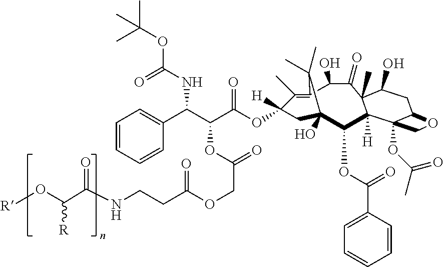

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate in the particle, e.g., the nanoparticle, has the following formula (III):

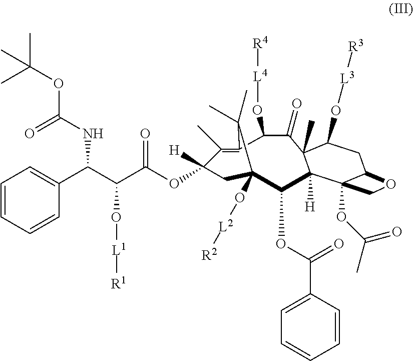

wherein L¹, L², L³ and L⁴ are each independently a bond or a linker, e.g., a linker described herein;

R¹, R², R³ and R⁴ are each independently hydrogen, C₁-C₆ alkyl, acyl, a hydroxy protecting group, or a polymer of formula (IV):

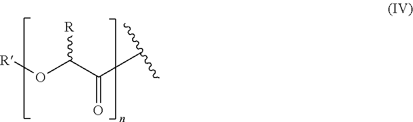

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R², R³ and R⁴ is a polymer of formula (IV).

In some embodiments, L² is a bond and R² is hydrogen.

In some embodiments, two agents are attached to a polymer via a multifunctional linker. In some embodiments, the two agents are the same agent. In some embodiments, the two agents are different agents. In some embodiments, the agent is docetaxel, and is covalently attached to the polymer via a glutamate linker.

In some embodiments, the polymer-agent conjugate in the particle, e.g., the nanoparticle, is:

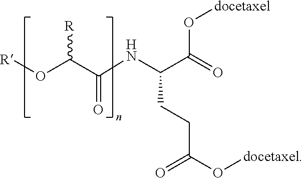

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., one docetaxel is attached via the hydroxyl group at the 2′ position and the other is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is docetaxel, and is covalently attached to the polymer via a tri(glutamate) linker.

In some embodiments, the polymer-agent conjugate in the particle, e.g., the nanoparticle, is:

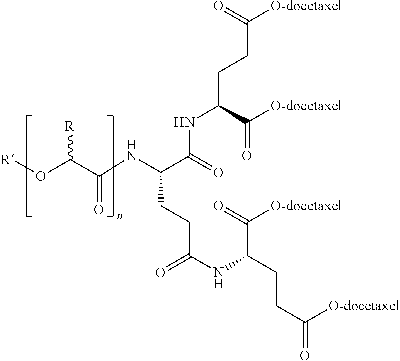

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, docetaxel molecules may be attached via different hydroxyl groups, e.g., three docetaxel molecules are attached via the hydroxyl group at the 2′ position and the other is attached via the hydroxyl group at the 7 position.

In some embodiments, the polymer-agent conjugate has the following formula:

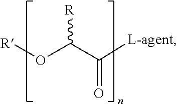

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is a taxane, e.g., docetaxel, paclitaxel, larotaxel or cabazitaxel.

In some embodiments, L is a bond.

In some embodiments, L is a linker, e.g., a linker described herein.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, the agent is paclitaxel, and the plurality of polymer-agent conjugates includes PLGA polymers attached to paclitaxel via the hydroxyl group at the 2′ position, and PLGA polymers attached to paclitaxel via the hydroxyl group at the 7 position. In some embodiments, the polymer is PLGA, the agent is paclitaxel, and the plurality of polymer-agent conjugates includes PLGA polymers attached to paclitaxel via the hydroxyl group at the 2′ position, PLGA polymers attached to paclitaxel via the hydroxyl group at the 7 position, and/or PLGA polymers attached to paclitaxel via the hydroxyl group at the 1 position. In some embodiments, the polymer is PLGA, the agent is paclitaxel, and the plurality of polymer-agent conjugates includes paclitaxel molecules attached to more than one polymer chain, e.g., paclitaxel molecules with PLGA polymers attached to the hydroxyl group at the 2′ position, the hydroxyl group at the 7 position and/or the hydroxyl group at the 1 position.

In some embodiments, the polymer is PLGA, the agent is docetaxel, and the plurality of polymer-agent conjugates includes PLGA attached to docetaxel via the hydroxyl group at the 2′ position and PLGA attached to docetaxel via the hydroxyl group at the 7 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, and/or PLGA polymers attached to docetaxel via the hydroxyl group at the 10 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, PLGA polymers attached to docetaxel via the hydroxyl group at the 10 position and/or PLGA polymers attached to docetaxel via the hydroxyl group at the 1 position. In some embodiments, the polymer is PLGA, the agent is docetaxel, and the plurality of polymer-agent conjugates includes docetaxel molecules attached to more than one polymer chain, e.g., docetaxel molecules with PLGA polymers attached to the hydroxyl group at the 2′ position, the hydroxyl group at the 7 position, the hydroxyl group at the 10 position and/or the hydroxyl group at the 1 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the first agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In some embodiments, the second agent is a diagnostic agent. In some embodiments, the second agent is a therapeutic agent. In some embodiments, the therapeutic agent is in the form of a salt (e.g., an insoluble salt). In some embodiments, the therapeutic agent is a salt of doxorubicin (e.g., a tosylate salt of doxorubicin). In some embodiments, the therapeutic agent is in the form of a prodrug (i.e., the prodrug releases the therapeutic agent in vivo). In some embodiments, the prodrug of the therapeutic agent is conjugated to a hydrophobic moiety that is cleaved in vivo (e.g., a polymer or oligomer).

In some embodiments, the second agent is a boronic acid containing drug. In some embodiments, the second agent is an anti-inflammatory agent. In some embodiments, the second agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine).

In some embodiments, the second agent is a boronic acid containing drug as described in structural formula A herein. In some embodiments, the second agent is a boronic acid containing drug described in the PATENTS. In some embodiments, the therapeutic agent is a bortezomib (Velcade®).

In some embodiments, the anti-cancer agent is paclitaxel. In some embodiments, the anti-cancer agent is docetaxel. In some embodiments, the anti-cancer agent is docetaxel-succinate. In some embodiments, the anti-cancer agent is selected from doxorubicin, doxorubicin hexanoate and doxorubicin hydrazone hexanoate. In some embodiments, the anti-cancer agent is larotaxel. In some embodiments, the anti-cancer agent is cabazitaxel. In some embodiments, the anti-cancer agent is selected from gemcitabine, 5FU and cisplatin or a prodrug thereof.

In some embodiments, the second agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the second agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the first agent is docetaxel and the second agent is doxorubicin.

In some embodiments, at least about 50% of the second agent is embedded in the particle (e.g., embedded in the first polymer, second polymer, and/or compound comprising at least one acidic moiety). In some embodiments, substantially all of the second agent is embedded in the particle (e.g., embedded in the first polymer, second polymer, and/or compound comprising at least one acidic moiety).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer,

a second polymer having a hydrophilic portion and a hydrophobic portion, and

an agent (e.g., a therapeutic or diagnostic agent) embedded in the particle.

In some embodiments, the agent embedded in the particle makes up from about 0.1 to about 10% by weight of the particle (e.g., about 0.5% wt., about 1% wt., about 2% wt., about 3% wt., about 4% wt., about 5% wt., about 6% wt., about 7% wt., about 8% wt., about 9% wt., about 10% wt.).

In some embodiments, the agent is substantially absent from the surface of the particle. In some embodiments, the agent is substantially uniformly distributed throughout the particle. In some embodiments, the agent is not uniformly distributed throughout the particle. In some embodiments, the particle includes hydrophobic pockets and the agent is concentrated in hydrophobic pockets of the particle.

In some embodiments, the agent forms one or more non-covalent interactions with a polymer in the particle. In some embodiments, the agent forms one or more hydrophobic interactions with a hydrophobic polymer in the particle. In some embodiments, the agent forms one or more hydrogen bonds with a polymer in the particle.

In some embodiments, the agent is not covalently bound to the first or second polymer.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the first polymer is a biodegradable polymer (e.g., PLA, PGA, PLGA, PCL, PDO, polyanhydrides, polyorthoesters or chitosan). In some embodiments, the first polymer is a hydrophobic polymer. In some embodiments, the percent by weight of the first polymer within the particle is from about 40% to about 90%. In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer is diblock copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio of the first and second polymer is from about 1:1 to about 20:1, e.g., about 1:1 to about 10:1, e.g., about 1:1 to 9:1, or about 1.2:to 8:1. In some embodiments, the ratio of the first and second polymer is from about 85:15 to about 55:45 percent by weight or about 84:16 to about 60:40 percent by weight.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a surfactant (if present), and the agent, e.g., an anti-cancer agent or other therapeutic or diagnostic agent, that targets the particle. Thus, in such embodiments, any contribution to localization by the first polymer, the second polymer, a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, the agent is a diagnostic agent. In some embodiments, the agent is a therapeutic agent. In some embodiments, the therapeutic agent is in the form of a salt (e.g., an insoluble salt). In some embodiments, the therapeutic agent is a salt of doxorubicin (e.g., a tosylate salt of doxorubicin). In some embodiments, the therapeutic agent is in the form of a prodrug (i.e., the prodrug releases the therapeutic agent in vivo).

In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent, or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine). In some embodiments, the anti-cancer agent is selected from gemcitabine, 5FU and cisplatin or a prodrug thereof. In some embodiments, the anti-cancer agent is docetaxel-succinate. In some embodiments, the anti-cancer agent is selected from doxorubicin hexanoate and doxorubicin hydrazone hexanoate.

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In some embodiments, at least about 50% of the agent is embedded in the particle (e.g., embedded in the first polymer and/or the second polymer). In some embodiments, substantially all of the agent is embedded in particle (e.g., embedded in the first polymer and/or the second polymer).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer and a second polymer;

a first agent and a second agent, wherein the first agent is attached to the first polymer to form a first polymer-agent conjugate, and the second agent is attached to the second polymer to form a second polymer-agent conjugate; and

a third polymer, the third polymer comprising a hydrophilic portion and a hydrophobic portion.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the first polymer is a PLGA polymer. In some embodiments, the second polymer is a PLGA polymer. In some embodiments, both the first and second polymers are PLGA polymers.

In some embodiments, the first agent is a therapeutic agent (e.g., an anti-cancer agent). In some embodiments, the second agent is a therapeutic agent (e.g., an anti-cancer agent). In some embodiments, the first and second agent have the same chemical structure. In some embodiments, the first agent and second agent have the same chemical structure and are attached to the respective polymers via the same point of attachment. In some embodiments, the first agent and second agent have the same chemical structure and are attached to the respective polymers through different points of attachment. In some embodiments, the first and second agent have different chemical structures. In some embodiments, the first agent and/or the second agent is a boronic acid containing drug as described in structural formula A herein. In some embodiments, the first agent and/or the second agent is a bortezomib (Velcade®).

In some embodiments, the particle has one or more of the following properties:

it further comprises a compound comprising at least one acidic moiety, wherein the compound is a polymer or a small molecule;

it further comprises a surfactant;

the first or second polymer is a PLGA polymer, wherein the ratio of lactic acid to glycolic acid is from about 25:75 to about 75:25;

the first or second polymer is a PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or

the ratio of the combined first and second polymer to the third polymer is such that the particle comprises at least 5%, 10%, 15%, 20%, 25% by weight of a polymer having a hydrophobic portion and a hydrophilic portion.

In an embodiment the first agent is attached to a first polymer, the second agent is attached to a second polymer and:

the first and second agents are the same, e.g., the same anti-cancer agent;

the first and second agents are the same, e.g., the same anti-cancer agent, and the first and second polymers are different from one another. E.g., the first and second polymers differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer;

the first and second agents are different agents, e.g., two different anti-cancer agents;

the first and second agents are different agents, e.g., two different anti-cancer agents, and the first and second polymers have the same structure, e.g., they are the same PLGA polymer;

the first and second agents are different agents, e.g., two different anti-cancer agents, and the first and second polymers are different from one another. E.g., the first and second polymers differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer.

In some embodiments, the first and/or second agent is a boronic acid containing drug as described in structural formula A herein. In some embodiments, the first agent and/or the second agent is a bortezomib (Velcade®).

In an embodiment the first agent is released from the first polymer-agent conjugate with a first release profile and the second agent is released from the second polymer-agent conjugate with a second release profile. E.g., a bond between the first agent and the first polymer is more rapidly broken than a bond between the second agent and the second polymer. E.g., the first polymer-agent conjugate can comprise a first linker (e.g., a linker or a bond) linking the first agent to the first polymer and the second polymer-agent conjugate can comprise a second linker (e.g., a linker or a bond) linking the second agent to the second polymer, wherein the linkers provide for different profiles for release of the first and second agents from their respective agent-polymer conjugates. As described above, the first and second agents can differ or be the same. Similarly, the first and second polymers can differ or be the same. Thus, the release profile of one or more agents can be optimized.

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In an embodiment the amount of first and second agent in the particle that is not attached to the first or second polymer is less than about 5% (e.g., less than about 2% or less than about 1%, e.g., in terms of w/w or number/number) of the amount of first or second agent attached to the first polymer or second polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the second polymer is a biodegradable polymer (e.g., PLA, PGA, PLGA, PCL, PDO, polyanhydrides, polyorthoesters, or chitosan). In some embodiments, the second polymer is a hydrophobic polymer. In some embodiments, the percent by weight of the second polymer within the particle is from about 20% to about 90% (e.g., from about 20% to about 80%, from about 25% to about 75%, or from about 30% to about 70%). In some embodiments, the second polymer is PLA. In some embodiments, the second polymer is PGA.

In some embodiments, the second polymer is a copolymer of lactic and glycolic acid (e.g., PLGA). In some embodiments, the second polymer is a PLGA-ester. In some embodiments, the second polymer is a PLGA-lauryl ester. In some embodiments, the second polymer comprises a terminal free acid. In some embodiments, the second polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers in PLGA to glycolic acid monomers is from about 75:25 to about 25:75, e.g., about 60:40 to about 40:60 (e.g., about 50:50), about 60:40, or about 75:25.

In some embodiments, the weight average molecular weight of the second polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the second polymer has a glass transition temperature of from about 20° C. to about 60° C. In some embodiments, the second polymer has a polymer polydispersity index of less than or equal to about 2.5 (e.g., less than or equal to about 2.2, or less than or equal to about 2.0). In some embodiments, the second polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the third polymer within the particle is up to about 50% by weight (e.g., from about 4 to any of about 50%, about 5%, about 10%, about 15%, about 20%, about 25%, about 30%, about 35%, about 40%, about 45% or about 50% by weight). In some embodiments, the third polymer has a hydrophilic portion and a hydrophobic portion. In some embodiments, the third polymer is a block copolymer. In some embodiments, the third polymer comprises two regions, the two regions together being at least about 70% by weight of the polymer (e.g., at least about 80%, at least about 90%, at least about 95%). In some embodiments, the third polymer is a block copolymer comprising a hydrophobic polymer and a hydrophilic polymer. In some embodiments, the third polymer, e.g., a diblock copolymer, comprises a hydrophobic polymer and a hydrophilic polymer. In some embodiments, the third polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the third polymer is a biodegradable polymer (e.g., PLA, PGA, PLGA, PCL, PDO, polyanhydrides, polyorthoesters, or chitosan). In some embodiments, the hydrophobic portion of the third polymer is PLA. In some embodiments, the hydrophobic portion of the third polymer is PGA. In some embodiments, the hydrophobic portion of the third polymer is a copolymer of lactic and glycolic acid (e.g., PLGA). In some embodiments, the hydrophobic portion of the third polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the third polymer is PEG. In some embodiments, the hydrophilic portion of the third polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the third polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the third polymer has a weight average molecular weight of from about 2 kDa to 3.5 kDa and the ratio of the weight average molecular weight of the hydrophilic to hydrophobic portions of the third polymer is from about 1:4 to about 1:6.5 (e.g., 1:4, 1:4.5, 1:5, 1:5.5, 1:6, 1:6.5). In one embodiment, the hydrophilic portion of the third polymer has a weight average molecular weight of from about 4 kDa to 6 kDa (e.g., 5 kDa) and the ratio of the weight average molecular weight of the hydrophilic to hydrophobic portions of the third polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the third polymer has a terminal hydroxyl moiety. In some embodiments, the hydrophilic polymer portion of the third polymer has a terminal alkoxy moiety. In some embodiments, the hydrophilic polymer portion of the third polymer is a methoxy PEG (e.g., a terminal methoxy PEG). In some embodiments, the hydrophilic polymer portion of the third polymer does not have a terminal alkoxy moiety. In some embodiments, the terminus of the hydrophilic polymer portion of the third polymer is conjugated to hydrophobic polymer, e.g., to make a triblock copolymer.

In some embodiments, the hydrophilic polymer portion of the third polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the third polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the combined first and second polymers to the third polymer is from about 1:1 to about 20:1, e.g., about 1:1 to about 10:1, e.g., about 1:1 to 9:1, or about 1.2:to 8:1. In some embodiments, the ratio of the first and second polymer is from about 85:15 to about 55:45 percent by weight or about 84:16 to about 60:40 percent by weight. In some embodiments, the ratio by weight of the combined first and second polymers to the compound comprising at least one acidic moiety is from about 1:3 to about 1000:1, e.g., about 1:1 to about 10:1, or about 1.5:1. In some embodiments, the ratio of the third polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer, a surfactant (if present), and the agent, e.g., an anti-cancer agent or other therapeutic or diagnostic agent, that targets the particle. Thus, in such embodiments, any contribution to localization by the first polymer, the second polymer, a third polymer, a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the third polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the combined first and second polymer to the third polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single first agent is attached to a single first polymer, e.g., to a terminal end of the polymer. In some embodiments, a plurality of first agents are attached to a single first polymer (e.g., 2, 3, 4, 5, 6, or more). In some embodiments, the agents are the same agent. In some embodiments, the agents are different agents. In some embodiments, a single second agent is attached to a single second polymer, e.g., to a terminal end of the polymer. In some embodiments, a plurality of second agents are attached to a single second polymer (e.g., 2, 3, 4, 5, 6, or more). In some embodiments, the agents are the same agent. In some embodiments, the agents are different agents.

In some embodiments, the first agent or the second agent is a diagnostic agent. In some embodiments, the first agent or the second agent is a therapeutic agent.

In some embodiments, the therapeutic agent is a boronic acid containing drug. In some embodiments, the therapeutic agent is an anti-inflammatory agent. In some embodiments, the therapeutic agent is an anti-cancer agent. In some embodiments, the anti-cancer agent is an alkylating agent, a vascular disrupting agent, a microtubule targeting agent, a mitotic inhibitor, a topoisomerase inhibitor, an anti-angiogenic agent or an anti-metabolite. In some embodiments, the anti-cancer agent is a taxane (e.g., paclitaxel, docetaxel, larotaxel or cabazitaxel). In some embodiments, the anti-cancer agent is an anthracycline (e.g., doxorubicin). In some embodiments, the anti-cancer agent is a platinum-based agent (e.g., cisplatin). In some embodiments, the anti-cancer agent is a pyrimidine analog (e.g., gemcitabine).

In some embodiments, the therapeutic agent is a boronic acid containing drug as described in structural formula A herein. In some embodiments, the therapeutic agent is a boronic acid containing drug described in the PATENTS. In some embodiments, the therapeutic agent is a bortezomib (Velcade®).

In some embodiments, the anti-cancer agent is paclitaxel, attached to the polymer via the hydroxyl group at the 2′ position, the hydroxyl group at the 1 position and/or the hydroxyl group at the 7 position. In some embodiments, the anti-cancer agent is paclitaxel, attached to the polymer via the hydroxyl group at the 2′ position and/or the hydroxyl group at the 7 position.

In some embodiments, the anti-cancer agent is docetaxel, attached to the polymer via the hydroxyl group at the 2′ position, the hydroxyl group at the 7 position, the hydroxyl group at the 10 position and/or the hydroxyl group at the 1 position. In some embodiments, the anti-cancer agent is docetaxel, attached to the polymer via the hydroxyl group at the 2′ position, the hydroxyl group at the 7 position and/or the hydroxyl group at the 10 position.

In some embodiments, the anti-cancer agent is docetaxel-succinate.

In some embodiments, the anti-cancer agent is a taxane that is attached to the polymer via the hydroxyl group at the 7 position and has an acyl group or a hydroxy protecting group on the hydroxyl group at the 2′ position (e.g., wherein the anti-cancer agent is a taxane such as paclitaxel, docetaxel, larotaxel or cabazitaxel). In some embodiments, the anti-cancer agent is larotaxel. In some embodiments, the anti-cancer agent is cabazitaxel.

In some embodiments, the anti-cancer agent is doxorubicin.

In some embodiments, the therapeutic agent is an agent for the treatment or prevention of cardiovascular disease, for example as described herein. In some embodiments, the therapeutic agent is an agent for the treatment of cardiovascular disease, for example as described herein. In some embodiments, the therapeutic agent is an agent for the prevention of cardiovascular disease, for example as described herein.

In some embodiments, the therapeutic agent is an agent for the treatment or prevention of an inflammatory or autoimmune disease, for example as described herein. In some embodiments, the therapeutic agent is an agent for the treatment of inflammatory or autoimmune disease, for example as described herein. In some embodiments, the therapeutic agent is an agent for the prevention of an inflammatory or autoimmune disease, for example as described herein.

In some embodiments, the first agent is attached directly to the first polymer, e.g., through a covalent bond. In some embodiments, the first agent is attached to a terminal end of the first polymer via an amide, ester, ether, amino, carbamate or carbonate bond. In some embodiments, the first agent is attached to a terminal end of the first polymer. In some embodiments, the first polymer comprises one or more side chains and the first agent is directly attached to the first polymer through one or more of the side chains.

In some embodiments, the second agent is attached directly to the second polymer, e.g., through a covalent bond. In some embodiments, the second agent is attached to a terminal end of the second polymer via an amide, ester, ether, amino, carbamate or carbonate bond. In some embodiments, the second agent is attached to a terminal end of the second polymer. In some embodiments, the second polymer comprises one or more side chains and the second agent is directly attached to the second polymer through one or more of the side chains.

In some embodiments, the agent is doxorubicin, and is covalently attached to the first polymer through an amide bond.

In some embodiments, the first or second polymer-agent conjugate in the particle, e.g., the nanoparticle, is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the first or second polymer-agent conjugate in the particle, e.g., the nanoparticle, is:

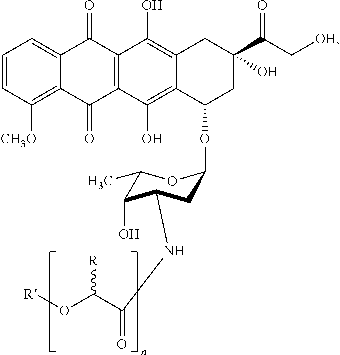

wherein about 30% to about 70%, 35% to about 65%, 40% to about 60%, 45% to about 55% of R substituents are hydrogen (e.g., about 50%) and about 30% to about 70%, 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is covalently attached to the polymer through an ester bond. In some embodiments, the agent is paclitaxel, and is attached to the polymer via the hydroxyl group at the 2′ position.

In some embodiments, the first or second polymer-agent conjugate in the particle, e.g., the nanoparticle, is:

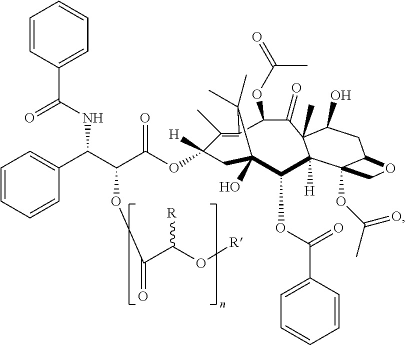

wherein about 30% to about 70%, about 35% to about 65%, about 40% to about 60%, about 45% to about 55% of R substituents are hydrogen (e.g., about 50%) and about 30% to about 70%, about 35% to about 65%, 40% to about 60%, 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is paclitaxel, and is attached to the polymer via the hydroxyl group at the 7 position.

In some embodiments, the first or second polymer-agent conjugate in the particle, e.g., the nanoparticle, is:

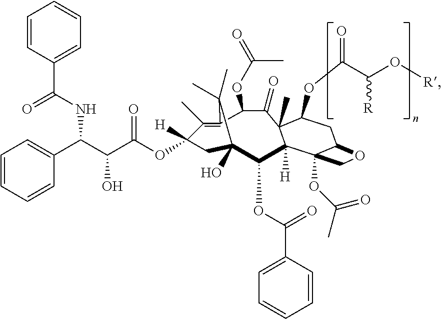

wherein about 30% to about 70%, about 35% to about 65%, about 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the particle includes a combination of polymer-paclitaxel conjugates described herein, e.g., polymer-paclitaxel conjugates illustrated above.

In some embodiments, the polymer-agent conjugate in the particle, e.g., the nanoparticle, has the following formula (I):

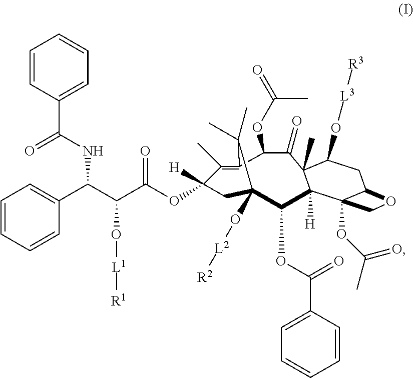

wherein L¹, L² and L³ are each independently a bond or a linker, e.g., a linker described herein;

wherein R¹, R² and R³ are each independently hydrogen, C₁-C₆ alkyl, acyl, or a polymer of formula (II):

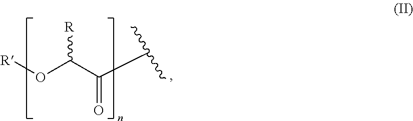

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R² and R³ is a polymer of formula (II).

In some embodiments, L² is a bond and R² is hydrogen.

In some embodiments, the agent is paclitaxel, and is covalently attached to the polymer via a carbonate bond.

In some embodiments, the agent is docetaxel, and is covalently attached to the polymer through an ester bond.

In some embodiments, the agent is docetaxel, and is attached to the polymer via the hydroxyl group at the 2′ position.

In some embodiments, the first or second polymer-agent conjugate in the particle, e.g., the nanoparticle, is:

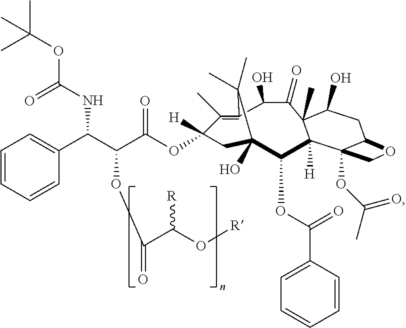

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 7 position.

In some embodiments, the polymer-agent conjugate in the particle, e.g., the nanoparticle, is:

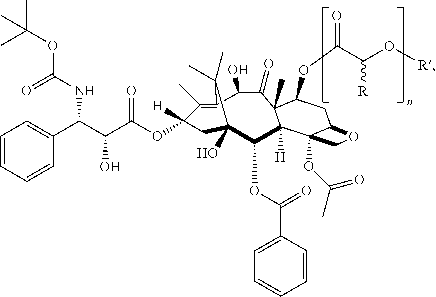

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is attached to the polymer via the hydroxyl group at the 10 position.

In some embodiments, the polymer-agent conjugate in the particle, e.g., the nanoparticle, is:

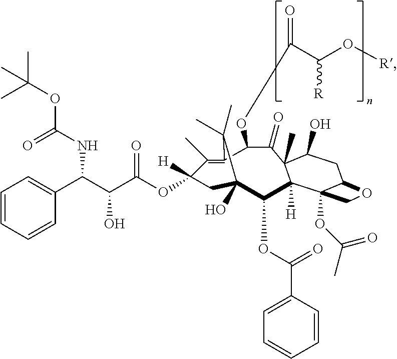

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is docetaxel, and is covalently attached to the polymer through a carbonate bond.

In some embodiments, the particle includes a combination of polymer-docetaxel conjugates described herein, e.g., polymer-docetaxel conjugates illustrated above.

In some embodiments, the agent is attached to the polymer through a linker. In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, the first or second polymer-agent conjugate in the particle, e.g., the nanoparticle, is:

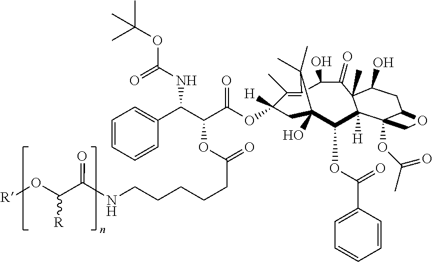

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate is:

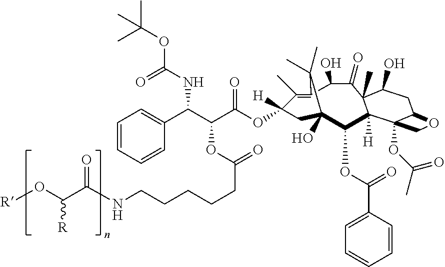

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the polymer-agent conjugate in the particle, e.g., the nanoparticle, has the following formula (III):

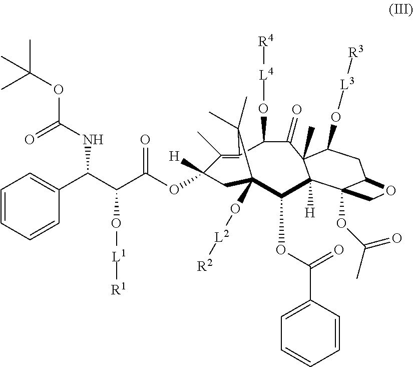

wherein L¹, L², L³ and L⁴ are each independently a bond or a linker, e.g., a linker described herein;

R¹, R², R³ and R⁴ are each independently hydrogen, C₁-C₆ alkyl, acyl, a hydroxy protecting group, or a polymer of formula (IV):

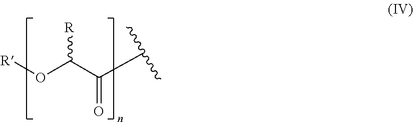

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)); and

wherein at least one of R¹, R², R³ and R⁴ is a polymer of formula (IV).

In some embodiments, L² is a bond and R² is hydrogen.

In some embodiments, two agents are attached to a polymer via a multifunctional linker. In some embodiments, the two agents are the same agent. In some embodiments, the two agents are different agents. In some embodiments, the agent is docetaxel, and is covalently attached to the polymer via a glutamate linker.

In some embodiments, the first or second polymer-agent conjugate in the particle, e.g., the nanoparticle, is:

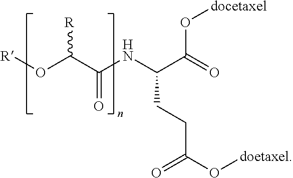

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, at least one docetaxel is attached to the polymer via the hydroxyl group at the 2′ position. In some embodiments, at least one docetaxel is attached to the polymer via the hydroxyl group at the 7 position. In some embodiments, at least one docetaxel is attached to the polymer via the hydroxyl group at the 10 position. In some embodiments, at least one docetaxel is attached to the polymer via the hydroxyl group at the 1 position. In some embodiments, each docetaxel is attached via the same hydroxyl group, e.g., the hydroxyl group at the 2′ position, the hydroxyl group at the 7 position, the hydroxyl group at the 1 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., one docetaxel is attached via the hydroxyl group at the 2′ position and the other is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is docetaxel, and is covalently attached to the polymer via a tri(glutamate) linker.

In some embodiments, the first or second polymer-agent conjugate in the particle, e.g., the nanoparticle, is:

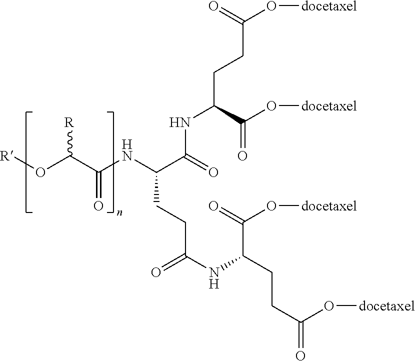

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each docetaxel is attached via the same hydroxyl group, e.g., the hydroxyl group at the 2′ position, the hydroxyl group at the 7 position or the hydroxyl group at the 10 position. In some embodiments, each docetaxel is attached via the hydroxyl group at the 2′ position. In some embodiments, each docetaxel is attached via the hydroxyl group at the 7 position. In some embodiments, each docetaxel is attached via the hydroxyl group at the 10 position. In some embodiments, each docetaxel is attached via a different hydroxyl group, e.g., three docetaxel molecules are attached via the hydroxyl group at the 2′ position and the other is attached via the hydroxyl group at the 7 position.

In some embodiments, the polymer-agent conjugate has the following formula:

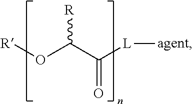

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the agent is a taxane, e.g., docetaxel, paclitaxel, larotaxel or cabazitaxel.

In some embodiments, L is a bond.

In some embodiments, L is a linker, e.g., a linker described herein.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, the agent is paclitaxel, and the plurality of polymer-agent conjugates includes PLGA polymers attached to paclitaxel via the hydroxyl group at the 2′ position, and PLGA polymers attached to paclitaxel via the hydroxyl group at the 7 position. In some embodiments, the polymer is PLGA, the agent is paclitaxel, and the plurality of polymer-agent conjugates includes PLGA polymers attached to paclitaxel via the hydroxyl group at the 2′ position, PLGA polymers attached to paclitaxel via the hydroxyl group at the 7 position, and/or PLGA polymers attached to paclitaxel via the hydroxyl group at the 1 position. In some embodiments, the polymer is PLGA, the agent is paclitaxel, and the plurality of polymer-agent conjugates includes paclitaxel molecules attached to more than one polymer chain, e.g., paclitaxel molecules with PLGA polymers attached to the hydroxyl group at the 2′ position, the hydroxyl group at the 7 position and/or the hydroxyl group at the 1 position.

In some embodiments, the polymer is PLGA, the agent is docetaxel, and the plurality of polymer-agent conjugates includes PLGA attached to docetaxel via the hydroxyl group at the 2′ position and PLGA attached to docetaxel via the hydroxyl group at the 7 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, and/or PLGA polymers attached to docetaxel via the hydroxyl group at the 10 position. In some embodiments, the polymer is PLGA, the agent is docetaxel, and the plurality of polymer-agent conjugates includes PLGA polymers attached to docetaxel via the hydroxyl group at the 2′ position, PLGA polymers attached to docetaxel via the hydroxyl group at the 7 position, PLGA polymers attached to docetaxel via the hydroxyl group at the 10 position and/or PLGA polymers attached to docetaxel via the hydroxyl group at the 1 position. In some embodiments, the polymer is PLGA, the agent is docetaxel, and the plurality of polymer-agent conjugates includes docetaxel molecules attached to more than one polymer chain, e.g., docetaxel molecules with PLGA polymers attached to the hydroxyl group at the 2′ position, the hydroxyl group at the 7 position, the hydroxyl group at the 10 position and/or the hydroxyl group at the 1 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker (e.g., a linker or a bond) linking the first agent to the first polymer and the second polymer-agent conjugate can comprise a second linker (e.g., a linker or a bond) linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the first agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In some embodiments, the second agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In yet another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa) with an agent attached thereto,

providing a polymer comprising a hydrophilic portion and a hydrophobic portion to form a mixture, and

subjecting the mixture to conditions sufficient to form a particle comprising the agent attached to the hydrophobic polymer and the polymer having a hydrophilic portion and a hydrophobic portion.

In some embodiments, the method further comprises attaching the agent to the hydrophobic polymer.

In some embodiments, the method further comprises providing a compound comprising at least one acidic moiety in the mixture.

In some embodiments, the method further comprises providing a surfactant in the mixture.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from the mixture.

In another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa) having a first agent attached thereto,

providing a polymer comprising a hydrophilic portion and a hydrophobic portion,

providing a second agent to form a mixture, and

subjecting the mixture to conditions sufficient to form a particle comprising the first agent attached to the hydrophobic polymer, the polymer comprising a hydrophilic portion and a hydrophobic portion, and a second agent.

In some embodiments, the hydrophilic polymer with the agent attached thereto is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the method further comprises attaching the first agent to the hydrophobic polymer.

In some embodiments, the method further comprises providing a compound comprising at least one acidic moiety in the mixture.

In some embodiments, the method further comprises providing a surfactant in the mixture.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from the mixture.

In another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa),

providing a polymer comprising a hydrophilic portion and a hydrophobic portion,

providing an agent to form a mixture, and

subjecting the mixture to conditions sufficient to form a particle comprising the hydrophobic polymer, the polymer comprising a hydrophilic portion and a hydrophobic portion, and the agent.

In some embodiments, the method further comprises providing a surfactant in the mixture.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from of the mixture.

In another aspect, the invention features a method of making a particle described herein, the method comprising:

dissolving a hydrophobic polymer-agent conjugate and polymer comprising a hydrophilic portion and a hydrophobic portion in an organic solvent to provide an organic solution;

combining the organic solution with an aqueous solution, the aqueous solution comprising a surfactant; and

mixing the resulting combination to provide a mixture comprising a particle described herein.

In some embodiments, the polymer-agent conjugate is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the method further comprises providing a compound comprising at least one acidic moiety in the organic solution.

In some embodiments, the organic solution is filtered (e.g., through a 0.22 micron filter) prior to mixing. In some embodiments, the aqueous solution is filtered (e.g., through a 0.22 micron filter) prior to mixing.

In some embodiments, the organic solvent is miscible with water. In some embodiments, the solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the organic solvent is immiscible with water.

In some embodiments, the ratio of the hydrophobic polymer-agent conjugate and polymer comprising a hydrophilic portion and a hydrophobic portion in the organic solution is from about 90:10 to about 55:45 weight % (e.g., from about 85:15 to about 60:40 weight %).

In some embodiments, the concentration of the surfactant in the aqueous solution is from about 0.1 to about 3.0 weight/volume. In one embodiment, the surfactant is a polymer (e.g., PVA).

In some embodiments, the mixture is purified. In some embodiments, the mixture is concentrated. In some embodiments, the mixture is subjected to tangential flow filtration or dialysis.

In some embodiments, the resulting particle is lyophilized. In one embodiment, the resulting particle is lyophilized in the presence of a lyoprotectant (e.g., a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether).

In some embodiments, the method provides a plurality of particles. In one embodiment, the particles are filtered (e.g., though a 0.22 micron filter). In some embodiments, subsequent to filtering a composition of a plurality of particles, the particles have a Dv90 of less than about 200 nm.

In another aspect, the invention features a mixture, the mixture comprising:

a hydrophobic polymer-agent conjugate;

a polymer comprising a hydrophilic portion and a hydrophobic portion; and

a liquid, wherein the polymer-agent conjugate and polymer comprising a hydrophilic portion and a hydrophobic portion are each independently suspended or dissolved in the liquid.

In some embodiments, the polymer-agent conjugate as in the mixture defined above, is as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the liquid is water. In some embodiments, the liquid is an organic solvent. In some embodiments, the organic solvent is miscible with water. In some embodiments, the organic solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the liquid is a mixture of water and an organic solvent.

In some embodiments, the mixture further comprises a surfactant (e.g., PVA). In some embodiments, the mixture further comprises a compound comprising at least one acidic moiety.

In some embodiments, the hydrophobic polymer-agent conjugate and polymer comprising a hydrophilic portion and a hydrophobic portion are in the mixture as a particle (e.g., a particle described herein).

In another aspect, the invention features a mixture, the mixture comprising:

a first hydrophobic polymer;

a second polymer comprising a hydrophilic portion and a hydrophobic portion;

a first agent attached to the first or second polymer;

a second agent; and

a liquid, wherein the first polymer, the second polymer, the first agent, and the second agent are each independently suspended or dissolved in the liquid.

In some embodiments, the first hydrophilic polymer, second polymer comprising a hydrophilic portion and a hydrophobic portion, first agent attached to the first or second polymer, and second agent are in the mixture as a particle (e.g., a particle described herein).

In some embodiments, the liquid is water. In some embodiments, the liquid is an organic solvent. In some embodiments, the organic solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the liquid is a mixture of water and an organic solvent.

In yet another aspect, the invention features a composition (e.g., a pharmaceutical composition) comprising a plurality of particles described herein. In some embodiments, the composition further comprises an additional component. In some embodiments, the additional component is a pharmaceutically acceptable carrier. In some embodiments, the additional component is a surfactant or a polymer, e.g., a surfactant or a polymer not associated with a particle. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the composition further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the composition further comprises a solvent or suspending liquid (e.g., dextrose). In some embodiments, the composition further comprises one or more of the following: antioxidant, antibacterial, buffer, bulking agent, chelating agent, inert gas, tonicity agent or viscosity agent.

In yet another aspect, the invention features, a composition, e.g., a pharmaceutical composition, that comprises at least two structurally distinct types of particles described herein. The first and second type of particle can differ, e.g., by: the agent, the first polymer, the second polymer, or an additional component, e.g., a surfactant.

E.g., the composition can comprise a first particle comprising a first polymer-agent conjugate, and a second, structurally distinct polymer-agent conjugate. In an embodiment the first polymer-agent conjugate comprises a first agent, e.g., a first anti-cancer drug, and the second polymer-agent conjugate comprises a second agent, e.g., a second anti-cancer drug.

In an embodiment the first or second polymer of the first type of particle and the corresponding polymer of the second type of particle can differ. E.g., they can differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer.

In an embodiment the first type of particle provides for a different profile for release of its agent as compared with the second type of particle, e.g., agent is released from the first type of particle with a first release profile and agent is released from the second type of particle with a second (different) release profile (the agent can be the same or different, e.g., two different anti-cancer agents). E.g., a bond between the agent and polymer in the first type of particle is more rapidly broken than a bond between the agent and polymer in the second type of particle. Thus, the release profile of one or more agents can be optimized.

In yet another aspect, the invention features a kit comprising a polymer-agent conjugate, particle or composition described herein and a device for delivery of the polymer-agent conjugate, particle or composition to a subject. In some embodiments, the above noted kit comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the device for delivery is an IV admixture bag, an IV infusion set, or a piggy back set.

In another aspect, the invention features a kit comprising a polymer-agent conjugate, particle or composition described herein and a container. In some embodiments, the foregoing kit comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the container is a vial. In some embodiments, the vial is a sealed vial (e.g., under inert atmosphere). In some embodiments, the vial is sealed with a flexible seal, e.g., a rubber or silicone closure (e.g., polybutadiene or polyisoprene). In some embodiments, the vial is a light blocking vial. In some embodiments, the vial is substantially free of moisture.

In another aspect, the invention features a kit comprising a polymer-agent conjugate, particle or composition described herein and instructions for reconstituting the polymer-agent conjugate, particle or composition into a pharmaceutically acceptable composition. In some embodiments, the above noted kit comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In embodiments the kit comprises a liquid for reconstitution, e.g., in a single or multi dose formant.

In another aspect, the invention features a kit comprising a polymer-agent conjugate, particle or composition described herein and pharmaceutically acceptable carrier.

In some embodiments, the foregoing kit comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the kit comprises a single dosage unit of a polymer-agent conjugate, particle or composition described herein.

In another aspect, the invention features a method of storing a polymer-agent conjugate, particle or composition described herein, the method comprising providing a polymer-agent conjugate, article or composition described herein in a container, and storing the container for at least about 24 hours. In some embodiments, the foregoing method of storing comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib. In some embodiments, the container is stored at ambient conditions. In some embodiments, the container is stored at a temperature of less than or equal to about 4° C. In some embodiments, the container is a light blocking container. In some embodiments, the container is maintained under inert atmosphere. In some embodiments, the container is substantially free of moisture. In some embodiments, the container is a vial. In some embodiments, the vial is a sealed vial (e.g., under inert atmosphere). In some embodiments, vial is sealed with a rubber or silicone closure (e.g., polybutadiene or polyisoprene). In some embodiments, the vial is a light blocking vial. In some embodiments, the vial is substantially free of moisture.

In some embodiments, the invention features a dosage form comprising a polymer-agent conjugate, particle or composition described herein. In some embodiments, the dosage form is an oral dosage form. In some embodiments, the dosage form is a parenteral dosage form.

In some embodiments, the dosage form further comprises one or more of the following: antioxidant, antibacterial, buffer, bulking agent, chelating agent, inert gas, tonicity agent or viscosity agent.

In some embodiments, the dosage form is a parenteral dosage form (e.g., an intravenous dosage form). In some embodiments, the dosage form is an oral dosage form. In some embodiments, the dosage form is an inhaled dosage form. In some embodiments, the inhaled dosage form is delivered via nebulzation, propellant or a dry powder device). In some embodiments, the dosage form is a topical dosage form. In some embodiments, the dosage form is a mucosal dosage form (e.g., a rectal dosage form or a vaginal dosage form). In some embodiments, the dosage form is an ophthalmic dosage form.

In some embodiments, the dosage form is a solid dosage form. In some embodiments, the dosage form is a liquid dosage form.

In yet another aspect, the invention features a single dosage unit comprising a polymer-agent conjugate, particle or composition described herein. In some embodiments, the single dosage unit is an intravenous dosage unit.

In another aspect, the invention features a method of preparing a liquid dosage form, the method comprising:

providing a polymer-agent conjugate, particle or composition described herein; and

dissolving or suspending the polymer-agent conjugate, particle or composition in a pharmaceutically acceptable carrier.

In some embodiments, the foregoing method of preparing a liquid dosage form comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In one aspect, the invention features a method of instructing a user to prepare a liquid dosage form, the method comprising:

providing a polymer-agent conjugate, particle or composition described herein; and

instructing a user to dissolve or suspend the polymer-agent conjugate, particle or composition in a pharmaceutically acceptable carrier.

In some embodiments, the foregoing method of instructing a user comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In one aspect, the invention features a method of evaluating a polymer-agent conjugate, particle or composition described herein, the method comprising:

subjecting a polymer-agent conjugate, particle or composition described herein to an analytical measurement and evaluating the particle or composition based on that measurement.

In some embodiments, the foregoing method of evaluation comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In some embodiments, the analytical measurement is evaluation of the presence or amount of an impurity or residual solvent. In some embodiments, the analytical measurement is a measurement of the polymer polydispersity index. In some embodiments, the analytical measurement is a measurement of the average particle size. In some embodiments, the analytical measurement is a measurement of the median particle size (Dv50). In some embodiments, the analytical measurement is a measurement of the particle size below which 90% of the volume of particles exists (Dv90). In some embodiments, the analytical measurement is a measurement of the particle polydispersity index.

In another aspect, the invention features a method of treating a disorder or disease described herein, the method comprising administering to a subject a polymer-agent conjugate, particle or composition described herein.

In some embodiments, the foregoing method of treating a disorder or a disease comprises a polymer-agent conjugate as described in any one of the 1^{st} to the 12^{th} embodiments defined below. In another embodiment, the polymer-agent conjugate is as described in any one of the 1^{st} to the 12^{th} embodiments and the boronic acid containing drug is represented by Formula A. Alternatively, the polymer-agent conjugate is as described in any one of the 1^{st} to the 12^{th} embodiments and the boronic acid containing drug is as described in the PATENTS. In another alternative, the polymer-agent conjugate is as described in any one of the 1^{st} to the 12^{th} embodiments and the boronic acid containing drug is bortezomib.

In an embodiment, the method further comprises administering agent not disposed in a particle, e.g., a particle described herein and/or not conjugated to a polymer, referred to herein as a “free” agent. In an embodiment, the agent disposed in a particle and the free agent are both anti-cancer agents, both agents for treating or preventing a cardiovascular disease, or both anti-inflammatory agents.

In an embodiment, the agent disposed in a particle and the free agent are the same anti-cancer agent. E.g., the agent is a taxane (e.g., paclitaxel, docetaxel, larotaxel or cabazitaxel). In an embodiment, the agent is an anthracycline (e.g., doxorubicin). In an embodiment, the agent is bortezomib.

In an embodiment, the agent disposed in a particle and the free agent are different anti-cancer agents.

In an embodiment, the agent disposed in a particle and the free agent are the same agent for treating or preventing a cardiovascular disease.

In an embodiment, the agent disposed in a particle and the free agent are different agents for treating or preventing a cardiovascular disease.

In an embodiment, the agent disposed in a particle and the free agent are different anti-inflammatory agents.

In yet another aspect, the invention features a method of treating a proliferative disorder, e.g., a cancer, in a subject, e.g., a human, the method comprises: administering a composition that comprises a polymer-agent conjugate, particle or composition, e.g., a polymer-agent conjugate, particle or composition described herein, to a subject in an amount effective to treat the disorder, to thereby treat the proliferative disorder. In some embodiments, the polymer-agent conjugate, particle or composition is a polymer-anticancer agent conjugate, particle or composition. In some embodiments, the foregoing method of treating a proliferative disorder comprises a polymer-agent conjugate as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as bortezomib, docetaxel, paclitaxel, larotaxel, cabazitaxel or doxorubicin, coupled, e.g., via a linker, to a polymer described herein. In an embodiment, the polymer-anticancer agent conjugate comprises an anticancer agent, coupled via a linker shown in FIG. 1 or FIG. 2 to a polymer. In an embodiment, the polymer-anticancer agent conjugate is a polymer-anticancer agent conjugate shown in FIG. 1 or FIG. 2.

In one embodiment, the polymer-anticancer agent conjugate, particle or composition is administered in combination with one or more additional chemotherapeutic agent, e.g., a chemotherapeutic agent or combination of chemotherapeutic agents described herein.

In an embodiment, the method further comprises administering an anti-cancer agent as a free agent.

In an embodiment, the agent disposed in a particle and the free agent are the same anti-cancer agent. E.g., the agent is a taxane (e.g., paclitaxel, docetaxel, larotaxel or cabazitaxel). In an embodiment, the agent is an anthracycline (e.g., doxorubicin).

In an embodiment, the agent disposed in a particle and the free agent are different anti-cancer agents.

In one embodiment, the cancer is a cancer described herein. For example, the cancer can be a cancer of the bladder (including accelerated, locally advanced and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer (including lung adenocarcinoma, bronchoalveolar cancer and squamous cell cancer)), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer and small cell lung cancer (including lung adenocarcinoma, bronchoalveolar cancer and squamous cell cancer) e.g., unresectable, locally advanced or metastatic non-small cell lung cancer and small cell lung cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the conjugate, particle or composition is administered by intravenous administration, e.g., an intravenous administration that is completed in a period equal to or less than 2 hours, 1.5 hours, 1 hour, 45 minutes or 30 minutes. In one embodiment, the composition is administered as a bolus infusion or intravenous push, e.g., over a period of 15 minutes, 10 minutes, 5 minutes or less.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein, and e.g., the polymer-docetaxel conjugate, particle or composition is administered to the subject in an amount that includes 60 mg/m² or greater (e.g., 65 mg/m², 70 mg/m², 75 mg/m², 80 mg/m², 85 mg/m², 90 mg/m², 95 mg/m², 100 mg/m², 105 m g/m², 110 mg/m², 115 mg/m², 120 mg/m², 125 mg/m², 130 mg/m², 135 mg/m², 140 mg/m², 145 mg/m², or 150 mg/m²) of docetaxel, to thereby treat the disorder. In one embodiment, the conjugate, particle or composition is administered by intravenous administration over a period of about 30 minutes, 45 minutes, 60 minutes, 90 minutes, 120 minutes, 150 minutes or 180 minutes. In one embodiment, the subject is administered at least one additional dose of the conjugate, particle or composition, e.g., the subject is administered at least two, three, four, five, six, seven, eight, nine, ten or eleven additional doses of the conjugate, particle or composition. In one embodiment, the conjugate, particle or composition is administered once every one, two, three, four, five, six weeks. In another embodiment, the polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein, and e.g., the polymer-docetaxel conjugate, particle or composition is administered to the subject in an amount that includes 30 mg/m² or greater (e.g., 31 mg/m², 33 mg/m², 35 mg/m², 37 mg/m², 40 mg/m², 43 mg/m², 45 mg/m², 47 mg/m², 50 mg/m², 55 mg/m², 60 mg/m²) of docetaxel, to thereby treat the disorder. In one embodiment, the conjugate, particle or composition is administered by intravenous administration over a period of about 30 minutes, 45 minutes, 60 minutes, 90 minutes, 120 minutes, 150 minutes or 180 minutes. In one embodiment, the subject is administered at least one additional dose of the conjugate, particle or composition, e.g., the subject is administered at least two, three, four, five, six, seven, eight, nine, ten or eleven additional doses of the conjugate, particle or composition. In one embodiment, the conjugate, particle or composition is administered once a week for three, four, five six, seven weeks, e.g., followed by one, two or three weeks without administration of the polymer-docetaxel conjugate, particle or composition. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount such that the conjugate, particle or composition includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 m g/m², 145 mg/m², or 150 mg/m²) of docetaxel. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein, and the conjugate, particle or composition is administered to the subject in an amount of the composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², or 150 mg/m²) of docetaxel, administered by intravenous administration over a period equal to or less than about 30 minutes, 45 minutes, 60 minutes, 90 minutes, 120 minutes, 150 minutes or 180 minutes, for at least one, two, three, fours, five or six doses, wherein the subject is administered a dose of the conjugate, particle or composition once every two, three, four, five or six weeks.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein, and the conjugate, particle or composition is administered to the subject in an amount of the composition that includes 30 mg/m² or greater (e.g., 31 m g/m², 33 mg/m², 35 mg/m², 37 mg/m², 40 mg/m², 43 mg/m², 45 mg/m², 47 mg/m², 50 mg/m², 55 mg/m², 60 mg/m²) of docetaxel, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the conjugate, particle or composition once a week for two, three four, five, six doses, e.g., followed by one, two or three weeks without administration of the polymer-docetaxel conjugate, particle or composition.

In one embodiment, the composition includes a polymer-docetaxel conjugate, particle or composition e.g., a polymer-docetaxel conjugate, particle or composition described herein, e.g., a polymer-docetaxel conjugate comprising docetaxel, coupled, e.g., via linkers, to a polymer described herein, and at least two, three, four, five, six, seven, eight, nine, ten or eleven doses are administered to the subject and each dose is an amount of the composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², or 150 mg/m²) of docetaxel, to thereby treat the disorder. In one embodiment, the dose is administered once every one, two, three, four, five, six, seven or eight weeks. In one embodiment, a dose is administered once every three weeks. In one embodiment, the composition includes a polymer-docetaxel conjugate, particle or composition e.g., a polymer-docetaxel conjugate, particle or composition described herein, e.g., a polymer-docetaxel conjugate comprising docetaxel, coupled, e.g., via linkers, to a polymer described herein, and at least two, three, four, five, six, seven, eight, nine, ten or eleven doses are administered to the subject and each dose is an amount of the composition that includes 30 mg/m² or greater (e.g., 31 mg/m², 33 mg/m², 35 mg/m², 37 mg/m², 40 mg/m², 43 mg/m², 45 mg/m², 47 mg/m², 50 mg/m², 55 mg/m², 60 mg/m²) of docetaxel, to thereby treat the disorder. In one embodiment, the dose is administered once a week for two, three, four, five, six, seven weeks, e.g., followed by one, two, three weeks without administration of the polymer-docetaxel conjugate, particle or composition. In one embodiment, each dose is administered by intravenous administration over a period of about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-anticancer agent conjugate, particle or composition is a polymer-paclitaxel conjugate, particle or composition, e.g., a polymer-paclitaxel conjugate, particle or composition described herein and, e.g., a polymer-paclitaxel conjugate comprising paclitaxel, coupled, e.g., via linkers, to a polymer described herein, and, e.g., the conjugate, particle or composition is administered in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 225 mg/m², 230 mg/m², 240 mg/m², 250 mg/m², 260 mg/m², 270 mg/m², 280 mg/m², 290 mg/m², 300 mg/m²) of paclitaxel, to thereby treat the disorder. In one embodiment, the polymer-paclitaxel conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the conjugate, particle or composition, e.g., the subject is administered at least two, three, four, five, six, seven, eight, nine or ten additional doses of the conjugate, particle or composition. In one embodiment, the polymer-paclitaxel conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m², 270 mg/m², 280 mg/m², 290 mg/m², 300 mg/m²) of paclitaxel. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2.

In one embodiment, the polymer-anticancer agent conjugate, particle or composition includes a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein, and the conjugate, particle or composition is administered to the subject in an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m², 270 mg/m², 280 mg/m², 290 mg/m², 300 mg/m²) of paclitaxel, administered by intravenous administration over a period equal to or less than about 30 minutes, 45 minutes, 60 minutes, 90 minutes, 120 minutes, 150 minutes or 180 minutes, for at least two, three, fours, five, six, seven or eight doses, wherein the subject is administered a dose of the composition once every one, two, three, four, five or six weeks.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein, and at least two, three, four, five, six, seven, eight, nine or ten doses are administered to the subject and each dose is an amount that includes 135 mg/m² or greater (e.g., 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 210 mg/m², 220 mg/m², 230 mg/m², 240 mg/m², 250 mg/m², 260 mg/m², 270 mg/m², 280 mg/m², 290 mg/m², 300 mg/m²) of paclitaxel, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein, and, e.g., the conjugate, particle or composition is administered in an amount that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of the doxorubicin, to thereby treat the disorder. In another embodiment, the polymer-doxorubicin conjugate, particle or composition is administered with one or more additional chemotherapeutic agent and the conjugate, particle or composition is administered in an amount that includes 40 mg/m² or greater (e.g., 45 mg/m², 50 mg/m², 55 mg/m², 60 mg/m², 65 mg/m², 70 mg/m², 75 mg/m², 80 mg/m²) of the doxorubicin, to thereby treat the disorder. In one embodiment, the conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, an additional dose (or additional doses) is administered in an amount of the conjugate, particle or composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of the doxorubicin, or 40 mg/m² or greater (e.g., 45 m g/m², 50 mg/m², 55 mg/m², 60 mg/m², 65 mg/m², 70 mg/m², 75 mg/m², 80 mg/m²) of the doxorubicin when administered in combination with an additional chemotherapeutic agent. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes. In an embodiment, the polymer-doxorubicin conjugate comprises doxorubicin, coupled via a linker shown in FIG. 1 or FIG. 2 to a polymer described herein. In an embodiment, the polymer-doxorubicin conjugate is a polymer-doxorubicin conjugate shown in FIG. 1.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein, and the conjugate, particle or composition is administered to the subject in an amount that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of the doxorubicin, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks. In another embodiment, the conjugate, particle or composition is administered in combination with an additional chemotherapeutic agent and the conjugate, particle or composition is administered to the subject in an amount that includes 40 mg/m² or greater (e.g., 45 mg/m², 50 mg/m², 55 mg/m², 60 mg/m², 65 mg/m², 70 mg/m², 75 mg/m², 80 mg/m²) of the doxorubicin, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate, particle or composition comprising doxorubicin, coupled, e.g., via linkers, to a polymer described herein, and at least two, three, four, five, six, seven or eight doses are administered to the subject and each dose is an amount of the composition that includes 60 mg/m² or greater (e.g., 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m²) of the doxorubicin, to thereby treat the disorder. In one embodiment, at least two, three, four, five, six, seven or eight doses of the polymer-doxorubicin conjugate, particle or composition are administered to the subject in combination with an additional chemotherapeutic agent and each dose of the conjugate, particle or composition is an amount that includes 40 mg/m² or greater (e.g., 45 mg/m², 50 mg/m², 55 mg/m², 60 mg/m², 65 mg/m², 70 mg/m², 75 mg/m², 80 mg/m²) of the doxorubicin, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-anticancer agent conjugate, particle or composition, e.g., a polymer-anticancer agent conjugate, particle or composition comprising an anticancer agent coupled, e.g., via linkers, to a polymer described herein, is administered once every three weeks in combination with one or more additional chemotherapeutic agent that is also administered once every three weeks. In one embodiment, the polymer-anticancer agent conjugate, particle or composition is administered once every three weeks in combination with one or more of the following chemotherapeutic agents: a vinca alkaloid (e.g., vinblastine, vincristine, vindesine and vinorelbine); an alkylating agent (e.g., cyclophosphamide, dacarbazine, melphalan, ifosfamide, temozolomide); a topoisomerase inhibitor (e.g., topotecan, irinotecan, etoposide, teniposide, lamellarin D, SN-38, camptothecin (e.g., IT-101)); a platinum-based agent (e.g., cisplatin, carboplatin, oxaliplatin); an antibiotic (e.g., mitomycin, actinomycin, bleomycin), an antimetabolite (e.g., an antifolate (e.g., pemetrexed, floxuridine, raltitrexed) and a pyrimidine analogue (e.g., capecitabine, cytarabine, gemcitabine, 5FU)); an anthracycline (e.g., doxorubicin, daunorubicin, epirubicin, idarubicin, mitoxantrone, valrubicin); and a taxane (e.g., paclitaxel, docetaxel, larotaxel or cabazitaxel).

In one embodiment, the polymer-anticancer agent conjugate, e.g., a polymer-anticancer agent conjugate, particle or composition comprising an anticancer agent coupled, e.g., via linkers, to a polymer described herein, is administered once every two weeks in combination with one or more additional chemotherapeutic agent that is administered orally. In one embodiment, the polymer-anticancer agent conjugate, particle or composition is administered once every two weeks in combination with one or more of the following chemotherapeutic agents: capecitabine, estramustine, erlotinib, rapamycin, SDZ-RAD, CP-547632; AZD2171, sunitinib, sorafenib and everolimus.

In some embodiments, the polymer-anticancer agent conjugate in the foregoing eleven paragraphs is as described in any one of the 1^{st} to the 12^{th} embodiments defined below and the boronic acid containing drug is bortezomib. Alternatively, the polymer-agent conjugate is a polymer-bortezomib conjugate.

In another aspect, the invention features a method of treating an unresectable cancer, a chemotherapeutic sensitive cancer, a chemotherapeutic refractory cancer, a chemotherapeutic resistant cancer, and/or a relapsed cancer. The method comprises: administering a polymer-anticancer agent conjugate, particle or composition, e.g., a polymer-anticancer agent conjugate, particle or composition described herein, to a subject, e.g., a human, in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the cancer is refractory to, resistant to and/or relapsed during or after, treatment with, one or more of: an anthracycline (e.g., doxorubicin, daunorubicin, epirubicin, idarubicin, mitoxantrone, valrubicin), an alkylating agent (e.g., cyclophosphamide, dacarbazine, melphalan, ifosfamide, temozolomide), an antimetabolite (e.g., an antifolate (e.g., pemetrexed, floxuridine, raltitrexed) and a pyrimidine analogue (e.g., capecitabine, cytarabine, gemcitabine, 5FU)), a vinca alkaloid (e.g., vinblastine, vincristine, vindesine, vinorelbine), a topoisomerase inhibitor (e.g., topotecan, irinotecan, etoposide, teniposide, lamellarin D, SN-38, camptothecin (e.g., IT-101)) and a platinum-based agent (e.g., cisplatin, carboplatin, oxaliplatin). In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid.

In one embodiment, the polymer-anticancer agent conjugate, particle or composition is administered in combination with a second chemotherapeutic agent, e.g., a chemotherapeutic agent described herein. For example, the polymer-anticancer agent conjugate, particle or composition can be administered in combination with a vinca alkaloid (e.g., vinblastine, vincristine, vindesine, vinorelbine) and/or a platinum-based agent (e.g., cisplatin, carboplatin, oxaliplatin).

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer (including lung adenocarcinoma, bronchoalveolar cancer and squamous cell cancer)), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer and small cell lung cancer (including lung adenocarcinoma, bronchoalveolar cancer and squamous cell cancer) e.g., unresectable, locally advanced or metastatic non-small cell lung cancer and small cell lung cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 or FIG. 2 to a polymer described herein. In an embodiment, the polymer-doxorubicin conjugate is a polymer-doxorubicin conjugate shown in FIG. 1.

In one embodiment, the polymer-doxorubicin conjugate, particle or composition is administered at a dose and/or dosing schedule described herein.

In yet another aspect, the invention features a method of treating metastatic or locally advanced breast cancer in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer conjugate shown in FIG. 1 or FIG. 2.

In one embodiment, the breast cancer is estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer) or inflammatory breast cancer.

In one embodiment, the polymer-anticancer agent conjugate, particle or composition is administered in combination with a HER-2 pathway inhibitor, e.g., a HER-2 inhibitor or a HER-2 receptor inhibitor. For example, the polymer-anticancer agent conjugate, particle or composition is administered with trastuzumab.

In some embodiments, the polymer-anticancer agent conjugate, particle or composition is administered in combination with a second chemotherapeutic agent. For example, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an anthracycline (e.g., daunorubicin, doxorubicin, epirubicin, valrubicin and idarubicin). In some embodiments, the polymer-anticancer agent conjugate, particle or composition is a polymer-taxane conjugate, particle or composition that is administered in combination with an anthracycline (e.g., daunorubicin, doxorubicin, epirubicin, valrubicin and idarubicin).

In some embodiments, the polymer-anticancer agent conjugate, particle or composition is administered in combination with an anti-metabolite, e.g., an antifolate (e.g., floxuridine, pemetrexed) or pyrimidine analogue (e.g., 5FU)).

In some embodiments, the polymer-anticancer agent conjugate, particle or composition is administered in combination with an anthracycline (e.g., daunorubicin, doxorubicin, epirubicin, valrubicin and idarubicin) and an anti-metabolite (e.g., floxuridine, pemetrexed, 5FU). In some embodiments, the polymer-anticancer agent conjugate, particle or composition is a polymer-taxane conjugate, particle or composition that is administered in combination with an anthracycline (e.g., daunorubicin, doxorubicin, epirubicin, valrubicin and idarubicin) and an anti-metabolite (e.g., floxuridine, pemetrexed, 5FU).

In some embodiments, the polymer-anticancer agent conjugate, particle or composition is administered in combination with a platinum-based agent (e.g., cisplatin, carboplatin, oxaliplatin).

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In some embodiments, the polymer-anticancer agent conjugate, particle or composition is administered in combination with a poly ADP-ribose polymerase (PARP) inhibitor (e.g., BSI 201, Olaparib (AZD-2281), ABT-888, AG014699, CEP 9722, MK 4827, KU-0059436 (AZD2281), LT-673, 3-aminobenzamide).

In some embodiments, the polymer-anticancer agent conjugate, particle or composition is administered in combination with a vinca alkaloid (e.g., vinblastine, vincristine, vindesine, vinorelbine).

In some embodiments, the polymer-anticancer agent conjugate, particle or composition is administered in combination with an antibiotic (e.g., mitomycin, actinomycin, bleomycin).

In some embodiments, the polymer-anticancer agent conjugate, particle or composition is administered in combination with an alkylating agent (e.g., cyclophosphamide, dacarbazine, melphalan, ifosfamide, temozolomide).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating metastatic or locally advanced breast cancer, e.g. a breast cancer described herein, in a subject, e.g., a human. The method comprises:

providing a subject who has metastatic or locally advanced breast cancer and has been treated with a chemotherapeutic agent which did not effectively treat the cancer (e.g., the subject has a chemotherapeutic refractory, a chemotherapeutic resistant and/or a relapsed cancer) or which had an unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer. In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the cancer is refractory to, resistant to, and/or relapsed with treatment with one or more of: a taxane, an anthracycline, a vinca alkaloid (e.g., vinblastine, vincristine, vindesine and vinorelbine), an alkylating agent (e.g., cyclophosphamide, dacarbazine, melphalan, ifosfamide, temozolomide) and a platinum-based agent (e.g., cisplatin, carboplatin, oxaliplatin). In one embodiment, the cancer is refractory to, resistant to, and/or relapsed with treatment with one or more of: an anthracycline and an alkylating agent, and a polymer-taxane conjugate, particle or composition is administered to the subject.

In one embodiment, the cancer is a multidrug resistant cancer.

In one embodiment, the composition is administered in combination with a pyrimidine analogue, e.g., a pyrimidine analogue described herein (e.g., capecitabine).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating hormone refractory prostate cancer in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer. In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is administered in combination with prednisone.

In one embodiment, the polymer-anticancer agent conjugate, particle or composition is administered in combination with estramustine.

In one embodiment, the polymer-anticancer agent conjugate, particle or composition is administered in combination with an anthracenedione (e.g., mitoxantrone) and prednisone.

In one embodiment, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632; AZD2171, AV-951, sunitinib and sorafenib).

In one embodiment, the polymer-anticancer agent conjugate, particle or composition is administered in combination with an mTOR inhibitor. Non-limiting examples of mTOR inhibitors include rapamycin, everolimus, AP23573, CCI-779, and SDZ-RAD.

In one embodiment, the polymer-anticancer agent conjugate, particle or composition is administered in combination with abiraterone.

In one embodiment, the polymer-anticancer agent conjugate, particle or composition is administered in combination with a platinum-based agent (e.g., cisplatin, carboplatin, oxaliplatin).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating hormone refractory prostate cancer in a subject, e.g., a human. The method comprises:

providing a subject who has hormone refractory prostate cancer and has been treated with a chemotherapeutic agent that did not effectively treat the cancer (e.g., the subject has a chemotherapeutic refractory, chemotherapeutic resistant and/or relapsed cancer) or who had unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate, particle or composition comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating metastatic or advanced ovarian cancer (e.g., peritoneal or fallopian tube cancer) in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is administered in combination with a platinum-based agent (e.g., cisplatin, carboplatin, oxaliplatin).

In one embodiment, the polymer-anticancer agent conjugate, particle or composition is administered in combination with an alkylating agent (e.g., cyclophosphamide, dacarbazine, melphalan, ifosfamide, temozolomide).

In one embodiment, the polymer-anticancer agent conjugate, particle or composition is administered in combination with a platinum-based agent (e.g., cisplatin, carboplatin, oxaliplatin) and an alkylating agent (e.g., cyclophosphamide, dacarbazine, melphalan, ifosfamide, temozolomide).

In one embodiment, the polymer-anticancer agent conjugate, particle or composition is administered in combination with one or more of: an anti-metabolite, e.g., an antifolate (e.g., pemetrexed, floxuridine, raltitrexed) or pyrimidine analog (e.g., capecitabine, cytarabine, gemcitabine, 5-fluorouracil); an alkylating agent (e.g., cyclophosphamide, dacarbazine, melphalan, ifosfamide, temozolomide); a topoisomerase inhibitor (e.g., etoposide, topotecan, irinotecan, teniposide, lamellarin D, SN-38); a platinum based agent (carboplatin, cisplatin, oxaliplatin); a vinca alkaloid (e.g., vinblastine, vincristine, vindesine, vinorelbine). In one embodiment, the composition is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the polymer-anticancer agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632, AZD2171, sorafenib and sunitinib.

In one embodiment, the polymer-anticancer agent conjugate, particle or composition is administered in combination with an mTOR inhibitor, e.g., rapamycin, everolimus, AP23573, CCI-779 or SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating metastatic or advanced ovarian cancer (e.g., peritoneal or fallopian tube cancer) in a subject, e.g., a human. The method comprises:

providing a subject who has advanced ovarian cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a composition comprising a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the subject has been treated with a platinum-based agent that did not effectively treat the cancer (e.g., the subject has been treated with cisplatin, carboplatin or oxaliplatin which did not effectively treat the cancer). In one embodiment, the subject has been treated with cisplatin or carboplatin which did not effectively treat the cancer.

In one embodiment, the polymer-anticancer agent conjugate, particle or composition is administered in combination with a pyrimidine analog, e.g., capecitabine or gemcitabine.

In one embodiment, the polymer-anticancer agent conjugate, particle or composition is administered in combination with capecitabine and gemcitabine.

In one embodiment, the polymer-anticancer agent conjugate, particle or composition is administered in combination with an anthracycline, e.g., daunorubicin, doxorubicin, epirubicin, valrubicin and idarubicin. In one embodiment, the anthracycline is doxorubicin, e.g., liposomal doxorubicin.

In one embodiment, the polymer-anticancer agent conjugate, particle or composition is administered in combination with a topoisomerase I inhibitor, e.g., irinotecan, topotecan, teniposide, lamellarin D, SN-38, camptothecin (e.g., IT-101). In one embodiment the topoisomerase I inhibitor is topotecan. In another embodiment, the topoisomerase I inhibitor is irinotecan or etoposide.

In one embodiment, the polymer-anticancer agent conjugate, particle or composition is administered in combination with one or more of: an anti-metabolite, e.g., an antifolate (e.g., pemetrexed, floxuridine, raltitrexed) or pyrimidine analog (e.g., capecitabine, cytarabine, gemcitabine, 5FU); an alkylating agent (e.g., cyclophosphamide, dacarbazine, melphalan, ifosfamide, temozolomide); a platinum based agent (carboplatin, cisplatin, oxaliplatin); and a vinca alkaloid (e.g., vinblastine, vincristine, vindesine, vinorelbine). In one embodiment, the polymer-anticancer agent conjugate, particle or composition is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating non small cell lung cancer or small cell lung cancer (e.g., unresectable, locally advanced or metastatic non small cell lung cancer or small cell lung cancer) in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer. The lung cancer can be a lung adenocarcinoma, a bronchoalveolar cancer, or a squamous cell cancer. In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion), codon 13 of the KRAS gene, codon 61 of the KRAS gene.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is administered in combination with a vascular endothelial (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632, AZD2171, sorafenib and sunitinib.

In one embodiment, the polymer-anticancer agent conjugate, particle or composition is administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In one embodiment, the polymer-anticancer agent conjugate, particle or composition is administered in combination with a platinum-based agent (e.g., cisplatin, carboplatin, oxaliplatin). In one embodiment, the polymer-anticancer agent conjugate, particle or composition is administered in combination with a platinum-based agent (e.g., cisplatin, carboplatin, oxaliplatin) and a nucleoside analog (e.g., gemcitabine). In one embodiment, the polymer-anticancer agent conjugate, particle or composition is administered in combination with a platinum-based agent (e.g., cisplatin, carboplatin, oxaliplatin) and an anti-metabolite, e.g., an antifolate (e.g., floxuridine, pemetrexed) or pyrimidine analogue (e.g., 5FU). In one embodiment, the polymer-anticancer agent conjugate, particle or composition is administered in combination with a platinum-based agent (e.g., cisplatin, carboplatin, oxaliplatin) and a vinca alkaloid (e.g., vinblastine, vincristine, vindesine, vinorelbine).

In one embodiment, the polymer-anticancer agent conjugate, particle or composition is administered in combination with a vinca alkaloid (e.g., vinblastine, vincristine, vindesine, vinorelbine).

In one embodiment, the polymer-anticancer agent conjugate, particle or composition is administered in combination with an alkylating agent (e.g., cyclophosphamide, dacarbazine, melphalan, ifosfamide, temozolomide).

In one embodiment, the polymer-anticancer agent conjugate, particle or composition is administered in combination with an mTOR inhibitor, e.g., rapamycin, everolimus, AP23573, CCI-779 or SDZ-RAD.

In one embodiment, the polymer-anticancer agent conjugate, particle or composition, either alone or with any of the combinations described herein, is administered in combination with radiation.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating unresectable, advanced or metastatic non small cell lung cancer in a subject, e.g., a human. The method comprises:

providing a subject who has unresectable, advanced or metastatic non small cell lung cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the subject has been treated with a vascular endothelial growth factor (VEGF) pathway inhibitor (e.g., a VEGF inhibitor or VEGF receptor inhibitor) which did not effectively treat the cancer (e.g., the subject has been treated with bevacizumab CP-547632, AZD2171, sorafenib and sunitinib which did not effectively treat the cancer).

In one embodiment, the subject has been treated with an endothelial growth factor (EGF) pathway inhibitor (e.g., an EGF inhibitor or an EGF receptor inhibitor) which did not effectively treat the cancer (e.g., the subject has been treated with cetuximab, erlotinib, gefitinib which did not effectively treat the cancer).

In one embodiment, the subject has been treated with a platinum-based agent which did not effectively treat the cancer (e.g., the subject has been treated with cisplatin, carboplatin or oxaliplatin which did not effectively treat the cancer).

In one embodiment, the polymer-anticancer agent conjugate, particle or composition is administered in combination with an anti-metabolite, e.g., an antifolate, e.g., floxuridine, pemetrexed or pyrimidine analogue (e.g., 5FU).

In one embodiment, the polymer-anticancer agent conjugate, particle or composition is administered in combination with an EGF pathway inhibitor, e.g., an EGF inhibitor or EGF receptor inhibitor. The EGF receptor inhibitor can be, e.g., cetuximab, erlotinib or gefitinib.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating multiple myeloma in a subject, e.g., a human. The method comprises: administering a composition comprising a polymer-anticancer agent conjugate, particle or composition, e.g., a polymer-anticancer agent conjugate, particle or composition described herein, to a subject in an amount effective to treat the myeloma, to thereby treat the myeloma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is administered as a primary treatment for multiple myeloma.

In one embodiment, the polymer-anticancer agent conjugate, particle or composition is administered in combination with dexamethasone. In one embodiment, the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), thalidomide or thalidomide derivative (e.g., lenalidomide). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), thalidomide or thalidomide derivative (e.g., lenalidomide). In another embodiment, the polymer-anticancer agent conjugate, particle or composition is a polymer-doxorubicin conjugate, particle or composition that is further administered in combination with thalidomide or thalidomide derivative (e.g., lenalidomide).

In one embodiment, the polymer-anticancer agent conjugate, particle or composition is administered in combination with a proteasome inhibitor (e.g., bortezomib) and dexamethasone. In one embodiment, the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), thalidomide or thalidomide derivative (e.g., lenalidomide). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), thalidomide or thalidomide derivative (e.g., lenalidomide). In another embodiment, the polymer-anticancer agent conjugate, particle or composition is a polymer-doxorubicin conjugate, particle or composition that is further administered in combination with thalidomide or thalidomide derivative (e.g., lenalidomide).

In one embodiment, the polymer-anticancer agent conjugate, particle or composition is administered in combination with a vinca alkaloid (e.g., vinblastine, vincristine, vindesine and vinorelbine) and dexamethasone. In one embodiment, the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), thalidomide or thalidomide derivative (e.g., lenalidomide).

In one embodiment, the polymer-anticancer agent conjugate, particle or composition is administered in combination with thalidomide or thalidomide derivative (e.g., lenalidomide).

In one embodiment, after the subject has received a primary treatment, e.g., a primary treatment described herein, the subject is further administered a high dose treatment. For example, the subject can be administered a high dose treatment of dexamethasone, an alkylating agent (e.g., cyclophosphamide or melphalan) and/or a polymer-anticancer agent conjugate, particle or composition described herein.

In one embodiment, after the primary treatment, e.g., after the primary treatment and the high dose treatment, stem cells are transplanted into the subject. In one embodiment, a subject who has received a stem cell transplant is administered thalidomide. In one embodiment, the subject is further administered a corticosteroid (e.g., prednisone).

In one embodiment, the polymer-anticancer agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632, AZD2171, sorafenib and sunitinib.

In some embodiments, the composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating multiple myeloma in a subject, e.g., a human, the method comprising:

providing a subject who has multiple myeloma and has been treated with a chemotherapeutic agent that did not effectively treat the myeloma (e.g., the subject has a chemotherapeutic refractory myeloma, a chemotherapeutic resistant myeloma and/or a relapsed myeloma) or who had an unacceptable side effect (e.g., the subject has a chemotherapeutic sensitive myeloma), and

administering a polymer-anticancer agent conjugate, particle or composition, e.g., a polymer-anticancer agent conjugate, particle or composition described herein, to a subject in an amount effective to treat the myeloma, to thereby treat the myeloma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as bortezomib, docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the subject has been treated with a proteasome inhibitor, e.g., bortezomib, which did not effectively treat the myeloma (e.g., the subject has a bortezomib refractory, a bortezomib resistant and/or relapsed myeloma).

In one embodiment, the subject has been treated with an anthracycline (e.g., daunorubicin, doxorubicin, epirubicin, valrubicin or idarubicin) which did not effectively treat the cancer (e.g., the subject has a doxorubicin refractory, a doxorubicin resistant and/or a relapsed myeloma).

In one embodiment, the subject has been treated with a thalidomide or thalidomide derivative (e.g., lenalidomide) which did not effectively treat the myeloma (e.g., the subject has thalidomide or thalidomide derivative refractory, thalidomide or thalidomide derivative resistant and/or a relapsed myeloma).

In one embodiment, the polymer-anticancer agent conjugate, particle or composition is administered in combination with an anthracycline (e.g., daunorubicin, doxorubicin (e.g., liposomal doxorubicin or a polymer-doxorubicin conjugate, particle or composition described herein), epirubicin, valrubicin and idarubicin). In one embodiment, the polymer-anticancer agent conjugate, particle or composition is administered in combination with an anthracycline (e.g., daunorubicin, doxorubicin (e.g., liposomal doxorubicin or a polymer-doxorubicin conjugate, particle or composition described herein), epirubicin, valrubicin and idarubicin) and a proteasome inhibitor, e.g., bortezomib.

In another embodiment, the polymer-anticancer agent conjugate, particle or composition is administered in combination with a proteasome inhibitor, e.g., bortezomib.

In one embodiment, the polymer-anticancer agent conjugate, particle or composition is administered in combination with thalidomide or a thalidomide derivative (e.g. lenalidomide) and dexamethasone.

In one embodiment, the polymer-anticancer agent conjugate, particle or composition is administered in combination with dexamethasone and cyclophosphamide. In one embodiment, the polymer-anticancer agent conjugate, particle or composition is further administered in combination with a topoisomerase inhibitor (e.g., etoposide, topotecan, irinotecan, teniposide, SN-38, lamellarin D) and/or a platinum based agent (carboplatin, cisplatin, oxaliplatin). In one embodiment, the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating AIDS-related Kaposi's Sarcoma in a subject, e.g., a human. The method comprises: administering a polymer-anticancer agent conjugate, particle or composition, e.g., a polymer-anticancer agent conjugate, particle or composition described herein, to a subject in an amount effective to treat the sarcoma, to thereby treat the sarcoma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as bortezomib, docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer conjugate shown in FIG. 1 or FIG. 2.

In one embodiment, the polymer-anticancer agent conjugate, particle or composition is administered in combination with an antiviral agent, e.g., a nucleoside or a nucleotide reverse transcriptase inhibitor, a non-nucleoside reverse transcriptase inhibitor, a protease inhibitor, an integrase inhibitor, and entry or fusion inhibitor, a maturation inhibitor, or a broad spectrum inhibitor. Examples of nucleoside reverse transcriptase inhibitors include zidovudine, didanosine, zalcitabine, stavudine, lamivudine, abacavir, emtricitabine and apricitabine. Nucleotide reverse transcriptase include, e.g., tenofovir and adefovir. Examples of a non-nucleoside reverse transcriptase inhibitor include efavirenz, nevirapine, delavirdine and etravirine. Protease inhibitors include, e.g., saquinavir, ritonavir, indinavir, nelfinavir and amprenavir. An exemplary integrase inhibitor is raltegravir. Examples of entry inhibitors and fusion inhibitors include maraviroc and enfuvirtide. Maturation inhibitors include, e.g., bevirimat and vivecon.

In one embodiment, the polymer-anticancer agent conjugate, particle or composition is administered in combination with cryosurgery. In one embodiment, polymer-anticancer agent conjugate, particle or composition is administered in combination with alitretinoin.

In one embodiment, the polymer-anticancer agent conjugate, particle or composition is administered in combination with an anthracycline (e.g., daunorubicin, doxorubicin (e.g., liposomal doxorubicin or a polymer-doxorubicin conjugate, particle or composition described herein), epirubicin, valrubicin and idarubicin). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin). In one embodiment, the polymer-anticancer agent conjugate, particle or composition is further administered with a vinca alkaloid (e.g., vinblastine, vincristine, vindesine and vinorelbine) and an antibiotic (e.g., actinomycin, bleomycin, hydroxyurea and mitomycin).

In one embodiment, the polymer-anticancer agent conjugate, particle or composition is administered in combination with a taxane (e.g., paclitaxel (e.g., a polymer-paclitaxel conjugate, particle or composition described herein) or docetaxel (e.g., a polymer-docetaxel conjugate, particle or composition described herein)). For example, in one embodiment, the polymer-anticancer agent conjugate, particle or composition is a polymer-doxorubicin conjugate, particle or composition and the polymer-doxorubicin agent conjugate, particle or composition is further administered in combination with a taxane (e.g., paclitaxel (e.g., a polymer-paclitaxel conjugate, particle or composition described herein) or docetaxel (e.g., a polymer-docetaxel conjugate, particle or composition described herein)). In one embodiment, the polymer-anticancer agent conjugate, particle or composition is further administered with a vinca alkaloid (e.g., vinblastine, vincristine, vindesine and vinorelbine).

In one embodiment, the polymer-anticancer agent is administered in combination with a vinca alkaloid (e.g., vinblastine, vincristine, vindesine and vinorelbine).

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating AIDS-related Kaposi's Sarcoma, in a subject, e.g., a human. The method comprises:

providing a subject who has AIDS-related Kaposi's Sarcoma and has been treated with a chemotherapeutic agent which did not effectively treat the sarcoma (e.g., the subject has a chemotherapeutic refractory, a chemotherapeutic resistant and/or a relapsed sarcoma) or which had an unacceptable side effect (e.g., the subject has a chemotherapeutic sensitive sarcoma), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the sarcoma is refractory to, resistant to, and/or relapsed with treatment with one or more of: a taxane (e.g., paclitaxel and docetaxel), an anthracycline, a vinca alkaloid (e.g., vinblastine, vincristine, vindesine and vinorelbine) and an anthracycline (e.g., daunorubicin, doxorubicin, epirubicin, valrubicin and idarubicin).

In one embodiment, the cancer is a multidrug resistant sarcoma.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating gastric cancer in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer conjugate shown in FIG. 1 or FIG. 2.

In one embodiment, the gastric cancer is gastroesophageal junction adenocarcinoma.

In one embodiment, the polymer-anticancer agent conjugate, particle or composition is administered prior to surgery, after surgery or before and after surgery to remove the cancer.

In one embodiment, the polymer-anticancer agent conjugate, particle or composition is administered in combination with one or more of an anthracycline (e.g., daunorubicin, doxorubicin (e.g., liposomal doxorubicin or a polymer-doxorubicin conjugate, particle or composition described herein), epirubicin, valrubicin and idarubicin), a platinum-based agent (e.g., cisplatin, carboplatin, oxaliplatin) and an anti-metabolite, e.g., an antifolate (e.g., floxuridine, pemetrexed) or pyrimidine analogue (e.g., 5FU)). For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline (e.g., daunorubicin, doxorubicin (e.g., liposomal doxorubicin or a polymer-doxorubicin conjugate, particle or composition described herein), epirubicin, valrubicin and idarubicin), a platinum-based agent (e.g., cisplatin, carboplatin, oxaliplatin) and an anti-metabolite, e.g., an antifolate (e.g., floxuridine, pemetrexed) or pyrimidine analogue (e.g., 5FU)). In another embodiment, the polymer-anticancer agent conjugate, particle or composition is a polymer-doxorubicin conjugate, particle or composition and the polymer-doxorubicin conjugate, particle or composition is further administered in combination with a platinum-based agent (e.g., cisplatin, carboplatin, oxaliplatin) and an anti-metabolite, e.g., an antifolate (e.g., floxuridine, pemetrexed) or pyrimidine analogue (e.g., 5FU)).

In some embodiments, the polymer-anticancer agent conjugate, particle or composition is administered in combination with an anti-metabolite, e.g., an antifolate (e.g., floxuridine, pemetrexed) or pyrimidine analogue (e.g., capecitabine, 5FU)). In one embodiment, the polymer-anticancer agent conjugate, particle or composition is further administered with a taxane (e.g., paclitaxel (e.g., a polymer-paclitaxel conjugate, particle or composition described herein) or docetaxel (e.g., a polymer-docetaxel conjugate, particle or composition described herein)). For example, in one embodiment, the polymer-anticancer agent conjugate, particle or composition is a polymer-doxorubicin conjugate, particle or composition and the polymer-doxorubicin conjugate, particle or composition is further administered in combination with an anti-metabolite, e.g., an antifolate (e.g., floxuridine, pemetrexed) or pyrimidine analogue (e.g., capecitabine, 5FU)) and a taxane (e.g., paclitaxel (e.g., a polymer-paclitaxel conjugate, particle or composition described herein) or docetaxel (e.g., a polymer-docetaxel conjugate, particle or composition described herein)).

In one embodiment, the polymer-anticancer agent conjugate, particle or composition is administered in combination with radiation.

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating gastric cancer, e.g. a gastric cancer described herein such as gastroesophageal junction adenocarcinoma, in a subject, e.g., a human. The method comprises:

providing a subject who has gastric cancer and has been treated with a chemotherapeutic agent which did not effectively treat the cancer (e.g., the subject has a non-resectable cancer, a chemotherapeutic refractory, a chemotherapeutic resistant and/or a relapsed cancer) or which had an unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the cancer is refractory to, resistant to, and/or relapsed with treatment with one or more of: a taxane (e.g., paclitaxel and docetaxel), an anthracycline (e.g., daunorubicin, doxorubicin, epirubicin, valrubicin and idarubicin), an anti-metabolite, e.g., an antifolate (e.g., floxuridine, pemetrexed) or pyrimidine analogue (e.g., capecitabine, 5FU)), and a platinum-based agent (e.g., cisplatin, carboplatin, oxaliplatin).

In one embodiment, the cancer is a multidrug resistant cancer.

In one embodiment, the polymer-anticancer agent conjugate, particle or composition is administered in combination with a pyrimidine analogue, e.g., a pyrimidine analogue described herein (e.g., capecitabine and 5FU).

In one embodiment, the polymer-anticancer agent conjugate, particle or composition is administered in combination with a platinum-based agent (e.g., cisplatin, carboplatin, oxaliplatin). In one embodiment, the polymer-anticancer agent conjugate, particle or composition is further administered in combination with a pyrimidine analogue, e.g., a pyrimidine analogue described herein (e.g., capecitabine and 5FU). In another embodiment, the polymer-anticancer agent conjugate, particle or composition is further administered in combination with a topoisomerase inhibitor (e.g., etoposide, topotecan, irinotecan, teniposide, SN-38, lamellarin D).

In one embodiment, the polymer-anticancer agent conjugate, particle or composition is administered in combination with a topoisomerase inhibitor (e.g., etoposide, topotecan, irinotecan, teniposide, SN-38, lamellarin D). In one embodiment, the polymer-anticancer agent conjugate, particle or composition is further administered in combination with a pyrimidine analogue, e.g., a pyrimidine analogue described herein (e.g., capecitabine and 5FU).

In some embodiments, the polymer-anticancer agent conjugate, particle or composition is administered in combination with a taxane (e.g., paclitaxel and docetaxel). In one embodiment, the polymer-anticancer agent conjugate, particle or composition is further administered in combination with a pyrimidine analogue, e.g., a pyrimidine analogue described herein (e.g., capecitabine and 5FU). For example, in one embodiment, the polymer-anticancer agent conjugate, particle or composition is a polymer-doxorubicin conjugate, particle or composition and the polymer-doxorubicin conjugate, particle or composition is administered in combination with a taxane (e.g., paclitaxel (e.g., a polymer-paclitaxel conjugate, particle or composition described herein) and docetaxel (e.g., a polymer-docetaxel conjugate, particle or composition described herein)) and a pyrimidine analogue, e.g., a pyrimidine analogue described herein (e.g., capecitabine and 5FU).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating a soft tissue sarcoma (e.g., non-resectable, advanced, metastatic or relapsed soft tissue sarcoma) in a subject, e.g., a human. The method comprises: administering a polymer-anticancer agent conjugate, particle or composition, e.g., a polymer-anticancer agent conjugate, particle or composition described herein, to a subject in an amount effective to treat the sarcoma, to thereby treat the sarcoma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer conjugate shown in FIG. 1 or FIG. 2.

In one embodiment, the soft tissue sarcoma is rhabdomyosarcoma, leiomyosarcoma, hemangiosarcoma, lymphangiosarcoma, synovial sarcoma, neurofibrosarcoma, liposarcoma, fibrosarcoma, malignant fibrous histiocytoma and dermatofibrosarcoma.

In one embodiment, the polymer-anticancer agent conjugate, particle or composition is administered in combination with an anthracycline, e.g., daunorubicin, doxorubicin (e.g., liposomal doxorubicin or a polymer-doxorubicin conjugate, particle or composition described herein), epirubicin, valrubicin and idarubicin. For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is administered in combination with an anthracycline, e.g., daunorubicin, doxorubicin (e.g., liposomal doxorubicin or a polymer-doxorubicin conjugate, particle or composition described herein), epirubicin, valrubicin and idarubicin.

In one embodiment, the polymer-anticancer agent conjugate, particle or composition is administered in combination with an alkylating agent (e.g., cyclophosphamide, dacarbazine, melphalan, ifosfamide, temozolomide). In one embodiment, the polymer-anticancer agent conjugate, particle or composition is further administered in combination with mesna. In one embodiment, the polymer-anticancer agent conjugate, particle or composition is further administered in combination with an anthracycline, e.g., daunorubicin, doxorubicin (e.g., liposomal doxorubicin or a polymer-doxorubicin conjugate, particle or composition described herein), epirubicin, valrubicin and idarubicin. For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline, e.g., daunorubicin, doxorubicin (e.g., liposomal doxorubicin or a polymer-doxorubicin conjugate, particle or composition described herein), epirubicin, valrubicin and idarubicin.

In one embodiment, the polymer-anticancer agent conjugate, particle or composition is administered in combination with an anti-metabolite, e.g., an antifolate (e.g., pemetrexed, floxuridine, raltitrexed) or pyrimidine analog (e.g., capecitabine, cytarabine, gemcitabine, 5FU). In one embodiment, the polymer-anticancer agent conjugate, particle or composition is further administered in combination with a taxane.

In one embodiment, the polymer-anticancer agent conjugate, particle or composition is administered in combination with a taxane (e.g., paclitaxel (e.g., a polymer-paclitaxel conjugate, particle or composition described herein) and docetaxel (e.g., a polymer-docetaxel conjugate, particle or composition described herein)). For example, in one embodiment, the polymer-anticancer agent conjugate, particle or composition is a polymer-doxorubicin conjugate, particle or composition and the polymer-doxorubicin conjugate, particle or composition is administered in combination with a taxane (e.g., paclitaxel (e.g., a polymer-paclitaxel conjugate, particle or composition described herein) and docetaxel (e.g., a polymer-docetaxel conjugate, particle or composition described herein)).

In one embodiment, the polymer-anticancer agent conjugate, particle or composition is administered in combination with a vinca alkaloid (e.g., vinblastine, vincristine, vindesine, vinorelbine).

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating a soft tissue sarcoma, in a subject, e.g., a human. The method comprises:

providing a subject who has a soft tissue sarcoma and has been treated with a chemotherapeutic agent which did not effectively treat the sarcoma (e.g., the subject has a chemotherapeutic refractory, a chemotherapeutic resistant and/or a relapsed sarcoma) or which had an unacceptable side effect (e.g., the subject has a chemotherapeutic sensitive sarcoma), and

administering a polymer-anticancer agent conjugate, particle or composition, e.g., a polymer-anticancer agent conjugate, particle or composition described herein, to a subject in an amount effective to treat the sarcoma, to thereby treat the sarcoma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the sarcoma is refractory to, resistant to, and/or relapsed with treatment with one or more of: a taxane (e.g., paclitaxel and docetaxel), an anthracycline (e.g., doxorubicin, daunorubicin, epirubicin, idarubicin, mitoxantrone, valrubicin), a vinca alkaloid (e.g., vinblastine, vincristine, vindesine and vinorelbine) and an alkylating agent (e.g., cyclophosphamide, dacarbazine, melphalan, ifosfamide, temozolomide).

In one embodiment, the sarcoma is a multidrug resistant cancer.

In one embodiment, the soft tissue sarcoma is rhabdomyosarcoma, leiomyosarcoma, hemangiosarcoma, lymphangiosarcoma, synovial sarcoma, neurofibrosarcoma, liposarcoma, fibrosarcoma, malignant fibrous histiocytoma and dermatofibrosarcoma.

In one embodiment, the polymer-anticancer agent conjugate, particle or composition is administered in combination with an anthracycline, e.g., daunorubicin, doxorubicin (e.g., liposomal doxorubicin or a polymer-doxorubicin conjugate, particle or composition described herein), epirubicin, valrubicin and idarubicin. For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is administered in combination with an anthracycline, e.g., daunorubicin, doxorubicin (e.g., liposomal doxorubicin or a polymer-doxorubicin conjugate, particle or composition described herein), epirubicin, valrubicin and idarubicin.

In one embodiment, the polymer-anticancer agent conjugate, particle or composition is administered in combination with an alkylating agent (e.g., cyclophosphamide, dacarbazine, melphalan, ifosfamide, temozolomide). In one embodiment, the polymer-anticancer agent conjugate, particle or composition is further administered in combination with mesna. In one embodiment, the polymer-anticancer agent conjugate, particle or composition is further administered in combination with an anthracycline, e.g., daunorubicin, doxorubicin (e.g., liposomal doxorubicin or a polymer-doxorubicin conjugate, particle or composition described herein), epirubicin, valrubicin and idarubicin. For example, in one embodiment, the polymer-anticancer agent conjugate, particle or composition is a polymer-docetaxel conjugate, particle or composition and/or a polymer-paclitaxel conjugate, particle or composition and the polymer-anticancer agent conjugate, particle or composition is further administered in combination with an anthracycline, e.g., daunorubicin, doxorubicin (e.g., liposomal doxorubicin or a polymer-doxorubicin conjugate, particle or composition described herein), epirubicin, valrubicin and idarubicin.

In one embodiment, the polymer-anticancer agent conjugate, particle or composition is administered in combination with an anti-metabolite, e.g., an antifolate (e.g., pemetrexed, floxuridine, raltitrexed) or pyrimidine analog (e.g., capecitabine, cytarabine, gemcitabine, 5FU). In one embodiment, the polymer-anticancer agent conjugate, particle or composition is further administered in combination with a taxane.

In one embodiment, the polymer-anticancer agent conjugate, particle or composition is administered in combination with a taxane (e.g., paclitaxel (e.g., a polymer-paclitaxel conjugate, particle or composition described herein) and docetaxel (e.g., a polymer-docetaxel conjugate, particle or composition described herein)). For example, in one embodiment, the polymer-anticancer agent conjugate, particle or composition is a polymer-doxorubicin conjugate, particle or composition and the polymer-doxorubicin conjugate, particle or composition is administered in combination with a taxane (e.g., paclitaxel (e.g., a polymer-paclitaxel conjugate, particle or composition described herein) and docetaxel (e.g., a polymer-docetaxel conjugate, particle or composition described herein)).

In one embodiment, the polymer-anticancer agent conjugate, particle or composition is administered in combination with a vinca alkaloid (e.g., vinblastine, vincristine, vindesine, vinorelbine).

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In one aspect, the disclosure features a method of treating pancreatic cancer (e.g., locally advanced or metastatic pancreatic cancer) in a subject, e.g., a human. The method comprises: administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel, doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer conjugate shown in FIG. 1 or FIG. 2. In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion), codon 13 of the KRAS gene, codon 61 of the KRAS gene.

In one embodiment, the polymer-anticancer agent conjugate, particle or composition is administered after surgery or before and after surgery to remove the cancer.

In one embodiment, the polymer-anticancer agent conjugate, particle or composition is administered in combination with one or more of an anti-metabolite, e.g., an antifolate, e.g., floxuridine, a pyrimidine analogue, e.g., 5FU, capecitabine, and/or a nucleoside analog, e.g., gemcitabine. For example, in one embodiment, the polymer-anticancer agent conjugate, particle or composition is administered in combination with a nucleoside analog, e.g., gemcitabine. In one embodiment, the polymer-anticancer agent conjugate, particle or composition is a polymer-doxorubicin conjugate, particle or composition is further administered in combination with a platinum-based agent (e.g., cisplatin, carboplatin, oxaliplatin) and a pyrimidine analogue (e.g., 5FU and/or capecitabine). In one embodiment, the polymer anticancer agent conjugate, particle or composition is further administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In some embodiments, the polymer-anticancer agent conjugate, particle or composition is administered in combination with an anti-metabolite, e.g., 5FU, and leucovorin. In one embodiment, the polymer-anticancer agent conjugate, particle or composition is administered in combination with radiation.

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632, AZD2171, sorafenib and sunitinib). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is administered in combination with a poly ADP-ribose polymerase (PARP) inhibitor (e.g., BSI 201, Olaparib (AZD-2281), ABT-888, AG014699, CEP 9722, MK 4827, KU-0059436 (AZD2281), LT-673, 3-aminobenzamide).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In one aspect, the disclosure features a method of treating pancreatic cancer, e.g. locally advanced or metastatic pancreatic cancer, in a subject, e.g., a human. The method comprises:

providing a subject who has pancreatic cancer and has been treated with a chemotherapeutic agent which did not effectively treat the cancer (e.g., the subject has a non-resectable cancer, a chemotherapeutic refractory, a chemotherapeutic resistant and/or a relapsed cancer) or which had an unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2. In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion), codon 13 of the KRAS gene, codon 61 of the KRAS gene.

In one embodiment, the cancer is refractory to, resistant to, and/or relapsed with treatment with one or more of: a taxane (e.g., paclitaxel, docetaxel, larotaxel, cabazitaxel), an anthracycline (e.g., daunorubicin, doxorubicin, epirubicin, valrubicin and idarubicin), an anti-metabolite, e.g., an antifolate (e.g., floxuridine, pemetrexed) or pyrimidine analogue (e.g., capecitabine, 5FU)), and a platinum-based agent (e.g., cisplatin, carboplatin, oxaliplatin).

In one embodiment, the cancer is a multidrug resistant cancer.

In one embodiment, the polymer-anticancer agent conjugate, particle or composition is administered in combination with a pyrimidine analogue, e.g., a pyrimidine analogue described herein (e.g., capecitabine and/or 5FU). In one embodiment, the polymer-anticancer agent conjugate, particle or composition is administered in combination with a pyrimidine analogue, e.g., 5FU, and leucovorin. In one embodiment, the polymer-anticancer agent conjugate, particle or composition is further administered in combination with a platinum-based agent (e.g., cisplatin, carboplatin, oxaliplatin).

In one embodiment, the polymer-anticancer agent conjugate, particle or composition is administered in combination with a poly ADP-ribose polymerase (PARP) inhibitor (e.g., BSI 201, Olaparib (AZD-2281), ABT-888, AG014699, CEP 9722, MK 4827, KU-0059436 (AZD2281), LT-673, 3-aminobenzamide).

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of treating advanced or metastatic colorectal cancer in a subject, e.g., a human. The method comprises: administering a composition comprising a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2. In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion), codon 13 of the KRAS gene, codon 61 of the KRAS gene.

In one embodiment, the polymer-anticancer agent conjugate, particle or composition is administered in combination with an antimetabolite, e.g., an antifolate (e.g., pemetrexed, raltitrexed). In one embodiment, the polymer-anticancer agent conjugate, particle or composition is administered in combination with an antimetabolite, e.g., 5FU, and leucovorin. In one embodiment, the polymer-anticancer agent conjugate, particle or composition is further administered in combination with a platinum-based agent (e.g., cisplatin, carboplatin, oxaliplatin). For example, in one embodiment, the polymer-anticancer agent conjugate, particle or composition is administered in combination with an antimetabolite, e.g., 5FU, leucovorin, and a platinum-based agent, e.g., oxaliplatin. In another embodiment, the antimetabolite is a pyrimidine analog, e.g., capecitabine.

In one embodiment, the polymer-anticancer agent conjugate, particle or composition is administered in combination with a platinum-based agent (e.g., cisplatin, carboplatin, oxaliplatin).

In one embodiment, the polymer-anticancer agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632, AZD2171, sorafenib and sunitinib. In one embodiment, the polymer-anticancer agent conjugate, particle or composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the polymer-anticancer agent conjugate, particle or composition is administered with a VEGF pathway inhibitor, e.g., bevacizumab, an antimetabolite, e.g., a pyrimidine analogue (e.g., 5FU), and leucovorin. In another embodiment, the polymer-anticancer agent conjugate, particle or composition is administered with a VEGF pathway inhibitor, e.g., bevacizumab, an antimetabolite, e.g., a pyrimidine analogue (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, SN-38, camptothecin (e.g., IT-101)). For example, in one embodiment, the polymer-anticancer agent conjugate, particle or composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the polymer-anticancer agent conjugate, particle or composition is administered in combination with a VEGF pathway inhibitor, e.g., bevacizumab, and an antimetabolite wherein the antimetabolite is a pyrimidine analog, e.g., capecitabine. In one embodiment, the polymer-anticancer agent conjugate, particle or composition is further administered in combination with a platinum-based agent (e.g., cisplatin, carboplatin, oxaliplatin) or a topoisomerase inhibitor (e.g., irinotecan, topotecan, etoposide, teniposide, lamellarin D, SN-38, camptothecin (e.g., IT-101)). For example, in one embodiment, the polymer-anticancer agent conjugate, particle or composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase I inhibitor (e.g., irinotecan).

In one embodiment, the polymer-anticancer agent conjugate, particle or composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the polymer-anticancer agent conjugate, particle or composition is administered in combination with an EGF pathway inhibitor, e.g., cetuximab or panitumumab, and a VEGF pathway inhibitor, e.g., bevacizumab.

In one embodiment, the polymer-anticancer agent conjugate, particle or composition is administered in combination with a topoisomerase inhibitor (e.g., irinotecan, topotecan, etoposide, teniposide, lamellarin D, SN-38, camptothecin (e.g., IT-101)). In one embodiment, the polymer-anticancer agent conjugate, particle or composition is administered in combination with a topoisomerase I inhibitor (e.g., irinotecan) and a VEGF pathway inhibitor, e.g., bevacizumab.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1.

In one embodiment, the polymer-doxorubicin conjugate, particle or composition is administered at a dose and/or dosing schedule described herein. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.gate, particle or composition is administered at a dose and/or dosing schedule described herein.

In yet another aspect, the invention features a method of treating advanced or metastatic colorectal cancer in a subject, e.g., a human, the method comprising:

providing a subject who has advanced or metastatic colorectal cancer and has been treated with a chemotherapeutic agent that did not effectively treat the cancer (e.g., the subject has a chemotherapeutic refractory cancer, a chemotherapeutic resistant cancer and/or a relapsed cancer) or who had an unacceptable side effect (e.g., the subject has a chemotherapeutic sensitive cancer), and

administering a polymer-anticancer agent conjugate, particle or composition, e.g., a polymer-anticancer agent conjugate, particle or composition described herein, to a subject in an amount effective to treat the cancer, to thereby treat the cancer.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion), codon 13 of the KRAS gene, codon 61 of the KRAS gene.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the subject has been treated with an anti-metabolite, e.g., a pyrimidine analogue which did not effectively treat the cancer (e.g., the subject has a capecitabine and/or 5FU refractory, a capecitabine and/or 5FU resistant and/or relapsed cancer).

In one embodiment, the subject has been treated with a pyrimidine analog which did not effectively treat the cancer (e.g., the subject has a capecitabine refractory, a capecitabine resistant and/or a relapsed cancer).

In one embodiment, the polymer-anticancer agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632, AZD2171, sorafenib and sunitinib. In one embodiment, the polymer-anticancer agent conjugate, particle or composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the polymer-anticancer agent conjugate, particle or composition is administered with a VEGF pathway inhibitor, e.g., bevacizumab, an antimetabolite (e.g., 5FU) and leucovorin. In another embodiment, the polymer-anticancer agent conjugate, particle or composition is administered with a VEGF pathway inhibitor, e.g., bevacizumab, an antimetabolite (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, SN-38, camptothecin (e.g., IT-101)). For example, in one embodiment, the polymer-anticancer agent conjugate, particle or composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase I inhibitor (e.g., irinotecan); or a VEGF pathway inhibitor, e.g., bevacizumab, an antimetabolite (e.g., 5FU), leucovorin and a topoisomerase I inhibitor (e.g., irinotecan).

In another embodiment, the polymer-anticancer agent conjugate, particle or composition is administered in combination with a VEGF pathway inhibitor, e.g., bevacizumab, and an antimetabolite wherein the antimetabolite is a pyrimidine analog, e.g., capecitabine. In one embodiment, the polymer-anticancer agent conjugate, particle or composition is further administered in combination with a platinum-based agent (e.g., cisplatin, carboplatin, oxaliplatin) or a topoisomerase inhibitor (e.g., irinotecan, topotecan, etoposide, teniposide, lamellarin D, SN-38, camptothecin (e.g., IT-101)). For example, in one embodiment, the polymer-anticancer agent conjugate, particle or composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase I inhibitor (e.g., irinotecan).

In one embodiment, the polymer-anticancer agent conjugate, particle or composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the polymer-anticancer agent conjugate, particle or composition is administered in combination with an EGF pathway inhibitor, e.g., cetuximab or panitumumab, and a VEGF pathway inhibitor, e.g., bevacizumab.

In one embodiment, the polymer-anticancer agent conjugate, particle or composition is administered in combination with a topoisomerase inhibitor (e.g., irinotecan, topotecan, etoposide, teniposide, lamellarin D, SN-38, camptothecin (e.g., IT-101)). In one embodiment, the polymer-anticancer agent conjugate, particle or composition is administered in combination with a topoisomerase I inhibitor (e.g., irinotecan) and a VEGF pathway inhibitor, e.g., bevacizumab.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In yet another aspect, the invention features a method of identifying a subject, e.g., a human, having a proliferative disorder, e.g., cancer, for treatment with a polymer-anticancer agent conjugate, particle or composition, e.g., a polymer-anticancer agent conjugate, particle or composition described herein, the method comprising

identifying a subject having a proliferative disorder who has received an anticancer agent (e.g., docetaxel, paclitaxel, larotaxel, cabazitaxel or doxorubicin) and has a neutrophil count less than a standard; and

identifying the subject as suitable for treatment with a polymer-anticancer agent conjugate, particle or composition, e.g., a polymer-anticancer agent conjugate, particle or composition described herein.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the method further comprising administering a polymer-anticancer agent conjugate, particle or composition, e.g., a polymer-anticancer agent conjugate, particle or composition described herein in an amount effective to treat the disorder.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In another aspect, the invention features a method of treating a subject, e.g., a human, with a proliferative disorder, e.g., cancer, the method comprising

selecting a subject having a proliferative disease who has received an anticancer agent (e.g., docetaxel, paclitaxel, larotaxel, cabazitaxel or doxorubicin) and has a neutrophil count less than a standard; and

administering a polymer-anticancer agent conjugate, particle or composition, e.g., a polymer-anticancer agent conjugate, particle or composition described herein, to the subject in an amount effective to treat the proliferative disorder, to thereby treat the disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the standard is a neutrophil count below or equal to 1500 cells/mm³. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In yet another aspect, the invention features a method for selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining whether a subject with a proliferative disorder has moderate to severe neutropenia; and

selecting a subject for treatment with a polymer-anticancer agent conjugate, particle or composition on the basis that the subject has moderate to severe neutropenia.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-docetaxel conjugate, particle or composition is administered in an amount such that the conjugate, particle or composition includes 60 mg/m² of docetaxel, an additional dose is administered in an amount such that the conjugate, particle or composition includes 60 mg/m² or greater of docetaxel.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-paclitaxel conjugate, particle or composition is administered in an amount such that the conjugate, particle or composition includes 135 mg/m² of paclitaxel, an additional dose is administered in an amount such that the conjugate, particle or composition includes 135 mg/m² or greater of paclitaxel.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-doxorubicin conjugate, particle or composition is administered in an amount such that the conjugate, particle or composition includes 40 mg/m² of doxorubicin, an additional dose is administered in an amount such that the conjugate, particle or composition includes 40 mg/m² or greater of doxorubicin.

In one embodiment, the method further comprises administering a polymer-anticancer agent conjugate, particle or composition, e.g., a polymer-anticancer agent conjugate, particle or composition described herein, to the subject.

In one embodiment, the subject experienced moderate to severe neutropenia from treatment with an anticancer agent. In one embodiment, the subject has one or more symptom of febrile neutropenia.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In one embodiment, the standard for moderate neutropenia is a neutrophil count of 1000 to 500 cells/mm³. In one embodiment, the standard for severe neutropenia is a neutrophil count of less than 500 cells/mm³.

In yet another aspect, the invention features a method for treating a subject, e.g., a human, with a proliferative disorder, e.g., cancer, comprising:

selecting a subject with a proliferative disorder, e.g., cancer, who has moderate to severe neutropenia; and

administering a polymer-anticancer agent conjugate, particle or composition, e.g., a polymer-anticancer agent conjugate, particle or composition described herein, to the subject in an amount effective to treat the disorder, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-docetaxel conjugate, particle or composition is administered in an amount such that the conjugate, particle or composition includes 60 mg/m² of docetaxel, an additional dose is administered in an amount such that the conjugate, particle or composition includes 60 mg/m² or greater of docetaxel.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2.

In one embodiment, the polymer-paclitaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-paclitaxel conjugate, particle or composition is administered in an amount such that the conjugate, particle or composition includes 135 mg/m² of paclitaxel, an additional dose is administered in an amount such that the conjugate, particle or composition includes 135 mg/m² or greater of paclitaxel.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-doxorubicin conjugate, particle or composition is administered in an amount such that the conjugate, particle or composition includes 40 mg/m² of doxorubicin, an additional dose is administered in an amount such that the conjugate, particle or composition includes 40 mg/m² or greater of doxorubicin.

In one embodiment, the method further comprises administering a polymer-anticancer agent conjugate, particle or composition, e.g., a polymer-anticancer agent conjugate, particle or composition described herein, to the subject.

In one embodiment, the subject experienced moderate to severe neutropenia from treatment with an anticancer agent. In one embodiment, the subject has one or more symptom of febrile neutropenia.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In one embodiment, the standard for moderate neutropenia is a neutrophil count of 1000 to 500 cells/mm³. In one embodiment, the standard for severe neutropenia is a neutrophil count of less than 500 cells/mm³.

In yet another aspect, the invention features a method for selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining whether a subject with a proliferative disorder, e.g., cancer, has experienced neuropathy from treatment with an anticancer agent, e.g., a taxane, a vinca alkaloid, an alkylating agent, a platinum-based agent or an epothilone; and

selecting a subject for treatment with a polymer-anticancer agent conjugate, particle or composition, e.g., a polymer-anticancer agent conjugate, particle or composition described herein, on the basis that the subject has experienced neuropathy from treatment with a chemotherapeutic agent, e.g., a taxane, a vinca alkaloid, an alkylating agent, a platinum-based agent or an epothilone.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-docetaxel conjugate, particle or composition is administered in an amount such that the conjugate, particle or composition includes 60 mg/m² of docetaxel, an additional dose is administered in an amount such that the conjugate, particle or composition includes 60 mg/m² or greater of docetaxel.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-paclitaxel conjugate, particle or composition is administered in an amount such that the conjugate, particle or composition includes 135 mg/m² of paclitaxel, an additional dose is administered in an amount such that the conjugate, particle or composition includes 135 mg/m² or greater of paclitaxel.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-doxorubicin conjugate, particle or composition is administered in an amount such that the conjugate, particle or composition includes 40 mg/m² of doxorubicin, an additional dose is administered in an amount such that the conjugate, particle or composition includes 40 mg/m² or greater of doxorubicin.

In one embodiment, the neuropathy is peripheral neuropathy. In one embodiment, the neuropathy is sensory neuropathy, motor neuropathy or both.

In one embodiment, the cancer is a cancer described herein. In one embodiment, the subject is selected for treatment with the polymer-anticancer agent conjugate, particle or composition in combination with one or more additional chemotherapeutic agent, e.g., a chemotherapeutic agent or combination of chemotherapeutic agents described herein.

In yet another aspect, the invention features a method for treating a subject, e.g., a human, with a proliferative disorder, e.g., cancer, comprising:

selecting a subject with a proliferative disorder, e.g., cancer, who has experienced one or more symptom of neuropathy from treatment with a chemotherapeutic agent, e.g., a taxane, a vinca alkaloid, an alkylating agent, a platinum-based agent or an epothilone; and administering a polymer-anticancer agent conjugate, particle or composition, e.g., a polymer-anticancer agent conjugate, particle or composition described herein, to the subject in an amount effective to treat the disorder, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel, cabazitaxel or doxorubicin, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-docetaxel conjugate, particle or composition is administered in an amount such that the conjugate, particle or composition includes 60 mg/m² of docetaxel, an additional dose is administered in an amount such that the conjugate, particle or composition includes 60 mg/m² or greater of docetaxel.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional dose (or doses). For example, when a dose of the polymer-paclitaxel conjugate, particle or composition is administered in an amount such that the conjugate, particle or composition includes 135 mg/m² of paclitaxel, an additional dose is administered in an amount such that the conjugate, particle or composition includes 135 mg/m² or greater of paclitaxel.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-doxorubicin conjugate, particle or composition is administered at a dose and/or dosing schedule described herein. In one embodiment, the dosing schedule is not changed between doses. For example, when the dosing schedule is every three weeks, an additional dose is administered in three weeks. In one embodiment, the dose does not change or is increased for an additional doses (or doses). For example, when a dose of the polymer-doxorubicin conjugate, particle or composition is administered in an amount such that the conjugate, particle or composition includes 40 mg/m² of doxorubicin, an additional dose is administered in an amount such that the conjugate, particle or composition includes 40 mg/m² or greater of doxorubicin.

In one embodiment, the subject experienced moderate to severe neuropathy from treatment with a chemotherapeutic agent. In one embodiment, the neuropathy is peripheral neuropathy. In one embodiment, the neuropathy is sensory neuropathy, motor neuropathy or both.

In one embodiment, the subject has experienced neuropathy after two, three fours, five cycles of treatment with an anticancer agent.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In another aspect, the invention features a method for selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining whether a subject with a proliferative disorder, e.g., cancer, has experienced an infusion site reaction (e.g., during or within 12 hours of infusion of an anticancer agent (e.g., a taxane)) or has or is at risk for having hypersensitivity to treatment with an anticancer agent (e.g., a taxane),

selecting a subject for treatment with a polymer-anticancer agent conjugate, particle or composition on the basis that the subject is in need of a reduced infusion site reaction (e.g., reduced as compared to the reaction associated with or caused by the treatment with an anticancer agent (e.g., taxane)) or the subject has or is at risk for having hypersensitivity to treatment with an anticancer agent (e.g., a taxane).

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel or cabazitaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the subject has exhibited one or more symptom of infusion site reaction to a previous treatment with the anticancer agent (e.g., taxane). Symptoms of infusion site reaction include: phlebitis, cellulitis, induration, skin exfoliation, necrosis, fibrosis, hyperpigmentation, inflammation and extravasation.

In one embodiment, the subject has exhibited one or more symptom of hypersensitivity to a previous treatment with the anticancer agent (e.g., the taxane) or to a treatment formulated with Cremaphor and/or polysorbate. Symptoms hypersensitivity include: dyspnea, hypotension, angioedema, urticaria, bronchospasm and erythema.

In one embodiment, the cancer is a cancer described herein. In one embodiment, the polymer-anticancer conjugate, particle or composition is selected for administration in combination with one or more additional chemotherapeutic agent, e.g., a chemotherapeutic agent or combination of chemotherapeutic agents described herein.

In yet another aspect, the invention features a method of treating a subject, e.g., a human, with a proliferative disorder, e.g., cancer, comprising:

selecting a subject with a proliferative disorder, e.g., cancer, who has experienced an infusion site reaction to treatment with an anticancer agent (e.g., a taxane) or has or is at risk for having hypersensitivity to an anticancer agent (e.g., a taxane); and administering a polymer-anticancer agent conjugate, particle or composition, e.g., a polymer-anticancer agent conjugate, particle or composition described herein, to the subject in an amount effective to treat the disorder, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel or cabazitaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the subject has exhibited one or more symptom of infusion site reaction to a previous treatment with the anticancer agent (e.g., taxane). Symptoms of infusion site reaction include: phlebitis, cellulitis, induration, skin exfoliation, necrosis, fibrosis, hyperpigmentation, inflammation and extravasation.

In one embodiment, the subject has exhibited one or more symptom of hypersensitivity to a previous treatment with the anticancer agent (e.g., the taxane) or a treatment formulated with Cremaphor and/or polysorbate. Symptoms hypersensitivity include: dyspnea, hypotension, angioedema, urticaria, bronchospasm and erythema.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In yet another aspect, the invention features a method of treating a subject, e.g., a human, with a proliferative disorder, e.g., cancer, comprising:

administering a polymer-anticancer agent conjugate, particle or composition, e.g., a polymer-anticancer agent conjugate, particle or composition described herein, to a subject with a proliferative disorder, e.g., cancer, in an amount effective to treat the disorder and in the absence of administration of one or more of a corticosteroid, an H1 antagonist and an H2 antagonist, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel or cabazitaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the polymer-anticancer agent conjugate, particle or composition is administered in the absence of administration of dexamethasone. In one embodiment, the polymer-anticancer agent conjugate, particle or composition is administered in the absence of administration of diphenhydramine. In one embodiment, the polymer-anticancer agent conjugate, particle or composition is administered in the absence of administration of cimetidine and/or ranitidine.

In one embodiment, the cancer is a cancer described herein. In one embodiment, the polymer-anticancer agent conjugate, particle or composition is administered in combination with one or more additional chemotherapeutic agent, e.g., a chemotherapeutic agent or combination of chemotherapeutic agents described herein.

In yet another aspect, the invention features a method of treating a subject, e.g., a human, with a proliferative disorder, e.g., cancer, comprising:

administering a polymer-anticancer agent conjugate, particle or composition, e.g., a polymer-anticancer agent conjugate, particle or composition described herein, to a subject with a proliferative disorder, e.g., cancer, in an amount effective to treat the disorder and in combination with a corticosteroid (e.g., dexamethasone), wherein the corticosteroid (e.g., dexamethasone) is administered at a dose less than 60 mg, 55 mg, 50 mg, 45 mg, 40 mg, 35 mg, 30 mg, to thereby treat the disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In yet another aspect, the invention features a method of treating a subject, e.g., a human, with a proliferative disorder, e.g., cancer, comprising:

administering a polymer-anticancer agent conjugate, particle or composition, e.g., a polymer-anticancer agent conjugate, particle or composition described herein, to a subject with a proliferative disorder, e.g., cancer, in an amount effective to treat the disorder and in combination with a corticosteroid (e.g., dexamethasone), an H1 antagonist (e.g., diphenhydramine) and/or an H2 antagonist (e.g., cimetidine and/or ranitidine), wherein the corticosteroid (e.g., dexamethasone) is administered at a dose less than 20 mg, 15 mg, 10 mg, 5 mg; the H1 antagonist (e.g., diphenhydramine) is administered at a dose of less than 50 mg, 45 mg, 30 mg, 20 mg, 15 mg, 10 mg, 5 mg; and/or the H2 antagonist (e.g., cimetidine) is administered at a dose of less than 300 mg, 275 mg, 250 mg, 225 mg, 200 mg, 175 mg, 150 mg, 125 mg, 100 mg and/or the H2 antagonist (e.g., ranitidime) is administered at a dose less than 50 mg, 45 mg, 40 mg, 35 mg, 30 mg, 25 mg, 20 mg, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-docetaxel conjugate shown in FIG. 1 or FIG. 2.

In one embodiment, the polymer-paclitaxel conjugate, particle or composition is administered at a dose and/or dosing schedule described herein.

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In yet another aspect, the invention features a method of selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining alanine aminotransferase (ALT), aspartate aminotransferase (AST) and/or bilirubin levels in a subject having a proliferative disorder; and

selecting a subject having ALT and/or AST levels greater than 2.5 times the upper limit of normal (ULN) and/or bilirubin levels greater than 2 times the ULN for treatment with a polymer-anticancer agent conjugate, particle or composition, e.g., a polymer-anticancer agent conjugate, particle or composition described herein.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1.

In one embodiment, the polymer-doxorubicin conjugate, particle or composition is administered at a dose and/or dosing schedule described herein.

In one embodiment, the cancer is a cancer described herein. In one embodiment, the subject is selected for treatment with the polymer-anticancer agent conjugate, particle or composition in combination with one or more additional chemotherapeutic agent, e.g., a chemotherapeutic agent or combination of chemotherapeutic agents described herein.

In yet another aspect, the invention features a method of treating a subject, e.g., a human, having a proliferative disorder, e.g., cancer, comprising:

selecting a subject with a proliferative disorder who has alanine aminotransferase (ALT) and/or aspartate aminotransferase (AST) levels greater than 2.5 times the upper limit of normal (ULN) and/or bilirubin levels greater than 2 times the ULN; and

administering a polymer-anticancer agent conjugate, particle or composition, e.g., a polymer-anticancer agent conjugate, particle or composition described herein, to the subject in an amount effective to treat the disorder, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2.

In one embodiment, the polymer-paclitaxel conjugate, particle or composition is administered at a dose and/or dosing schedule described herein.

In one embodiment, the polymer-anticancer agent conjugate, particle or composition is a polymer-doxorubicin conjugate, particle or composition, e.g., a polymer-doxorubicin conjugate, particle or composition described herein, e.g., a polymer-doxorubicin conjugate comprising doxorubicin, coupled, e.g., via linkers, to a polymer described herein. In an embodiment, the polymer-doxorubicin conjugate comprises doxorubicin, coupled via a linker shown in FIG. 1 to a polymer described herein. In an embodiment, the polymer-doxorubicin conjugate is a polymer-doxorubicin conjugate shown in FIG. 1.

In one embodiment, the polymer-doxorubicin conjugate, particle or composition is administered at a dose and/or dosing schedule described herein.

In one embodiment, the cancer is a cancer described herein. In one embodiment, the subject is selected for treatment with the polymer-anticancer agent conjugate, particle or composition in combination with one or more additional chemotherapeutic agent, e.g., a chemotherapeutic agent or combination of chemotherapeutic agents described herein.

In yet another aspect, the invention features a method of selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining alkaline phosphatase (ALP), serum glutamate oxaloacetate transaminase (SGOT), serum glutamate pyruvate transaminase (SGPT) and/or bilirubin levels in a subject having a proliferative disorder; and

selecting a subject having ALP levels greater than 2.5 times the upper limit of normal (ULN), SGOT and/or SGPT levels greater than 1.5 times the upper limit of normal (ULN) and/or bilirubin levels greater than the ULN for treatment with an anticancer agent (e.g., docetaxel), e.g., a polymer-anticancer agent conjugate, particle or composition, e.g., a polymer-anticancer agent conjugate, particle or composition described herein.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2, to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the cancer is a cancer described herein. In one embodiment, the subject is selected for treatment with the polymer-anticancer agent conjugate, particle or composition in combination with one or more additional chemotherapeutic agent, e.g., a chemotherapeutic agent or combination of chemotherapeutic agents described herein.

In yet another aspect, the invention features a method of treating a subject, e.g., a human, having a proliferative disorder, e.g., cancer, comprising:

selecting a subject with a proliferative disorder who has alkaline phosphatase (ALP) levels greater than 2.5 times the upper limit of normal (ULN), serum glutamate oxaloacetate transaminase (SGOT) and/or serum glutamate pyruvate transaminase (SGPT) levels greater than 1.5 times the ULN and/or bilirubin levels greater than the ULN; and

administering a polymer-anticancer agent conjugate, particle or composition, e.g., a polymer-anticancer agent conjugate, particle or composition described herein, to the subject in an amount effective to treat the disorder, to thereby treat the proliferative disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the cancer is a cancer described herein. In one embodiment, the subject is selected for treatment with the polymer-anticancer agent conjugate, particle or composition in combination with one or more additional chemotherapeutic agent, e.g., a chemotherapeutic agent or combination of chemotherapeutic agents described herein.

In yet another aspect, the invention features a method of selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining if a subject having a proliferative disorder is currently being administered (e.g., the subject has been administered a cytochrome P450 isoenzyme inhibitor, e.g., a CYP3A4 inhibitor or a CYP2C8 inhibitor, the same day as chemotherapy treatment or within 1, 2, 3, 4, 5, 6, or 7 days before chemotherapy treatment) or will be administered (e.g., will be administered on the same day as the chemotherapy treatment or within 1, 2, 3, 4, 5, 6, or 7 days after chemotherapy treatment) a cytochrome P450 isoenzyme inhibitor, e.g., CYP3A4 inhibitor (e.g., ketoconazole, itraconazole, clarithromycin, atazanavir, nefazodone, saquinavir, telithromycin, ritonavir, amprenavir, indinavir, nelfinavir, delavirdine or voriconazole) and/or a CYP2C8 inhibitor (e.g., quercetin); and

selecting a subject with a proliferative disorder, e.g., cancer, who is currently being administered or will be administered a cytochrome P450 isoenzyme, e.g., a CYP3A4 inhibitor and/or a CYP2C8 inhibitor, for treatment with a polymer-anticancer agent conjugate, particle or composition, e.g., a polymer-anticancer agent conjugate, particle or composition described herein, at a dose described herein.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel or cabazitaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In another aspect, the invention features a method of treating a subject, e.g., a human, having a proliferative disorder, e.g., cancer, comprising:

selecting a subject with a proliferative disorder, e.g., cancer, who is currently being administered or will be, administered a cytochrome P450 isoenzyme, e.g., a CYP3A4 inhibitor and/or a CYP2C8 inhibitor;

administering a polymer-anticancer agent conjugate, particle or composition, e.g., a polymer-anticancer agent conjugate, particle or composition, described herein, to the subject at a dose described herein, to thereby treat the disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, paclitaxel, larotaxel or cabazitaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the polymer-anticancer agent conjugate, particle or composition is a polymer-paclitaxel conjugate, particle or composition, e.g., a polymer-paclitaxel conjugate, particle or composition described herein, e.g., a polymer-paclitaxel conjugate comprising paclitaxel, coupled, e.g., via linkers, to a polymer described herein. In an embodiment, the polymer-paclitaxel conjugate comprises paclitaxel, coupled via a linker shown in FIG. 1 or FIG. 2 to a polymer described herein. In an embodiment, the polymer-paclitaxel conjugate is a polymer-paclitaxel conjugate shown in FIG. 1 or FIG. 2. Alternatively, the polymer-anticancer agent conjugate is as described in any one of the 1^{st} to the 12^{th} embodiments defined below and the boronic acid containing drug is bortezomib. Alternatively, the polymer-agent conjugate is a polymer-bortezomib conjugate.

In one embodiment, the polymer-paclitaxel conjugate, particle or composition is administered at a dose and/or dosing schedule described herein.

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In yet another aspect, the invention features a method of selecting a subject, e.g., a human, with a proliferative disorder, e.g., cancer, for treatment with a polymer-anticancer agent conjugate, particle or composition, e.g., a polymer-anticancer agent conjugate, particle or composition described herein, comprising:

determining if a subject having a proliferative disorder has or is at risk for having fluid retention and/or effusion and

selecting a subject with a proliferative disorder, e.g., cancer, who has or is at risk for having fluid retention, for treatment with a polymer-anticancer agent conjugate, particle or composition, e.g., a polymer-anticancer agent conjugate, particle or composition described herein, at a dose described herein.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the subject has one or more of the following symptoms of fluid retention: edema (e.g., peripheral, localized, generalized, lymphedema, pulmonary edema, or unspecified edema) and effusion (e.g., pleural, pericardial and ascites).

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In another aspect, the invention features a method of treating a subject, e.g., a human, having a proliferative disorder, e.g., cancer, comprising:

selecting a subject with a proliferative disorder, e.g., cancer, who has or is at risk for having fluid retention;

administering a polymer-anticancer agent conjugate, particle or composition, e.g., a polymer-anticancer agent conjugate, particle or composition, described herein, to the subject at a dose described herein, to thereby treat the disorder.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as docetaxel, coupled, e.g., via linkers, to a polymer described herein. In an embodiment, the polymer-anticancer agent conjugate comprises an anticancer agent, coupled via a linker shown in FIG. 1 or FIG. 2 to a polymer described herein. In an embodiment, the polymer-anticancer agent conjugate is a polymer-anticancer agent conjugate shown in FIG. 1 or FIG. 2.

In one embodiment, the polymer-anticancer agent conjugate, particle or composition is a polymer-docetaxel conjugate, particle or composition, e.g., a polymer-docetaxel conjugate, particle or composition described herein, e.g., a polymer-docetaxel conjugate comprising docetaxel, coupled, e.g., via linkers, to a polymer described herein. In an embodiment, the polymer-docetaxel conjugate comprises docetaxel, coupled via a linker shown in FIG. 1 or FIG. 2 to a polymer described herein. In an embodiment, the polymer-docetaxel conjugate is a polymer-docetaxel conjugate shown in FIG. 1.

In one embodiment, the polymer-docetaxel conjugate, particle or composition is administered at a dose and/or dosing schedule described herein.

In one embodiment, the subject has one or more of the following symptoms of fluid retention: edema (e.g., peripheral, localized, generalized, lymphedema, pulmonary edema, or unspecified edema) and effusion (e.g., pleural, pericardial and ascites).

In one embodiment, the cancer is a cancer described herein. In one embodiment, the polymer-anticancer conjugate, particle or composition is administered in combination with one or more additional chemotherapeutic agent, e.g., a chemotherapeutic agent or combination of chemotherapeutic agents described herein.

In one aspect, the disclosure features a method of treating a disorder, e.g., a cardiovascular disorder or an autoimmune disorder in a subject, e.g., a human, the method comprises: administering a polymer-agent conjugate, particle or composition, e.g., a polymer-agent conjugate, particle or composition described herein, to a subject in an amount effective to treat the disorder, to thereby treat the disorder.

In an embodiment, the polymer-anticancer agent conjugate comprises an agent coupled, e.g., via linkers, to a polymer described herein. In an embodiment, the polymer-agent conjugate comprises an agent, coupled via a linker shown in FIG. 1 or FIG. 2 to a polymer described herein.

In some embodiments, the polymer-agent conjugate, particle or composition is administered orally, parenterally, or intravenously. In some embodiments, the polymer-agent conjugate, particle or composition is administered to a subject once a day. In some embodiments, the polymer-agent conjugate particle or composition is administered to a subject once a week. In some embodiments, the polymer-agent conjugate, particle or composition is administered to a subject every 21 or every 28 days. In some embodiments, the polymer-agent conjugate, particle or composition is administered over a course of at least about 1 month. In some embodiments, the polymer-agent conjugate, particle or composition is administered over a course of from about 6 months to about 1 year.

In some embodiments, the method further comprises monitoring the subject for one or more toxicities or side effects. In some embodiments, the method further comprises administering at least one additional agent in combination with the polymer-agent conjugate, particle or composition.

In one aspect, the disclosure features a method of treating multiple myeloma in a subject, e.g., a human. The method comprises: administering a composition comprising a polymer-anticancer agent conjugate, particle or composition, e.g., a polymer-anticancer agent conjugate, particle or composition described herein, to a subject in an amount effective to treat the myeloma, to thereby treat the myeloma.

In some embodiments, the polymer-anticancer agent conjugate in the foregoing paragraph is as described in any one of the 1^{st} to the 12^{th} embodiments defined below and the boronic acid containing drug is bortezomib. Alternatively, the polymer-agent conjugate is a polymer-bortezomib conjugate.

In an embodiment, the polymer-anticancer agent conjugate comprises an anticancer agent such as bortezomib, coupled, e.g., via linkers, to a polymer described herein. In an embodiment, the polymer-anticancer agent conjugate comprises an anticancer agent, coupled via a linker described herein to a polymer described herein. In an embodiment, the polymer-anticancer agent conjugate is represented by structural formulas (I)-(X).

In one embodiment, the polymer-anticancer agent conjugate, particle or composition is administered as a primary treatment for multiple myeloma.

In one embodiment, the polymer-anticancer agent conjugate, particle or composition is administered in combination with dexamethasone. In one embodiment, the polymer-anticancer agent conjugate, particle or composition is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide). For example, in one embodiment, the polymer-anticancer agent conjugate, particle or composition is a polymer-bortezomib conjugate, particle or composition and the polymer-bortezomib conjugate, particle or composition is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide).

In one embodiment, the polymer-anticancer agent conjugate, particle or composition is administered in combination with a vinca alkaloid (e.g., vinblastine, vincristine, vindesine and vinorelbine) and dexamethasone. In one embodiment, the polymer-anticancer agent conjugate, particle or composition is further administered in combination with an anthracycline (e.g., daunorubicin, doxorubicin (e.g., liposomal doxorubicin), epirubicin, valrubicin and idarubicin). For example, in one embodiment, the polymer-anticancer agent conjugate, particle or composition is a polymer-bortezomib conjugate, particle or composition and the polymer-bortezomib conjugate, particle or composition is administered in combination with a vinca alkaloid (e.g., vinblastine, vincristine, vindesine and vinorelbine), dexamethasone, and an anthracycline (e.g., daunorubicin, doxorubicin (e.g., liposomal doxorubicin), epirubicin, valrubicin and idarubicin).

In one embodiment, the polymer-anticancer agent conjugate, particle or composition is administered in combination with thalidomide or thalidomide derivative (e.g., lenalidomide). In one embodiment, the polymer-anticancer agent conjugate, particle or composition is further administered in combination with dexamethasone.

In one embodiment, after the subject has received a primary treatment, e.g., a primary treatment described herein, the subject is further administered a high dose treatment. For example, the subject can be administered a high dose treatment of dexamethasone, an alkylating agent (e.g., cyclosposphamide or melphalan) and/or a polymer-anticancer agent conjugate, particle or composition described herein.

In one embodiment, after the primary treatment, e.g., after the primary treatment and the high dose treatment, stem cells are transplanted into the subject. In one embodiment, a subject who has received a stem cell transplant is administered thalidomide. In one embodiment, the subject is further administered a corticosteroid (e.g., prednisone).

In one embodiment, the polymer-anticancer agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171.

In some embodiments, the composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-bortezomib conjugate, particle or composition, e.g., a polymer-bortezomib conjugate, particle or composition described herein, e.g., a polymer-bortezomib conjugate comprising bortezomib, coupled, e.g., via linkers, to a polymer described herein. In an embodiment, the polymer-bortezomib conjugate comprises bortezomib, coupled via a linker described herein to a polymer described herein. In an embodiment, the polymer-bortezomib conjugate is represented by structural formulas (I)-(X).

In one embodiment, the polymer-bortezomib conjugate, particle or composition is administered at a dose and/or dosing schedule described herein.

In one aspect, the disclosure features a method of treating multiple myeloma in a subject, e.g., a human, the method comprising:

providing a subject who has multiple myeloma and has been treated with a chemotherapeutic agent that did not effectively treat the myeloma (e.g., the subject has a chemotherapeutic refractory myeloma, a chemotherapeutic resistant myeloma and/or a relapsed myeloma) or who had an unacceptable side effect (e.g., the subject has a chemotherapeutic sensitive myeloma), and

administering a polymer-anticancer agent conjugate, particle or composition, e.g., a polymer-anticancer agent conjugate, particle or composition described herein, to a subject in an amount effective to treat the myeloma, to thereby treat the myeloma.

In an embodiment, the polymer-anticancer agent conjugate comprises an anticancer agent such as bortezomib, coupled, e.g., via linkers, to a polymer described herein. In an embodiment, the polymer-anticancer agent conjugate comprises an anticancer agent, coupled via a linker described herein to a polymer described herein. In an embodiment, the polymer-anticancer agent conjugate is represented by structural formula (I)-(X).

In one embodiment, the subject has been treated with a proteasome inhibitor, e.g., bortezomib, which did not effectively treat the myeloma (e.g., the subject has a bortezomib refractory, a bortezomib resistant and/or relapsed myeloma).

In one embodiment, the subject has been treated with an anthracycline (e.g., daunorubicin, doxorubicin, epirubicin, valrubicin or idarubicin) which did not effectively treat the cancer (e.g., the subject has a doxorubicin refractory, a doxorubicin resistant and/or a relapsed myeloma).

In one embodiment, the subject has been treated with a thalidomide or thalidomide derivative (e.g., lenalidomide) which did not effectively treat the myeloma (e.g., the subject has thalidomide or thalidomide derivative refractory, thalidomide or thalidomide derivative resistant and/or a relapsed myeloma).

In one embodiment, the polymer-anticancer agent conjugate, particle or composition is administered in combination with an anthracycline (e.g., daunorubicin, doxorubicin (e.g., liposomal doxorubicin), epirubicin, valrubicin and idarubicin). In one embodiment, the polymer-anticancer agent conjugate, particle or composition is administered in combination with an anthracycline (e.g., daunorubicin, doxorubicin (e.g., liposomal doxorubicin), epirubicin, valrubicin and idarubicin).

In one embodiment, the polymer-anticancer agent conjugate, particle or composition is administered in combination with thalidomide or a thalidomide derivative (e.g. lenalidomide) and dexamethasone.

In one embodiment, the polymer-anticancer agent conjugate, particle or composition is administered in combination with dexamethaxone and cyclophosphamide. In one embodiment, the polymer-anticancer agent conjugate, particle or composition is further administered in combination with a topoisomerase inhibitor (e.g., etoposide, topotecan, irinotecan, tenoposide, SN-38, lamellarin D) and/or a platinum based agent (carboplatin, cisplatin, oxaliplatin). In one embodiment, the polymer-anticancer agent conjugate, particle or composition is further administered in combination with an anthracycline (e.g., daunorubicin, doxorubicin (e.g., liposomal doxorubicin), epirubicin, valrubicin and idarubicin).

In an embodiment, the polymer-anticancer agent conjugate comprises an anticancer agent such as bortezomib, coupled, e.g., via linkers, to a polymer described herein. In an embodiment, the polymer-anticancer agent conjugate comprises an anticancer agent, coupled via a linker described herein to a polymer described herein. In an embodiment, the polymer-anticancer agent conjugate is represented by structural formula (I)-(X).

In one embodiment, the polymer-bortezomib conjugate, particle or composition is administered at a dose and/or dosing schedule described herein.

In one aspect, the disclosure features a method of treating mantle cell lymphoma in a subject, e.g., a human. The method comprises: administering a polymer-anticancer agent conjugate, particle or composition, e.g., a polymer-anticancer agent conjugate, particle or composition described herein, to a subject in an amount effective to treat the lymphoma, to thereby treat the lymphoma.

In an embodiment, the polymer-anticancer agent conjugate comprises an anticancer agent such as bortezomib, coupled, e.g., via linkers, to a polymer described herein. In an embodiment, the polymer-anticancer agent conjugate comprises an anticancer agent, coupled via a linker described herein to a polymer described herein. In an embodiment, the polymer-anticancer agent conjugate is represented by structural formula (I)-(X).

In one embodiment, the polymer-anticancer agent conjugate, particle or composition is administered in combination with an anthracycline (e.g., daunorubicin, doxorubicin (e.g., liposomal doxorubicin), epirubicin, valrubicin and idarubicin) and a vinca alkaloid (e.g., vinblastine, vincristine, vindesine and vinorelbine). For example, in one embodiment, the polymer-anticancer agent conjugate, particle or composition is a polymer-bortezomib conjugate, particle or composition and the polymer-bortezomib conjugate, particle or composition is further administered in combination with an anthracycline (e.g., doxorubicin (e.g., liposomal doxorubicin)) and a vinca alkaloid (e.g., vincristine). In one embodiment, the polymer-anticancer agent conjugate, particle or composition is further administered with one or more of an alkylating agent (e.g., cyclophosphamide, dacarbazine, melphalan, ifosfamide, temozolomide), prednisone, demethasone and rituximab. For example, in one embodiment, the polymer-anticancer agent conjugate, particle or composition is administered in one of the following combinations: an alkylating agent (e.g., cyclophosphamide), an anthracycline (e.g., doxorubicin (e.g., liposomal doxorubicin)), a vinca alkaloid (e.g., vincristine) and prednisone; an alkylating agent (e.g., cyclophosphamide), an anthracycline (e.g., doxorubicin (e.g., liposomal doxorubicin)), a vinca alkaloid (e.g., vincristine), prednisone and rituximab; an alkylating agent (e.g., cyclophosphamide), an anthracycline (e.g., doxorubicin (e.g., liposomal doxorubicin)), a vinca alkaloid (e.g., vincristine) and demethasone; an alkylating agent (e.g., cyclophosphamide), an anthracycline (e.g., doxorubicin (e.g., liposomal doxorubicin)), a vinca alkaloid (e.g., vincristine), demethasone and rituximab; an anthracycline (e.g., doxorubicin (e.g., liposomal doxorubicin)), a vinca alkaloid (e.g., vincristine) and prednisone; an anthracycline (e.g., doxorubicin (e.g., liposomal doxorubicin)), a vinca alkaloid (e.g., vincristine), prednisone and rituximab; an anthracycline (e.g., doxorubicin (e.g., liposomal doxorubicin)), a vinca alkaloid (e.g., vincristine) and demethasone; and an anthracycline (e.g., doxorubicin (e.g., liposomal doxorubicin)), a vinca alkaloid (e.g., vincristine), demethasone and rituximab.

In one embodiment, the polymer-anticancer agent conjugate, particle or composition is administered in combination with an alkylating agent (e.g., cyclophosphamide, dacarbazine, melphalan, ifosfamide, temozolomide) and a vinca alkaloid (e.g., vinblastine, vincristine, vindesine and vinorelbine). For example, in one embodiment, the polymer-anticancer agent conjugate, particle or composition is a polymer-bortezomib conjugate, particle or composition and the polymer-bortezomib conjugate, particle or composition is further administered in combination with an alkylating agent (e.g., cyclophosphamide) and a vinca alkaloid (e.g., vincristine). In one embodiment, the polymer-anticancer agent conjugate, particle or composition is further administered with one or more of prednisone, demethasone and rituximab. For example, in one embodiment, the polymer-anticancer agent conjugate, particle or composition is administered in one of the following combinations: an alkylating agent (e.g., cyclophosphamide), a vinca alkaloid (e.g., vincristine) and prednisone; an alkylating agent (e.g., cyclophosphamide), a vinca alkaloid (e.g., vincristine), prednisone and rituximab; an alkylating agent (e.g., cyclophosphamide), a vinca alkaloid (e.g., vincristine) and demethasone; and an alkylating agent (e.g., cyclophosphamide), a vinca alkaloid (e.g., vincristine), demethasone and rituximab.

In one embodiment, the polymer-anticancer agent conjugate, particle or composition is administered in combination with an anthracycline (e.g., daunorubicin, doxorubicin (e.g., liposomal doxorubicin), epirubicin, valrubicin and idarubicin) and an alkylating agent (e.g., cyclophosphamide, dacarbazine, melphalan, ifosfamide, temozolomide). For example, in one embodiment, the polymer-anticancer agent conjugate, particle or composition is a polymer-bortezomib conjugate, particle or composition and the polymer-bortezomib conjugate, particle or composition is further administered in combination with an anthracycline (e.g., doxorubicin (e.g., liposomal doxorubicin)) and an alkylating agent (e.g., cyclophosphamide). In one embodiment, the polymer-anticancer agent conjugate, particle or composition is further administered with one or more of prednisone, demethasone and rituximab. For example, in one embodiment, the polymer-anticancer agent conjugate, particle or composition is administered in one of the following combinations: an alkylating agent (e.g., cyclophosphamide), an anthracycline (e.g., doxorubicin (e.g., liposomal doxorubicin)) and prednisone; an alkylating agent (e.g., cyclophosphamide), an anthracycline (e.g., doxorubicin (e.g., liposomal doxorubicin)), prednisone and rituximab; an alkylating agent (e.g., cyclophosphamide), an anthracycline (e.g., doxorubicin (e.g., liposomal doxorubicin)) and demethasone; an alkylating agent (e.g., cyclophosphamide), an anthracycline (e.g., doxorubicin (e.g., liposomal doxorubicin)), demethasone and rituximab.

In one embodiment, a topoisomerase inhibitor (e.g., etoposide, topotecan, irinotecan, tenoposide, SN-38, lamellarin D) can be further administered with any of the combinations described above. For example, in one embodiment, the polymer-anticancer agent conjugate, particle or composition is administered in one of the following combinations: an alkylating agent (e.g., cyclophosphamide), an anthracycline (e.g., doxorubicin (e.g., liposomal doxorubicin)), a vinca alkaloid (e.g., vincristine) and prednisone; an alkylating agent (e.g., cyclophosphamide), an anthracycline (e.g., doxorubicin (e.g., liposomal doxorubicin)), a vinca alkaloid (e.g., vincristine), prednisone and rituximab.

In one embodiment, the method further includes administering an additional chemotherapeutic treatment, wherein the additional chemotherapeutic treatment includes a combination of rituximab, an immunosuppressive agent (e.g., methotrexate) and cytarabine.

In one embodiment, the polymer-anticancer agent conjugate, particle or composition is administered in combination with cladribine.

In some embodiments, the polymer-anticancer agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the polymer-anticancer agent conjugate, particle or composition is administered in combination with bevacizumab.

In some embodiments, the polymer-anticancer agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-anticancer agent conjugate, particle or composition is a polymer-bortezomib conjugate, particle or composition, e.g., a polymer-bortezomib conjugate, particle or composition described herein, e.g., a polymer-bortezomib conjugate comprising bortezomib, coupled, e.g., via linkers, to a polymer described herein. In an embodiment, the polymer-bortezomib conjugate comprises bortezomib, coupled via a linker described herein to a polymer described herein. In an embodiment, the polymer-bortezomib conjugate is represented by structural formula (I)-(X).¹⁹⁶

In one embodiment, the polymer-bortezomib conjugate, particle or composition is administered at a dose and/or dosing schedule described herein.

In one aspect, the disclosure features a method of treating mantle cell lymphoma, in a subject, e.g., a human. The method comprises:

providing a subject who has mantle cell lymphoma and has been treated with a chemotherapeutic agent which did not effectively treat the lymphoma (e.g., the subject has a chemotherapeutic refractory, a chemotherapeutic resistant and/or a relapsed lymphoma) or which had an unacceptable side effect (e.g., the subject has a chemotherapeutic sensitive lymphoma), and

administering a polymer-anticancer agent conjugate, particle or composition, e.g., a polymer-anticancer agent conjugate, particle or composition described herein, to a subject in an amount effective to treat the cancer, to thereby treat the cancer. In an embodiment, the polymer-anticancer agent conjugate comprises an anticancer agent such as bortezomib, coupled, e.g., via linkers, to a polymer described herein. In an embodiment, the polymer-anticancer agent conjugate comprises an anticancer agent, coupled via a linker described herein to a polymer described herein. In an embodiment, the polymer-anticancer agent conjugate is represented by structural formula (I)-(X).

In one embodiment, the lymphoma is refractory to, resistant to, and/or relapsed with treatment with one or more of: an alkylating agent (e.g., cyclophosphamide, dacarbazine, melphalan, ifosfamide, temozolomide), a vinca alkaloid (e.g., vinblastine, vincristine, vindesine and vinorelbine) and an anthracycline (e.g., daunorubicin, doxorubicin, epirubicin, valrubicin and idarubicin).

In one embodiment, the cancer is a multidrug resistant lymphoma.

In one embodiment, the polymer-anticancer agent conjugate, particle or composition can be administered in combination with one or more of: bendamustine, cladribine, fludarabine, thalidomide, a thalidomide derivative (e.g., lenalidomide), pentostatin and an mTOR inhibitor (e.g., temsirolimus). In one embodiment, the polymer-anticancer agent conjugate, particle or composition can further be administered in combination with an alkylating agent (e.g., cyclophosphamide, dacarbazine, melphalan, ifosfamide, temozolomide). For example, in one embodiment, the polymer-anticancer agent conjugate, particle or composition is administered in one of the following combinations: fludarabine and an alkylating agent (e.g., cyclophosphamide); fludarabine, an alkylating agent (e.g., cyclophosphamide) and mitoxantrone; fludarabine and mitoxantrone; and pentostatin and an alkylating agent (e.g., cyclophosphamide).

In one embodiment, the polymer-anticancer agent conjugate, particle or composition is administered in combination with topoisomerase inhibitor (e.g., topotecan, irinotecan, etoposide, teniposide, SN-38, lamellarin D, camptothecin (e.g., IT-101)) and an alkylating agent (e.g., cyclophosphamide, dacarbazine, melphalan, ifosfamide, temozolomide). In one embodiment, the polymer-anticancer agent conjugate, particle or composition is further administered in combination with prednisone and/or procarbazine.

In one embodiment, the polymer-anticancer agent conjugate, particle or composition is a polymer-bortezomib conjugate, particle or composition, e.g., a polymer-bortezomib conjugate, particle or composition described herein, e.g., a polymer-bortezomib conjugate comprising bortezomib, coupled, e.g., via linkers, to a polymer described herein. In an embodiment, the polymer-bortezomib conjugate comprises bortezomib, coupled via a linker described herein to a polymer described herein. In an embodiment, the polymer-bortezomib conjugate is represented by structural formula (I)-(X).

In one embodiment, the polymer-bortezomib conjugate, particle or composition is administered at a dose and/or dosing schedule described herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts a table of polymer-drug conjugates.

FIG. 2 depicts a table of polymer-drug conjugates.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Polymer-agent conjugates, particles, and compositions are described herein. Also disclosed are dosage forms containing the polymer-agent conjugates, particles and compositions; methods of using the polymer-agent conjugates, particles and compositions (e.g., to treat a disorder); kits including the polymer-agent conjugates, particles and compositions; methods of making the polymer-agent conjugates, particles and compositions; methods of storing the polymer-agent conjugates, particles and compositions; and methods of analyzing the particles.

### DEFINITIONS

The term “ambient conditions,” as used herein, refers to surrounding conditions at about one atmosphere of pressure, 50% relative humidity and about 25° C.

The term “attach,” as used herein with respect to the relationship of a first moiety to a second moiety, e.g., the attachment of an agent to a polymer, refers to the formation of a covalent bond between a first moiety and a second moiety. In the same context, “attachment” refers to the covalent bond. For example, a therapeutic agent attached to a polymer is a therapeutic agent covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). E.g., where an attachment is through a linker, a first moiety (e.g., a drug) is covalently bonded to a linker, which in turn is covalently bonded to a second moiety (e.g., a hydrophobic polymer described herein).

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradation,” as used herein, encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

An “effective amount” or “an amount effective” refers to an amount of the polymer-agent conjugate, compound or composition which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition is outweighed by the therapeutically beneficial effects.

The term “embed,” as used herein, refers to the formation of a non-covalent interaction between a first moiety and a second moiety, e.g., an agent and a polymer (e.g., a therapeutic or diagnostic agent and a hydrophobic polymer). An embedded moiety, e.g., an agent embedded in a polymer or a particle, is associated with a polymer or other component of the particle through one or more non-covalent interactions such as van der Waals interactions, hydrophobic interactions, hydrogen bonding, dipole-dipole interactions, ionic interactions, and pi stacking. An embedded moiety has no covalent linkage to the polymer or particle in which it is embedded. An embedded moiety may be completely or partially surrounded by the polymer or particle in which it is embedded.

The term “hydrophilic,” as used herein, refers to a moiety that has a solubility in aqueous solution of at least about 0.05 mg/mL or greater (e.g., at least about 1.0 mg/mL or greater).

The term “hydrophobic,” as used herein, refers to a moiety that can be dissolved in an aqueous solution at physiological ionic strength only to the extent of about 0.05 mg/mL or less (preferably about 0.001 mg/mL or less).

A “hydroxy protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, acyl (e.g., acetyl), triethylsilyl (TES), t-butyldimethylsilyl (TBDMS), 2,2,2-trichloroethoxycarbonyl (Troc), and carbobenzyloxy (Cbz).

“Inert atmosphere,” as used herein, refers to an atmosphere composed primarily of an inert gas, which does not chemically react with the polymer-agent conjugates, particles, compositions or mixtures described herein. Examples of inert gases are nitrogen (N₂), helium, and argon.

“Linker,” as used herein, is a moiety having at least two functional groups. One functional group is capable of reacting with a functional group on a polymer described herein, and a second functional group is capable of reacting with a functional group on agent described herein. In some embodiments the linker has just two functional groups. A linker may have more than two functional groups (e.g., 3, 4, 5, 6, 7, 8, 9, 10 or more functional groups), which may be used, e.g., to link multiple agents to a polymer. Depending on the context, linker can refer to a linker moiety before attachment to either of a first or second moiety (e.g., agent or polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. It can be used to protect nanoparticles, liposomes, and/or micelles during lyophilization, for example to reduce or prevent aggregation, particle collapse and/or other types of damage. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that can not be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as α cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2 hydroxy propyl-beta cyclodextrin, e.g., partially etherified cyclodextrins (e.g., partially etherified cyclodextrins) disclosed in U.S. Pat. No. 6,407,079, the contents of which are incorporated herein by this reference.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis—the term “derivative” implies no process-based limitation.

The term “nanoparticle” is used herein to refer to a material structure whose size in any dimension (e.g., x, y, and z Cartesian dimensions) is less than about 1 micrometer (micron), e.g., less than about 500 nm or less than about 200 nm or less than about 100 nm, and greater than about 5 nm. A nanoparticle can have a variety of geometrical shapes, e.g., spherical, ellipsoidal, etc. The term “nanoparticles” is used as the plural of the term “nanoparticle.”

As used herein, “particle polydispersity index (PDI)” or “particle polydispersity” refers to the width of the particle size distribution. Particle PDI can be calculated from the equation PDI=2a₂/a₁² where a₁ is the 1^{st} Cumulant or moment used to calculate the intensity weighted Z average mean size and a₂ is the 2^{nd} moment used to calculate a parameter defined as the polydispersity index (PdI). A particle PDI of 1 is the theoretical maximum and would be a completely flat size distribution plot. Compositions of particles described herein may have particle PDIs of less than 0.5, less than 0.4, less than 0.3, less than 0.2, or less than 0.1. Particle PDI is further defined in the document “What does polydispersity mean (Malvern)”, which is incorporated herein by reference. (Available at http://www.malvern.com/malvern/kbase.nsf/allbyno/KB000780/$file/FAQ%20-%20What%20does%20polydispersity%20mean.pdf).

“Pharmaceutically acceptable carrier or adjuvant,” as used herein, refers to a carrier or adjuvant that may be administered to a patient, together with a polymer-agent conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. In some cases, the polymer is biologically derived, i.e., a biopolymer. Non-limiting examples of biopolymers include peptides or proteins (i.e., polymers of various amino acids), or nucleic acids such as DNA or RNA.

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

As used herein, the term “prevent” or “preventing” as used in the context of the administration of an agent to a subject, refers to subjecting the subject to a regimen, e.g., the administration of a polymer-agent conjugate, particle or composition, such that the onset of at least one symptom of the disorder is delayed as compared to what would be seen in the absence of the regimen.

The term “prodrug” is intended to encompass compounds that, under physiological conditions, are converted into therapeutically active agents. A common method for making a prodrug is to include selected moieties that are hydrolyzed under physiological conditions to reveal the desired molecule, such as an ester or an amide. In some embodiments, the prodrug is converted by an enzymatic activity of the host animal. Exemplary prodrugs include hexanoate conjugates.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a polymer-agent conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl (CH₃C(O)—), benzoyl (C₆H₅C(O)—), and acetylamino acids (e.g., acetylglycine, CH₃C(O)NHCH₂C(O)—.

The term “alkoxy” refers to an alkyl group, as defined below, having an oxygen radical attached thereto. Representative alkoxy groups include methoxy, ethoxy, propyloxy, tert-butoxy and the like.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure. The term “alkylenyl” refers to a divalent alkyl, e.g., —CH₂—, —CH₂CH₂—, and —CH₂CH₂CH₂—.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

### Polymer-Agent Conjugates

A polymer-agent conjugate described herein includes a polymer (e.g., a hydrophobic polymer or a polymer containing a hydrophilic portion and a hydrophobic portion) and an agent (e.g., a therapeutic or diagnostic agent). An agent described herein may be attached to a polymer described herein, e.g., directly or through a linker. An agent may be attached to a hydrophobic polymer (e.g., PLGA), or a polymer having a hydrophobic portion and a hydrophilic portion (e.g., PEG-PLGA). An agent may be attached to a terminal end of a polymer, to both terminal ends of a polymer, or to a point along a polymer chain. In some embodiments, multiple agents may be attached to points along a polymer chain, or multiple agents may be attached to a terminal end of a polymer via a multifunctional linker.

Polymers

A wide variety of polymers and methods for forming polymer-agent conjugates and particles therefrom are known in the art of drug delivery. Any polymer may be used in accordance with the present invention. Polymers may be natural or unnatural (synthetic) polymers. Polymers may be homopolymers or copolymers containing two or more monomers. Polymers may be linear or branched.

If more than one type of repeat unit is present within the polymer, then the polymer is said to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

Hydrophobic Polymers

A polymer-agent conjugate or particle described herein may include a hydrophobic polymer. The hydrophobic polymer may be attached to an agent. Exemplary hydrophobic polymers include the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers (see generally, Svenson, S (ed.)., Polymeric Drug Delivery: Volume I: Particulate Drug Carriers. 2006; ACS Symposium Series; Amiji, M. M (ed.)., Nanotechnology for Cancer Therapy. 2007; Taylor & Francis Group, LLP; Nair et al. Prog. Polym. Sci. (2007) 32: 762-798); hydrophobic peptide-based polymers and copolymers based on poly(L-amino acids) (Lavasanifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

Hydrophobic polymers useful in preparing the polymer-agent conjugates or particles described herein also include biodegradable polymers. Examples of biodegradable polymers include polylactides, polyglycolides, caprolactone-based polymers, poly(caprolactone), polydioxanone, polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), poly(vinylpyrrolidone), polyethylene glycol, polyhydroxycellulose, polysaccharides, chitin, chitosan and hyaluronic acid, and copolymers, terpolymers and mixtures thereof. Biodegradable polymers also include copolymers, including caprolactone-based polymers, polycaprolactones and copolymers that include polybutylene terephthalate.

In some embodiments, the polymer is a polyester synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid.

A copolymer may also be used in a polymer-agent conjugate or particle described herein. In some embodiments, a polymer may be PLGA, which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers is 50:50, 60:40 or 75:25.

In particular embodiments, by optimizing the ratio of lactic acid to glycolic acid monomers in the PLGA polymer of the polymer-agent conjugate or particle, parameters such as water uptake, agent release (e.g., “controlled release”) and polymer degradation kinetics may be optimized. Furthermore, tuning the ratio will also affect the hydrophobicity of the copolymer, which may in turn affect drug loading.

In certain embodiments wherein the biodegradable polymer also has an agent or other material attached to it, the biodegradation rate of such polymer may be characterized by a release rate of such materials. In such circumstances, the biodegradation rate may depend on not only the chemical identity and physical characteristics of the polymer, but also on the identity of material(s) attached thereto. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts. In some embodiments, the release can be affected by an additional component in the particle, e.g., a compound having at least one acidic moiety (e.g., free-acid PLGA).

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 4 and 8 having a temperature of between 25° C. and 37° C. In other embodiments, the polymer degrades in a period of between about one hour and several weeks, depending on the desired application.

When polymers are used for delivery of pharmacologically active agents in vivo, it is important that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue (D. F. Williams, J. Mater. Sci. 1233 (1982)). To minimize the toxicity of the intact polymer carrier and its degradation products, polymers have been designed based on naturally occurring metabolites. Exemplary polymers include polyesters derived from lactic and/or glycolic acid and polyamides derived from amino acids.

A number of biodegradable polymers are known and used for controlled release of pharmaceuticals. Such polymers are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety.

A hydrophobic polymer described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer.

A hydrophobic polymer may have a weight average molecular weight ranging from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 6 kDa to about 13 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

A hydrophobic polymer described herein may have a polymer polydispersity index (PDI) of less than or equal to about 2.5 (e.g., less than or equal to about 2.2, or less than or equal to about 2.0). In some embodiments, a hydrophobic polymer described herein may have a polymer PDI of about 1.0 to about 2.5, about 1.0 to about 2.0, about 1.0 to about 1.7, or from about 1.0 to about 1.6.

A particle described herein may include varying amounts of a hydrophobic polymer, e.g., from about 20% to about 90% by weight (e.g., from about 20% to about 80%, from about 25% to about 75%, or from about 30% to about 70%).

A hydrophobic polymer described herein may be commercially available, e.g., from a commercial supplier such as BASF, Boehringer Ingelheim, Durcet Corporation, Purac America and SurModics Pharmaceuticals. A polymer described herein may also be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization.

A commercially available or synthesized polymer sample may be further purified prior to formation of a polymer-agent conjugate or incorporation into a particle or composition described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

Polymers Containing a Hydrophilic Portion and a Hydrophobic Portion

A polymer-agent conjugate or particle described herein may include a polymer containing a hydrophilic portion and a hydrophobic portion. A polymer containing a hydrophilic portion and a hydrophobic portion may be a copolymer of a hydrophilic block coupled with a hydrophobic block. These copolymers may have a weight average molecular weight between about 5 kDa and about 30 kDa (e.g., from about 5 kDa to about 25 kDa, from about 10 kDa to about 22 kDa, from about 10 kDa to about 15 kDa, from about 12 kDa to about 22 kDa, from about 7 kDa to about 15 kDa, from about 15 kDa to about 19 kDa, or from about 11 kDa to about 13 kDa, e.g., about 9 kDa, about 10 kDa, about 11 kDa, about 12 kDa, about 13 kDa, about 14 kDa about 15 kDa, about 16 kDa, about 17 kDa, about 18 kDa or about 19 kDa). The polymer containing a hydrophilic portion and a hydrophobic portion may be attached to an agent.

Examples of suitable hydrophobic portions of the polymers include those described above. The hydrophobic portion of the copolymer may have a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

Examples of suitable hydrophilic portions of the polymers include the following: carboxylic acids including acrylic acid, methacrylic acid, itaconic acid, and maleic acid; polyoxyethylenes or polyethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, poly(vinylpyrrolidone), starches and starch derivatives, dextran and dextran derivatives; polypeptides, such as polylysines, polyarginines, polyglutamic acids; polyhyaluronic acids, alginic acids, polylactides, polyethyleneimines, polyionenes, polyacrylic acids, and polyiminocarboxylates, gelatin, and unsaturated ethylenic mono or dicarboxylic acids. A listing of suitable hydrophilic polymers can be found in Handbook of Water-Soluble Gums and Resins, R. Davidson, McGraw-Hill (1980).

The hydrophilic portion of the copolymer may have a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa).

A polymer containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same hydrophobic polymer or different hydrophobic polymers. The block copolymers used herein may have varying ratios of the hydrophilic portion to the hydrophobic portion, e.g., ranging from 1:1 to 1:40 by weight (e.g., about 1:1 to about 1:10 by weight, about 1:1 to about 1:2 by weight, or about 1:3 to about 1:6 by weight).

A polymer containing a hydrophilic portion and a hydrophobic portion may have a variety of end groups. In some embodiments, the end group may be a hydroxy group or an alkoxy group. In some embodiments, the end group of the polymer is not further modified. In some embodiments, the end group may be further modified. For example, the end group may be capped with an alkyl group, to yield an alkoxy-capped polymer (e.g., a methoxy-capped polymer), or may be derivatized with a targeting agent (e.g., folate) or a dye (e.g., rhodamine).

A polymer containing a hydrophilic portion and a hydrophobic portion may include a linker between the two blocks of the copolymer. Such a linker may be an amide, ester, ether, amino, carbamate or carbonate linkage, for example.

A polymer containing a hydrophilic portion and a hydrophobic portion described herein may have a polymer polydispersity index (PDI) of less than or equal to about 2.5 (e.g., less than or equal to about 2.2, or less than or equal to about 2.0, or less than or equal to about 1.5). In some embodiments, the polymer PDI is from about 1.0 to about 2.5, e.g., from about 1.0 to about 2.0, from about 1.0 to about 1.8, from about 1.0 to about 1.7, or from about 1.0 to about 1.6.

A particle described herein may include varying amounts of a polymer containing a hydrophilic portion and a hydrophobic portion, e.g., up to about 50% by weight (e.g., from about 4 to about 50%, about 5%, about 10%, about 15%, about 20%, about 25%, about 30%, about 35%, about 40%, about 45% or about 50% by weight). For example, the percent by weight of the second polymer within the particle is from about 3% to 30%, from about 5% to 25% or from about 8% to 23%.

A polymer containing a hydrophilic portion and a hydrophobic portion described herein may be commercially available, or may be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl) carbodiimide hydrochloride). Following conjugation, the two blocks may be linked via an amide, ester, ether, amino, carbamate or carbonate linkage.

A commercially available or synthesized polymer sample may be further purified prior to formation of a polymer-agent conjugate or incorporation into a particle or composition described herein. In some embodiments, purification may remove lower molecular weight polymers that may lead to unfilterable polymer samples. A polymer may be purified by precipitation from solution, or precipitation onto a solid such as Celite. A polymer may also be further purified by size exclusion chromatography (SEC).

Agents

An agent to be delivered using a polymer-agent conjugate, particle or composition described herein may be a therapeutic, diagnostic, prophylactic or targeting agent. The agent may be a small molecule, organometallic compound, nucleic acid, protein, peptide, metal, isotopically labeled chemical compound, drug, vaccine, immunological agent, etc.

In some embodiments, the agent is a compound with pharmaceutical activity. In another embodiment, the agent is a clinically used or investigated drug. In another embodiment, the agent has been approved by the U. S. Food and Drug Administration for use in humans or other animals. In some embodiments, the agent is an antibiotic, anti-viral agent, anesthetic, steroidal agent, anti-cancer agent, anti-inflammatory agent (e.g., a non-steroidal anti-inflammatory agent), anti-neoplastic agent, antigen, vaccine, antibody, decongestant, antihypertensive, sedative, birth control agent, progestational agent, anti-cholinergic, analgesic, anti-depressant, anti-psychotic, p-adrenergic blocking agent, diuretic, cardiovascular active agent, vasoactive agent, nutritional agent, vitamin (e.g., riboflavin, nicotinic acid, pyridoxine, pantothenic acid, biotin, choline, inositol, carnitine, vitamin C, vitamin A, vitamin E, vitamin K), gene therapy agent (e.g., DNA-protein conjugates, anti-sense agents); or targeting agent.

In some embodiments, the agent is an anti-cancer agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®)).

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™)

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the agent is an anti-cancer agent. An anti-cancer agent may be an alkylating agent (e.g., nitrogen mustards, nitrosoureas, platinum, alkyl sulfonates, hydrazines, triazenes, aziridines, spindle poison, cytotoxic agents, topoisomerase inhibitors and others), a cytotoxic agent, an anti-angiogenic agent, a vascular disrupting agent, a microtubule targeting agent, a mitotic inhibitor, a topoisomerase inhibitor, or an anti-metabolite (e.g., folic acid, purine, and pyrimidine derivatives). Exemplary anti-cancer agents include aclarubicin, actinomycin, alitretinon, altretamine, aminopterin, aminolevulinic acid, amrubicin, amsacrine, anagrelide, arsenic trioxide, asparaginase, atrasentan, belotecan, bexarotene, endamustine, bleomycin, busulfan, camptothecin, capecitabine, carboplatin, carboquone, carmofur, carmustine, celecoxib, chlorambucil, chlormethine, cisplatin, cladribine, clofarabine, crisantaspase, cyclophosphamide, cytarabine, dacarbazine, dactinomycin, daunorubicin, decitabine, demecolcine, docetaxel, doxorubicin, efaproxiral, elesclomol, elsamitrucin, enocitabine, epirubicin, estramustine, etoglucid, etoposide, floxuridine, fludarabine, fluorouracil (5FU), fotemustine, gemcitabine, Gliadel implants, hydroxycarbamide, hydroxyurea, idarubicin, ifosfamide, irinotecan, irofulven, larotaxel, leucovorin, liposomal doxorubicin, liposomal daunorubicin, lonidamine, lomustine, lucanthone, mannosulfan, masoprocol, melphalan, mercaptopurine, mesna, methotrexate, methyl aminolevulinate, mitobronitol, mitoguazone, mitotane, mitomycin, mitoxantrone, nedaplatin, nimustine, oblimersen, omacetaxine, ortataxel, oxaliplatin, paclitaxel, pegaspargase, pemetrexed, pentostatin, pirarubicin, pixantrone, plicamycin, porfimer sodium, prednimustine, procarbazine, raltitrexed, ranimustine, rubitecan, sapacitabine, semustine, sitimagene ceradenovec, strataplatin, streptozocin, talaporfin, tamoxifen, tegafur-uracil, temoporfin, temozolomide, teniposide, tesetaxel, testolactone, tetranitrate, thiotepa, tiazofurine, tioguanine, tipifarnib, topotecan, trabectedin, triaziquone, triethylenemelamine, triplatin, tretinoin, treosulfan, trofosfamide, uramustine, valrubicin, verteporfin, vinblastine, vincristine, vindesine, vinflunine, vinorelbine, vorinostat, zorubicin, and combinations thereof, or other cytostatic or cytotoxic agents described herein.

In some embodiments, the agent is an anti-inflammatory/autoimmune agent. An anti-inflammatory/autoimmune agent may be a steroid, nonsteroidal anti-inflammatory drug (NSAID), PDE4 inhibitor, antihistamine, or COX-2 inhibitor. Exemplary anti-inflammatory/autoimmune agents include [alpha]-bisabolol, 1-naphthyl salicylate, 2-amino-4-picoline, 3-amino-4-hydroxybutyric acid, 5-bromosalicylic acid acetate, 5′-nitro-2′-propoxyacetanilide, 6[alpha]-methylprednisone, aceclofenac, acemetacin, acetaminophen, acetaminosalol, acetanilide, acetylsalicylic acid, alclofenac, alclometasone, alfentanil, algestone, allylprodine, alminoprofen, aloxiprin, alphaprodine, aluminum bis(acetylsalicylate), amcinonide, amfenac, aminochlorthenoxazin, aminopropylon, aminopyrine, amixetrine, ammonium salicylate, ampiroxicam, amtolmetin guacil, anileridine, antipyrine, antrafenine, apazone, artemether, artemisinin, artsunate, aspirin, atovaquone, beclomethasone, bendazac, benorylate, benoxaprofen, benzpiperylon, benzydamine, benzylmorphine, bermoprofen, betamethasone, betamethasone-17-valerate, bezitramide, bromfenac, bromosaligenin, bucetin, bucloxic acid, bucolome, budesonide, bufexamac, bumadizon, buprenorphine, butacetin, butibufen, and butorphanol.

Other exemplary anti-inflammatory/autoimmune agents include caiprofen, carbamazepine, carbiphene, carsalam, celecoxib, chlorobutanol, chloroprednisone, chloroquine phosphate, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisol, cortisone, cortivazol, cropropamide, crotethamide, cyclazocine, cyclizine, deflazacort, dehydrotestosterone, deoxycorticosterone, deracoxib, desomorphine, desonide, desoximetasone, dexamethasone, dexamethasone-21-isonicotinate, dexoxadrol, dextromoramide, dextropropoxyphene, dezocine, diamorphone, diampromide, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, diphenhydramine, dipipanone, diprocetyl, dipyrone, ditazol, doxycycline hyclate, drotrecogin alfa, droxicam, e-acetamidocaproic acid, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, etoricoxib, and eugenol.

Other exemplary anti-inflammatory/autoimmune agents include felbinac, fenbufen, fenclozic acid, fendosal, fenoprofen, fentanyl, fentiazac, fepradinol, feprazone, floctafenine, fluazacort, flucloronide, fludrocortisone, flufenamic acid, flumethasone, flunisolide, flunixin, flunoxaprofen, fluocinolone acetonide, fluocinonide, fluocoitolone, fluocortin butyl, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halofantrine, halometasone, haloprednone, heroin, hydro cortamate, hydrocodone, hydrocortisone, hydrocortisone 21-lysinate, hydrocortisone acetate, hydrocortisone cypionate, hydrocortisone hemisuccinate, hydrocortisone succinate, hydromorphone, hydroxypethidine, hydroxyzine, ibufenac, ibuprofen, ibuproxam, imidazole salicylate, indomethacin, indoprofen, isofezolac, isoflupredone, isoflupredone acetate, isoladol, isomethadone, isonixin, isoxepac and isoxicam.

Other exemplary anti-inflammatory/autoimmune agents include ketobemidone, ketoprofen, ketorolac, lefetamine, levallorphan, levophenacyl-morphan, levorphanol, lofentanil, lonazolac, lornoxicam, loxoprofen, lumiracoxib, lysine acetylsalicylate, mazipredone, meclofenamic acid, medrysone, mefenamic acid, mefloquine hydrochloride, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, naproxen, narceine, nefopam, nicomorphine, nifenazone, niflumic acid, nimesulide, norlevorphanol, normethadone, normorphine, norpipanone, olsalazine, opium, oxaceprol, oxametacine, oxaprozin, oxycodone, oxymorphone and oxyphenbutazone.

Other exemplary anti-inflammatory/autoimmune agents include p-lactophenetide, papaveretum, paramethasone, paranyline, parecoxib, parsalmide, p-bromoacetanilide, pentazocine, perisoxal, phenacetin, phenadoxone, phenazocine, phenazopyridine hydrochloride, phenocoll, phenomorphan, phenoperidine, phenopyrazone, phenyl acetylsalicylate, phenyl salicylate, phenylbutazone, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proguanil hydrochloride, proheptazine, promedol, promethazine, propacetamol, properidine, propiram, propoxyphene, propyphenazone, proquazone, protizinic acid, proxazole, ramifenazone, remifentanil, rimazolium metilsulfate, rofecoxib, roflumilast, rolipram, S-adenosylmethionine, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, valdecoxib, viminol, xenbucin, ximoprofen, zaltoprofen, and zomepirac.

In some embodiments, the agent is an agent for the treatment of cardiovascular disease. An agent for the treatment of cardiovascular disease may be an [alpha]-receptor blocking drug, [beta]-adrenaline receptor blocking drug, AMPA antagonist, angiotensin converting enzyme inhibitor, angiotensin II antagonist, animal salivary gland plasminogen activator, anti-anginal agent, anti-arrhythmic agent, anti-hyperlipidemic drug, anti-hypertensive agent, anti-platelet drug, calcium antagonist, calcium channel blocking agent, cardioglycoside, cardioplegic solution, cardiotonic agent, catecholamine formulation, cerebral protecting drug, cyclooxygenase inhibitor, digitalis formulation, diuretic (e.g., a K+ sparing diuretic, loop diuretic, nonthiazide diuretic, osmotic diuretic, or thiazide diuretic), endothelin receptor blocking drug, fibrinogen antagonist, fibrinolytic agent, GABA agonist, glutamate antagonist, growth factor, heparin, K+ channel opening drug, kainate antagonist, naturiuretic agent, nitrate drug, nitric oxide donor, NMDA antagonist, nonsteroidal anti-inflammatory drug, opioid antagonist, PDE III inhibitor, phosphatidylcholine precursor, phosphodiesterase inhibitor, platelet aggregation inhibitor, potassium channel blocking agent, prostacyclin derivative, sclerosing solution, sedative, serotonin agonist, sodium channel blocking agent, statin, sympathetic nerve inhibitor, thrombolytic agent, thromboxane receptor antagonist, tissue-type plasminogen activator, vasoconstrictor agent, vasodilator agent, or xanthine formulation.

Exemplary agents for the treatment of cardiovascular disease include acebutolol, adenosine, alacepril, alprenolol, alteplase, amantadine, amiloride, amiodarone, amlodipine, amosulalol, anisoylated plasminogen streptokinase activator complex, aranidipine, argatroban, arotinolol, artilide, aspirin, atenolol, azimilide, bamidipine, batroxobin, befunolol, benazepril, bencyclane, bendrofluazide, bendroflumethiazide, benidipine, benzthiazide, bepridil, beraprost sodium, betaxolol, bevantolol, bisoprolol, bopindolol, bosentan, bretylium, bucumolol, buferalol, bumetanide, bunitrolol, buprandolol, butofilolol, butylidine, candesartan, captopril, carazolol, carteolol, carvedilol, celiprolol, ceronapril, cetamolol, chlorothiazide, chlorthalidone, cilazapril, cilnidipine, cilostazol, cinnarizine, citicoline, clentiazem, clofilium, clopidogrel, cloranolol, cyclandelate, cyclonicate, dalteparin calcium, dalteparin sodium, danaparoid sodium, delapril, diazepam, digitalis, digitoxin, digoxin, dilazep hydrochloride, dilevalol, diltiazem, dipyridamole, disopyramide, dofetilide, and dronedarone.

Other exemplary agents for the treatment of cardiovascular disease include ebumamonine, edaravone, efonidipine, elgodipine, Eminase, enalapril, encainide, enoxaparin, eprosartan, ersentilide, esmolol, etafenone, ethacrynic acid, ethyl icosapentate, felodipine, fiunarizine, flecainide, flumethiazide, flunarizine, flurazepam, fosinopril, furosemide, galopamil, gamma-aminobutyric acid, glyceryl trinitrate, heparin calcium, heparin potassium, heparin sodium, hydralazine, hydrochlorothiazide, hydroflumethiazide, ibudilast, ibutilide, ifenprodil, ifetroban, iloprost, imidapril, indenolol, indobufene, indomethacin, irbesartan, isobutilide, isosorbide nitrate, isradipine, labetalol, lacidipine, lercanidipine, lidocaine, lidoflazine, lignocaine, lisinopril, lomerizine, losartan, magnesium ions, manidipine, methylchlorthiazide, metoprolol, mexiletine, mibefradil, mobertpril, monteplase, moricizine, musolimine, nadolol, naphlole, nasaruplase, nateplase, nicardipine, nickel chloride, nicorandil, nifedipine, nikamate, nilvadipine, nimodipine, nipradilol, nisoldipine, nitrazepam, nitrendipine, nitroglycerin, nofedoline and nosergoline.

Other agents for the treatment of cardiovascular disease include pamiteplase, papaverine, parnaparin sodium, penbutolol, pentaerythritol tetranitrate, pentifylline, pentopril, pentoxifylline, perhexiline, perindopril, phendilin, phenoxezyl, phenytoin, pindolol, polythiazide, prenylamine, procainaltide, procainamide, propafenone, propranolol, prostaglandin 12, prostaglandin El, prourokinase, quinapril, quinidine, ramipril, randolapril, rateplase, recombinant tPA, reviparin sodium, sarpogrelate hydrochloride, semotiadil, sodium citrate, sotalol, spirapril, spironolactone, streptokinase, tedisamil, temocapril, terodiline, tiapride, ticlopidene, ticrynafen, tilisolol, timolol, tisokinase, tissue plasminogen activator (tPA), tocainide, trandolapril, trapidil, trecetilide, triamterene, trichloromethiazide, urokinase, valsartan, verapamil, vichizyl, vincamin, vinpocetine, vitamin C, vitamin E, warfarin, and zofenopril.

In some embodiments, the agent is a derivative of a compound with pharmaceutical activity, such as an acetylated derivative or a pharmaceutically acceptable salt. In some embodiments, the agent is a prodrug such as a hexanoate conjugate.

Agent may mean a combination of agents that have been combined and attached to a polymer and/or loaded into the particle. Any combination of agents may be used. For example, pharmaceutical agents may be combined with diagnostic agents, pharmaceutical agents may be combined with prophylactic agents, pharmaceutical agents may be combined with other pharmaceutical agents, diagnostic agents may be combined with prophylactic agents, diagnostic agents may be combined with other diagnostic agents, and prophylactic agents may be combined with other prophylactic agents. In certain embodiments for treating cancer, at least two traditional chemotherapeutic agents are attached to a polymer and/or loaded into the particle.

In certain embodiments, the agent may be attached to a polymer to form a polymer-agent conjugate.

In certain embodiments, the agent in the particle is attached to a polymer of the particle. The agent may be attached to any polymer in the particle, e.g., a hydrophobic polymer or a polymer containing a hydrophilic and a hydrophobic portion.

In certain embodiments, an agent is embedded in the particle. The agent may be associated with a polymer or other component of the particle through one or more non-covalent interactions such as van der Waals interactions, hydrophobic interactions, hydrogen bonding, dipole-dipole interactions, ionic interactions, and pi stacking.

An agent may be present in varying amounts of a polymer-agent conjugate, particle or composition described herein. When present in a particle, the agent may be present in an amount, e.g., from about 1 to about 30% by weight (e.g., from about 2 to about 30% by weight, from about 4 to about 25% by weight, or from about 5 to about 13%, 14%, 15%, 16%, 17%, 18%, 19% or 20% by weight).

Modes of Attachment

An agent described herein may be directly attached to a polymer described herein. A reactive functional group of an agent may be directly attached to a functional group on a polymer. An agent may be attached to a polymer via a variety of linkages, e.g., an amide, ester, succinimide, carbonate or carbamate linkage. For example, in one embodiment, hydroxy group of an agent may be reacted with a carboxylic acid group of a polymer, forming a direct ester linkage between the agent and the polymer. In another embodiment, an amino group of an agent may be linked to a carboxylic acid group of a polymer, forming an amide bond.

In some embodiments, an agent may be directly attached to a terminal end of a polymer. For example, a polymer having a carboxylic acid moiety at its terminus may be covalently attached to a hydroxy or amino moiety of an agent, forming an ester or amide bond.

In certain embodiments, suitable protecting groups may be required on the other polymer terminus or on other reactive substituents on the agent, to facilitate formation of the specific desired conjugate. For example, a polymer having a hydroxy terminus may be protected, e.g., with an alkyl group (e.g., methyl) or an acyl group (e.g., acetyl). An agent such as a taxane (e.g., paclitaxel, docetaxel, larotaxel or cabazitaxel) may be protected, e.g., with an acetyl group, on the 2′ hydroxyl group, such that the docetaxel may be attached to a polymer via the 7-hydroxyl group, the 10 hydroxyl group or the 1 hydroxyl group.

In some embodiments, the process of attaching an agent to a polymer may result in a composition comprising a mixture of polymer-agent conjugates having the same polymer and the same agent, but which differ in the nature of the linkage between the agent and the polymer. For example, when an agent has a plurality of reactive moieties that may react with a polymer, the product of a reaction of the agent and the polymer may include a polymer-agent conjugate wherein the agent is attached to the polymer via one reactive moiety, and a polymer-agent conjugate wherein the agent is attached to the polymer via another reactive moiety. For example, taxanes have a plurality of hydroxyl moieties, all of which may react with a polymer. Thus, when the agent is a taxane, the resulting composition may include a plurality of polymer-taxane conjugates including polymers attached to the agent via different hydroxyl groups present on the taxane. In the case of paclitaxel, the plurality of polymer-agent conjugates may include polymers attached to paclitaxel via the hydroxyl group at the 2′ position, polymers attached to paclitaxel via the hydroxyl group at the 7 position, and/or polymers attached to paclitaxel via the hydroxyl group at the 1 position. The plurality of polymer-agent conjugates may also include paclitaxel molecules linked to 2 or more hydroxyl groups. For example, the plurality may include paclitaxel molecules linked to 2 polymers via the hydroxyl group at the 2′ position and the hydroxyl group at the 7 position; the hydroxyl group at the 2′ position and hydroxyl group at the 10 position; or the hydroxyl group at the 7 position and the hydroxyl group at the 10 position. In the case of docetaxel, the plurality of polymer-agent conjugates may include polymers attached to docetaxel via the hydroxyl group at the 2′ position, polymers attached to docetaxel via the hydroxyl group at the 7 position, polymers attached to docetaxel via the hydroxyl group at the 10 position and/or polymers attached to docetaxel via the hydroxyl group at the 1 position. The plurality of polymer-agent conjugates may also include docetaxel molecules linked to 2 or more hydroxyl groups. For example, the plurality may include docetaxel molecules linked to 2 polymers via the hydroxyl group at the 2′ position and the hydroxyl group at the 7 position, the hydroxyl group at the 2′ position and the hydroxyl group at the 10 position; or the hydroxyl group at the 7 position and the hydroxyl group at the 10 position.

In some embodiments, the process of attaching an agent to a polymer may involve the use of protecting groups. For example, when an agent has a plurality of reactive moieties that may react with a polymer, the agent may be protected at certain reactive positions such that a polymer will be attached via a specified position. In one embodiment, when the agent is a taxane, the agent may be selectively coupled to the polymer, e.g., via the 2′-hydroxyl group, by protecting the remaining hydroxyl groups with suitable protecting groups. For example, when the agent is docetaxel, the 2′ hydroxyl group may be protected, e.g., with a Cbz group. After purification of the product that is selectively protected at the 2′ positions, the 7 and 10 positions may then be orthogonally protected, e.g., with a silyl protecting group. The 2′ hydroxyl group may then be deprotected, e.g., by hydrogenation, and the polymer may be coupled to the 2′ hydroxyl group. The 7 and 10 hydroxyl groups may then be deprotected, e.g., using fluoride, to yield the polymer-docetaxel conjugate in which the polymer is attached to docetaxel via the 2′ hydroxyl group.

Alternatively, docetaxel may be reacted with two equivalents of a protecting group such that a mixture of products is formed, e.g., docetaxel protected on the hydroxyl groups at the 2′ and 7 positions, and docetaxel protected on the hydroxyl groups at the 2′ and 10 positions. These products may be separated and purified, and the polymer may be coupled to the free hydroxyl group (the 10-OH or the 7-OH respectively). The product may then be deprotected to yield the product polymer-docetaxel conjugate in which the polymer is attached to docetaxel via the hydroxyl group at the 7 position, or polymer attached to docetaxel via the hydroxyl group at the 10 position.

In some embodiments, selectively-coupled products such as those described above may be combined to form mixtures of polymer-agent conjugates. For example, PLGA attached to docetaxel via the 2′-hydroxyl group, and PLGA attached to docetaxel via the 7-hydroxyl group, may be combined to form a mixture of the two polymer-agent conjugates, and the mixture may be used in the preparation of a particle.

A polymer-agent conjugate may comprise a single agent attached to a polymer. The agent may be attached to a terminal end of a polymer, or to a point along a polymer chain.

In some embodiments, the polymer-agent conjugate may comprise a plurality of agents attached to a polymer (e.g., 2, 3, 4, 5, 6 or more agents may be attached to a polymer). The agents may be the same or different. In some embodiments, a plurality of agents may be attached to a multifunctional linker (e.g., a polyglutamic acid linker). In some embodiments, a plurality of agents may be attached to points along the polymer chain.

Linkers

An agent may be attached to a polymer via a linker, such as a linker described herein. In certain embodiments, a plurality of the linker moieties are attached to a polymer, allowing attachment of a plurality of agents to the linker. The agent may be released from the linker under biological conditions. In another embodiment a single linker is attached to a polymer, e.g., at a terminus of the polymer.

The linker may be, for example, an alkylenyl (divalent alkyl) group. In some embodiments, one or more carbon atoms of the alkylenyl linker may be replaced with one or more heteroatoms. In some embodiments, one or more carbon atoms may be substituted with a substituent (e.g., alkyl, amino, or oxo substituents).

In some embodiments, the linker, prior to attachment to the agent and the polymer, may have one or more of the following functional groups: amine, amide, hydroxyl, carboxylic acid, ester, halogen, thiol, maleimide, carbonate, or carbamate.

In some embodiments, the linker may comprise an amino acid linker or a peptide linker. Frequently, in such embodiments, the peptide linker is cleavable by hydrolysis, under reducing conditions, or by a specific enzyme.

When the linker is the residue of a divalent organic molecule, the cleavage of the linker may be either within the linker itself, or it may be at one of the bonds that couples the linker to the remainder of the conjugate, i.e. either to the agent or the polymer.

In some embodiments, a linker may be selected from one of the following:

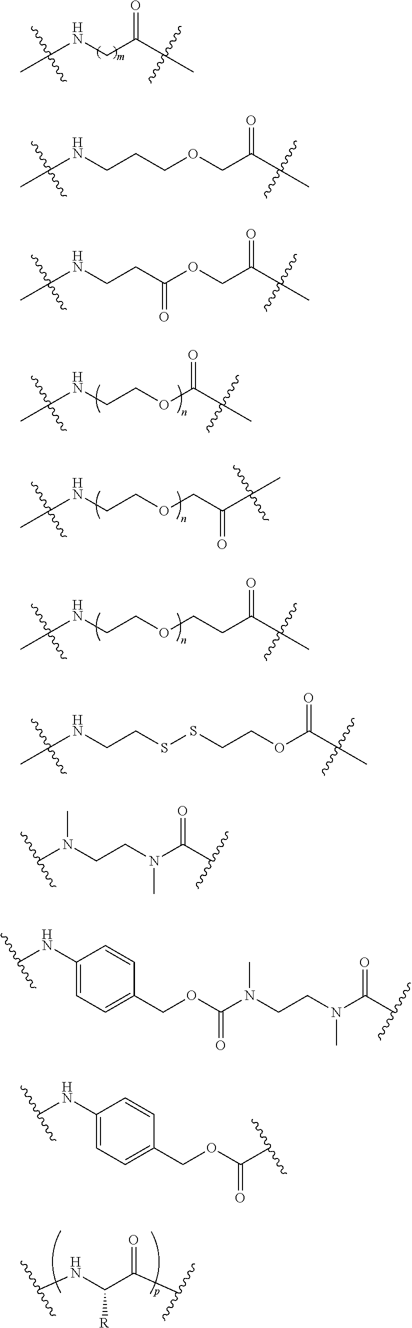

wherein m is 1-10, n is 1-10, p is 1-10, and R is an amino acid side chain.

A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids.

In some embodiments, a linker may be a covalent bond.

Methods of making polymer-agent conjugates

The polymer-agent conjugates may be prepared using a variety of methods known in the art, including those described herein. In some embodiments, to covalently link the agent to a polymer, the polymer or agent may be chemically activated using any technique known in the art. The activated polymer is then mixed with the agent, or the activated agent is mixed with the polymer, under suitable conditions to allow a covalent bond to form between the polymer and the agent. In some embodiments, a nucleophile, such as a thiol, hydroxyl group, or amino group, on the agent attacks an electrophile (e.g., activated carbonyl group) to create a covalent bond. An agent may be attached to a polymer via a variety of linkages, e.g., an amide, ester, succinimide, carbonate or carbamate linkage.

In some embodiments, an agent may be attached to a polymer via a linker. In such embodiments, a linker may be first covalently attached to a polymer, and then attached to an agent. In other embodiments, a linker may be first attached to an agent, and then attached to a polymer.

Exemplary Polymer-Agent Conjugates

Polymer-agent conjugates can be made using many different combinations of components described herein. For example, various combinations of polymers (e.g., PLGA, PLA or PGA), linkers attaching the agent to the polymer, and agents are described herein.

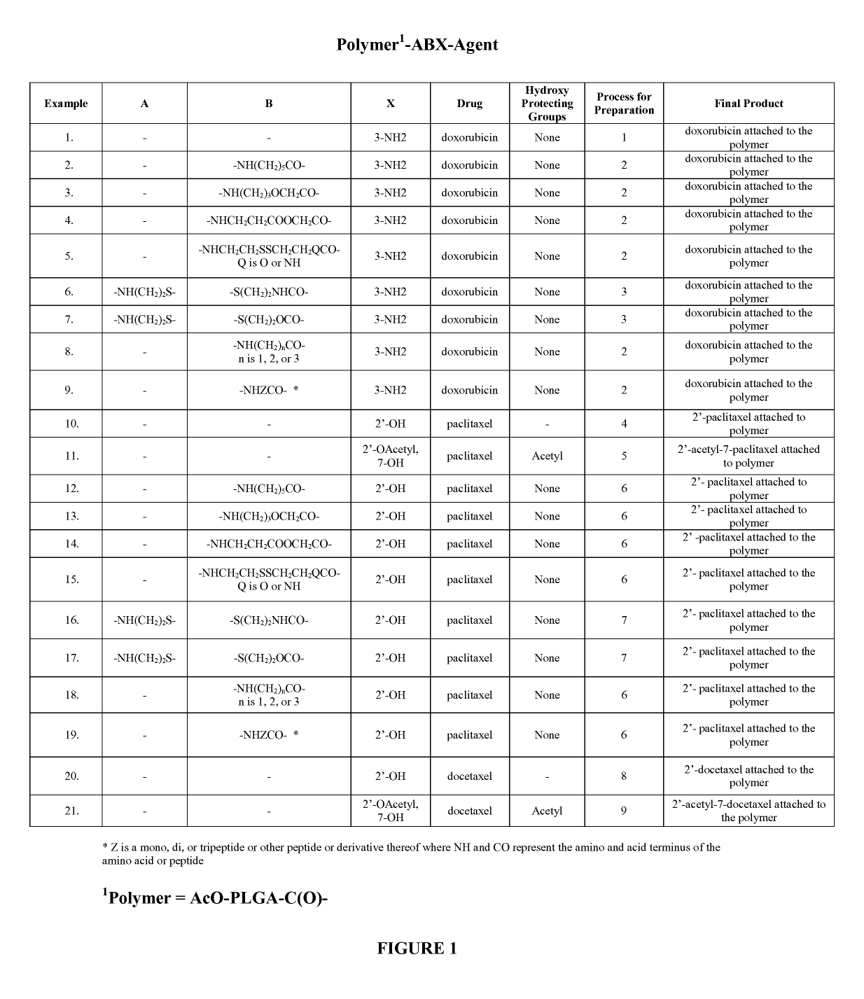

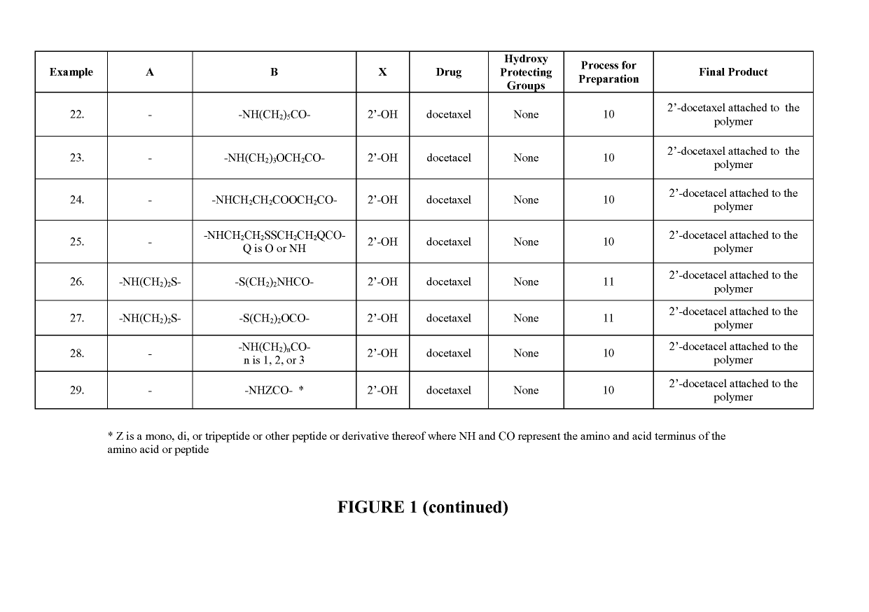

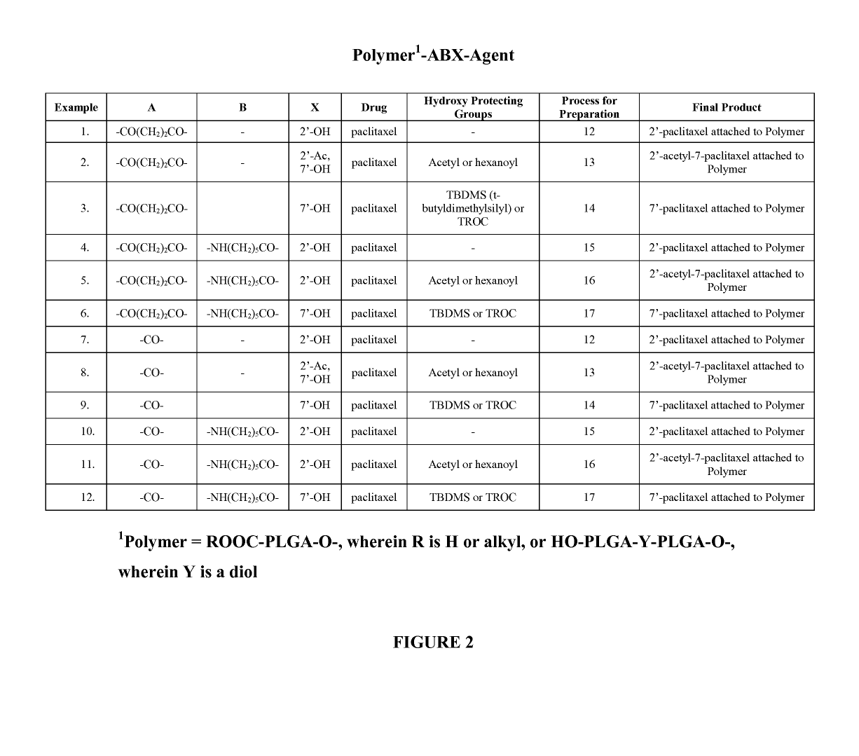

FIG. 1 and FIG. 2. are tables depicting examples of different polymer-agent conjugates. The polymer-agent conjugates in FIG. 1 and FIG. 2 are represented by the following formula:

Polymer-ABX-Agent

“Polymer” in this formula represents the polymer portion of the polymer-agent conjugate. The polymer can be further modified on the end not conjugated with the agent. For example in instances where the polymer terminates with an —OH, the —OH can be capped, for example with an acyl group, as depicted in FIG. 1. In instances where the polymer terminates with a —COOH, the polymer may be capped, e.g., with an alkyl group to provide an ester.

A and B represent the connection between the polymer and the agent. Position A is either a bond between linker B and the carbonyl of the polymer (represented as a “-” in FIG. 1 and FIG. 2), a bond between the agent and the carbonyl of the polymer (represented as a “-” in FIG. 1 and FIG. 2) or depicts a portion of the linker that is attached via a bond to the carbonyl of the polymer. Position B is either not occupied (represented by “-” in FIG. 2) or represents the linker or the portion of the linker that is attached via a bond to the agent; and

X represents the heteroatom on the agent through which the linker or polymer is coupled to the agent.

As provided in FIG. 1 and FIG. 2, the column with the heading “drug” indicates which agent is included in the polymer-agent conjugate.

The three columns on the right of the table in FIG. 1 and FIG. 2 indicate respectively, what, if any, protecting groups are used to protect a hydroxy group on the agent, the process for producing the polymer-agent conjugate, and the final product of the process for producing the polymer-agent conjugate.

The processes referred to in FIG. 1 are given a numerical representation, e.g., Process 1, Process 2, Process 3 etc. as seen in the second column from the right. The steps for each these processes respectively are provided below.

Process 1: Couple the polymer directly to doxorubicin to afford doxorubicin linked to polymer.

Process 2: Couple the protected linker of position B to doxorubicin, deprotect the linker and couple to polymer via the carboxylic acid group of the polymer to afford the doxorubicin linked to the polymer.

Process 3: Couple the activated linker of position B to doxorubicin, couple to polymer containing linker of position A via the linker of A to afford doxorubicin linked to polymer.

Process 4: Couple the polymer directly to paclitaxel to afford 2′-linked paclitaxel to polymer

Process 5: Acetylate the 2′OH group of paclitaxel, couple the polymer directly to 7-OH group of paclitaxel and isolate the 2′acetyl-7-paclitaxel linked to polymer

Process 6: Couple the protected linker of position B to the paclitaxel, deprotect the linker and couple to polymer via the carboxylic acid group of the polymer to afford the 2′-paclitaxel linked to the polymer

Process 7: Couple the activated linker of position B to the 2′-hydroxyl of paclitaxel, and couple to polymer containing linker of position A via the linker of A to afford 2′-paxlitaxel linked to polymer.

Process 8: Couple the polymer directly to docetaxel to afford 2′docetaxel linked to polymer

Process 9: Acetylate the 2′OH group of docetaxel, couple the polymer directly to 7-OH group of docetaxel and isolate the 2′acetyl-7-docetaxel linked to polymer

Process 10: Couple the protected linker of position B to the docetaxel, deprotect the linker and couple to polymer via the carboxylic acid group of the polymer to afford the 2′-docetaxel linked to the polymer

Process 11: Couple the activated linker of position B to the 2′-hydroxyl of docetaxel, and couple to polymer containing linker of position A via the linker of A to afford 2′-docetacel linked to polymer.

The processes referred to in FIG. 2 (terminal alcohol containing polymers) are given a numerical representation, e.g., Process 12, Process 13, Process 14 etc. as seen in the second column from the right. The steps for each these processes respectively are provided below.

Process 12: Couple paclitaxel directly to polymer containing linker of position A via the linker of A to afford 2′-paclitaxel linked to polymer.

Process 13: Protect the 2′-alcohol of paclitaxel, couple paclitaxel directly to polymer containing linker of position A via the linker of A to afford 2′-protected-7-paclitaxel linked to polymer. The protecting group is removed in vivo.

Process 14: Protect the 2′-alcohol of paclitaxel, couple paclitaxel directly to polymer containing linker of position A via the linker of A, deprotect the 2′-hydroxyl group to afford 7-paclitaxel linked to polymer.

Process 15: Couple the protected linker of position B to the 2′-hydroxyl of paclitaxel, deprotect, and couple to polymer containing linker of position A via the linker of A to afford 2′-paclitaxel linked to polymer.

Process 16: Protect the 2′-alcohol of paclitaxel, couple the protected paclitaxel to the protected linker of position B to the 7′-hydroxyl of paclitaxel, deprotect the linker protecting group and couple to polymer containing linker of position A via the linker of A to afford 2′-protected-7-paclitaxel linked to polymer.

Process 17: Protect the 2′-alcohol of paclitaxel, couple the protected paclitaxel to the protected linker of position B to the 7′-hydroxyl of paclitaxel, deprotect both the amino and the hydroxyl groups, and couple to polymer containing linker of position A via the linker of A or deprotect the linker protecting group, couple to polymer containing linker of position A via the linker of A and deprotect the hydroxyl group to afford 7′-paclitaxel linked to polymer.

Exemplary polymer-agent conjugates include the following.

1) Docetaxel-5050-PLGA-O-acetyl

One exemplary polymer-agent conjugate is docetaxel-5050-PLGA-O-acetyl, which is a conjugate of PLGA and docetaxel. This conjugate has the formula shown below:

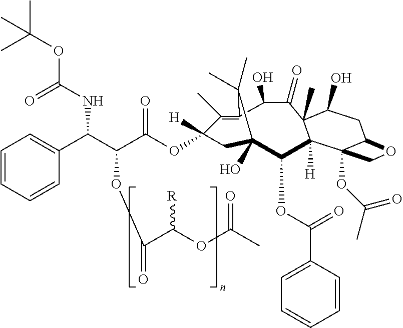

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from glc-monomers and lac-monomers (as opposed to dimers) can be used as well.

The terminal hydroxyl (OH) group of PLGA is acetylated prior to conjugation of docetaxel to the terminal carboxylic acid (COOH) group. Docetaxel is attached to PLGA via an ester bond, primarily via the 2′ hydroxyl group. The product may include docetaxel attached to the polymer via the 2′, 7, 10 and/or 1 positions, and docetaxel attached to multiple polymer chains (e.g., via both the 2′ and 7 positions).

The weight loading of docetaxel on the PLGA polymer ranges from 5-16 weight %.

2) Doxorubicin-5050 PLGA-amide

Another exemplary polymer-agent conjugate is doxorubicin-5050 PLGA-amide, which is a conjugate of PLGA and doxorubicin. This conjugate has the formula shown below:

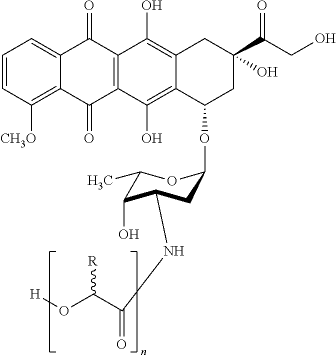

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

The PLGA was synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from glc-monomers and lac-monomers (as opposed to dimers) can be used as well.

Doxorubicin is attached to PLGA via an amide bond. The weight loading of doxorubicin on the PLGA polymer ranges from 8-12 weight %.

3) Paclitaxel-5050-PLGA-O-acetyl

Another exemplary polymer-agent conjugate is paclitaxel-5050-PLGA-O-acetyl, which is a conjugate of PLGA and paclitaxel. This conjugate has the structure shown below:

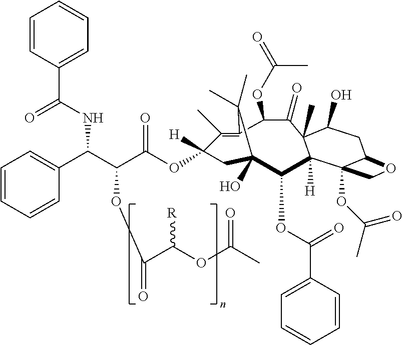

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA was synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from glc-monomers and lac-monomers (as opposed to dimers) can be used as well.

The terminal hydroxyl (OH) group of PLGA is acetylated prior to conjugation of paclitaxel to the terminal carboxylic acid (COOH) group. Paclitaxel is attached to PLGA via an ester bond, primarily via the 2′ hydroxyl group. The product may include paclitaxel attached to the polymer via the 2′, 7 and/or 1 positions, and paclitaxel attached to multiple polymer chains (e.g., via both the 2′ and 7 positions). The weight loading of paclitaxel on the PLGA polymer ranges from 7-9 weight %.

4) Docetaxel-hexanoate-5050 PLGA-O-acetyl

Another exemplary polymer-agent conjugate is docetaxel-hexanoate-5050 PLGA-O-acetyl, which is a conjugate of PLGA and docetaxel with a hexanoate linker. This conjugate has the formula shown below:

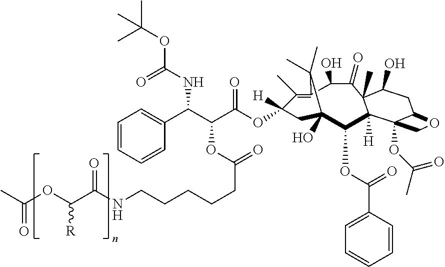

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA was synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from glc-monomers and lac-monomers (as opposed to dimers) can be used as well.

There is a hexanoate linker between the PLGA polymer and the drug docetaxel. Docetaxel-hexanoate is attached to the polymer primarily via the 2′ hydroxyl group of docetaxel. The product may include docetaxel-hexanoate attached to the polymer via the 2′, 7, 10 and/or 1 positions, and docetaxel attached to multiple polymer chains (e.g., via both the 2′ and 7 positions). The weight loading of docetaxel on the PLGA polymer ranges from 10-11 weight %.

5) Bis(docetaxel) glutamate-5050 PLGA-O-acetyl

Another exemplary polymer-agent conjugate is bis(docetaxel) glutamate-5050 PLGA-O-acetyl, which is a conjugate of docetaxel and PLGA, with a bifunctional glutamate linker. This conjugate has the formula shown below:

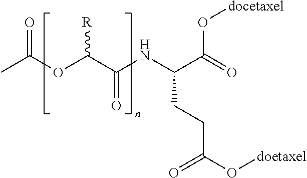

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from glc-monomers and lac-monomers (as opposed to dimers) can be used as well.

Each docetaxel is attached to the glutamate linker via an ester bond, primarily via the 2′ hydroxyl groups. The product may include polymers in which one docetaxel is attached via the hydroxyl group at the 2′ position and the other is attached via the hydroxyl group at the 7 position; one docetaxel is attached via the hydroxyl group at the 2′ position and the other is attached via the hydroxyl group at the 10 position; one docetaxel is attached via the hydroxyl group at the 7 position and the other is attached via the hydroxyl group at the 10 position; and/or polymers in which only one docetaxel is linked to the polymer, via the hydroxyl group at the 2′ position, the hydroxyl group at the 7 position or the hydroxyl group at the 10 position; and/or docetaxel molecules attached to multiple polymer chains (e.g., via both the hydroxyl groups at the 2′ and 7 positions). The weight loading of docetaxel on the PLGA polymer ranges from 10-16 weight %.

6) Tetra-(docetaxel) triglutamate-5050 PLGA-O-acetyl

Another exemplary polymer-agent conjugate is tetra-(docetaxel) triglutamate-5050 PLGA-O-acetyl, which is a conjugate of PLGA and docetaxel, with a tetrafunctional tri(glutamate) linker. This conjugate has the formula shown below:

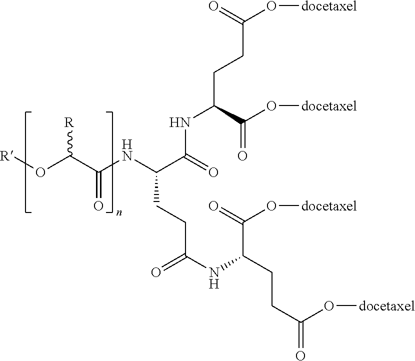

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

Each docetaxel is attached to the tri(glutamate) linker via an ester bond, primarily via the 2′ hydroxyl groups. The product may include polymers in which docetaxel is attached via the 2′, 7, 10 and/or 1 positions, in any combination; or polymers in which 0, 1, 2 or 3 docetaxel molecules are attached, via the 2′, 7, 10 and/or 1 positions; and/or docetaxel molecules attached to multiple polymer chains (e.g., via both the 2′ and 7 positions). The weight loading of docetaxel on the PLGA polymer ranges from 19-21 weight %.

In some embodiments, the polymer-agent conjugate of the present invention is a polymer-anticancer agent conjugate described below.

In a 1^{st} embodiment of the present invention, the polymer-anticancer agent conjugate is represented by any one of the following structural formulas:

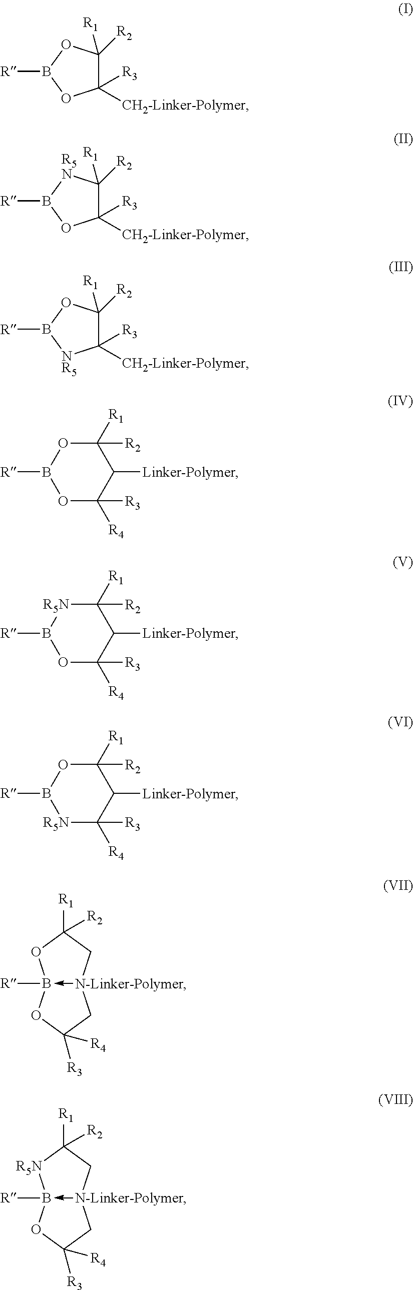

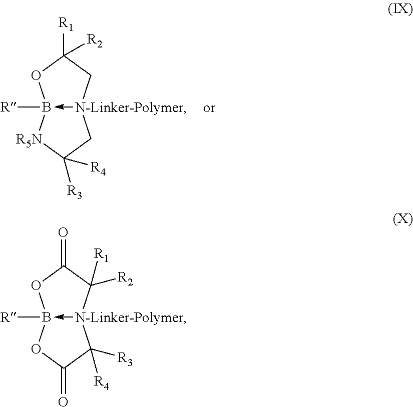

wherein:

R″ is the residue of a boronic acid containing pharmaceutically active agent. Specifically, R″ is portion of the pharmaceutically active agent that is attached to the boron atom of the drug and together with the boronic acid group (or —BZ₁Z₂ group) forms the entire drug molecule,

R₁, R₂, R₃ and R₄ are each independently —H or a (C₁-C₅)alkyl; R₅ is —H or (C₁-C₆) alkyl; and

the polymer is a polymer described herein and is optionally functionalized at the terminus.

In some embodiments, the polymer-anticancer agent conjugate is represented by any one of the following structural formulas:

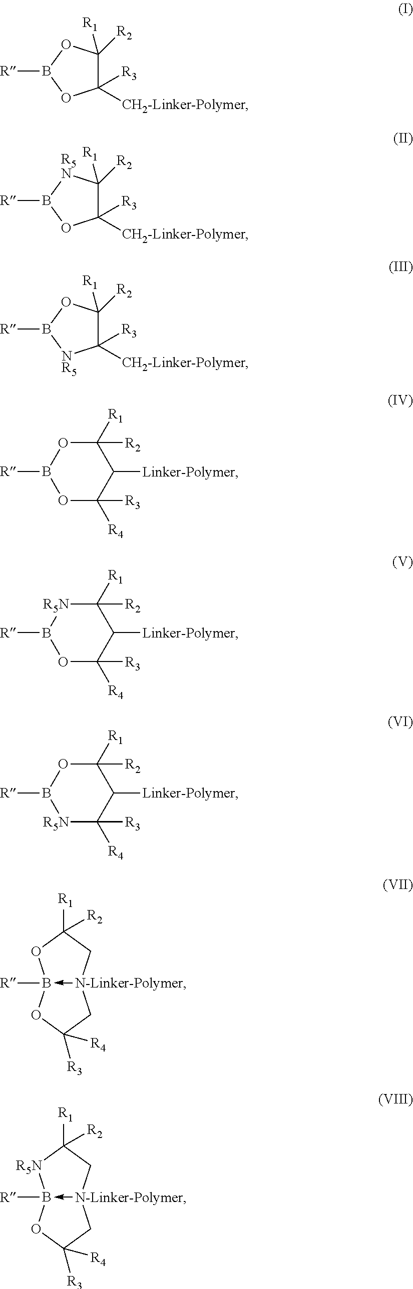

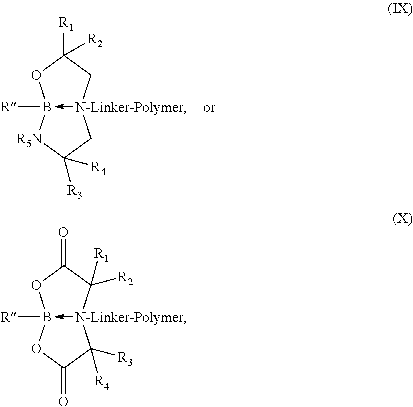

wherein:

R″ is the residue of bortezomib which, together with —B(OH)₂ forms bortezomib;

R₁, R₂, R₃ and R₄ are each independently —H or a (C₁-C₅)alkyl; R₅ is —H or (C₁-C₆) alkyl; and

the polymer is a polymer described herein and is optionally functionalized at the terminus.

In a 2^{nd} embodiment, for polymer-anticancer agent conjugate represented by structural formulas (I)-(X), the linker is represented by —W—X—Y—Z₁-A₁- and the polymer comprises a hydroxyl terminal and is attached to the linker at the hydroxyl terminal, wherein:

W is —(CH₂)ₘ—, —O— or —N(R₅)—, when the polymer-agent conjugate is represented by structural formulas (I)-(VI); or

W is —(CH₂)ₘ—, when the polymer-agent conjugate is represented by structural formulas (VII)-(X);

X is a bond when W is —(CH₂)ₘ— and X is —C(═O)— when W is —O—, or —N(R₅);

Y is a bond, —O—, or —N(R₅)—;

Z₁ is represented by the following structural formula:

-D-(CH₂)ₚ-Q₁-(CH₂)_{q}—;

Q₁ is a bond, —O—, —N(R₅)—, —C(═O)O—, —O—C(═O)—,

—O—C(═O)—N(R₅)—, —N(R₅)—C(═O)—O—, —S—S—, —(CH₂—CH₂—O)ₙ—, or

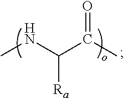

D is a bond, aryl or heteroaryl;

A₁ is —C(═O)—, —O—C(═O)— or —N(R₅)—C(═O)—, or A₁ is a bond when Q₁ is

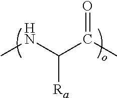

and q is 0;

Rₐ is a side chain of a naturally occurring amino acid or an analog thereof;

R₅ is —H or (C₁-C₆)alkyl,

m, p, q are each an integer from 0 to 10;

n is an integer from 2 to 10; and

o is an integer from 1 to 10, provided when Y is —O— or —N(R₅)—, Q₁ is —O—, —N(R₅)—, —O—C(═O)—N(R₅)—, —N(R₅)—C(═O)—O—, —O—C(═O)— or —S—S—, and D is a bond, then p≧2; when Q₁ is —O—, —N(R₅)—, —(CH₂—CH₂—O)ₙ—, —O—C(═O)—N(R₅)—, —N(R₅)—C(═O)—O—, —O—C(═O)—, —C(═O)—O— or —S—S— and A₁ is —O—C(═O)— or —N(R₅)—C(═O)—, q≧2; when Y is —O— or —N(R₅)—, D and Q₁ are both a bond, and p and q are both 0, then A₁ is —C(═O)—; when Y is —O— or —N(R₅)—, D and Q₁ are both a bond, A₁ is —O—C(═O)— or —N(R₅)—C(═O)—, then p+q≧2; and when W is —O— or —N(R₅)—, Y, D and Q₁ are all a bond, then p+q≧2.

In a 3^{rd} embodiment, for polymer-anticancer agent conjugate represented by structural formulas (I)-(X), the linker is represented by —W—X—Y—Z₂-A₂- and the polymer comprises a carboxyl terminal and is attached to the linker at the carboxyl terminal, wherein:

Z₂ is represented by the following structural formula:

—(CH₂)ₚ-Q₂-(CH₂)_{q}-D-;

Q₂ is a bond, —O—, —N(R₅)—, —N(R₅)—C(═O)—O—, —O—C(═O)—N(R₅)—, —OC(═O)—, —C(═O)—O—, —S—S—, —(O—CH₂—CH₂)ₙ— or

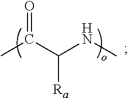

A₂ is —O— or —N(R₅)—, or A₂ is a bond when Q₂ is

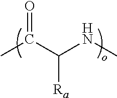

and q is 0, and the remainder of the variables are as described above in the 2^{nd} embodiment, provided when Y is —O— or —N(R₅)— and Q₂ is —O—, —N(R₅)—, —(O—CH₂—CH₂)ₙ—, —N(R₅)—C(═O)—O—, —O—C(═O)—N(R₅)—, —OC(═O)— or —S—S—, then p≧2; when Q₂ is —O—, —N(R₅)—, —N(R₅)—C(═O)—O—, —O—C(═O)—N(R₅)—, —OC(═O)—, —C(═O)—O—, or —S—S— and D is a bond, then q≧2; when Y is —O— or —N(R₅)—, Q₂ and D are both a bond, then p+q≧2; when W is —O— or —N(R₅)—, Y, Q₂ and D are all bond, then p+q≧1; and when W is —O— or —N(R₅)—, Y is a bond, and Q₂ is —N(R₅)—C(═O)—O—, —O—C(═O)—N(R₅)—, —OC(═O)—, —C(═O)—O—, —S—S— or —(O—CH₂—CH₂)ₙ—, then p≧2.

In one embodiment, the biodegradable polymer described in the 1^{st}, 2^{nd} or 3^{rd} embodiment above is a polyhydroxyalkanoate polymer optionally derivatized at terminus. More specifically, the polyhydroxyalkanoate polymer is selected from PLA, PGA, PLGA and PCL.

In a 4^{th} embodiment, the polymer-anticancer agent conjugate is represented by any one of the following structural formulas:

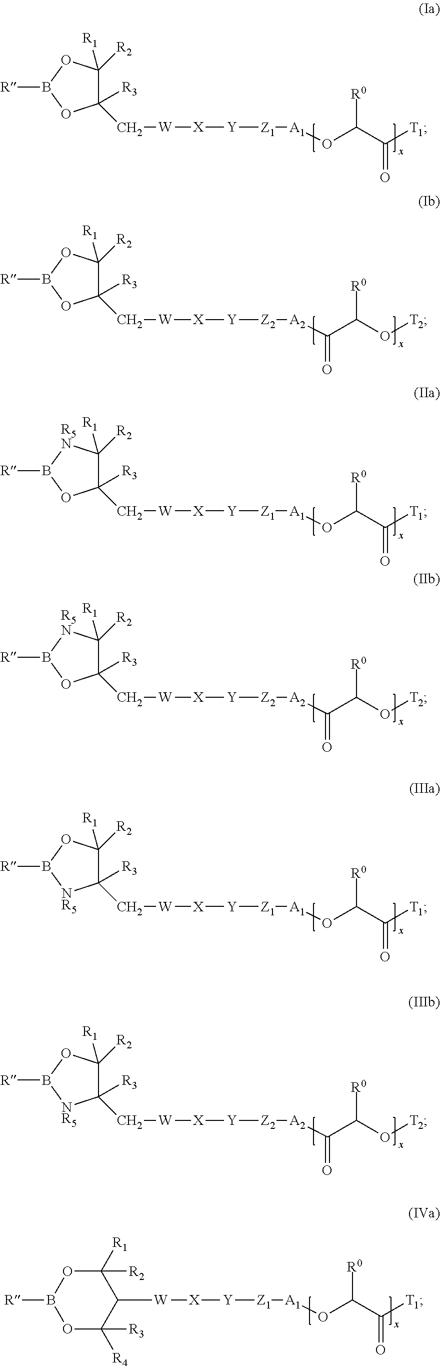

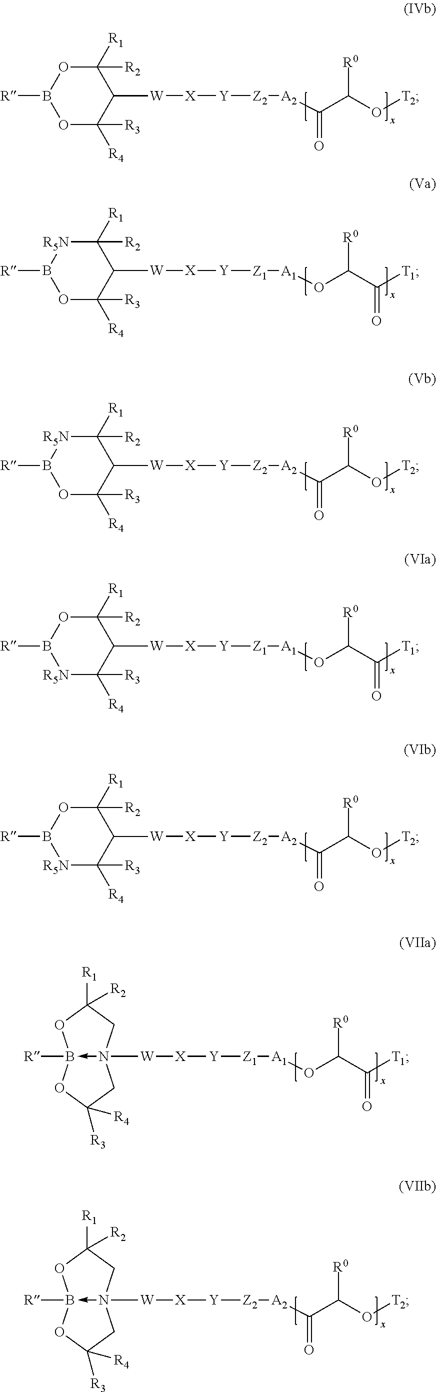

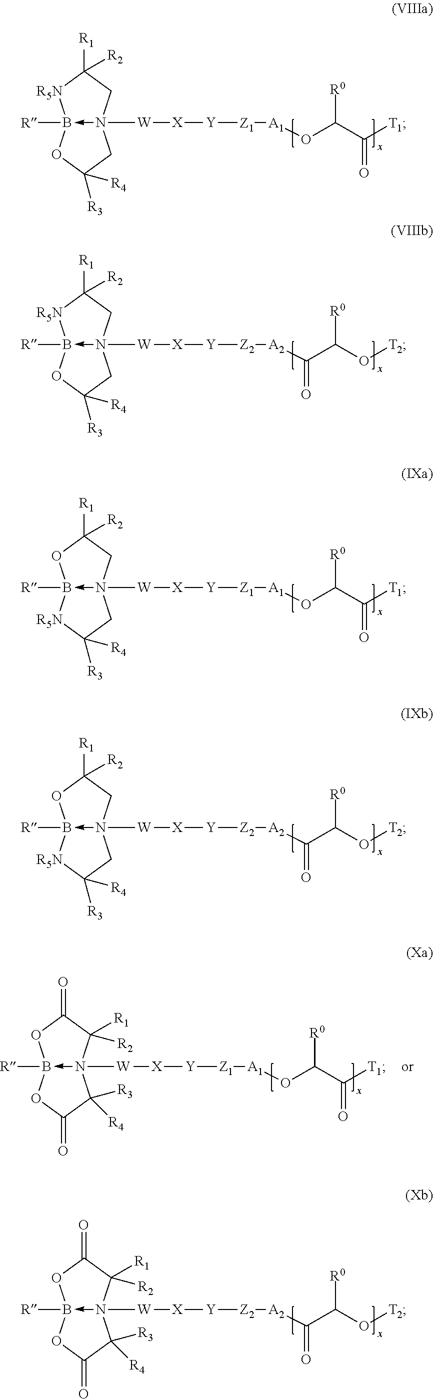

wherein:

each R⁰ is independently H or a (C₁-C₆)alkyl;

T₁ is OH or OR₆;

T₂ is H, —C(O)R₇ or alkoxy PEG (e.g. —(CH₂—CH₂—O)_{y}—CH₂—CH₂—O—CH₃, wherein y is an integer from 1 to 20);

R₆ is a (C₁-C₆)alkyl or alkoxy PEG (e.g. —(CH₂—CH₂—O)_{y}—CH₂—CH₂—O—CH₃, wherein y is an integer from 1 to 20);

R₇ is a (C₁-C₆)alkyl; and

x is an integer from 1 to 1000;

and the remainder of the variables are as described in the first, second or third embodiment.

In one embodiment, for the polymer-anticancer agent conjugates described in the 4^{th} embodiment, T₁ is OH or —O—(CH₂—CH₂—O)_{y}—CH₂—CH₂—O—CH₃; T₂ is H, or —C(O)—CH₃.

In another embodiment, for the polymer-anticancer agent conjugates described in the 4^{th} embodiment, the ratio of R⁰ is methyl to R⁰ is H is from about 0.1:99.9 to 99.9:0.1. In some embodiments, the ratio R⁰ is methyl to R⁰ is H is from about 75:25 to about 25:75 (e.g. from about 60:40 to about 40:60, or from about 55:45 to about 45:55). In some embodiments, the ratio of R⁰ is methyl to R⁰ is H is from about 50:50 to about 75:25.

In another embodiment, for the polymer-anticancer agent conjugates described in the 4^{th} embodiment, about 50% of the groups represented by R⁰ are H and about 50% of the groups represented by R⁰ is methyl.

In another embodiment, for the polymer-anticancer agent conjugates described in the 4^{th} embodiment, about 50% of the groups represented by R⁰ are H and about 50% of the groups represented by R⁰ are methyl; and T₁ is OH and T₂ is H or —C(O)—CH₃.

In a 5^{th} embodiment, the polymer-anticancer agent conjugate is represented by any one of the following structural formulas:

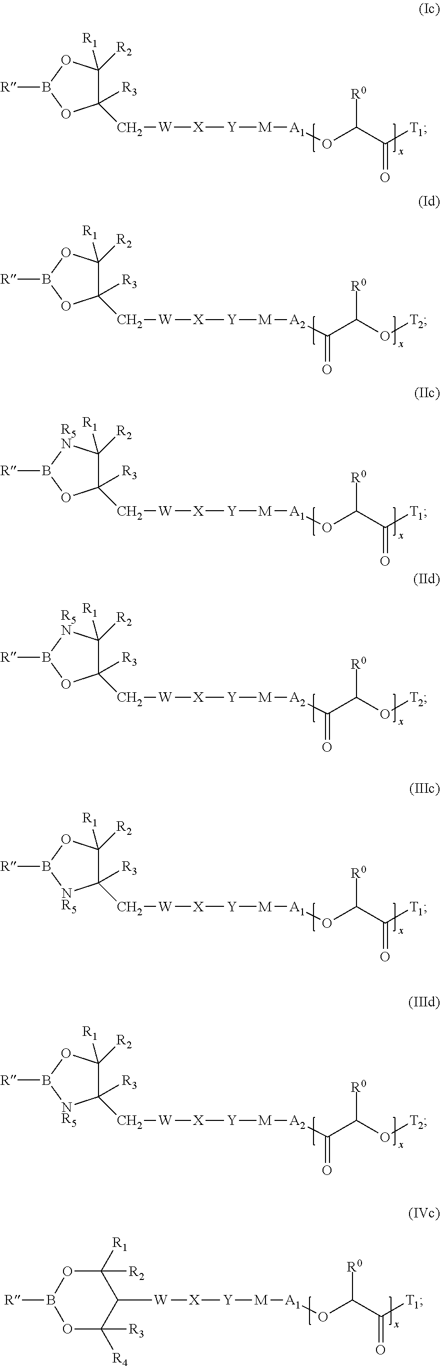

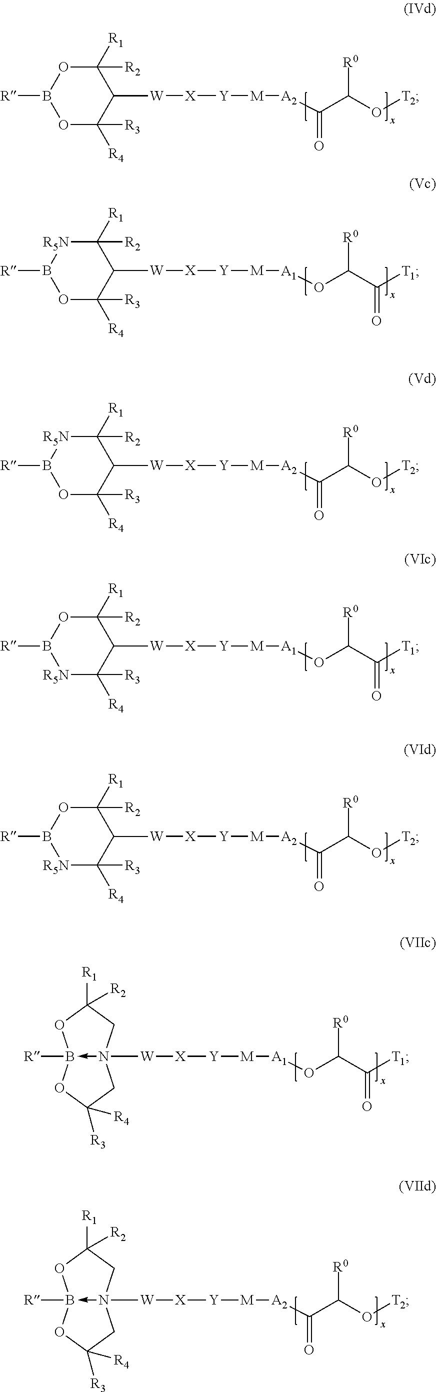

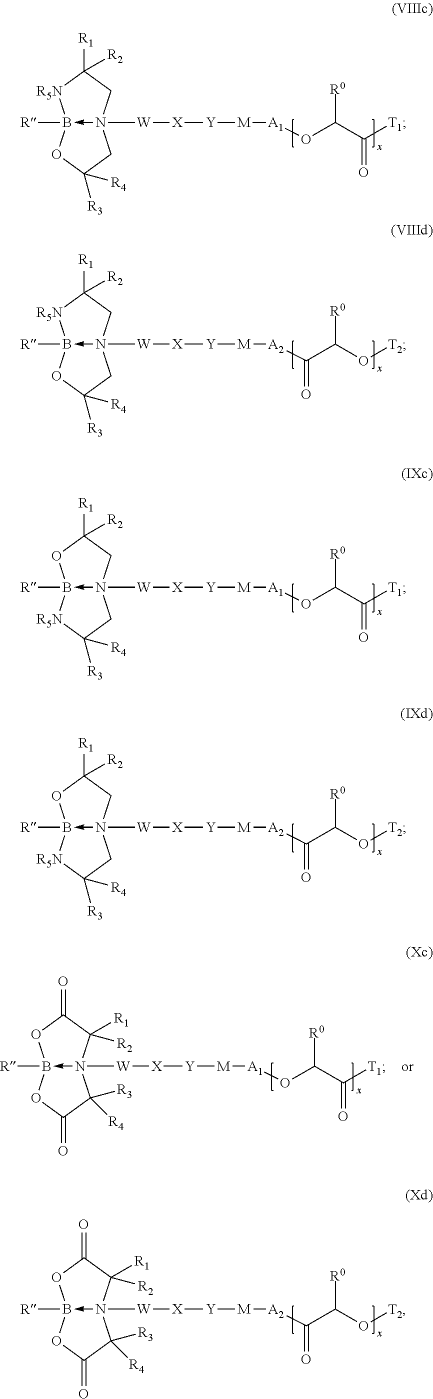

wherein M is a bond or —(CH₂)ᵣ—; r is an integer from 1 to 10; and the remainder of the variables are as described above in the 4^{th} embodiment. In one embodiment, r is 2, 3, 4 or 5.

In a 6^{th} embodiment, for polymer-anticancer agent conjugates described in the 2^{nd} embodiment and polymer-anticancer agent conjugates of structural formulas (Ia)-(Xa) in the 4^{th} embodiment, the linker (i.e., —W—X—Y—Z-A₁-) is represented by any one of the following structural formulas:

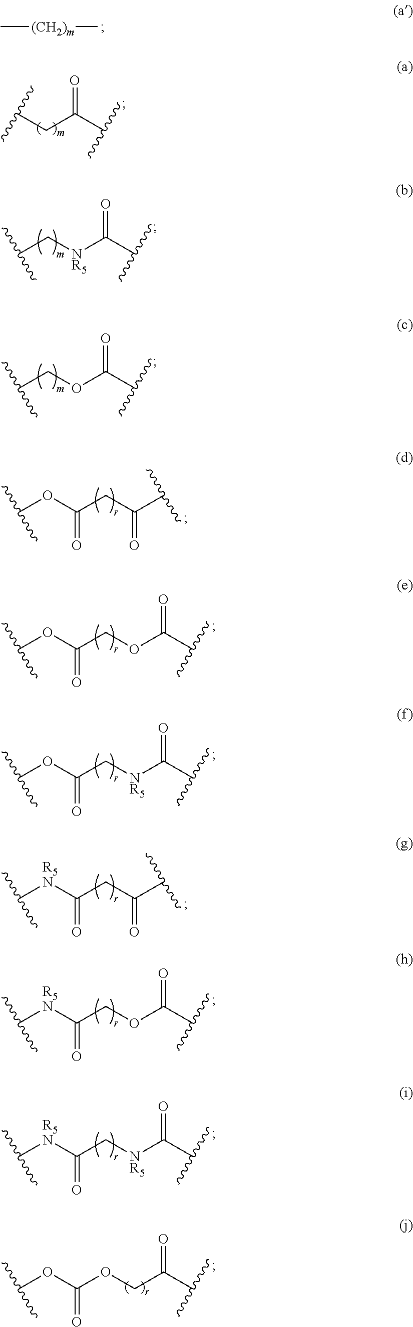

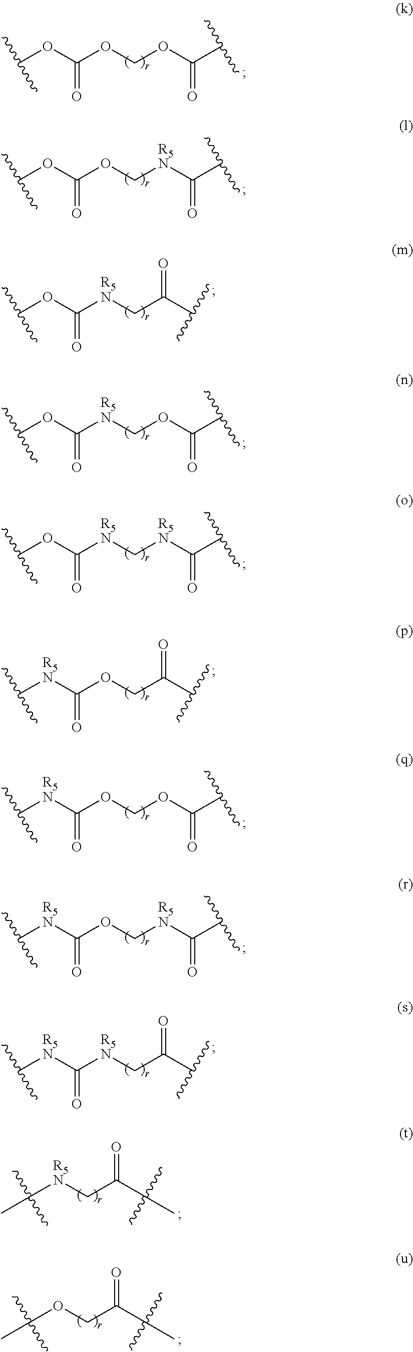

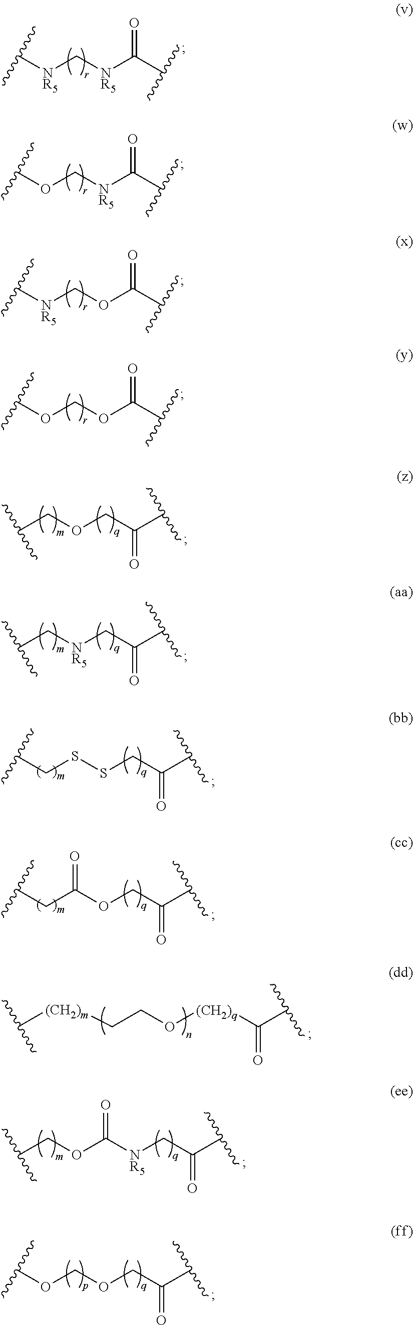

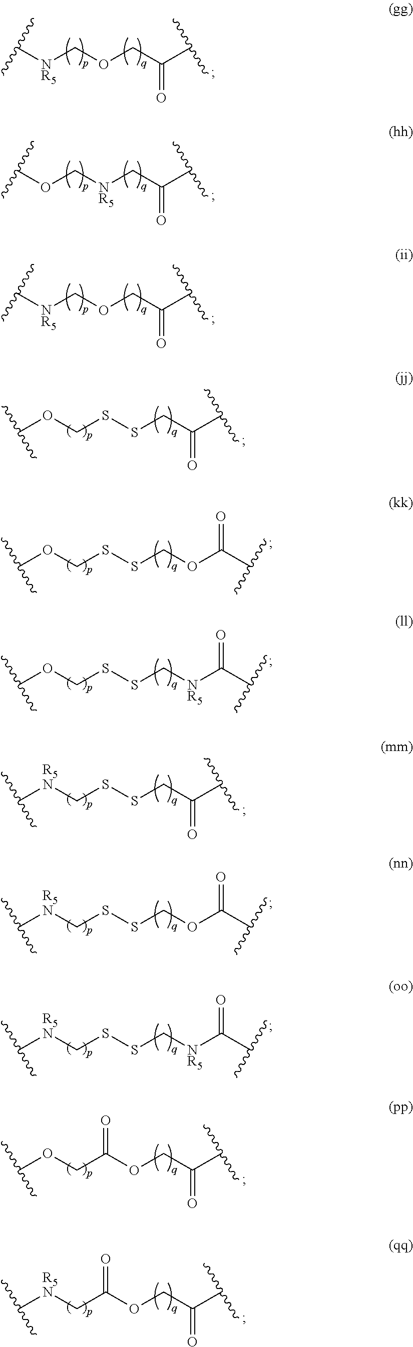

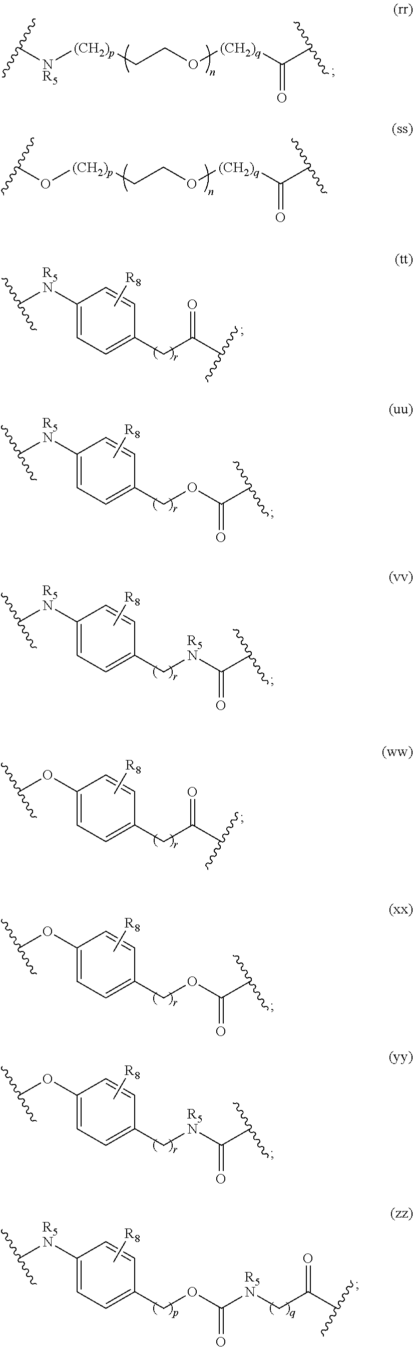

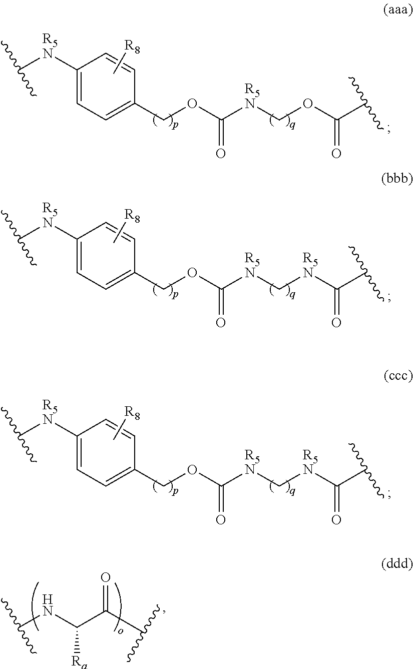

wherein R₈ is a substituent; r is an integer from 1 to 10; m, n, p and q are each an integer from 0 to 10; and o is an integer from 1 to 10. More specifically, R₈ is selected from H, halo, —CN, —NO₂, —OH, (C₁-C₆)alkyl, halo(C₁-C₆)alkyl, hydroxy(C₁-C₆)alkyl, (C₁-C₆)alkoxy, halo(C₁-C₆)alkoxy, (C₁-C₃)alkoxy(C₁-C₃)alkyl and —NR₉R₁₀. R₉ and R₁₀ are each independently H, (C₁-C₆)alkyl, halo(C₁-C₆)alkyl, (C₁-C₆)alkoxy, halo(C₁-C₆)alkoxy, (C₁-C₃)alkoxy(C₁-C₃)alkyl. For structural formulas (k), (l), (n), (o), (q), (r), (v), (w), (x) and (γ), r≧2. For structural formulas (z) and (aa)-(ee), m≧1 and q≧1. For structural formulas (ff)-(oo), p≧2 and q≧1. For structural formulas (pp)-(qq), p≧1 and q≧1. For structural formulas (zz), q≧1. For structural formulas (aaa)-(ccc), q≧2.

In a 7^{th} embodiment, for polymer-anticancer agent conjugates described in 2^{nd} embodiment and polymer-anticancer agent conjugates of structural formulas (Ia)-(Xa) in the 4^{th} embodiment, the linker (i.e., —W—X—Y—Z-A₁-) is represented by any one of the following structural formulas:

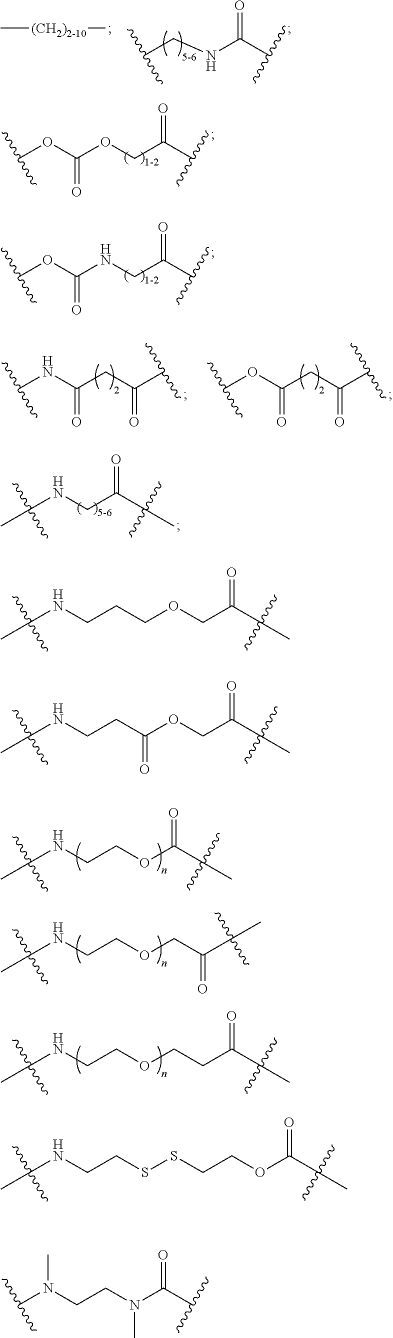

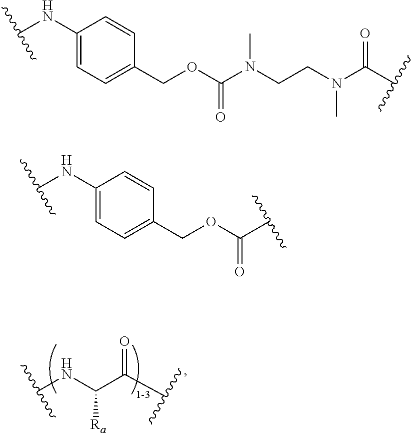

wherein n is an integer from 2 to 5.

In a 8^{th} embodiment, for polymer-anticancer agent conjugates described in 3^{rd} embodiment and polymer-anticancer agent conjugates of structural formulas (Ib)-(Xb) in the 4^{th} embodiment, the linker (i.e., —W—X—Y—Z-A₂-) is represented by any one of the following structural formulas:

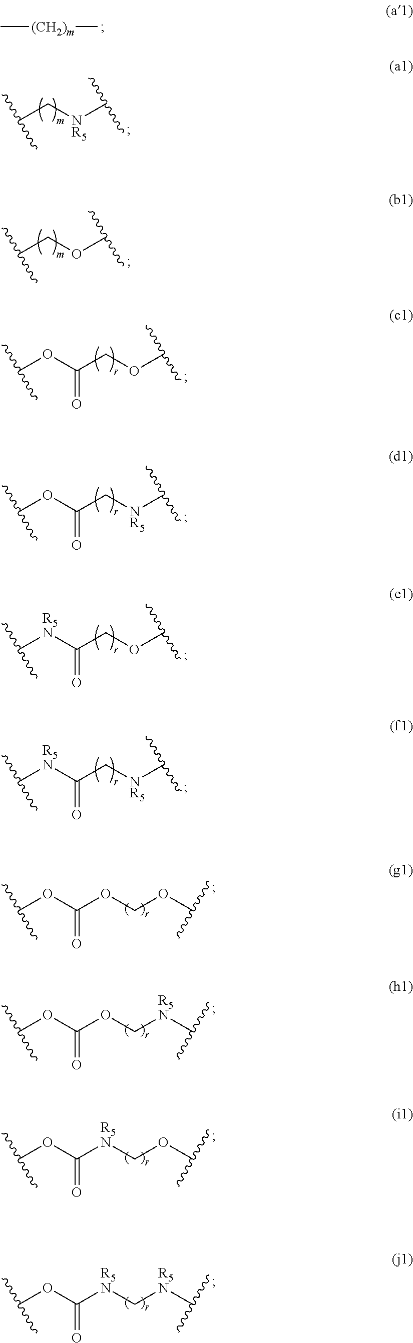

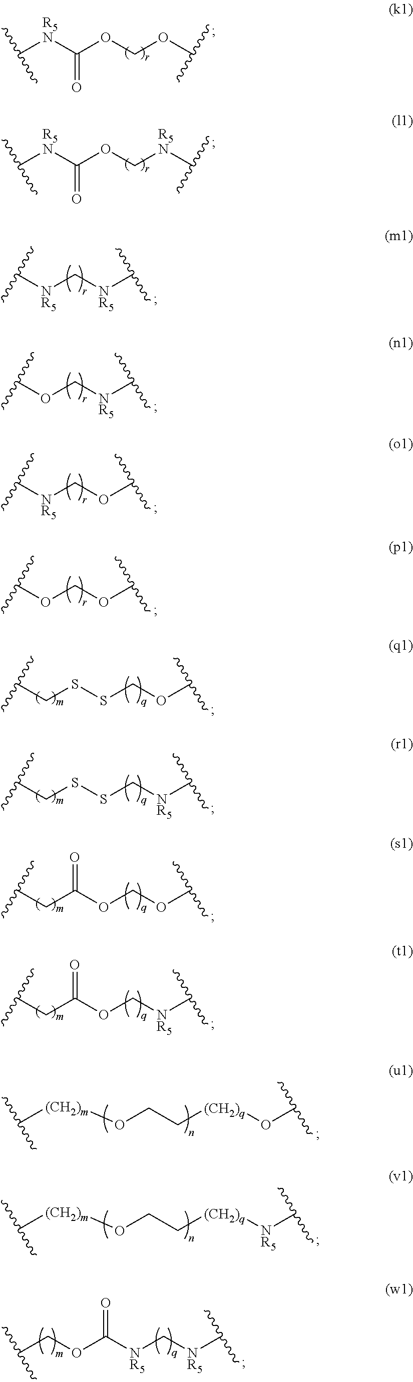

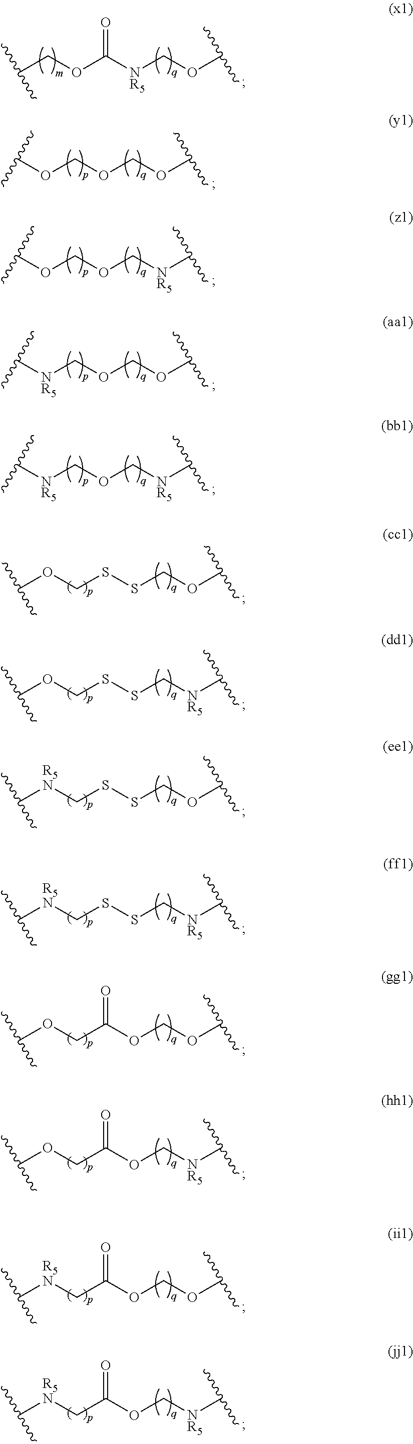

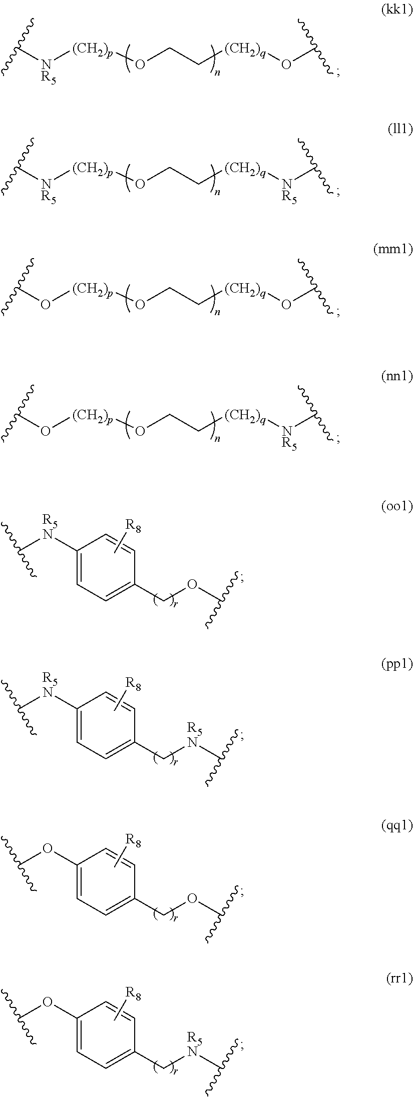

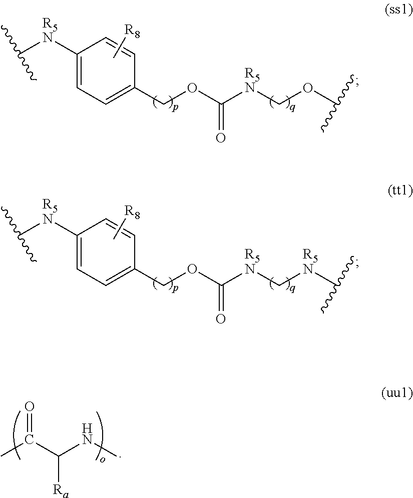

R₈, o, m, n, p, q and r are as described in the 6^{th} embodiment. For structural formulas (g1)-(p1), r≧2. For structural formulas (q1)-(t1), (w1) and (x1), q≧2. For structural formulas (y1), (z1), (aa1)-(ff1), p≧2 and q≧2. For structural formulas (gg1)-(jj1), p≧1 and q≧2. For structural formulas (kk1)-(nn1), p≧2. For structural formulas (ss1)-(tt1), q≧2.⁴³³

In a 9^{th} embodiment, for polymer-anticancer agent conjugates described in 3^{rd} embodiment and polymer-anticancer agent conjugates of structural formulas (Ib)-(Xb) in the 4^{th} embodiment, the linker (i.e., —W—X—Y—Z-A₂-) is —(CH₂)_{w}— (w is an integer from 2-6) or is represented by any one of the following structural formulas:

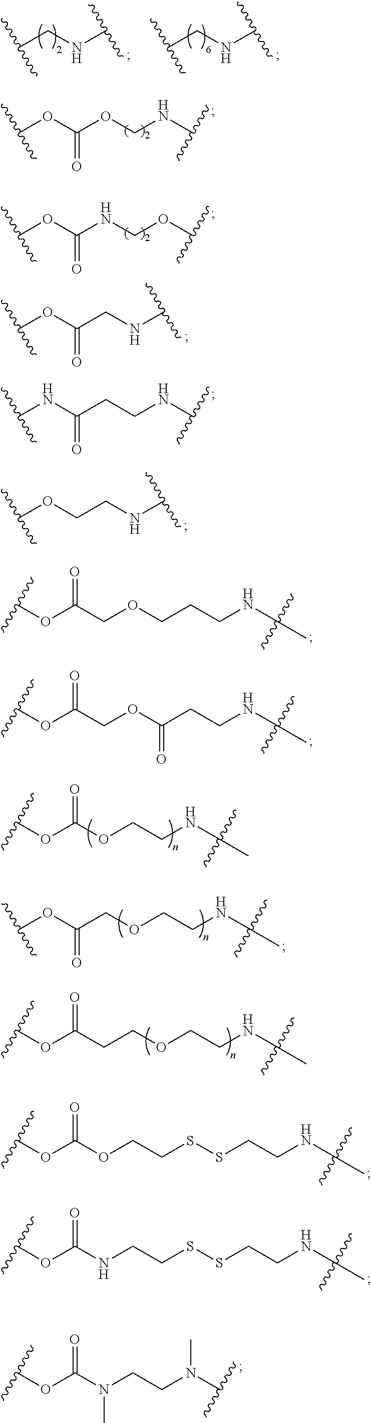

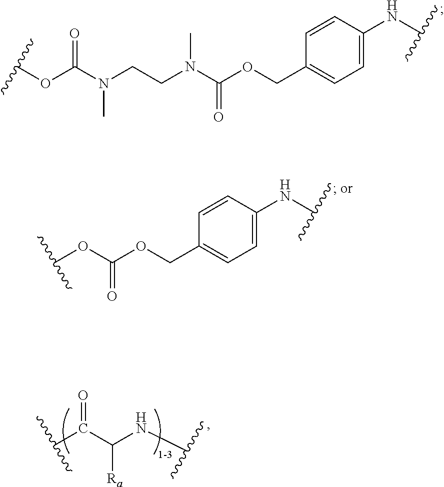

n is an integer from 2 to 5.

In a 10^{th} embodiment, for polymer-anticancer agent conjugate represented by structural formulas (I)-(X), the linker is represented by structural formula (AA) and the polymer comprises a hydroxyl terminal and is attached to the linker at the hydroxyl terminal:

—(CH₂)ₘ—O—CH₂—O—(CH₂)_{q}-A₁ (AA);

wherein A₁ is —C(═O)—, —N(R₅)—C(═O)—, or —O—C(═O)—; and m is an integer from 0 to 10, provided when A₁ is —N(R₅)—C(═O)—, or —O—C(═O)—, then q≧2.

In a 11^{th} embodiment, for polymer-anticancer agent conjugate represented by structural formulas (I)-(X), the linker is represented by structural formula (AA1), (BB 1), (CC1) or (DD1) and the polymer comprises a carboxyl terminal and is attached to the linker at the carboxyl terminal:

—(CH₂)ₘ—O—CH₂—O—(CH₂)_{q}-A₂- (AA1),

—(CH₂)ₘ—O—(CH₂)ₚ—O—CH₂—N(R₅)— (BB1)

—(CH₂)ₘ—(CH₂)ₚ—O—CH₂—N(R₅)— (CC1)

wherein A₂ is —O— or —N(R₅)—; m is an integer from 0 to 10 and q is an integer from 2 to 10; p is an integer from 0 to 10 for structural formula (CC1) and p is an integer from 2 to 10 for structural formula (BB1).

In a 12^{th} embodiment, the polymer-agent conjugate is represented by structural formula (3):

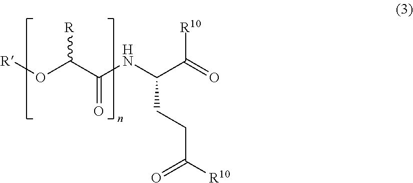

or a pharmaceutically acceptable salt thereof, wherein
each R¹⁰ is independently —OH, -L′-pharmaceutically active agent or;

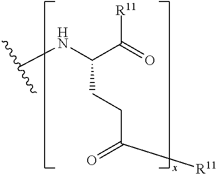

each R¹¹ is —OH or -L′-pharmaceutically active agent, provided that the conjugate comprises at least one -L′-pharmaceutically active agent group;

each x is an integer from 0-5; and

L′ is a linker

Exemplary conjugates of formula (3) are as shown below as formulas (4) and (5):

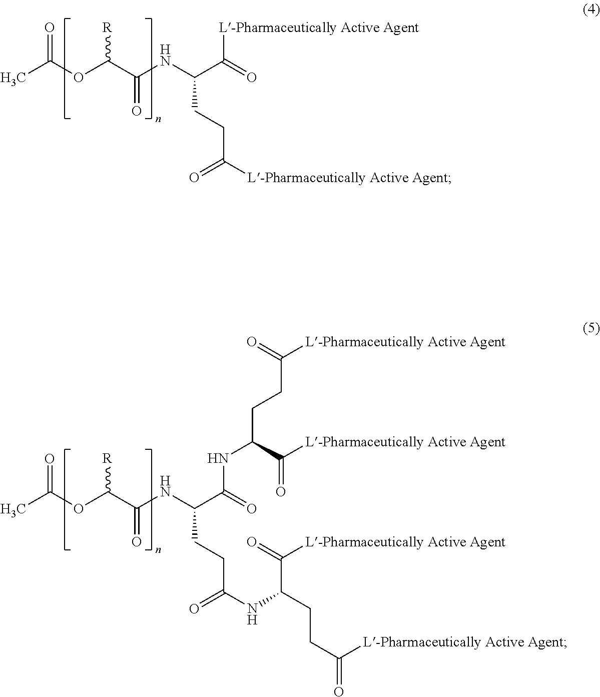

and

wherein each L′ is a linker and the “pharmaceutically active agent” is a pharmaceutically active agent. Exemplary values for each -L′-pharmaceutically active agent are as described for the following structural formulas:

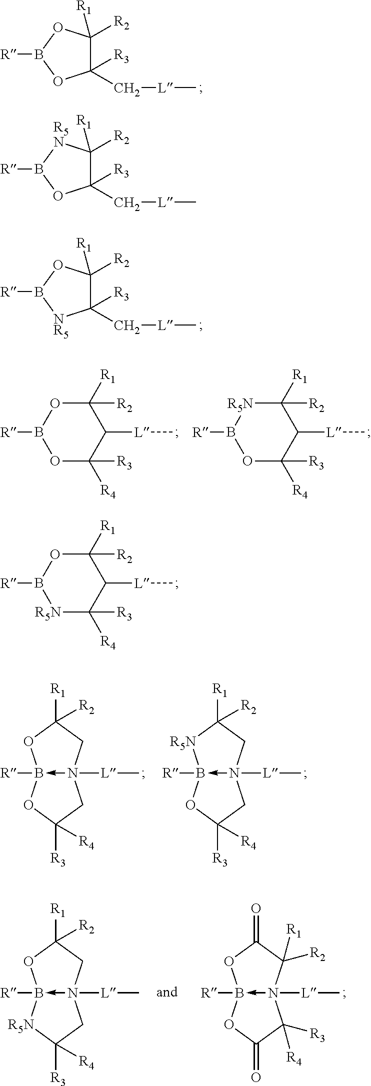

R″ is the residue of the pharmaceutically active agent of Formula (A) or (2A) that is connected to the boronic acid group or the —BZ₁Z₂ group and together with —B(OH)₂ or —BZ₁Z₂ form the boronic acid containing pharmaceutically active agent (or analogue thereof); and L″ is a linker; and R₁, R₂, R₃, R₄ and R₅ are each independently —H or a (C₁-C₅)alkyl. Bortezomib is a preferred pharmaceutically active agent. Exemplary linkers for Formulas (3), (4) and (5) are as described for any one of the third, eighth or ninth embodiments, provided that -L′-pharmaceutically active agent moiety contains no oxygen-oxygen or oxygen-nitrogen bonds. Preferably, bortezomib is the pharmaceutically active agent and the linker represented by L′ is any one of the linker in the third, eighth or ninth embodiments.

An exemplary -L′-pharmaceutically active agent for formulas (3), (4) and (5) is shown below as formula (6) or formula (7):

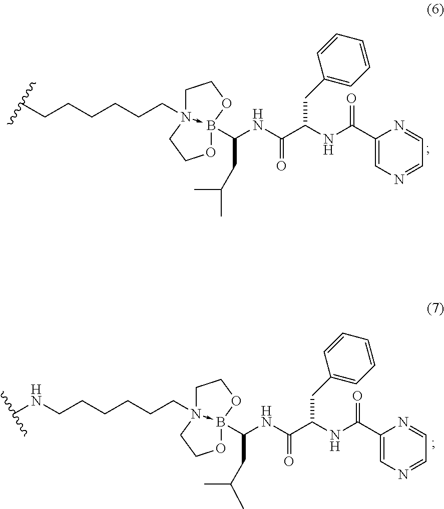 [SMILES: *CCCCCC[N+]12CCO[B-]1([C@H](CC(C)C)NC(=O)[C@H](Cc1ccccc1)NC(=O)c1cnccn1)OCC2 ; *NCCCCCC[N+]12CCO[B-]1([C@H](CC(C)C)NC(=O)[C@H](Cc1ccccc1)NC(=O)c1cnccn1)OCC2]

Optionally, the —(CH₂)₆— linker shown in structural formula (6) can be replaced with any linkers described in the 3^{rd}, 8^{th} and 9^{th} embodiments.

In one embodiment, for polymer-anticancer agent conjugates described in any one of 1^{st} to 12^{th} embodiments, R″B(OH)₂ or its analog is represented by the following structural formula:

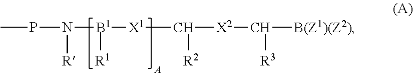

wherein:

P is R or R⁷—C(═O)— or R⁷—SO₂—, wherein R⁷ selected from the group consisting of

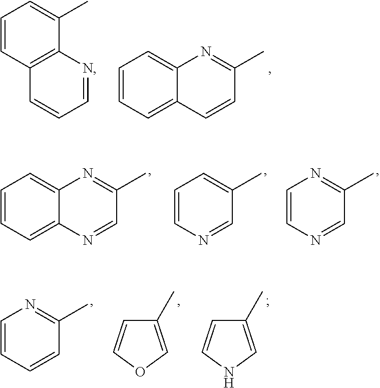

or P is;

X₂ is selected from the group consisting of

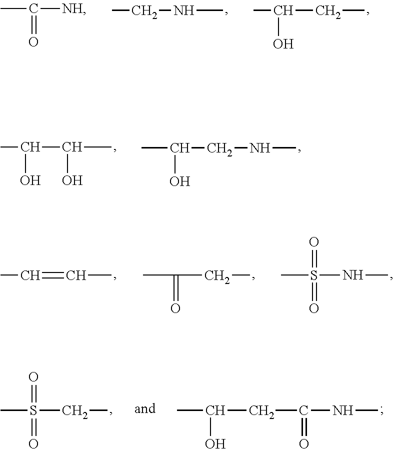

R′ is hydrogen or alkyl;

R₂ and R₃ are independently selected from the group consisting of hydrogen, alkyl, cycloalkyl, aryl, heterocycle and —CH₂—R₅, where R₅ is aryl, aralkyl, alkaryl, cycloalkyl, heterocycle or —Y—R₆,

where Y is a chalcogen, and R₆ is alkyl;

Z₁ and Z₂ are independently alkyl, hydroxy, alkoxy, aryloxy, or together form a dihydroxy compound having at least two hydroxy groups separated by at least two connecting atoms in a chain or ring, said chain or ring comprising carbon atoms, and optionally, a heteroatom or heteroatoms which can be N, S, or O; and A is 0.

In another embodiment, for structural Formula (A):

P is R₇—C(O)— or R₇—SO₂—, where R₇ is pyrazinyl;

X₂ is —C(O)—NH—;

R′ is hydrogen or alkyl;

R₂ and R₃ are independently hydrogen, alkyl, cycloalkyl, aryl, or —CH₂—R₅;

R₅ in each instance, is one of aryl, aralkyl, alkaryl, cycloalkyl, or —W—R₆, where W is a chalcogen and R₆ is alkyl;

where the ring portion of any of said aryl, aralkyl, or alkaryl in R₂, R₃ and R₅ can be optionally substituted by one or two substituents independently selected from the group consisting of C₁₋₆ alkyl, C₃₋₈ cycloalkyl, C₁₋₆ alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₆₋₁₀ aryl, C₁₋₆alkyl(C₆₋₁₀) aryl, and halo(C₆₋₁₀)aryl;

Z₁ and Z₂ are independently one of hydroxy, alkoxy, or aryloxy, or together Z₁ and Z₂ form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or O; and

A is zero.

In another embodiment, for Formula (A):

P is R⁷—C(O)—, where R⁷ is heteroaryl or heteroarylalkyl;

X² is —C(O)—NH—;

R′ is hydrogen or alkyl, or R′ forms together with the adjacent R¹, or when A is zero, forms together with the adjacent R², a nitrogen-containing mono-, bi- or tri-cyclic, saturated or partially saturated ring system having 4-14 ring members, that can be optionally substituted by one or two of keto, hydroxy, alkyl, aryl, aralkyl, alkoxy or aryloxy;

R¹, at each occurrence, is independently one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted; R² is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;

R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;

R⁵, in each instance, is one of aryl, aralkyl, alkaryl, cycloalkyl, a 5-10 membered saturated, partially unsaturated or aromatic heterocycle or —W—R⁶, where W Is a chalcogen; and

R⁶ is alkyl, where the ring portion of any of said aryl, aralkyl, alkaryl or heterocycle can be optionally substituted;

Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or 0; and

A is zero.

In another embodiment, for Formula (A):

P is hydrogen or an amino group protecting moiety;

A is zero;

X² is —C(O)—NH—;

R′ is hydrogen or C₁₋₈ alkyl;

R² is —CH₂—R⁵;

R³ is C₄ alkyl;

R⁵ is aryl or cycloalkyl, wherein R⁵ is optionally substituted by one or two substituents independently selected from the group consisting of C₁₋₆ alkyl, C₃₋₈ cycloalkyl, C₁₋₆ alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₁₋₆ alkyl(C₆₋₁₀)aryl, and halo(C₆₋₁₀)aryl;

Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or 0; and

A is zero.

In another embodiment for the compound of Formula (A):

P is hydrogen or an amino-group protecting moiety;

R′ is hydrogen or alkyl;

A is 0, 1, or 2;

R¹, R², and R³ are each independently hydrogen, alkyl, cycloalkyl, aryl, or —CH₂—R⁵; R⁵, in each instance, is aryl, aralkyl, alkaryl, cycloalkyl, heterocyclyl, heteroaryl, or —W—R⁶, where W is a chalcogen and R⁶ is alkyl;

wherein the ring portion of any said aryl, aralkyl, alkaryl, cycloalkyl, heterocyclyl, or heteroaryl in R¹, R², R³, or R⁵ can be optionally substituted; and

Z¹ and Z² together form a moiety derived from a sugar, wherein the atom attached to boron in each case is an oxygen atom.

In another embodiment for the compound of Formula (A):

P is R′ or R⁷—C(═O)— or R⁷—SO₂—, wherein R⁷ selected from the group consisting of

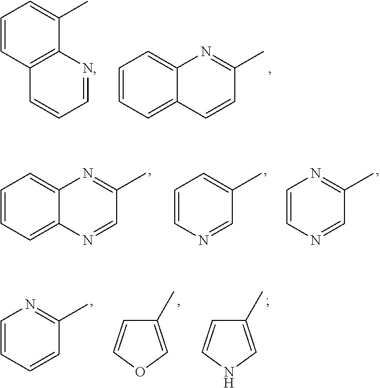

or P is

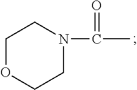

X₂ is selected from the group consisting of

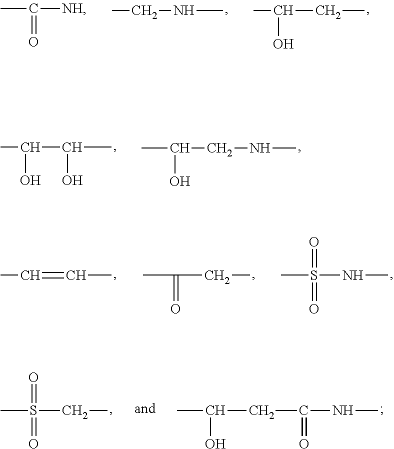

R′ is hydrogen or alkyl;

R₂ and R₃ are independently selected from the group consisting of hydrogen, alkyl, cycloalkyl, aryl, heterocycle and —CH₂—R₅, where R₅ is aryl, aralkyl, alkaryl, cycloalkyl, heterocycle or —Y—R₆,

where Y is a chalcogen, and R₆ is alkyl; and

Z₁ and Z₂ are independently alkyl, hydroxy, alkoxy, aryloxy, or together form a dihydroxy compound having at least two hydroxy groups separated by at least two connecting atoms in a chain or ring, said chain or ring comprising carbon atoms, and optionally, a heteroatom or heteroatoms which can be N, S, or O.

In another embodiment for the compound of Formula (A):

P is R₇—C(O)— or R₇—SO₂—, where R₇ is pyrazinyl;

X₂ is —C(O)—NH—;

R′ is hydrogen or alkyl;

R₂ and R₃ are independently hydrogen, alkyl, cycloalkyl, aryl, or —CH₂—R₅;

R₅ in each instance, is one of aryl, aralkyl, alkaryl, cycloalkyl, or —W—R₆, where W is a chalcogen and R₆ is alkyl;

where the ring portion of any of said aryl, aralkyl, or alkaryl in R₂, R₃ and R₅ can be optionally substituted by one or two substituents independently selected from the group consisting of C₁₋₆ alkyl, C₃₋₈ cycloalkyl, C₁₋₆ alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₆₋₁₀ aryl, C₁₋₆ alkyl(C₆₋₁₀) aryl, and halo(C₆₋₁₀)aryl;

A is zero; and

Z₁ and Z₂ are independently one of hydroxy, alkoxy, or aryloxy, or together Z₁ and Z₂ form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or 0.

In another embodiment, for polymer-anticancer agent conjugates described in any one of 1^{st} to 12^{th} embodiments, R″B(OH)₂ is represented by Formula (2A):

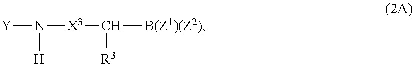

or a pharmaceutically acceptable salts thereof, wherein:

Y is one of R⁸—C(O)—, R⁸—SO₂—, R⁸—NH—C(O)— or R⁸—O—C(O)—, where R⁸ is one of alkyl, aryl, alkaryl, aralkyl, any of which can be optionally substituted, or when Y is R⁸—C(O)— or R⁸—SO₂—, then R⁸ can also be an optionally substituted 5-10 membered, saturated, partially unsaturated or aromatic heterocycle;

X³ is a covalent bond or —C(O)—CH₂—;

R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;

R⁵, in each instance, is one of aryl, aralkyl, alkaryl, cycloalkyl, a 5-10 membered saturated, partially unsaturated or aromatic heterocycle or —W—R⁶, where W is a chalcogen and R⁶ is alkyl, where the ring portion of any of said aryl, aralkyl, alkaryl or heterocycle can be optionally substituted; and

Z¹ and Z² are independently alkyl, hydroxy, alkoxy, aryloxy, or together form a moiety derived from dihydroxy compound having at least two hydroxy groups separated by at least two connecting atoms in a chain or ring, said chain or ring comprising carbon atoms, and optionally, a heteroatom or heteroatoms which can be N, S, or O;

provided that when Y is R⁸—C(O)—, R⁸ is other than phenyl, benzyl or C₁-C₃ alkyl.

In another embodiment, for polymer-anticancer agent conjugates described in any one of 1^{st} to 12^{th} embodiments, R″B(OH)₂ and its analogs are as described in U.S. Pat. Nos. 5,780,454, 6,083,903, 6,297,217, 6,617,317, 6,713,446, 6,747,150, 6,958,319, 7,119,080, 7,582,621, 7,465,836, 7,393,856, and 7,390,806, and U.S. Published Applications US2009/0239824, US2009/0227541, US2008/0293675, US2007/0286822, US2007/0265226, US2007/0179296, US2007/0155699 and US2006/0234981, all of which are incorporated by reference.

In another embodiment, for polymer-anticancer agent conjugates described in any one of 1^{st} to 12^{th} embodiments, R is represented by the following structural formula:

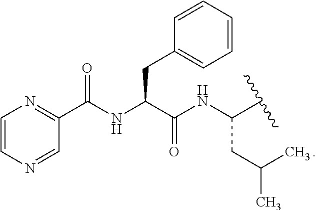 [SMILES: *[C@H](CC(C)C)NC(=O)[C@H](Cc1ccccc1)NC(=O)c1cnccn1]

The present invention is directed to polymer conjugates of proteasome inhibitors and boronic acid containing drugs (particles and composition comprising the same) including drugs that are both proteasome inhibitors and contain boronic acid groups. The boronic acid containing drugs are represented herein by R″—B(OH)₂. The conjugate is formed by reaction of the boronic acid group with a reactive functional group on the polymer to form covalent linkages, typically boronic esters or amides. Therefore, after the conjugate is formed, the pharmaceutically active moiety no longer contains a boronic acid, but is still present as the residue of the boronic acid containing drug R″—B<, wherein “<” indicates that the boron atom contains two bonds to the polymer. The boronic acid containing drug can then be released in vivo by, for example, hydrolysis of the boronic ester and/or boronic amide linkages. For ease of reference in the subject application and in the 1^{st} through 12^{th} embodiments, the variable “R” is defined in terms of the boronic acid containing drug of which it is a part, i.e., “R” is the residue of the boronic acid containing drug R″—B—(OH)₂. By way of example, the “R” group corresponding to the drugs of formulas (A) and (2A) is shown below as formulas (A′) and (2A′), respectively:

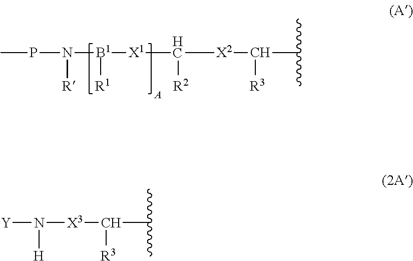

As such, “R” is the portion of the boronic acid containing drug that is attached to the boron atom of the drug and together with the boronic acid group forms the entire drug molecule

In some embodiments, the polymer described in the 1^{st}, 2^{nd}, 3^{rd} or 11^{th} or 12^{th} embodiment is a biogradable polymer (e.g., polylactic acid (PLA), polyglycolic acid (PGA), poly(lactic-co-glycolic acid) (PLGA), polycaprolactone (PCL), polydioxanone (PDO), polyanhydrides, polyorthoesters, or chitosan). In some embodiments, the polymer is a hydrophobic polymer. In some embodiments, the polymer is PLA. In some embodiments, the polymer is PGA.

In some embodiments, the polymer described in the 1^{st}, 2^{nd}, 3^{rd} or 11^{th} or 12^{th} embodiment is a copolymer of lactic and glycolic acid (poly(lactic-co-glycolic acid) (PLGA)). In some embodiments, the polymer is a PLGA-ester. In some embodiments, the polymer is a PLGA-lauryl ester. In some embodiments, the polymer comprises a terminal free acid prior to conjugation to an agent. In some embodiments, the polymer comprises a terminal acyl group (e.g., an acetyl group). In some embodiments, the ratio of lactic acid monomers to glycolic acid monomers is from about 0.1:99.9 to about 99.9:0.1. In some embodiments, the ratio of lactic acid monomers to glycolic acid monomers is from about 75:25 to about 25:75 (e.g., about 50:50 or about 75:25).

In some embodiments, the average molecular weight of the polymer in any one of the 1^{st} to 12^{th} embodiment is from about 1 kDa to about 20 kDa (e.g., from about 1 kDa to about 15 kDa, from about 2 kDa to about 12 kDa, from about 6 kDa to about 20 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, or about 9 kDa). In some embodiments, the polymer has a glass transition temperature of about 20° C. to about 60° C. In some embodiments, the polymer has a polymer polydispersity index of less than or equal to about 2.5 (e.g., less than or equal to about 2.2, or less than or equal to about 2.0).

In some embodiments, the polymer in the 1^{st}, 2^{nd}, 3^{rd} or 11^{th} or 12^{th} embodiment has a hydrophilic portion and a hydrophobic portion. In some embodiments, the polymer is a block copolymer. In some embodiments, the polymer comprises two regions, the two regions together being at least about 70% by weight of the polymer (e.g., at least about 80%, at least about 90%, at least about 95%). In some embodiments, the polymer is a block copolymer comprising a hydrophobic polymer and a hydrophilic polymer. In some embodiments, the polymer, e.g., a diblock copolymer, comprises a hydrophobic polymer and a hydrophilic polymer. In some embodiments, the polymer, e.g., a triblock copolymer, comprises a hydrophobic polymer, a hydrophilic polymer and a hydrophobic polymer.

In some embodiments, the hydrophobic portion of the polymer in the 1^{st}, 2^{nd}, 3^{rd} or 11^{th} or 12^{th} embodiment is a biodegradable polymer (e.g., PLA, PGA, PLGA, PCL, PDO, polyanhydrides, polyorthoesters, or chitosan). In some embodiments, the hydrophobic portion of the polymer is PLA. In some embodiments, the hydrophobic portion of the polymer is PGA. In some embodiments, the hydrophobic portion of the polymer is a copolymer of lactic and glycolic acid (e.g., PLGA).

In some embodiments, the hydrophilic portion of the polymer 1^{st}, 2^{nd}, 3^{rd} or 11^{th} or 12^{th} embodiment is polyethylene glycol (PEG). In some embodiments, the hydrophilic portion of the polymer has a molecular weight of from about 1 kDa to about 20 kDa (e.g., from about 1 kDa to about 15 kDa, from about 2 kDa to about 12 kDa, from about 6 kDa to about 20 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, or about 9 kDa). In some embodiments, the ratio of molecular weight of the hydrophilic to hydrophobic portions of the polymer is from about 1:20 to about 1:1 (e.g., about 1:10 to about 1:1, about 1:2 to about 1:1, or about 1:6 to about 1:3).

In some embodiments, the hydrophilic portion of the polymer 1^{st}, 2^{nd}, 3^{rd} or 11^{th} or 12^{th} embodiment terminates in a hydroxyl moiety prior to conjugation to an agent. In some embodiments, the hydrophilic portion of the polymer terminates in an alkoxy moiety. In some embodiments, the hydrophilic portion of the polymer is a methoxy PEG (e.g., a terminal methoxy PEG).

In some embodiments, the polymer-agent conjugate or polymer-anticancer agent conjugate described above can be the first polymer or the second polymer in a particle described herein. In some embodiments, the polymer-agent conjugate or polymer-anticancer agent conjugate described above can be the first polymer or the second polymer in a nanoparticle described herein.

### Compositions of Polymer-Agent Conjugates

Compositions of polymer-agent conjugates described above may include mixtures of products. For example, the conjugation of an agent to a polymer may proceed in less than 100% yield, and the composition comprising the polymer-agent conjugate may thus also include unconjugated polymer.

Compositions of polymer-agent conjugates may also include polymer-agent conjugates that have the same polymer and the same agent, and differ in the nature of the linkage between the agent and the polymer. For example, in some embodiments, when the agent is a taxane, the composition may include polymers attached to the agent via different hydroxyl groups present on the agent. In the case of paclitaxel, the composition may include polymers attached to paclitaxel via the hydroxyl group at the 2′ position, polymers attached to paclitaxel via the hydroxyl group at the 7 position, and/or polymers attached to paclitaxel via the hydroxyl group at the 1 position. In the case of docetaxel, the composition may include polymers attached to docetaxel via the hydroxyl group at the 2′ position, polymers attached to docetaxel via the hydroxyl group at the 7 position, polymers attached to docetaxel via the hydroxyl group at the 10 position and/or polymers attached to docetaxel via the hydroxyl group at the 1 position. The polymer-agent conjugates may be present in the composition in varying amounts. For example, when an agent having a plurality of available attachment points (e.g., taxane) is reacted with a polymer, the resulting composition may include more of a product conjugated via a more reactive hydroxyl group, and less of a product attached via a less reactive hydroxyl group.

Additionally, compositions of polymer-agent conjugates may include agents that are attached to more than one polymer chain. For example, in the case of paclitaxel, the composition may include: paclitaxel attached to one polymer chain via the hydroxyl group at the 2′ position and a second polymer chain via the hydroxyl group at the 7 position; paclitaxel attached to one polymer chain via the hydroxyl group at the 2′ position and a second polymer chain via the hydroxyl group at the 10 position; paclitaxel attached to one polymer chain via the hydroxyl group at the 7 position and a second polymer chain via the hydroxyl group at the 10 position; and/or paclitaxel attached to one polymer chain via the hydroxyl group at the 2′ position; a second polymer chain via the hydroxyl group at the 7 position and a third polymer chain via the hydroxyl group at the 10 position. In the case of docetaxel, the composition may include: docetaxel attached to one polymer chain via the hydroxyl group at the 2′ position and a second polymer chain via the hydroxyl group at the 7 position; docetaxel attached to one polymer chain via the hydroxyl group at the 2′ position and a second polymer chain via the hydroxyl group at the 10 position; docetaxel attached to one polymer chain via the hydroxyl group at the 2′ position and a second polymer chain via the hydroxyl group at the 1 position; docetaxel attached to one polymer chain via the hydroxyl group at the 7 position and a second polymer chain via the hydroxyl group at the 10 position; docetaxel attached to one polymer chain via the hydroxyl group at the 7 position and a second polymer chain via the hydroxyl group at the 1 position; docetaxel attached to one polymer chain via the hydroxyl group at the 10 position and a second polymer chain via the hydroxyl group at the 1 position; docetaxel attached to one polymer chain via the hydroxyl group at the 2′ position, a second polymer chain via the hydroxyl group at the 7 position and a third polymer chain via the hydroxyl group at the 10 position; docetaxel attached to one polymer chain via the hydroxyl group at the 2′ position, a second polymer chain via the hydroxyl group at the 10 position and a third polymer chain via the hydroxyl group at the 1 position; docetaxel attached to one polymer chain via the hydroxyl group at the 2′ position, a second polymer chain via the hydroxyl group at the 7 position and a third polymer chain via the hydroxyl group at the 1 position; docetaxel attached to one polymer chain via the hydroxyl group at the 7 position, a second polymer chain via the hydroxyl group at the 10 position and a third polymer chain via the hydroxyl group at the 1 position; and/or docetaxel attached to one polymer chain via the hydroxyl group at the 2′ position, a second polymer chain via the hydroxyl group at the 7 position, a third polymer chain via the hydroxyl group at the 10 position and a fourth polymer chain via the hydroxyl group at the 1 position.

### Particles

In general, a particle described herein includes a hydrophobic polymer, a polymer containing a hydrophilic portion and a hydrophobic portion, and one or more agents (e.g., therapeutic or diagnostic agents). In some embodiments, an agent may be attached to a polymer (e.g., a hydrophobic polymer or a polymer containing a hydrophilic and a hydrophobic portion), and in some embodiments, an additional agent may be embedded in the particle. In some embodiments, an agent may not be attached to a polymer and may be embedded in the particle. The additional agent may be the same as the agent attached to a polymer, or may be a different agent. A particle described herein may also include a compound having at least one acidic moiety, such as a carboxylic acid group. The compound may be a small molecule or a polymer having at least one acidic moiety. In some embodiments, the compound is a polymer such as PLGA. A particle described herein may also include one or more excipients, such as surfactants, stabilizers or lyoprotectants. Exemplary stabilizers or lyoprotectants include carbohydrates (e.g., a carbohydrate described herein, such as, e.g., sucrose, cyclodextrin or a derivative of cyclodextrin (e.g. 2-hydroxypropyl-β-cyclodextrin)), salt, PEG, PVP, crown either or polyol (e.g., trehalose, mannitol, sorbitol or lactose).

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

A composition of a plurality of particles described herein may have an average diameter of about 50 nm to about 500 nm (e.g., from about 50 nm to about 200 nm). A composition of a plurality of particles particle may have a median particle size (Dv50) is from about 50 nm to about 220 nm (e.g., from about 75 nm to about 200 nm). A composition of a plurality of particles particle may have a Dv90 (particle size below which 90% of the volume of particles exists) of about 50 nm to about 500 nm (e.g., about 75 nm to about 220 nm).

A particle described herein may have a surface zeta potential ranging from about −80 mV to about 50 mV, when measured in water. Zeta potential is a measurement of surface potential of a particle. In some embodiments, a particle may have a surface zeta potential, when measured in water, ranging between about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., 0 to −20 mV.

A particle described herein may include a small amount of a residual solvent, e.g., a solvent used in preparing the particles such as acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate. In some embodiments, the particle may include less than 5000 ppm of a solvent (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

A particle described herein may include varying amounts of a hydrophobic polymer, e.g., from about 20% to about 90% (e.g., from about 20% to about 80%, from about 25% to about 75%, or from about 30% to about 70%). A particle described herein may include varying amounts of a polymer containing a hydrophilic portion and a hydrophobic portion, e.g., up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%.

A particle described herein may be substantially free of a targeting agent (e.g., of a targeting agent covalently linked to the particle, e.g., to the first or second polymer or agent), e.g., a targeting agent able to bind to or otherwise associate with a target biological entity, e.g., a membrane component, a cell surface receptor, prostate specific membrane antigen, or the like. A particle described herein may be substantially free of a targeting agent that causes the particle to become localized to a tumor, a disease site, a tissue, an organ, a type of cell, e.g., a cancer cell, within the body of a subject to whom a therapeutically effective amount of the particle is administered. A particle described herein may be substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer within the particle is conjugated to a targeting moiety. In an embodiment substantially free of a targeting agent means substantially free of any moiety other than the first polymer, the second polymer, a third polymer (if present), a surfactant (if present), and the agent, e.g., an anti-cancer agent or other therapeutic or diagnostic agent, that targets the particle. Thus, in such embodiments, any contribution to localization by the first polymer, the second polymer, a third polymer (if present), a surfactant (if present), and the agent is not considered to be “targeting.” A particle described herein may be free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. A particle described herein may be substantially free of an amphiphilic layer that reduces water penetration into the nanoparticle. A particle described herein may comprise less than 5 or 10% (e.g., as determined as w/w, v/v) of a lipid, e.g., a phospholipid. A particle described herein may be substantially free of a lipid layer, e.g., a phospholipid layer, e.g., that reduces water penetration into the nanoparticle. A particle described herein may be substantially free of lipid, e.g., is substantially free of phospholipid.

A particle described herein may be substantially free of a radiopharmaceutical agent, e.g., a radiotherapeutic agent, radiodiagnostic agent, prophylactic agent, or other radioisotope. A particle described herein may be substantially free of an immunomodulatory agent, e.g., an immunostimulatory agent or immunosuppressive agent. A particle described herein may be substantially free of a vaccine or immunogen, e.g., a peptide, sugar, lipid-based immunogen, B cell antigen or T cell antigen.

A particle described herein may be substantially free of a water-soluble hydrophobic polymer such as PLGA, e.g., PLGA having a molecular weight of less than about 1 kDa.

In a particle described herein, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

Methods of Making Particles and Compositions

A particle described herein may be prepared using any method known in the art for preparing particles, e.g., nanoparticles. Exemplary methods include spray drying, emulsion (e.g., emulsion-solvent evaporation or double emulsion), precipitation (e.g., nanoprecipitation) and phase inversion.

In one embodiment, a particle described herein can be prepared by precipitation (e.g., nanoprecipitation). This method involves dissolving the components of the particle (i.e., one or more polymers, an optional additional component or components, and an agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In one set of embodiments, the particles are formed by providing one or more solutions containing one or more polymers and additional components, and contacting the solutions with certain solvents to produce the particle. In a non-limiting example, a hydrophobic polymer (e.g., PLGA), is conjugated to an agent to form a conjugate. This polymer-agent conjugate, a polymer containing a hydrophilic portion and a hydrophobic portion (e.g., PEG-PLGA), and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate a particle described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and active agent dissolved in a water-miscible solvent is mixed at high velocity with another jet stream containing water, an anti-solvent for the active agent and the hydrophobic block of the copolymer. The fast mixing and high energy dissipation involved in this process provide timescales that are shorter than the timescale for nucleation and growth of particles, which leads to the formation of nanoparticles with active agent loading contents and size distributions not provided by other technologies. When forming the nanoparticles via flash nanoprecipitation, mixing occurs fast enough to allow high supersaturation levels of all components to be reached prior to the onset of aggregation. Therefore, the active agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A particle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

A particle described herein may also be prepared using Microfluidics Reaction Technology (MRT). At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

A particle described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

After the particles are prepared, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See, e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

After purification of the particles, they may be sterile filtered (e.g., using a 0.22 micron filter) while in solution.

In certain embodiments, the particles are prepared to be substantially homogeneous in size within a selected size range. The particles are preferably in the range from 30 nm to 300 nm in their greatest diameter, (e.g., from about 30 nm to about 250 nm). The particles may be analyzed by techniques known in the art such as dynamic light scattering and/or electron microscopy, (e.g., transmission electron microscopy or scanning electron microscopy) to determine the size of the particles. The particles may also be tested for agent loading and/or the presence or absence of impurities.

Lyophilization

A particle described herein may be prepared for dry storage via lyophilization, commonly known as freeze-drying. Lyophilization is a process which extracts water from a solution to form a granular solid or powder. The process is carried out by freezing the solution and subsequently extracting any water or moisture by sublimation under vacuum. Advantages of lyophilization include maintenance of substance quality and minimization of therapeutic compound degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

Lyophilization may take place in the presence of a lyoprotectant, e.g., a lyoprotectant described herein. In some embodiments, the lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

Methods of Storing

A polymer-agent conjugate, particle or composition described herein may be stored in a container for at least about 1 hour (e.g., at least about 2 hours, 4 hours, 8 hours, 12 hours, 24 hours, 2 days, 1 week, 1 month, 2 months, 3 months, 4 months, 5 months, 6 months, 1 year, 2 years or 3 years). Accordingly, described herein are containers including a polymer-agent conjugate, particle or composition described herein.

A polymer-agent conjugate, particle or composition may be stored under a variety of conditions, including ambient conditions (e.g., at room temperature, ambient humidity, and atmospheric pressure). A polymer-agent conjugate, particle or composition may also be stored at low temperature, e.g., at a temperature less than or equal to about 5° C. (e.g., less than or equal to about 4° C. or less than or equal to about 0° C.). A polymer-agent conjugate, particle or composition may also be frozen and stored at a temperature of less than about 0° C. (e.g., between −80° C. and −20° C.). A polymer-agent conjugate, particle or composition may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

A polymer-agent conjugate, particle or composition described herein may be stored in a variety of containers, including a light-blocking container such as an amber vial. A container may be a vial, e.g., a sealed vial having a rubber or silicone enclosure (e.g., an enclosure made of polybutadiene or polyisoprene). A container may be substantially free of atmospheric oxygen and/or other reactive gases, and/or substantially free of moisture.

Methods of Evaluating Particles

A particle described herein may be subjected to a number of analytical methods. For example, a particle described herein may be subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

In some embodiments, a particle described herein may be evaluated using dynamic light scattering. Particles may be illuminated with a laser, and the intensity of the scattered light fluctuates at a rate that is dependent upon the size of the particles as smaller particles are “kicked” further by the solvent molecules and move more rapidly. Analysis of these intensity fluctuations yields the velocity of the Brownian motion and hence the particle size using the Stokes-Einstein relationship. The diameter that is measured in Dynamic Light Scattering is called the hydrodynamic diameter and refers to how a particle diffuses within a fluid. The diameter obtained by this technique is that of a sphere that has the same translational diffusion coefficient as the particle being measured.

In some embodiments, a particle described herein may be evaluated using cryo scanning electron microscopy (Cryo-SEM). SEM is a type of electron microscopy in which the sample surface is imaged by scanning it with a high-energy beam of electrons in a raster scan pattern. The electrons interact with the atoms that make up the sample producing signals that contain information about the sample's surface topography, composition and other properties such as electrical conductivity. For Cryo-SEM, the SEM is equipped with a cold stage for cryo-microscopy. Cryofixation may be used and low-temperature scanning electron microscopy performed on the cryogenically fixed specimens. Cryo-fixed specimens may be cryo-fractured under vacuum in a special apparatus to reveal internal structure, sputter coated and transferred onto the SEM cryo-stage while still frozen.

In some embodiments, a particle described herein may be evaluated using transmission electron microscopy (TEM). In this technique, a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a charge-coupled device (CCD) camera.

Exemplary Particles

1) Docetaxel-5050-PLGA-O-acetyl PEGylated Nanoparticles

One exemplary nanoparticle includes the polymer-agent conjugate docetaxel-5050-PLGA-O-acetyl, which is a conjugate of PLGA and docetaxel. This conjugate has the formula shown below:

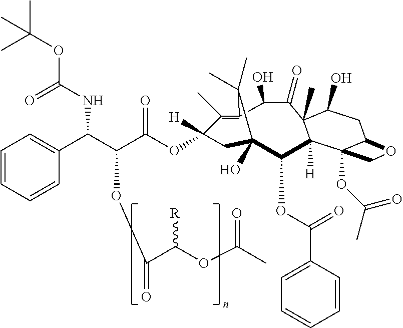

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

The terminal hydroxyl (OH) group of PLGA is acetylated prior to conjugation of docetaxel to the terminal carboxylic acid (COOH) group. Docetaxel is attached to PLGA via an ester bond, primarily via the 2′ hydroxyl group. The product may include docetaxel attached to the polymer via the 2′, 7, 10 and/or 1 positions; and/or docetaxel molecules attached to multiple polymer chains (e.g., via both the 2′ and 7 positions).

The weight loading of docetaxel on the PLGA polymer ranges from 5-16 weight %. This results in a mixture composed of docetaxel-5050 PLGA-O-acetyl and 5050 PLGA-O-acetyl in a ratio ranging from 99:1 to 60:40 weight %. The second component of the particle is thus 5050 PLGA-O-acetyl, having a free —COOH moiety at its terminus. Its structure is represented by the following formula:

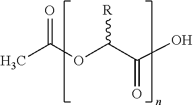

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the docetaxel-5050-PLGA-O-acetyl nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

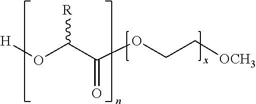

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to docetaxel-5050 PLGA-O-acetyl (preferably about 16 to 40 weight %), giving ratios of 85:15 to 55:45 weight % (preferably 84:16 to 60:40 weight %).

A fourth component of the docetaxel-5050-PLGA-O-acetyl nanoparticles is a surfactant, typically poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

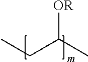

The polymer mixture of docetaxel-5050-PLGA-O-acetyl, 5050 PLGA-O-acetyl and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent) weight/volume. This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is from about 1:1 to about 1:10 volume/volume, preferably about 1:10 percent volume/volume. The resulting mixture contains PEGylated nanoparticles composed of the polymer-drug conjugate, free 5050 PLGA-O-acetyl, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 6.0 mg/mL (e.g., about 1, 2, 3, 4, 5 or 6 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

2) Doxorubicin-5050 PLGA-amide PEGylated Nanoparticles

Another exemplary nanoparticle includes the polymer-agent conjugate doxorubicin-5050 PLGA-amide, which is a conjugate of PLGA and doxorubicin. This conjugate has the formula shown below:

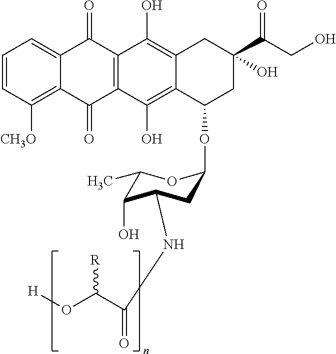

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

Doxorubicin is attached to PLGA via an amide bond. The weight loading of doxorubicin on the PLGA polymer ranges from 8-12 weight %. The conjugation of doxorubicin results in a mixture composed of doxorubicin-5050 PLGA-amide and 5050 PLGA in a ratio ranging from 100:0 to 70:30 weight %. The second component of the particle is thus 5050 PLGA, having a free —COOH moiety at its terminus. Its structure is represented by the following formula:

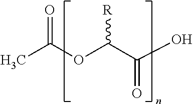

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the doxorubicin-5050 PLGA-amide nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

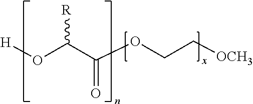

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to docetaxel-5050 PLGA-O-acetyl (preferably about 16 to 40 weight %), giving ratios of 85:15 to 55:45 weight % (preferably 84:16 to 60:40 weight %).

A fourth component of the doxorubicin-5050 PLGA-amide nanoparticles is a surfactant, poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

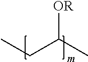

The polymer mixture of doxorubicin-5050 PLGA-amide, 5050 PLGA and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent). This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is from about 1:1 to about 1:10 volume/volume, preferably about 1:10 percent volume/volume. The resulting mixture contains PEGylated nanoparticles composed of the polymer-drug conjugate, free 5050 PLGA-O-acetyl acid, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 6.0 mg/mL (e.g., about 1, 2, 3, 4, 5 or 6 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

3) Paclitaxel-5050-PLGA-O-acetyl PEGylated Nanoparticles

One exemplary nanoparticle includes the polymer-agent conjugate paclitaxel-5050-PLGA-O-acetyl, which is a conjugate of PLGA and paclitaxel. This conjugate has the structure shown below:

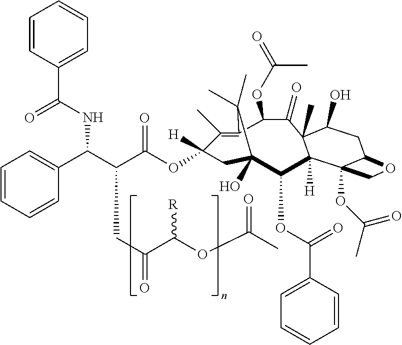

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

The terminal hydroxyl (OH) group of PLGA is acetylated prior to conjugation of paclitaxel to the terminal carboxylic acid (COOH) group. Paclitaxel is attached to PLGA via an ester bond, primarily via the 2′ hydroxyl group. The product may include paclitaxel attached to the polymer via the 2′, 7 and/or 1 positions; and/or paclitaxel molecules attached to multiple polymer chains (e.g., via both the 2′ and 7 positions). The weight loading of paclitaxel on the PLGA polymer ranges from about 5-16 weight %.

The conjugation of paclitaxel to PLGA results in a mixture composed of paclitaxel-5050 PLGA-O-acetyl and free 5050 PLGA-O-acetyl in a ratio ranging from 100:0 to 70:30 weight %. The second component of the particle is thus 5050 PLGA-O-acetyl, having a free —COOH moiety at its terminus. Its structure is represented by the following formula:

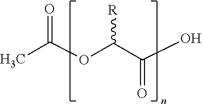

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the paclitaxel-5050-PLGA-O-acetyl nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

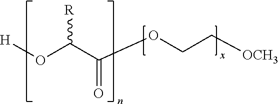

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to docetaxel-5050 PLGA-O-acetyl (preferably about 16 to 40 weight %), giving ratios of 85:15 to 55:45 weight % (preferably 84:16 to 60:40 weight %).

A fourth component of the paclitaxel-5050-PLGA-O-acetyl nanoparticles is surfactant, typically poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

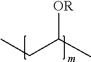

The polymer mixture of paclitaxel-5050-PLGA-O-acetyl, 5050 PLGA-O-acetyl and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent). This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is from about 1:1 to about 1:10 volume/volume, preferably about 1:10 percent volume/volume. The resulting mixture contains PEGylated nanoparticles composed of the polymer-drug conjugate, free 5050 PLGA-O-acetyl acid, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 6.0 mg/mL (e.g., about 1, 2, 3, 4, 5 or 6 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

4) Docetaxel-hexanoate-5050 PLGA-O-acetyl PEGylated Nanoparticles

Another exemplary nanoparticle includes the polymer-agent conjugate docetaxel-hexanoate-5050 PLGA-O-acetyl, which is a conjugate of PLGA and docetaxel with a hexanoate linker. This conjugate has the formula shown below:

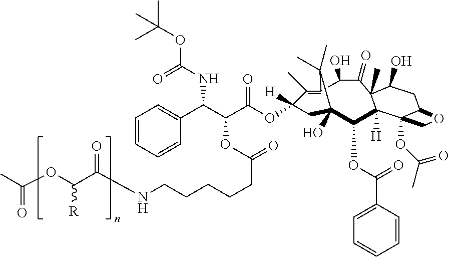

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

There is a hexanoate linker between the PLGA polymer and the drug docetaxel. Docetaxel-hexanoate is attached to the polymer primarily via the 2′ hydroxyl group of docetaxel. The product may include docetaxel-hexanoate attached to the polymer via the 2′, 7, 10 and/or 1 positions; and/or docetaxel-hexanoate molecules attached to multiple polymer chains (e.g., via both the 2′ and 7 positions). The weight loading of docetaxel on the PLGA polymer ranges from 10-11 weight %. The conjugation of docetaxel to PLGA results in a mixture composed of docetaxel-hexanoate-5050 PLGA-O-acetyl and free 5050 PLGA-O-acetyl in a ratio ranging from 100:0 to 70:30 weight %. The second component of the particle is thus 5050 PLGA-O-acetyl, having a free —COOH moiety at its terminus. Its structure is represented by the following formula:

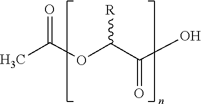

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the docetaxel-hexanoate-5050 PLGA-O-acetyl nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

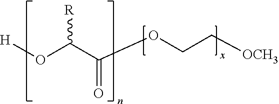

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to docetaxel-5050 PLGA-O-acetyl (preferably about 16 to 40 weight %), giving ratios of 85:15 to 55:45 weight % (preferably 84:16 to 60:40 weight %).

A fourth component of the docetaxel-hexanoate-5050 PLGA-O-acetyl nanoparticles is a surfactant, typically poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

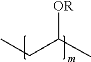

The polymer mixture of docetaxel-hexanoate-5050 PLGA-O-acetyl, 5050 PLGA-O-acetyl and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent). This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is 1:10 percent volume/volume. The resulting mixture contains PEGylated from about 1:1 to about 1:10 volume/volume, preferably about nanoparticles composed of the polymer-drug conjugate, free 5050 PLGA-O-acetyl acid, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 6.0 mg/mL (e.g., about 1, 2, 3, 4, 5 or 6 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

5) Bis(docetaxel) glutamate-5050 PLGA-O-acetyl PEGylated Nanoparticles

Another exemplary nanoparticle includes the polymer-agent conjugate bis(docetaxel) glutamate-5050 PLGA-O-acetyl, which is a conjugate of docetaxel and PLGA, with a bifunctional glutamate linker. This conjugate has the formula shown below:

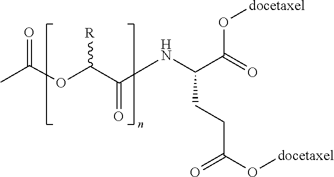

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

Each docetaxel is attached to the glutamate linker via an ester bond, primarily via the 2′ hydroxyl groups. The product may include polymers in which one docetaxel is attached via the hydroxyl group at the 2′ position and the other is attached via the hydroxyl group at the 7 position; one docetaxel is attached via the hydroxyl group at the 2′ position and the other is attached via the hydroxyl group at the 10 position; one docetaxel is attached via the hydroxyl group at the 7 position and the other is attached via the hydroxyl group at the 10 position; and/or polymers in which only one docetaxel is linked to the polymer, via the hydroxyl group at the 2′ position, the hydroxyl group at the 7 position or the hydroxyl group at the 10 position; and/or docetaxel molecules attached to multiple polymer chains (e.g., via both the hydroxyl groups at the 2′ and 7 positions). The weight loading of docetaxel on the PLGA polymer ranges from 10-16 weight %. The conjugation of docetaxel to PLGA results in a mixture composed of bis(docetaxel) glutamate-5050 PLGA-O-acetyl and 5050 PLGA-O-acetyl in a ratio ranging from 100:0 to 70:30 weight %. The second component of the particle is thus 5050 PLGA-O-acetyl, having a free —COOH moiety at its terminus. Its structure is represented by the following formula:

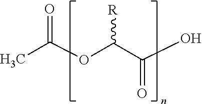

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the bis(docetaxel) glutamate-5050 PLGA-O-acetyl nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

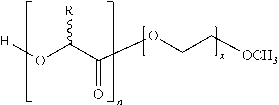

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to docetaxel-5050 PLGA-O-acetyl (preferably about 16 to 40 weight %), giving ratios of 85:15 to 55:45 weight % (preferably 84:16 to 60:40 weight %).

A fourth component of the bis(docetaxel) glutamate-5050 PLGA-O-acetyl nanoparticles is a surfactant, typically poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

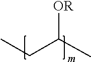

The polymer mixture of bis(docetaxel) glutamate-5050 PLGA-O-acetyl, 5050 PLGA-O-acetyl and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent). This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is from about 1:1 to about 1:10 volume/volume, preferably about 1:10 percent volume/volume. The resulting mixture contains PEGylated nanoparticles composed of the polymer-drug conjugate, free 5050 PLGA-O-acetyl acid, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 6.0 mg/mL (e.g., about 1, 2, 3, 4, 5 or 6 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

6) Tetra-(docetaxel) triglutamate-5050 PLGA-O-acetyl PEGylated Nanoparticles

Another exemplary nanoparticle includes the polymer-agent conjugate tetra-(docetaxel) triglutamate-5050 PLGA-O-acetyl, which is a conjugate of PLGA and docetaxel, with a tetrafunctional tri(glutamate) linker. This conjugate has the formula shown below:

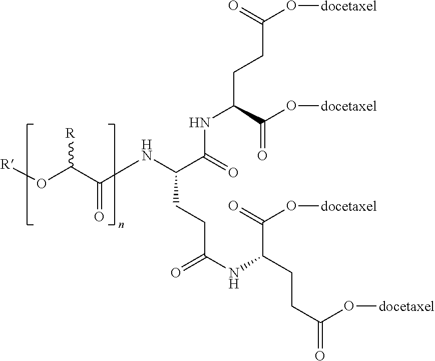

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

PLGA may be synthesized by ring opening polymerization of lactic acid (lac) lactones and glycolic acid (glc) lactones. Thus, the polymer consists of alternating dimers in random sequence, e.g., HO-[(lac-lac)-(lac-lac)-(glc-glc)-(glc-glc)-(lac-lac)-(glc-glc)-(lac-lac)-(glc-glc)]ₙ-COOH and so on. Alternatively, PLGA synthesized from of glc-monomers and lac-monomers (as opposed to dimers) can be used as well.

Each docetaxel is attached to the tri(glutamate) linker via an ester bond, primarily via the 2′ hydroxyl groups. The product may include polymers in which docetaxel is attached via the 2′, 7, 10 and/or 1 positions, in any combination; or polymers in which 0, 1, 2 or 3 docetaxel molecules are attached, via the 2′, 7, 10 and/or 1 positions; and/or docetaxel molecules attached to multiple polymer chains (e.g., via both the 2′ and 7 positions). The weight loading of docetaxel on the PLGA polymer ranges from 19-21 weight %. The conjugation of docetaxel to PLGA results in a mixture composed of tetra-(docetaxel) triglutamate-5050 PLGA-O-acetyl and 5050 PLGA-O-acetyl in a ratio ranging from 100:0 to 70:30 weight %. The second component of the particle is thus 5050 PLGA-O-acetyl, having a free —COOH moiety at its terminus. Its structure is represented by the following formula:

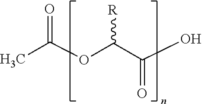

wherein R is H or CH₃; wherein about 40-60% of R substituents are H and about 40-60% are CH₃ (e.g., about 50% are H and 50% are CH₃); and n is an integer from about 75 to about 230, from about 80 to about 200, or from about 105 to about 170 (e.g., n is an integer such that the molecular weight of the polymer is from about 5 kDa to about 15 kDa or from about 6 kDa to about 13 kDa, or about 7 kDa to about 11 kDa). The polymer PDI ranges from 1.0 to 2.0 (preferably 1.0 to 1.7).

A third component of the tetra-(docetaxel) triglutamate-5050 PLGA-O-acetyl nanoparticles is the diblock copolymer methoxy-poly(ethylene glycol)-block-poly(lactide-co-glycolide) (“mPEG-PLGA”). The two blocks are linked via an ester bond, and the PEG block is capped with a methyl group. The structure is represented by the following formula:

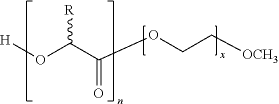

wherein R is H or CH₃; about 40-60% of R substituents are H and about 40-60% are CH₃ (e.g., about 50% are H and 50% are CH₃); n is an integer from about 100 to about 270 (e.g., n is an integer such that the molecular weight of the PLGA block is from about 7 kDa to about 17 kDa); and x is an integer from about 25 to about 500 (e.g., x is an integer such that the molecular weight of the PEG block is from about 1 kDa to about 21 kDa). The molecular weight of the PLGA block ranges from about 8 kDa to about 13 kDa (preferably about 9 kDa to about 11 kDa) when conjugated to PEG2000, giving a total molecular weight for mPEG-PLGA ranging from about 10 kDa to about 15 kDa (preferably about 11 to about 13 kDa), with a polymer PDI of about 1.0 to about 2.0 (preferably about 1.0 to about 1.7). The molecular weight of the PLGA block is from about 12 kDa to about 22 kDa when conjugated to PEG5000, giving a total molecular weight for mPEG-PLGA of about 17 kDa to about 27 kDa (preferably about 15 kDa to about 19 kDa), with a polymer PDI of about 1.0 to about 2.0 (preferably about 1.0 to about 1.7). mPEG-PLGA is added to the mixture in a range from 15 to 45 weight % with respect to tetra-(docetaxel) triglutamate-5050 PLGA-O-acetyl (preferably about 16 to 40 weight %), giving ratios of 85:15 to 55:45 weight % (preferably 84:16 to 60:40 weight %).

A fourth component of the tetra-(docetaxel) triglutamate-5050 PLGA-O-acetyl nanoparticles is a surfactant, typically poly(vinyl alcohol) (PVA). The structure of PVA is shown below; it is generated by hydrolysis of polyvinyl acetate. The PVA used in the particles described herein is about 80-90% hydrolyzed; thus, in the structure below, about 80-90% of R substituents are H and about 10-20% are (CH₃C═O). m is an integer from about 90 to about 1000 (e.g., m is an integer such that the molecular weight of the polymer is from about 5 kDa to about 45 kDa, preferably from about 9 kDa to about 30 kDa). The viscosity of poly(vinyl alcohol) ranges from 2.5-6.5 mPa·sec at 20° C.

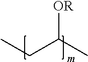

The polymer mixture of tetra-(docetaxel) triglutamate-5050 PLGA-O-acetyl, 5050 PLGA-O-acetyl and PEGylated block copolymer mPEG-PLGA are dissolved in a water-miscible organic solvent, typically acetone, in the desired mixing ratio to yield a solution composed of a total polymer concentration ranging from about 0.5 to about 5.0 percent (preferably 0.5-2.0 percent). This combined polymer solution is then added under vigorous mixing to the aqueous solution containing poly(vinyl alcohol) in a concentration of about 0.25 to about 2.0 percent weight/volume (preferably about 0.5 percent weight/volume). The mixing ratio between organic solvent and water is from about 1:1 to about 1:10 volume/volume, preferably about 1:10 percent volume/volume. The resulting mixture contains PEGylated nanoparticles composed of the polymer-drug conjugate, free 5050 PLGA-O-acetyl acid, mPEG-PLGA, PVA, and acetone. This mixing process is generally described as solvent-to-anti-solvent precipitation or nanoprecipitation.

This resulting mixture is subjected to tangential flow filtration or dialysis to remove the organic solvent, unbound mPEG-PLGA and PVA, and to concentrate the nanoparticles to an equivalent drug concentration up to about 9.0 mg/mL (e.g., about 1, 2, 3, 4, 5, 6, 7, 8 or 9 mg/mL). The resulting mixture contains PEGylated nanoparticles composed of the polymer-drug conjugate (about 20 to about 80 weight %), free 5050 PLGA-O-acetyl acid (about 0 to about 40 weight %), mPEG-PLGA (about 5 to about 30 weight %), and PVA (about 15 to about 35 weight %). In a composition of a plurality of PEGylated nanoparticles, the PEGylated nanoparticles have a Dv₉₀ less than 200 nm, with particle PDI of 0.05 to 0.15.

A lyoprotectant (typically sucrose or 2-hydroxypropyl-β-cyclodextrin) may be added in a ratio ranging from 1:1 to 15:1 (preferably 10:1) weight/weight of the entire solution, to the concentrated mixture in order to allow water removal by a freeze-drying process to produce a dry powder for storage purposes. This powder contains PEGylated nanoparticles composed of the polymer-drug conjugate, free 5050 PLGA-O-acetyl acid, mPEG-PLGA, PVA, and sucrose. The powder can be reconstituted in water, saline solution, phosphate-buffered saline (PBS) solution, or D5W for medical application, to a final equivalent drug concentration of up to about 6.0 mg/mL (e.g., about 1, 2, 3, 4, 5 or 6 mg/mL). In a composition of the reconstituted PEGylated nanoparticles, the PEGylated nanoparticles have a particle size of Dv₉₀ less than 200 nm, with a particle PDI of 0.15 to 0.2.

PEGylated nanoparticles can be sterile filtered (i.e., using a 0.22 micron Steriflip filter) while in solution prior to lyophilization or, alternatively, the organic and aqueous solutions can be sterile filtered prior to the mixing step and the nanoparticle process can be done aseptically. Another format would be to store the nanoparticles in a solution rather than a lyophilized cake. The lyophilized or solution PEGylated nanoparticle product would then be stored under appropriate conditions, e.g., refrigerated (2-8° C.), frozen (less than 0° C.), or controlled room temperature.

### Pharmaceutical Compositions

In another aspect, the present invention provides a composition, e.g., a pharmaceutical composition, comprising a plurality of particles described herein and a pharmaceutically acceptable carrier or adjuvant.

In some embodiments, a pharmaceutical composition may include a pharmaceutically acceptable salt of a compound described herein, e.g., a polymer-agent conjugate. Pharmaceutically acceptable salts of the compounds described herein include those derived from pharmaceutically acceptable inorganic and organic acids and bases.

Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a polymer-agent conjugate, particle or composition, which may be any liquid solution compatible with the polymer-agent conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, D5W and mixtures thereof.

A composition described herein may also include another component, such as an antioxidant, antibacterial, buffer, bulking agent, chelating agent, an inert gas, a tonicity agent and/or a viscosity agent.

In one embodiment, the polymer-agent conjugate, particle or composition is provided in lyophilized form and is reconstituted prior to administration to a subject. The lyophilized polymer-agent conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the particle and active from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxamers, polysorbates, polyoxyethylene esters, lecithins, saccharides, oligosaccharides, polysaccharides, carbohydrates, cyclodextrins (e.g. 2-hydroxypropyl-β-cyclodextrin) and polyols (e.g., trehalose, mannitol, sorbitol, lactose, sucrose, glucose and dextran), salts and crown ethers.

In some embodiments, the lyophilized polymer-agent conjugate, particle or composition is reconstituted with water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution of the polymer-agent conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject polymer-agent conjugate, particle or composition with a third vial containing sufficient parenteral diluent to prepare the final concentration for administration. A typical diluent is Lactated Ringer's Injection.

The final dilution of the reconstituted polymer-agent conjugate, particle or composition may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active agent which can be combined with a pharmaceutically acceptable carrier to produce a single dosage form will vary depending upon the host being treated, the particular mode of administration. The amount of active agent which can be combined with a pharmaceutically acceptable carrier to produce a single dosage form will generally be that amount of the compound which produces a therapeutic effect.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulzation, propellant or a dry powder device) or via an implanted reservoir.

Pharmaceutical compositions suitable for parenteral administration comprise one or more polymer-agent conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the polymer-agent conjugate, particle or composition then depends upon its rate of dissolution which, in turn, may depend upon crystal size and crystalline form. Alternatively, delayed absorption of a parenterally administered drug form is accomplished by dissolving or suspending the polymer-agent conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the polymer-agent conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the polymer-agent conjugate, particle or composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more polymer-agent conjugate, particle or composition described herein with one or more suitable non-irritating excipients which is solid at room temperature, but liquid at body temperature. The composition will therefore melt in the rectum or vaginal cavity and release the polymer-agent conjugate, particle or composition. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention. An ocular tissue (e.g., a deep cortical region, a supranuclear region, or an aqueous humor region of an eye) may be contacted with the ophthalmic formulation, which is allowed to distribute into the lens. Any suitable method(s) of administration or application of the ophthalmic formulations of the invention (e.g., topical, injection, parenteral, airborne, etc.) may be employed. For example, the contacting may occur via topical administration or via injection.

### Dosages and Dosage Regimens

The polymer-agent conjugate(s), particle(s) or composition(s) can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the polymer-agent conjugate, particle or composition is administered to a subject at a dosage of, e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the polymer-agent conjugate, particle or composition is administered as a bolus infusion or intravenous push, e.g., over a period of 15 minutes, 10 minutes, 5 minutes or less. In one embodiment, the polymer-agent conjugate, particle or composition is administered in an amount such the desired dose of the agent is administered. Preferably the dose of the polymer-agent conjugate, particle or composition is a dose described herein.

In one embodiment, the subject receives 1, 2, 3, up to 10, up to 12, up to 15 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The polymer, particle, or composition can be administered as a first line therapy, e.g., alone or in combination with an additional agent or agents. In other embodiments, a polymer-agent conjugate, particle or composition is administered after a subject has developed resistance to, has failed to respond to or has relapsed after a first line therapy. The polymer-agent conjugate, particle or composition may be administered in combination with a second agent. Preferably, the polymer-agent conjugate, particle or composition is administered in combination with a second agent described herein. The second agent may be the same or different as the agent in the particle.

### Kits

A polymer-agent conjugate, particle or composition described herein may be provided in a kit. The kit includes a polymer-agent conjugate, particle or composition described herein and, optionally, a container, a pharmaceutically acceptable carrier and/or informational material. The informational material can be descriptive, instructional, marketing or other material that relates to the methods described herein and/or the use of the particles for the methods described herein.

The informational material of the kits is not limited in its form. In one embodiment, the informational material can include information about production of the polymer-agent conjugate, particle or composition, physical properties of the polymer-agent conjugate, particle or composition, concentration, date of expiration, batch or production site information, and so forth. In one embodiment, the informational material relates to methods for administering the polymer-agent conjugate, particle or composition.

In one embodiment, the informational material can include instructions to administer a polymer-agent conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a polymer-agent conjugate, particle or composition described herein to a suitable subject, e.g., a human, e.g., a human having or at risk for a disorder described herein. In another embodiment, the informational material can include instructions to reconstitute a polymer-agent conjugate or particle described herein into a pharmaceutically acceptable composition.

In one embodiment, the kit includes instructions to use the polymer-agent conjugate, particle or composition, such as for treatment of a subject. The instructions can include methods for reconstituting or diluting the polymer-agent conjugate, particle or composition for use with a particular subject or in combination with a particular chemotherapeutic agent. The instructions can also include methods for reconstituting or diluting the polymer conjugate composition for use with a particular means of administration, such as by intravenous infusion.

In another embodiment, the kit includes instructions for treating a subject with a particular indication, such as a particular cancer, or a cancer at a particular stage. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid. In another embodiment, the instructions will describe treatment of selected subjects with the polymer-agent conjugate, particle or composition. For example, the instructions can describe treatment of one or more of: a subject who has received an anticancer agent (e.g., docetaxel, paclitaxel, larotaxel, cabazitaxel, doxorubicin) and has a neutrophil count less than a standard; a subject who has moderate to severe neutropenia; a subject who has experienced one or more symptom of neuropathy from treatment with an anticancer agent, e.g., a taxane, a vinca alkaloid, an alkylating agent, an anthracycline, a platinum-based agent or an epothilone; a subject who has experienced an infusion site reaction or has or is at risk for having hypersensitivity to treatment with an anticancer agent (e.g., a taxane); a subject having transaminase (ALT and/or AST levels) greater than the upper limit of normal (ULN) and/or bilirubin levels greater than ULN; a subject having ALP levels greater than the upper limit of normal (ULN), SGOT and/or SGPT levels greater the upper limit of normal (ULN) and/or bilirubin levels greater than the ULN; a subject who is currently being administered or will be administered a cytochrome P450 isoenzyme inhibitor; and a subject who has or is at risk for having fluid retention and/or effusion.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a particle described herein and/or its use in the methods described herein. The informational material can also be provided in any combination of formats.

In addition to a polymer-agent conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a polymer-agent conjugate, particle or composition described herein and the other ingredients, or for using a polymer-agent conjugate, particle or composition described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the polymer-agent conjugate, particle or composition and the second therapeutic agent are in separate containers, and in another embodiment, the polymer-agent conjugate, particle or composition and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A polymer-agent conjugate, particle or composition described herein can be provided in any form, e.g., liquid, frozen, dried or lyophilized form. It is preferred that a polymer-agent conjugate, particle or composition described herein be substantially pure and/or sterile. In an embodiment, the polymer-agent conjugate, particle or composition is sterile. When a polymer-agent conjugate, particle or composition described herein is provided in a liquid solution, the liquid solution preferably is an aqueous solution, with a sterile aqueous solution being preferred. In one embodiment, the polymer-agent conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a polymer-agent conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a polymer-agent conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Methods of Using Particles and Compositions

The polymer-agent conjugates, particles and compositions described herein can be administered to cells in culture, e.g. in vitro or ex vivo, or to a subject, e.g., in vivo, to treat or prevent a variety of disorders, including those described herein below. The polymer-agent conjugates, particles and compositions can be used as part of a first line, second line, or adjunct therapy, and can also be used alone or in combination with one or more additional treatment regimes.

### Cancer

The disclosed polymer-agent conjugates, particles and compositions are useful in treating proliferative disorders, e.g., treating a tumor and metastases thereof wherein the tumor or metastases thereof is a cancer described herein. In some embodiments, wherein the agent is a diagnostic agent, the polymer-agent conjugates, particles and compositions described herein can be used to evaluate or diagnose a cancer.

The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The polymer-agent conjugates, compounds or compositions described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a polymer-agent conjugate, compound or composition and a second therapeutic agent. Various combinations are described herein. The combination can reduce the development of tumors, reduces tumor burden, or produce tumor regression in a mammalian host.

Cancer Combination Therapy

The polymer-agent conjugate, compound or composition may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The polymer-agent conjugate, compound or composition and the at least one additional therapeutic agent can be administered simultaneously, in the same or in separate compositions, or sequentially. For sequential administration, the polymer-agent conjugate, compound or composition can be administered first, and the additional agent can be administered second, or the order of administration can be reversed.

In some embodiments, the polymer-agent conjugate, compound or composition is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the polymer-agent conjugate, compound or composition is administered with at least one additional therapeutic agent, such as a chemotherapeutic agent. In certain embodiments, the polymer-agent conjugate, compound or composition is administered in combination with one or more additional chemotherapeutic agent, e.g., with one or more chemotherapeutic agents described herein.

In some embodiments, the polymer-agent conjugate, compound or composition is administered in combination with a chemotherapeutic agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

In some embodiments, the polymer-agent conjugate, compound or composition is administered instead of another microtubule affecting agent, e.g., instead of a microtubule affecting agent as a first line therapy or a second line therapy. For example, the polymer-agent conjugate, compound or composition can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steroid can be administered in combination with a polymer-agent conjugate, compound or composition. Examples of hormones and steroids include: 17a-ethinylestradiol (Estinyl®, Ethinoral®, Feminone®, Orestralyn®), diethylstilbestrol (Acnestrol®, Cyren Deladumone®, Diastyl®, Domestrol®, Estrobene®, Estrobene®, Estrosyn®, Fonatol®, Makarol®, Milestrol®, Milestrol®, Neo-Oestronol Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the polymer-agent conjugate, compound or composition is administered in combination with an anti-microbial (e.g., leptomycin B).

In another embodiment, the polymer-agent conjugate, compound or composition is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the polymer-agent conjugate, compound or composition is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), calcineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a polymer-agent conjugate, compound or composition is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary chemotherapeutic agents that may be administered in combination with a polymer-agent conjugate, compound or composition include, bevacizumab (Avastin®), cisplatin (Platinol®), carboplatin (Paraplat®, Paraplatin®), irinotecan (Camptosar®), floxuridine (FUDF®), 5-fluorouracil (5FU) (Adrucil®, Efudex®, Fluoroplex®), leucovorin (Wellcovorin®), capecitabine (Xeloda®), gemcitabine (Gemzar®), oxaliplatin (Eloxatin®), mitoxantrone (Novantrone®), prednisone (Delta-Dome®, Deltasone®, Liquid Pred®, Lisacort®, Meticorten®, Orasone®, Prednicen-M®, Sk-Prednisone®, Sterapred®), estramustine (Emcyt®), sunitinib (Sutent®), temsirolimus (Torisel®), sorafenib (Nexavar®), everolimus (Afinitor®), cetuximab (Erbitux®), pemetrexed (ALIMTA®), erlotinib (Tarceva®), daunorubicin (Cerubidine®, Rubidomycin®), doxorubicin (Adriamycin®), trastuzumab (Herceptin®), or tamoxifen (Nolvadex®). Exemplary combinations of agents that can be administered with a polymer-agent conjugate, compound or composition include, e.g., bevacizumab (Avastin®) and interferon; 5FU (Adrucil®, Efudex®, Fluoroplex®) and leucovorin (Wellcovorin®); UFT (Uftoral®) and Leucovorin (Wellcovorin®); cisplatin (Platinol®) and pemetrexed (ALIMTA®); cisplastin (Platinol®) and vinorelbine (Navelbine®); cisplastin (Platinol®) and gemcitabine (Gemzar®); cisplastin (Platinol®) and vinblastine (Velban®, Velsar®); cisplastin (Platinol®), dacarbazine (DTIC-Dome®) and vinblastine (Velban®, Velsar®); cisplastin (Platinol®), temozolomide (Methazolastone®, Temodar®) and vinblastine (Velban®, Velsar®); cisplatin (Platinol®) and 5FU (Adrucil®, Efudex®, Fluoroplex®); oxaliplatin (Eloxatin®) and irinotecan (Camptosar®); 5FU (Adrucil®, Efudex®, Fluoroplex®), irinotecan (Camptosar®), and leucovorin (Wellcovorin®); 5FU (Adrucil®, Efudex®, Fluoroplex®), irinotecan (Camptosar®), oxaliplatin (Eloxatin®), and leucovorin (Wellcovorin®); 5FU (Adrucil®, Efudex®, Fluoroplex®) and radiation; 5FU (Adrucil®, Efudex®, Fluoroplex®), radiation and cisplatin (Platinol®); oxaliplatin (Eloxatin®), 5FU (Adrucil®, Efudex®, Fluoroplex®), and leucovorin (Wellcovorin®); capecitabine (Xeloda®), oxaliplatin (Eloxatin®), and bevacizumab (Avastin®); capecitabine (Xeloda®), irinotecan (Camptosar®), and bevacizumab (Avastin®); capecitabine (Xeloda®) and bevacizumab (Avastin®); irinotecan (Camptosar®) and bevacizumab (Avastin®); cetuximab (Erbutux®) and bevacizumab (Avastin®); cetuximab (Erbutux®), irinotecan (Camptosar®) and bevacizumab (Avastin®); panitumumab (Vectibix®) and bevacizumab (Avastin®); 5FU (Adrucil®, Efudex®, Fluoroplex®), leucovorin (Wellcovorin®) and bevacizumab (Avastin®); 5FU (Adrucil®, Efudex®, Fluoroplex®), leucovorin (Wellcovorin®), oxaliplatin (Eloxatin®) and bevacizumab (Avastin®); 5FU (Adrucil®, Efudex®, Fluoroplex®), leucovorin (Wellcovorin®), irinotecan (Camptosar®) and bevacizumab (Avastin®); 5FU (Adrucil®, Efudex®, Fluoroplex®), oxaliplatin (Eloxatin®), irinotecan (Camptosar®), leucovorin (Wellcovorin®) and bevacizumab (Avastin®); and UFT (Uftoral®), irinotecan (Camptosar®) and leucovorin (Wellcovorin®).

When formulating the pharmaceutical compositions featured in the invention the clinician may utilize preferred dosages as warranted by the condition of the subject being treated. For example, in one embodiment, a polymer-agent conjugate, compound or composition may be administered at a dosing schedule described herein, e.g., once every one, two three four, five, or six weeks.

Also, in general, a polymer-agent conjugate, compound or composition, and an additional chemotherapeutic agent(s) do not have to be administered in the same pharmaceutical composition, and may, because of different physical and chemical characteristics, have to be administered by different routes. For example, the polymer-agent conjugate, compound or composition may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a polymer-agent conjugate, compound or composition is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, SN-38, camptothecin (e.g., IT-101)); and a platinum-based agent (e.g., cisplatin (Platinol®), carboplatin (Paraplat®, Paraplatin®), oxaliplatin (Eloxatin®)).

In another embodiment, the polymer-agent conjugate, compound or composition is administered once every two weeks in combination with one or more additional chemotherapeutic agent that is administered orally. For example, the polymer-agent conjugate, compound or composition can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The actual dosage of the polymer-agent conjugate, compound or composition and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In one embodiment, the polymer-agent conjugate, compound or composition can be administered at a dose that includes 0.5 to 300 mg/m² of an agent, e.g., 2.5 mg/m² to 30 mg/m², 9 to 280 mg/m², 0.5 to 100 mg/m², 0.5 to 35 mg/m², 25 to 90 mg/m². Preferably, the polymer-agent conjugate, compound or composition is administered at a dosage described herein.

In some embodiments, when a polymer-agent conjugate, compound or composition is administered in combination with one or more additional chemotherapeutic agent, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml x min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

The disclosure also encompasses a method for the synergistic treatment of cancer wherein a polymer-agent conjugate, compound or composition is administered in combination with an additional chemotherapeutic agent or agents.

The particular choice of polymer conjugate and anti-proliferative cytotoxic agent(s) or radiation will depend upon the diagnosis of the attending physicians and their judgment of the condition of the subject and the appropriate treatment protocol.

If the polymer-agent conjugate, compound or composition and the chemotherapeutic agent(s) and/or radiation are not administered simultaneously or essentially simultaneously, then the initial order of administration of the polymer-agent conjugate, compound or composition, and the chemotherapeutic agent(s) and/or radiation, may be varied. Thus, for example, the polymer-agent conjugate, compound or composition may be administered first followed by the administration of the chemotherapeutic agent(s) and/or radiation; or the chemotherapeutic agent(s) and/or radiation may be administered first followed by the administration of the polymer-agent conjugate, compound or composition. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (polymer-agent conjugate, compound or composition, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### Cardiovascular Disease

The disclosed methods may be useful in the prevention and treatment of cardiovascular disease. Cardiovascular diseases that can be treated or prevented using polymer-agent conjugates, particles, compositions and methods described herein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using polymer-agent conjugates, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The polymer-agent conjugates, particles, compositions and methods described herein may also be used for increasing HDL levels in plasma of an individual.

Yet other disorders that may be treated with polymer-agent conjugates, particles, compositions and methods described herein include restenosis, e.g., following coronary intervention, and disorders relating to an abnormal level of high density and low density cholesterol.

The polymer-agent conjugate, particle or composition can be administered to a subject undergoing or who has undergone angioplasty. In one embodiment, the polymer-agent conjugate, particle or composition is administered to a subject undergoing or who has undergone angioplasty with a stent placement. In some embodiments, the polymer-agent conjugate, particle or composition can be used as a strut of a stent or a coating for a stent.

The polymer-agent conjugates, particles or compositions can be used during the implantation of a stent, e.g., as a separate intravenous administration, as coating for a stent or as the strut of a stent.

Stent

The polymer-agent conjugates, particles or compositions described herein can be used as or be part of a stent. As used herein, the term “stent” refers to a man-made ‘tube’ inserted into a natural passage or conduit in the body to prevent or counteract localized flow constriction. Types of stents include, e.g., coronary stent, urinary tract stent, urethral/prostatic stent, vascular stent (e.g., peripheral vascular stent, or stent graft), esophageal stent, duodenal stent, colonic stent, biliary stent, and pancreatic stent. Types of stents that can be used in coronary arteries include, e.g., bare-metal stent (BMS) and drug-eluting stent (DES). A coronary stent can be placed within the coronary artery during an angioplasty procedure.

Bare-Metal Stent (BMS)

In one embodiment, the polymer-agent conjugate, particle or composition can be used in combination with a BMS. As used herein, BMS refers to a stent without a coating that is made or a metal or combination of metals. BMS can be made from, e.g., stainless steel (e.g., BxVelocity™ stent, Express2™ stent, R Stent™, and Matrix® coronary stent), cobalt-chromium alloy (e.g., Driver® coronary stent, ML Vision® stent, and Coronnium® stent), or nickel titanium (Nitinol® stent). A polymer-agent conjugate, particle or composition described herein can be used as a coating of a BMS, e.g., to coat the luminal and/or abluminal surface of a BMS.

Drug-Eluting Stent (DES)

In one embodiment, the polymer-agent conjugate, particle or composition can be a DES or can be part of a DES. As used herein, DES refers to a stent placed into a natural passage or conduit of the body (e.g., a narrowed coronary artery) that releases (e.g., slowly releases) one or more agents to treat one or more symptoms associated with the constricted flow to the passage or conduit and/or one or more effect caused by or associated with the stent. For example, the DES can release one (or more) agent that reduces or inhibits the migration and/or proliferation of vascular smooth muscle cells (SMCs), that promotes or increases epithelialization, that reduces or inhibits a hypersensitivity reaction, that reduces or inhibits inflammation, that reduces or inhibits thrombosis, that reduces the risk of restenosis, and/or that reduces or inhibits other unwanted effects due to the stent.

One type of DES includes a stent strut and a polymer, on which an agent is loaded. Thus, in one embodiment, a polymer-agent conjugate, particle or composition described herein can be used in combination with other polymeric struts (e.g., other biocompatible or bioasorbable polymers). For example, a polymer-agent conjugate, particle or composition described herein can be coated on a polymeric strut, e.g., on the luminal and/or abluminal surface of a polymeric strut.

In another embodiment, the polymer-agent conjugates, particles and compositions described herein can be used as a polymeric strut, with out without an additional polymer and/or agent.

In one embodiment, the rate of major adverse cardiac events (MACE) of a subject having a stent made of a polymer-agent conjugate, particle or composition described herein or a strut coated with a polymer-agent conjugate, particle or composition described herein is reduced by at least 10, 20, 30, 40, 50, 60, 70, 80, 90, 95% or more, as compared to the rate of MACE of a subject having a stent made of a different material (e.g., a metal or polymer) or a stent not coated or coated with a polymer and/or agent other than the polymer-agent conjugate, particle or composition. In another embodiment, the need for target vessel revascularization (TVR) of a subject having a stent made of a polymer-agent conjugate, particle or composition described herein or a strut coated with a polymer-agent conjugate, particle or composition described herein is reduced by at least 10, 20, 30, 40, 50, 60, 70, 80, 90, 95% or more, compared to the TVR of a subject having a stent made of a different material (e.g., a metal or polymer) or a stent not coated or coated with a polymer and/or agent other than the polymer-agent conjugate, particle or composition. In yet another embodiment, the rate for target lesion revascularization (TLR) of a subject having a stent made of a polymer-agent conjugate, particle or composition described herein or a strut coated with a polymer-agent conjugate, particle or composition described herein is reduced by at least 10, 20, 30, 40, 50, 60, 70, 80, 90, 95% or more, compared to the TLR of a subject having a stent made of a different material (e.g., a metal or polymer) or a stent not coated or coated with a polymer and/or agent other than the polymer-agent conjugate, particle or composition.

Agents

Agents that can be loaded onto a DES include, for example, antiproliferative agents, e.g., anticancer agents (e.g., a taxane (e.g., docetaxel, paclitaxel, larotaxel and cabazitaxel) and an anthracycline (e.g., doxorubicin); pro-endothelial cell agents, anti-restenotic agents; anti-inflammatory agents; statins (e.g., simovastatin); immunosuppresants (e.g., mycophenolic acid); somatostatin receptor agonists (e.g., angiopeptin); and dimethyl sulfoxide.

Exemplary anti-proliferative agents include, e.g., an anticancer agent, e.g., a taxane (e.g., docetaxel, paclitaxel, larotaxel and cabazitaxel) and an anthracycline (e.g., doxorubicin); and an immunosuppressive agent, e.g., a rapamycin analogue (e.g., everolimus, zotarolimus, biolimus), pimecrolimus, or tacrolimus.

One or more of the pro-endothelial agents can be loaded on the stents, e.g., to promote, accelerate or increase endothelial healing. Exemplary pro-endothelial agents include, e.g., agents that diminish platelet adhesion and/or fibrinogen binding (e.g., titanium-nitride-oxide or titanium-nitride), agents that capture endothelial progenitor cells (EPCs) (e.g., antibodies (e.g., anti-CD34 antibody) or peptides (e.g., integrin-binding cyclic Arg-Gly-Asp peptide)), or estradiol.

One or more of anti-restenotic agent can also be loaded on or in the stents, e.g., anti-inflammmatory agents (e.g., dexamethasome), immunosuppressive agents (e.g., mycophenolic acid), antisense agents (e.g., an advanced six-ring morpholino backbone c-myc antisense (AVI-4126)), inhibitors of vascular smooth muscle cell proliferation and/or tissue factor expression (e.g., 3-hydroxy-3-methylglutaryl coenzyme A (HMG-CoA)-reductase-inhibitors (statins), simvastatin, angiopeptin or dimethyl sulfoxide (DMSO)), or anti-hyperlipidemic agents (e.g., probucol).

In one embodiment, the agent (or agents) is loaded on the luminal side of the stent. In another embodiment, the agent (or agents) is loaded on the abluminal side of the stent. In yet another embodiment, the agent (or agents) is loaded on both the luminal and abluminal sides of the stent. In another embodiment, an agent (or agents) is loaded on the luminal side of the stent and a different agent (or combination of agents) is loaded on the abluminal side of the stent. Thus, different agents (e.g., an anti-proliferation agent and a pro-endothelial agent) can be loaded on different sides (luminal or abluminal) of the stent, e.g., to allow for differential agent elution, or different agents can be loaded on the same side (luminal or abluminal side) of the stent, e.g., to allow for dual local agent elution.

In one embodiment, the agent is present at a concentration of at least about 1, 2, 3, 4, 5, 6, 7, 8, 9, 10, 20, 50, or 100 μg/mm. In one embodiment, more than about 50, 60, 70, 80, 90, 95, 99% of the agent is released over a period of one month. In one embodiment, the release of the agent (e.g., a pro-endothelial agent) is delayed for at least about 1, 2, 3, 4, 5, 6, 7, 8, 9, or 10 days. In one embodiment, the release of the agent sustains for at least 7, 14, 21, 28, 35, or 42 days.

Polymeric Stents

Stents described herein can be made of biocompatible and/or bioabsorbable polymers. A polymer-agent conjugate, particle or composition described herein can be the stent, the strut of a stent or the poly-agent conjugate, particle or composition can coat a strut made of a polymeric material.

An example of a biocompatible stent is the Endeavor Rsolute® stent. This system is composed of three elements: one hydrophobic polymer (‘C10’) to retain the drug and control drug release, another polymer (‘C19’) to provide improved biocompatibility, and finally (on the outer-most side of the stent) a polyvinyl pyrrolidinone (PVP) hydrophilic polymer which increases the initial drug burst and further enhances biocompatibility. Thus, in one embodiment, the polymer-agent conjugate, particle or composition can be coated on an Endeavor Rsolute® stent. In other embodiments, a polymer-agent conjugate, particle or composition described herein can replace one or more of the elements of the Endeavor Rsolute® stent.

Bioabsorbable polymers (e.g., inert bioabsorbable polymer) can also be used in a DES, e.g., to reduce prothrombogenic potential and/or allow non-invasive imaging. In some embodiments, the bioabsorbable polymer has a degradation time of at least about 14, 21, 28, 35, 42, 49, 56, 63, 70 days.

Exemplary bioasorbable stents include, e.g., a polymeric stent (e.g., a poly-L-lactide stent, a tyrosine poly(desaminotyrosyl-tyrosine ethyl ester) carbonate stent, and a poly(anhydride ester) salicyclic acid stent). For example, Igaki-Tamai stent is constructed from a poly-L-lactic acid polymer and contains either the tyrosine kinase antagonist ST638 or paclitaxel. REVA® stent is a tyrosine poly(desaminotyrosyl-tyrosine ethyl ester) carbonate stent. It is radio-opaque and has slide and lock mechanism designed to allow for substantial reductions in stent-strut thickness. IDEAL™ stent is a poly(anhydride ester) salicyclic acid stent Infinnium® stent is composed of two biodegradable polymers with different paclitaxel-release kinetics. Other exemplary bioasorbable stents include, e.g., BVS®, Sahajanand®, Infinnium®, BioMATRIX®, Champion®, and Infinnium®. In one embodiment, a polymer-agent conjugate, particle or composition described herein can be coated onto any of these bioabsorbable stents. In other embodiments, a polymer-agent conjugate, particle or composition described herein can replace one or more elements of one of these bioabsorbable stents.

Biosorbable Metallic Stents

The polymer-agent conjugates, particles and compositions described herein can be used to coat a bioabsorbable metallic stent. An exemplary bioabsorbable stent is the Absorbable Metal Stent (AMS®) which is an alloy stent made of 93% magnesium and 7% rare-earth metals.

Reservoir Stents

As described herein, reservoir stents can be used, e.g., to decrease the “thickness” of the stent or reduce the unwanted effect due to microfragmentation of the polymer and/or the agent. For example, the drug can be loaded in one or more reservoirs or wells in the stent, compared to, e.g., more or less uniformly spread over the stent.

In one embodiment, a polymer-agent conjugate, particle or composition described herein is loaded in the reservoirs or wells located on the stent, e.g., the polymer-agent conjugate, particle or composition described herein is loaded in the reservoirs or wells located on the luminal side or the abluminal side of the stent. In yet another embodiment, the polymer-agent conjugate, particle or composition described herein is loaded in the reservoirs or wells located on both the luminal and abluminal sides of the stent.

In one embodiment, different agents (e.g., an anti-proliferation agent and a pro-endothelial agent) can be loaded into the reservoirs or wells on different sides (luminal or abluminal) of the stent, e.g., to allow for differential agent elution. In another embodiment, different agents can be loaded into adjacent reservoirs or wells of the same side (luminal or abluminal side) of the stent, e.g., to allow for dual local drug elution.

Strut

In one embodiment, the strut thickness is at least about 25, 50, 100, 150, 200, 250 μm. In another embodiment, the strut wideness is at least about 0.002, 0.004, 0.006, 0.008, or 0.01 inch. In yet another embodiment, the number of struts is at least about 4, 8, 12, 16, or 18 in its cross-section.

Various shapes of struts such as a zig zag coil, a ratchet log design, circumferential loops, etc. are known in the art and can be employed in the stents described herein.

In one embodiment, the strut can be made of a polymer-agent conjugate particle or composition described herein.

Combination Therapy

In one embodiment, a polymer-agent conjugate, particle or composition described herein may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a polymer-agent conjugate, particle or composition may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocainide, procainamide, encainide, flecanide, tocainide, phenytoin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions.

In another embodiment, a polymer-agent conjugate, particle or composition may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; antiarrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex, or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, fiunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin El and prostaglandin 12), an endothelin receptor blocking drug (such as bosentan), diltiazem, nicorandil, and nitroglycerin. Examples of cerebral protecting drugs include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of brain metabolic stimulants include amantadine, tiapride, and gamma-aminobutyric acid. Examples of anticoagulants include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of antiplatelet drugs include ticlopidine hydrochloride, dipyridamole, cilostazol, ethyl icosapentate, sarpogrelate hydrochloride, dilazep hydrochloride, trapidil, a nonsteroidal anti-inflammatory agent (such as aspirin), beraprostsodium, iloprost, and indobufene.

Examples of thrombolytic drugs include urokinase, tissue-type plasminogen activators (such as alteplase, tisokinase, nateplase, pamiteplase, monteplase, and rateplase), and nasaruplase. Examples of antihypertensive drugs include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of antianginal drugs include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline) trimetazidine, dipyridamole, etafenone, dilazep, trapidil, nicorandil, enoxaparin, and aspirin.

Examples of diuretics include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, and potassiumcanrenoate), osmotic diuretics (such as isosorbide, D-mannitol, and glycerin), nonthiazide diuretics (such as meticrane, tripamide, chlorthalidone, and mefruside), and acetazolamide. Examples of cardiotonics include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as amrinone, olprinone, and milrinone), denopamine, ubidecarenone, pimobendan, levosimendan, aminoethylsulfonic acid, vesnarinone, carperitide, and colforsin daropate. Examples of antiarrhythmic drugs include ajmaline, pirmenol, procainamide, cibenzoline, disopyramide, quinidine, aprindine, mexiletine, lidocaine, phenyloin, pilsicainide, propafenone, flecainide, atenolol, acebutolol, sotalol, propranolol, metoprolol, pindolol, amiodarone, nifekalant, diltiazem, bepridil, and verapamil. Examples of antihyperlipidemic drugs include atorvastatin, simvastatin, pravastatin sodium, fluvastatin sodium, clinofibrate, clofibrate, simfibrate, fenofibrate, bezafibrate, colestimide, and colestyramine.

Yet other exemplary cardiovascular agents include, for example, anti-angiogenic agents and vascular disrupting agents.

### Inflammation and Autoimmune Disease

The polymer-agent conjugates, particles, compositions and methods described herein may be used to treat or prevent a disease or disorder associated with inflammation. A polymer-agent conjugate, particle or composition described herein may be administered prior to the onset of, at, or after the initiation of inflammation. When used prophylactically, the polymer-agent conjugate, particle or composition is preferably provided in advance of any inflammatory response or symptom. Administration of the polymer-agent conjugate, particle or composition may prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, a polymer-agent conjugate, particle, composition or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The polymer-agent conjugate, particle or composition may be used to treat chronic hepatitis infection, including hepatitis B and hepatitis C.

Additionally, a polymer-agent conjugate, particle, composition or method described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

Combination Therapy

In certain embodiments, a polymer-agent conjugate, particle or composition described herein may be administered alone or in combination with other compounds useful for treating or preventing inflammation. Exemplary anti-inflammatory agents include, for example, steroids (e.g., Cortisol, cortisone, fludrocortisone, prednisone, 6[alpha]-methylprednisone, triamcinolone, betamethasone or dexamethasone), nonsteroidal anti-inflammatory drugs (NSAIDS (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, [alpha]-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, caiprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide and cyclazocine.

Further examples of anti-inflammatory agents include deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocoitolone, fluoresone, fluorometholone, fluperolone, flupirtine, fluprednidene, fluprednisolone, fluproquazone, flurandrenolide, flurbiprofen, fluticasone, formocortal and fosfosal.

Further examples of anti-inflammatory agents include gentisic acid, glafenine, glucametacin, glycol salicylate, guaiazulene, halcinonide, halobetasol, halometasone, haloprednone, heroin, hydrocodone, hydro cortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate and myrophine.

Further examples of anti-inflammatory agents include nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In one embodiment, a polymer-agent conjugate, particle or composition described herein may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, and lumiracoxib.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### Examples

### Example 1

### Purification and Characterization of 5050 PLGA

Step A: A 3-L round-bottom flask equipped with a mechanical stirrer was charged with 5050PLGA (300 g, Mw: 7.8 KDa; Mn: 2.7 KDa) and acetone (900 mL). The mixture was stirred for 1 h at ambient temperature to form a clear yellowish solution.

Step B: A 22-L jacket reactor with a bottom-outlet valve equipped with a mechanical stirrer was charged with MTBE (9.0 L, 30 vol. to the mass of 5050 PLGA). Celite® (795 g) was added to the solution with overhead stirring at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by 1H NMR analysis.

Step C: A 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL.

Step D: A 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86%]. The ¹H NMR analysis was consistent with that of the desired product and Karl Fisher analysis showed 0.52 wt % of water. The product was analyzed by HPLC (AUC, 230 nm) and GPC (AUC, 230 nm). The process produced a more narrow polymer polydispersity, i.e. Mw: 8.8 kDa and Mn: 5.8 kDa.

### Example 2

### Purification and Characterization of 5050 PLGA Lauryl Ester

A 12-L round-bottom flask equipped with a mechanical stirrer was charged with MTBE (4 L) and heptanes (0.8 L). The mixture was agitated at ˜300 rpm, to which a solution of 5050 PLGA lauryl ester (65 g) in acetone (300 mL) was added dropwise. Gummy solids were formed over time and finally clumped up on the bottom of the flask. The supernatant was decanted off and the solid was dried under vacuum at 25° C. for 24 h to afford 40 g of purified 5050 PLGA lauryl ester as a white powder [yield: 61.5%]. ¹H NMR (CDCl₃, 300 MHz): δ 5.25-5.16 (m, 53H), 4.86-4.68 (m, 93H), 4.18 (m, 7H), 1.69-1.50 (m, 179H), 1.26 (bs, 37H), 0.88 (t, J=6.9 Hz, 6H). The ¹H NMR analysis was consistent with that of the desired product. GPC (AUC, 230 nm): 6.02-9.9 min, t_{R}=7.91 min.

### Example 3

### Purification and Characterization of 7525 PLGA

A 22-L round-bottom flask equipped with a mechanical stirrer was charged with 12 L of MTBE, to which a solution of 7525 PLGA (150 g, approximately 6.6 kD) in dichloromethane (DCM, 750 mL) was added dropwise over an hour with an agitation of ˜300 rpm, resulting in a gummy solid. The supernatant was decanted off and the gummy solid was dissolved in DCM (3 L). The solution was transferred to a round-bottom flask and concentrated to a residue, which was dried under vacuum at 25° C. for 40 h to afford 94 g of purified 7525 PLGA as a white foam [yield: 62.7%,]. ¹H NMR (CDCl₃, 300 MHz): δ 5.24-5.15 (m, 68H), 4.91-4.68 (m, 56H), 3.22 (s, 2.3H, MTBE), 1.60-1.55 (m, 206H), 1.19 (s, 6.6H, MTBE). The ¹H NMR analysis was consistent with that of the desired product. GPC (AUC, 230 nm): 6.02-9.9 min, t_{R}=7.37 min.

### Example 4

### Synthesis, Purification and Characterization of O-Acetyl-5050-PLGA

A 2000-mL, round-bottom flask equipped with an overhead stirrer was charged with purified 5050 PLGA [220 g, Mn of 5700] and DCM (660 mL). The mixture was stirred for 10 min to form a clear solution. Ac2O (11.0 mL, 116 mmol) and pyridine (9.4 mL, 116 mmol) were added to the solution, resulting in a minor exotherm of ˜0.5° C. The reaction was stirred at ambient temperature for 3 h and concentrated to ˜600 mL. The solution was added to a suspension of Celite® (660 g) in MTBE (6.6 L, 30 vol.) over 1 h with overhead stirring at ˜200 rpm. The suspension was filtered through a polypropylene filter and the filter cake was air-dried at ambient temperature for 1 day. It was suspended in acetone (1.6 L, ˜8 vol) with overhead stirring for 1 h. The slurry was filtered though a fitted funnel (coarse) and the filter cake was washed with acetone (3×300 mL). The combined filtrates were clarified though a Celite pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The 1H NMR analysis was consistent with that of the desired product. The product was analyzed by HPLC (AUC, 230 nm) and GPC (Mw: 9.0 kDa and Mn: 6.3 kDa).

### Example 5

### Synthesis, Purification and Characterization of Doxorubicin 5050 PLGA Amide

A 1000-ml round-bottom flask with a magnetic stirrer was charged with purified 5050 PLGA [55.0 g, 10.4 mmol, 1.0 equiv.], doxorubicin.HCl (6.7 g, 11.4 mmol, 1.1 equiv, 2-chloro-N-methyl pyridinium iodide (3.45 g, 13.5 mmol, 1.3 equiv, and DMF (250 mL, anhydrous) under N₂. The suspension was stirred for 15 min and triethylamine (4.6 mL, 32.2 mmol, 3.15 equiv.) was added dropwise over 10 min. The reaction mixture became a dark red solution after the addition of TEA and an exotherm from 23.2° C. to 26.2° C. was observed. The reaction was complete after 1.5 h as indicated by HPLC analysis. The mixture was filtered through a 0.5 μM PTFE membrane and the filtrate was added dropwise into water (5.50 L) containing 11 mL of AcOH over 20 min via addition funnels. The suspension was stirred for 1 h (pH ˜3-4), filtered over 30 min, and the filter cake was washed with water (3×300 mL). The solid was suspended in water (3.0 L) containing 0.1 vol % of AcOH and 5 vol % of acetone, stirred for 1 h, and filtered (pH ˜4-5) to afford 201.9 g of wet doxorubicin 5050 PLGA amide. The wet doxorubicin 5050 PLGA amide sample was transferred into a glass tray and dried under vacuum with nitrogen bleeding at 25° C. for 16 h to afford 162.9 g of semi-dry solid. The ¹H NMR analysis indicated ˜1.0 wt % of residual DMF. This sample was suspended in H₂O (3 L) containing 3 mL of AcOH and 15 mL of acetone and stirred for 6 h, filtered, washed with H₂O (0.5 L), and held for 0.5 h to afford 163.3 g of wet doxorubicin 5050 PLGA amide. The wet doxorubicin 5050 PLGA amide (155.8 g) was dried under vacuum with N₂ bleeding at 25° C. for 16 h to afford 120.3 g of semi-dry product, which was dried at ambient temperature with N₂ purge for 16 h to afford 54.4 g of doxorubicin 5050 PLGA amide [yield: 93%]. ¹H NMR (CDCl₃, 300 MHz): δ 14.00 (s, 1H), 13.27 (s, 1H), 8.05 (d, J=7.8 Hz, 1H), 7.80 (t, J=7.8 Hz, 1H), 7.40 (d, J=8.4 Hz, 1H), 6.44 (bs, 0.8H), 5.51 (bs, 1.2H), 5.22-5.17 (m, 40H), 4.91-4.72 (m, 81H), 4.31-4.08 (m, 7H), 3.64 (bs, 0.9H), 3.30 (d, J=20.4, 1H), 3.04 (d, J=18.9 Hz, 1H), 2.94 (s, 0.1H, DMF), 2.89 (s, 0.1H, DMF), 2.36 (d, J=14.4 Hz, 1H), 2.17 (d, J=14.1 Hz, 1H), 1.84 (bs, 5H), 1.60-1.55 (m, 120H), 1.28 (d, J=6.6 Hz). The ¹H NMR analysis was consistent with that of the desired product. HPLC (AUC, 480 nm): 13.00-17.80 min, t_{R} 16.8 min. GPC (AUC, 480 nm): 5.2-8.6 min, t_{R} 6.51 min. The product may also include free 5050 PLGA and/or a trace amount of doxorubicin.

### Example 6

### Synthesis, Purification and Characterization of Doxorubicin 7525 PLGA Amide

2-chloro-N-methyl pyridinium iodide (1.95 g, 7.63 mmol) and TEA (3.15 mL, 22.6 mmol) were added to a mixture of purified 7525 PLGA [25.0 g, 3.80 mmol] and doxorubicin.HCl (3.08 g, 5.32 mmol) in DMF (125 mL, anhydrous) and stirred at ambient temperature. After 1 h, the reaction was complete by HPLC (0.4% doxorubicin remaining); however, there was 5.2% of an impurity at 12.0 min by HPLC analysis. The mixture was added into 2.50 L of water (25 mL of acetone wash) and 5.0 mL of acetic acid was added (pH=4-5). The resulting slurry was stirred for 30 min and filtered (250 mL water wash). The isolated wet cake was found to have only 1.7% of the 12.0 min impurity by HPLC analysis. The wet cake was slurried in water (1.25 L) and 1.3 mL of acetic acid was added. The mixture was stirred for 45 min, filtered (washed with 250 mL of water), and dried under vacuum for 44 h to afford 25.2 g of doxorubicin 7525 PLGA amide as a red solid [Yield: 93%]. ¹H NMR (CDCl₃, 300 MHz): δ 13.99 (s, 1H), 13.26 (s, 1H), 8.04 (d, J=7.8 Hz, 1.2H), 7.79 (t, J=7.8 Hz, 1.1H), 7.40 (d, J=8.4 Hz, 1.1H), 6.44 (bs, 0.8H), 5.50 (bs, 1.3H), 5.22-5.17 (m, 60H), 4.91-4.72 (m, 53H), 4.31-4.08 (m, 8H), 3.64 (bs, 1.1H), 3.30 (d, J=20.4, 1.0H), 3.04 (d, J=18.9 Hz, 1.2H), 2.94 (s, ˜1.0H, DMF), 2.89 (s, 1.1H, DMF), 2.36 (d, J=14.4 Hz, 1.8H), 2.17 (m, 3.4H), 1.84 (bs, 3H), 1.60-1.55 (m, 184H), 1.28 (d, J=4.6 Hz, 6.6H). The ¹H NMR analysis was consistent with that of the desired product. HPLC (AUC, 480 nm): 13.15-18.50 min, t_{R} 17.6 min. GPC (AUC, 480 nm): 5.2-8.5 min, t_{R} 6.29 min. The product may also include free 7525 PLGA and/or a trace amount of doxorubicin.

### Example 7

### Synthesis, Purification and Characterization of Paclitaxel-5050 PLGA-O-acetyl

A 250-mL round-bottom flask equipped with an overhead stirrer was charged with 5050 PLGA-O-acetyl [20 g, 2.6 mmol], paclitaxel (1.85 g, 2.1 mmol, 0.8 equiv., N,N′-dicyclohexyl-carbodiimide (DCC, 0.66 g, 3.2 mmol, 1.3 equiv.), 4-dimethylaminopyridine (DMAP, 0.39 g, 3.2 mmol, 1.3 equiv.), and DCM (100 mL, 5 vol). The mixture was agitated at 20° C. for 16 h and filtered to remove the dicyclohexylurea (DCU). The filtrate was concentrated to a residue and the residue was dissolved in acetone (100 mL), resulting in a cloudy suspension. It was filtered to remove residual DCU byproduct. The filtrate was added dropwise to 5:1 MTBE/heptanes (1.2 L) with vigorously stirring. The white precipitates formed a gum shortly after precipitation. The supernatant was decanted off and the gummy solid was isolated. The precipitation was repeated twice and the gummy solid was dried under vacuum at 25° C. for 16 h to afford 15.7 g of paclitaxel-5050 PLGA-O-acetyl [yield: 72%]¹H NMR (CDCl₃, 300 MHz): δ 8.15 (d, J=7.5 Hz, 1H), 7.75 (d, J=6.6 Hz, 1H), 7.54-7.38 (m, 6H), 6.29-6.24 (a singlet overlaps with a triplet, 1H), 6.06 (bs, 0.5H), 5.69 (d, J=6.9 Hz, 0.4H), 5.58 (bs, 0.5H), 5.26-5.17 (m, 40H), 4.93 (d, J=7.8 Hz, 0.5H), 4.90-4.72 (m, 85H), 4.43 (t, J=3.9 Hz, 1H), 4.31 (d, J=8.1 Hz, 0.5H), 4.21 (d, J=8.1 Hz, 0.5H), 3.81 (d, J=6.6 Hz, 0.5H), 2.44 (bs, 2.5H), 2.23 (s, 1.5H), 2.17 (s, 19H, acetone), 1.8-1.7 (bs, 15H), 1.68 (s, 1.5H), 1.60-1.55 (m, 124H), 1.22 (bs, 2.5H), 1.14 (s, 1.5H). The ¹H NMR analysis was consistent with that of the desired product. HPLC (AUC, 230 nm): 13.00-16.50 min, t_{R} 15.60 min. GPC (AUC, 230 nm): 6.0-9.7 min, t_{R}=7.35 min. The major product is paclitaxel-2′-5050 PLGA-O-acetyl (wherein paclitaxel is attached to 5050 PLGA-O-acetyl via the 2′ hydroxyl group); the product may also include free 5050 PLGA-O-acetyl, 7 paclitaxel-conjugate, 1 paclitaxel-conjugate, product in which two or more polymer chains are linked to paclitaxel (e.g., via the 2′ and 7 positions) and/or a trace amount of paclitaxel.

### Example 8

### Synthesis, Purification and Characterization of Docetaxel-5050 PLGA-O-acetyl

A 250-mL round-bottom flask equipped with an overhead stirrer was charged with O-acetyl-5050 PLGA (16 g, 2.6 mmol), docetaxel (1.8 g, 2.1 mmol, 0.8 equiv.), DCC (0.66 g, 3.2 mmol, 1.3 equiv.), 4-dimethylaminopyridine (DMAP, 0.35 g, 3.2 mmol, 1.3 equiv.), and EtOAc (80 mL, 5 vol). The mixture was agitated at 20° C. for 2.5 h and an additional 0.5 equivalents of DCC (0.27 g) and DMAP (0.16 g) were added. The reaction was stirred at ambient temperature for 16 h and filtered to remove the dicyclohexylurea (DCU). The filtrate was diluted with EtOAc to 250 mL. It was washed with 1% HCl (2×60 mL) and brine (60 mL). The organic layer was separated, dried over Na₂SO₄, and filtered. The filtrate was concentrated to a residue and the residue was dissolved in acetone (100 mL), resulting in a cloudy suspension. It was filtered to remove residual DCU byproduct. The filtrate was added dropwise to 5:1 MTBE/heptanes (600 mL) with vigorously stirring. The white precipitates formed a gum shortly after precipitation. The supernatant was decanted off and the gummy solid was isolated. The precipitation was repeated three more times and the gummy solid was dissolved in acetone (300 mL). The solution was concentrated to a residue, which was dried under vacuum at 25° C. for 64 h to afford 14 g of docetaxel-5050 PLGA-O-acetyl [yield: 78%]. ¹H NMR (CDCl₃, 300 MHz): δ 8.11 (d, J=6.9 Hz, 1H), 7.61 (m, 0.6H), 7.50 (t, J=7.2 Hz, 6H), 7.39 (m, 1.3H), 6.22 (bs, 0.5H), 6.68 (d, J=7.5 Hz, 5.69-5.67 (m, 2.2H), 5.49-5.17 (m, 49H), 4.90-4.72 (m, 102H), 4.43 (m, 1.2; H), 3.92 (d, J=5.7 Hz, 0.5H), 2.42 (bs, 2.1H), 2.17 (s, 29.3H, acetone), 1.90 (s, 3H), 1.80 (bs, 3H), 1.72 (s, 2H), 1.64-1.55 (m, 164H), 1.34 (s, 7H), 1.22 (m, 4H), 1.12 (s, 2.4H). The ¹H NMR analysis was consistent with that of the desired product. HPLC (AUC, 230 nm): 15.50-18.00 min, t_{R} 17.34 min. GPC (AUC, 230 nm): 6.0-9.7 min, t_{R}=7.35 min. The major product is docetaxel-2′-5050 PLGA-O-acetyl (wherein docetaxel is attached to 5050 PLGA-O-acetyl via the 2′ hydroxyl group); the product may also include free 5050 PLGA-O-acetyl, 7 docetaxel-conjugate, 10 docetaxel-conjugate, 1 docetaxel-conjugate, product in which two or more polymer chains are linked to docetaxel (e.g., via the 2′ and 7 positions) and/or a trace amount of docetaxel.

### Example 9

### Synthesis, Purification and Characterization of bis(docetaxel) glutamate-5050 PLGA-O-acetyl

A 500-mL, round-bottom flask was charged with 5050 PLGA-O-acetyl [40 g, 5.88 mmol], dibenzyl glutamate (3.74 g, 7.35 mmol), and DMF (120 mL, 3 vol.) and allowed to mix for 10 min to afford a clear solution. CMPI (2.1 g, 8.23 mmol) and TEA (2.52 mL) were added and the solution was stirred at ambient temperature for 3 h. The yellowish solution was added to a suspension of Celite (120 g) in MTBE (2.0 L) over 0.5 h with overhead stirring. The solid was filtered, washed with MTBE (300 mL), and vacuum-dried at 25° C. for 16 h. The solid was then suspended in acetone (400 mL, 10 vol), stirred for 0.5 h, filtered and the filter cake was washed with acetone (3×100 mL). The combined filtrates were concentrated to 150 mL and added to cold water (3.0 L, 0-5° C.) over 0.5 h with overhead stirring. The resulting suspension was stirred for 2 h and filtered through a PP filter. The filter cake was air-dried for 3 h and then vacuum-dried at 28° C. for 16 h to afford the product, dibenzylglutamate 5050 PLGA-O-acetyl [40 g, yield: 95%]. The ¹H NMR analysis indicated that the ratio of benzyl aromatic protons to methine protons of lactide was 10:46. HPLC analysis indicated 96% purity (AUC, 227 nm) and GPC analysis showed Mw: 8.9 kDa and Mn: 6.5 kDa.

Dibenzylglutamate 5050 PLGA-O-acetyl (40 g) was dissolved in ethyl acetate (400 mL) to afford a yellowish solution. Charcoal (10 g) was added to the mixture and stirred for 1 h at ambient temperature. The solution was filtered through a pad of Celite (60 mL) to afford a colorless filtrate. The filter cake was washed with ethyl acetate (3×50 mL) and the combined filtrates were concentrated to 400 mL. Palladium on activated carbon (Pd/C, 5 wt %, 4.0 g) was added, the mixture was evacuated for 1 min, filled up with H₂ using a balloon and the reaction was stirred at ambient temperature for 3 h. The solution was filtered through a Celite pad (100 mL) and the filter cake was washed with acetone (3×50 mL). The combined filtrates had a grey color and were concentrated to 200 mL. The solution was added to a suspension of Celite (120 g) in MTBE (2.0 L) over 0.5 h with overhead stirring. The suspension was stirred at ambient temperature for 1 h and filtered through a PP filter. The filter cake was dried at ambient temperature for 16 h, suspended in acetone (400 mL), and stirred for 0.5 h. The solution was filtered through a PP filter and the filter cake was washed with acetone (3×50 mL). To remove any residual Pd, macroporous polystyrene-2,4,6-trimercaptotriazine resin (MP-TMT, 2.0 g, Biotage, capacity: 0.68 mmol/g) was added at ambient temperature for 16 h with overhead stirring. The solution was filtered through a Celite pad to afford a light grey solution. The solution was concentrated to 200 mL and added to cold water (3.0 L, 0-5° C.) over 0.5 h with overhead stirring. The resulting suspension was stirred at <5° C. for 1 h and filtered through a PP filter. The filter cake was air-dried for 12 h and vacuum-dried for 2 days to afford a semi-glassy solid [glutamic acid-PLGA5050-O-acetyl, 38 g, yield: 95%]. HPLC analysis showed 99.6% purity (AUC, 227 nm) and GPC analysis indicated Mw: 8.8 kDa and Mn: 6.6 kDa.

To remove any residual water, the glutamic acid-PLGA5050-O-acetyl [38 g] was dissolved in acetonitrile (150 mL) and concentrated to dryness. The residue was vacuum-dried at ambient temperature for 16 h to afford the desired product as a light grey powder [36 g]. A 1000-mL, round-bottom flask equipped with a magnetic stirrer was charged with glutamic acid-PLGA5050-O-acetyl [30 g, 4.5 mmol, Mn: 6.6 kDa], docetaxel (4.3 g, 2.9 mmol, 1.2 equiv), DMF (60 mL), and DCM (60 mL). The mixture was stirred for 10 min to afford a light brown solution. The first portion of EDC.HCl (1.6 g, 8.3 mmol) and DMAP (1.0 g, 8.3 mmol) was added and stirred at ambient temperature to yield a dark brown solution. After 2 h, a second portion of EDC.HCl (0.8 g, 4.2 mmol) and DMAP (0.50 g, 4.2 mmol) was added and stirred for an additional 2 to produce a darker solution. A third portion of EDC.HCl (0.3 g, 1.6 mmol) and DMAP (0.2 g, 1.6 mmol) was added. An additional portion of EDC.HCl (0.3 g, 1.6 mmol) and DMAP (0.2 g, 1.6 mmol) was added and stirred at ambient temperature for 2 h. The reaction mixture was added to a suspension of Celite (100 g) in MTBE (3.0 L) over 0.5 h with overhead stirring. The suspension was filtered through a PP filter and the filter cake was dried under vacuum at 25° C. for 12 h. The solid was suspended in acetone (250 mL) for 0.5 h with overhead stirring. The suspension was filtered and the filter cake was washed with acetone (3×60 mL). The combined filtrates were concentrated to 200 mL and added to cold water (3 L, 0° C.) over 0.5 h with overhead stirring. The suspension was filtered through a PP filter; the filter cake was washed with water (3×100 mL) and the solid was dried under vacuum at 25° C. for 16 h to afford a crude product [33 g]. To reduce any possible residual docetaxel, a second MTBE purification was conducted. The crude product was dissolved in acetone (150 mL) and added to a suspension of Celite (100 g) in MTBE (3 L). The suspension was filtered; the solid was vacuum-dried for 3 h, and suspended in acetone (500 mL) with overhead stirring. The suspension was filtered and the filter cake was washed with acetone (3×100 mL). The combined filtrates were concentrated to 200 mL and co-evaporated with acetonitrile (100 mL) to dryness. The residue was dissolved in acetone (200 mL) and the solution was precipitated into a suspension of Celite® (100 g)/MTBE (3 L) a third time. The mixture was stirred at ambient temperature for 1 h and filtered. The filter cake was washed with MTBE (2×200 mL) and vacuum-dried at ambient temperature overnight. The bis(docetaxel) glutamate-5050 PLGA-O-acetyl/Celite complex was suspended in acetone (300 mL) with overhead stirring. The suspension was filtered and added to cold water (3 L) over 0.5 h with overhead stirring. The suspension was stirred at <5° C. for 1 h and filtered through a PP filter. The filter cake was washed with water (3×200 mL); the filter cake was conditioned for 0.5 h and vacuum-dried for 2 days to afford the desired product as an off-white powder [30 g, yield: 88%;]. This product was purified by another MTBE precipitation without using Celite. The product was dissolved in acetone to afford a solution (200 mL) and added to cold MTBE (2 L, 0° C.) over 1 h with overhead stirring. The resulting suspension was filtered and the filter cake was vacuum-dried at 25° C. for 16 h to afford a product with a tan color [34 g]. This sample was further dried for another 24 h and the residual MTBE was not reduced. To remove the residual MTBE, the product was precipitated into water. The isolated solid was vacuum-dried for 2 days to constant weight to afford the desired product as an off-white powder [bis(docetaxel) glutamate-5050 PLGA-O-acetyl, 28.5 g, yield: 84%]. The ¹H NMR analysis indicated that the docetaxel loading was 10% and HPLC analysis showed >99.5% purity (AUC, 227 nm). GPC analysis indicated Mw: 9.9 kDa and Mn: 6.1 kDa. The major product is bis(2′-docetaxel) glutamate-5050 PLGA-O-acetyl (wherein each docetaxel is attached to the glutamate linker via the 2′ hydroxyl group); the product may also include free 5050 PLGA-O-acetyl, mono(2′-docetaxel) glutamate-5050 PLGA-O-acetyl, mono(7-docetaxel) glutamate-5050 PLGA-O-acetyl, mono(10-docetaxel) glutamate-5050 PLGA-O-acetyl, mono(1-docetaxel) glutamate-5050 PLGA-O-acetyl, (2′-docetaxel)(7-docetaxel) glutamate-5050 PLGA-O-acetyl, (2′-docetaxel)(10-docetaxel) glutamate-5050 PLGA-O-acetyl, (2′-docetaxel)(1-docetaxel) glutamate-5050 PLGA-O-acetyl, (7-docetaxel)(10-docetaxel) glutamate-5050 PLGA-O-acetyl, (7-docetaxel)(1-docetaxel) glutamate-5050 PLGA-O-acetyl, (10-docetaxel)(1-docetaxel) glutamate-5050 PLGA-O-acetyl, and/or a trace amount of docetaxel.

### Example 10

### Synthesis, Purification and Characterization of tetra-(docetaxel)triglutamate-5050 PLGA-O-acetyl

A 250-mL, round-bottom flask equipped with a magnetic stirrer was charged with N-(tert-butoxycarbonyl)-L-glutamic acid (20 g, 40 mmol), (S)-dibenzyl 2-aminopentanedioate (4.85 g, 19.5 mmol), and DMF (100 mL). The mixture was stirred for 5 min to afford a clear solution. EDC.HCl (8.5 g, 44.3 mmol) and DMAP (9.8 g, 80 mmol) were added. The reaction was stirred at ambient temperature for 3 h, at which time HPLC analysis indicated completion of the reaction. The reaction was concentrated to a syrup (˜75 g) and EtOAc (250 mL) was added with overhead stirring. The resulting suspension was filtered to remove the N,N-dimethyl pyridiniump-toluenesulfonate. The filtrate was concentrated to a yellowish oil and water (200 mL) was added with vigorous stirring. White solid was gradually formed and the suspension was filtered. The solid was washed with water (2×50 mL) and dried under vacuum for 24 h to afford the N-Boc-tetrabenzyl-triglutamate product as a white powder [16.5 g, yield: 95%]. The 1H NMR analysis showed the desired product and HPLC analysis indicated a 92% purity (AUC, 254 nm). This crude product was further purified by recrystallization as follows. N-Boc-tetrabenzyl-triglutamate (15 g) was dissolved in hot IPAc (15 mL, 1 vol) and the solution was allowed to cool down to ambient temperature. A hydrogel like solid was formed and it was slurried in MTBE (200 mL) for 1 h, filtered. The filtration was slow owing to the hydrogel-like particles. The hydrogel solid was vacuum-dried at ambient temperature to afford product as a white powder [12.5 g, recovery yield: 83%]. The 1H NMR analysis showed the desired product and HPLC analysis indicated ˜100% purity (AUC, 254 nm).

A 250-mL, round bottom flask was charged with N-tert-butyloxycarbonyl-tetrabenzyl-triglutamate [N-t-BOC-tetrabenzyl-triglutamate, 11 g, 12.7 mmol] and DCM (25 mL) to afford a clear solution. Trifluoroacetic acid (TFA, 25 mL) was added to the solution and the reaction was stirred at ambient temperature. The solution was concentrated to a residue, dissolved in DCM (200 mL) and washed with saturated sodium bicarbonate (NaHCO₃, 2×25 mL) and brine (30 mL). The organic layer was separated and dried over sodium sulfate (Na₂SO₄, 15 g). The solution was filtered and the filtrate was concentrated to a residue and vacuum-dried at ambient temperature for 16 h to afford the desired product (NH₂-tetrabenzyl-triglutamate) as a wax-like semi-solid product [9.3 g, yield: 96%]. HPLC analysis indicated a 97% purity (AUC, 254 nm).

A 1000-mL, round-bottom flask equipped with a magnetic stirrer was charged with NH₂-tetrabenzyl-triglutamate [4.0 g, 5.3 mmol], o-acetyl PLGA 5050 [30 g, 4.4 mmol, Mn: 6.8 kDa,], and DMF (100 mL). The mixture was stirred for a few minutes to afford a clear solution. 1-chloro-4-methylpyridinium iodide (CMPI, 1.7 g, 6.6 mmol) and trifluoroacetic acid (TEA, 1.3 mL, 8.8 mmol) were added and the reaction was stirred at ambient temperature for 3 h. The reaction mixture was added into cold water (2 L) over 1 h with overhead stirring. The generated suspension was filtered through a PP filter. The filter cake was washed with water (3×300 mL) and air-dried at ambient temperature for 16 h to afford a crude product. It was dissolved in acetonitrile (200 mL) and the solution concentrated to dryness. The residue was dissolved in acetone (100 mL) and the solution was added to cold MTBE (0° C., 2 L) over 0.5 h with overhead stirring to afford a suspension. It was filtered through a PP filter and the filter cake was vacuum-dried for 16 h to afford the product (tetrabenzyl-triglutamate-PLGA 5050-O-acetyl [30 g, yield: 88%]. The ¹H NMR analysis indicated the ratio of benzyl aromatic protons over methine protons of lactide was 20:45. HPLC analysis showed >95% purity (AUC, 227 nm) and GPC analysis indicated a Mw: 8.9 kDa and a Mn: 6.7 kDa.

The tetrabenzyl-triglutamate-PLGA 5050-O-acetyl [30 g, 1.5 mmol] was dissolved in ethyl acetate (300 mL) to afford a pale yellowish solution. Charcoal (10 g) was added and the mixture was stirred at ambient temperature for 1 h and filtered through a Celite pad (100 mL). The filtrate became colorless and was transferred to a 1000-mL, round bottom flask equipped with a magnetic stirrer. Palladium on activated carbon (Pd/C, 5 wt. %, 4.0 g) was added, the mixture was evacuated for 1 min, filled up with H₂ using a balloon and stirred at ambient temperature for 3 h. It was filtered through a Celite pad (100 mL) and the filter cake was washed with acetone (3×50 mL). The combined filtrates had a grey color and were filtered through multiple 0.45 μM polytetrafluoroethylene (PTFE) filters. The filtrate was concentrated to 150 mL and added to cold water (1.5 L, 0-5° C.) over 0.5 h with overhead stirring. The suspension was filtered and the filter cake was washed with water (3×100 mL), conditioned for 0.5 h, and vacuum-dried for 24 h to afford a white powder [triglutamate-PLGA5050-O-acetyl, 21 g, yield: 72%]. HPLC analysis indicated a 100% purity (AUC, 227 nm) and. GPC analysis showed a Mw: 9.2 kDa and Mn: 6.9 kDa.

A 1000-mL, round-bottom flask equipped with a magnetic stirrer was charged with triglutamate-PLGA5050-O-acetyl [20 g, 2.9 mmol, Mn 6.9 kDa,], docetaxel (5.7 g, 7.0 mmol, 2.4 equiv.), and DMF (75 mL). The mixture was stirred for 5 min to afford a clear solution. EDC.HCl (1.08 g, 5.6 mmol) and DMAP (0.72 g, 5.6 mmol) were added and the reaction was stirred at ambient temperature for 3 h. A second portion EDC.HCl (0.54 g, 2.8 mmol), and DMAP (0.54 g, 2.8 mmol) was added and the reaction was stirred for an additional 3 h. A third portion of EDC.HCl (0.36 g, 1.9 mmol) and DMAP (0.24 g, 1.9 mmol) was added and the reaction was stirred for 14 h. An additional portion of EDC.HCl (0.36 g, 1.9 mmol) and DMAP (0.24 g, 1.9 mmol) was added and the reaction was stirred for another 4 h. The reaction mixture was added to a suspension of Celite (60 g) in MTBE (2.0 L) over 0.5 h with overhead stirring. The suspension was filtered through a PP filter and the crude product/Celite complex was dried under vacuum at 25° C. for 12 h. The product/complex was suspended in acetone (200 mL) for 0.5 h with overhead stirring and filtered. The filter cake was washed with acetone (3×60 mL). The combined filtrates were concentrated to 100 mL. A second Celite/MTBE precipitation was conducted; the filtrate from the acetone extraction was concentrated to 100 mL, added to cold water (1.0 L, 0-5° C.) with overhead stirring and filtered. The solid was vacuum-dried for 2 days to afford crude product as a white powder [24 g]. The crude product was dissolved in acetone (120 mL) and added to a suspension of Celite (70 g, Aldrich, standard supercell, acid washed) in MTBE (2.0 L) at ambient temperature with overhead stirring. The suspension was stirred for 2 h and filtered through a fritted funnel. The filter cake was washed with MTBE (2×200 mL) and vacuum-dried at ambient temperature overnight. The solid was suspended in acetone (200 mL) with overhead stirring for 1 h. The suspension was filtered through a fritted funnel and the filter cake was rinsed with acetone (3×100 mL). The combined filtrates were concentrated to ˜150 mL and precipitated into Celite/MTBE a fourth time. To facilitate the purification, the filtrate was concentrated to ˜120 mL and added to MTBE (2.0 L) at ambient temperature with vigorous stirring. The suspension was filtered through a fritted funnel and the filter cake was vacuum-dried for 16 h to afford a crude product as a white powder containing ˜30 wt % of residual MTBE [30 g, >100% yield,]. The crude product was dissolved in acetone (120 mL) and the solution was precipitated into MTBE (2.0 L). The resultant suspension was stirred at ambient temperature for 3 h and filtered through a fritted funnel. The filter cake was vacuum-dried for 12 h to afford a white solid [30 g]. At this point, a third water precipitation was conducted to isolate the product and reduce the residual MTBE. The above crude product was dissolved in acetone (100 mL) and the solution was added to cold water (1.5 L, 0-5° C.) over 0.5 h with overhead stirring. The suspension was filtered through a fritted funnel. The filter cake was washed with water (3×200 mL), conditioned for 2 h, and vacuum-dried for 2 days to afford the desired product (tetra-(docetaxel) triglutamate-5050 PLGA-O-acetyl) as a white powder [20 g, yield: 78%;]. HPLC analysis showed a 99.5% purity along with 0.5% of residual docetaxel. GPC analysis indicated a Mw: 10.8 kDa and Mn: 6.6 kDa.

The major product is tetra(2′-docetaxel) triglutamate-5050 PLGA-O-acetyl (wherein each docetaxel is attached to the triglutamate linker via the 2′ hydroxyl group); the product may also include free 5050 PLGA-O-acetyl, monofunctionalized polymers (e.g., mono(2′-docetaxel) triglutamate-5050 PLGA-O-acetyl or monosubstituted products attached via the 7, 10 or 1 hydroxyl groups), difunctionalized polymers (e.g., bis(2′-docetaxel)triglutamate-5050 PLGA-O-acetyl, or disubstituted products with docetaxel molecules attached via other hydroxyl groups or mixtures thereof), trifunctionalized polymers (e.g., tris(2′-docetaxel)triglutamate-5050 PLGA-O-acetyl, or trisubstituted products with docetaxel molecules attached via other hydroxyl groups or mixtures thereof), and/or a trace amount of docetaxel.

### Example 11

### Synthesis, purification and characterization of folate-PEG-PLGA-lauryl ester

The synthesis of folate-PEG-PLGA-lauryl ester involves the direct coupling of folic acid to PEG bisamine (Sigma-Aldrich, n=75, MW 3350 Da). PEG bisamine was purified due to the possibility that small molecular weight amines were present in the product. 4.9 g of PEG bisamine was dissolved in DCM (25 mL, 5 vol) and then transferred into MTBE (250 mL, 50 vol) with vigorous agitation. The polymer precipitated as white powder. The mixture was then filtered and the solid was dried under vacuum to afford 4.5 g of the product [92%]. The ¹H NMR analysis of the solid gave a clean spectrum; however, not all alcohol groups were converted to amines based on the integration of α-methylene to the amine group (63% bisamine, 37% monoamine).

Folate-(γ)CO—NH-PEG-NH₂ was synthesized using the purified PEG bisamine. Folic acid (100 mg, 1.0 equiv.) was dissolved in hot DMSO (4.5 mL, 3 vol to PEG bisamine). The solution was cooled to ambient temperature and (2-(7-Aza-1H-benzotriazole-1-yl)-1,1,3,3-tetramethyluronium hexafluorophosphate) (HATU, 104 mg, 1.2 equiv.) and N,N-Diisopropylethylamine (DIEA, 80 μL, 2.0 equiv.) were added. The resulting yellow solution was stirred for 30 minutes and PEG bisamine (1.5 g, 2 equiv.) in DMSO (3 mL, 2 vol) was added. Excess PEG bisamine was used to avoid the possible formation of di-adduct of PEG bisamine and to improve the conversion of folic acid. The reaction was stirred at 20° C. for 16 h and directly purified by CombiFlash using a C18 column (RediSep, 43 g, C18). The fractions containing the product were combined and the CH₃CN was removed under vacuum. The remaining water solution (˜200 mL) was extracted with chloroform (200 mL×2). The combined chloroform phases were concentrated to approximately 10 mL and transferred into MTBE to precipitate the product as a yellow powder. In order to completely remove any unreacted PEG bisamine in the material, the yellow powder was washed with acetone (200 mL) three times. The remaining solid was dried under vacuum to afford a yellow semi-solid product (120 mg). HPLC analysis indicated a purity of 97% and the ¹H NMR analysis showed that the product was clean.

Folate-(γ)CO—NH-PEG-NH2 was reacted with p-nitrophenyl-COO-PLGA-CO₂-lauryl to provide folic acid-PEG-PLGA-lauryl ester. To prepare p-nitrophenyl-COO-PLGA-CO₂-lauryl, PLGA 5050 (lauryl ester) [10.0 g, 1.0 equiv.] and p-nitrophenyl chloroformate (0.79 g, 2.0 equiv.) were dissolved in DCM. To the dissolved polymer solution, one portion of TEA (3.0 equiv.) was added. The resulting solution was stirred at 20° C. for 2 h and the ¹H NMR analysis indicated complete conversion. The reaction solution was then transferred into a solvent mixture of 4:1 MTBE/heptanes (50 vol). The product precipitated and gummed up. The supernatant was decanted off and the solid was dissolved in acetone (20 vol). The resulting acetone suspension was filtered and the filtrate was concentrated to dryness to produce the product as a white foam [7.75 g, 78%, Mn=4648 based on GPC]. The ¹H NMR analysis indicated a clean product with no detectable p-nitrophenol.

Folate-(γ)CO—NH-PEG-NH2 (120 mg, 1.0 equiv.) was dissolved in DMSO (5 mL) and TEA (3.0 equiv.) was added. The pH of the reaction mixture was 8-9. p-nitrophenyl-COO-PLGA-CO₂-lauryl (158 mg, 1.0 equiv.) in DMSO (1 mL) was added and the reaction was monitored by HPLC. A new peak at 16.1 min (˜40%, AUC, 280 nm) was observed from the HPLC chromatogram in 1 h. A small sample of the reaction mixture was treated with excess 1,8-diazabicyclo[5.4.0]undec-7-ene (DBU) and the color instantly changed to dark yellow. HPLC analysis of this sample indicated complete disappearance of p-nitrophenyl-COO-PLGA-CO₂-lauryl and the 16.1 min peak. Instead, a peak on the right side of folate-(γ)CO—NH-PEG-NH2 appeared. It can be concluded that the p-nitrophenyl-COO-PLGA-CO₂-lauryl and the possible product were not stable under strong basic conditions. In order to identify the new peak at 16.1 min, ˜⅓ of the reaction mixture was purified by CombiFlash. The material was finally eluted with a solvent mixture of 1:4 DMSO/CH₃CN. It was observed that this material was yellow which could have indicated folate content. Due to the large amount of DMSO present, this material was not isolated from the solution. The fractions containing unreacted folate-(γ)CO—NH-PEG-NH2 was combined and concentrated to a residue. A ninhydrin test of this residue gave a negative result, which may imply the lack of amine group at the end of the PEG. This observation can also explain the incomplete conversion of the reaction.

The rest of reaction solution was purified by CombiFlash. Similarly to the previous purification, the suspected yellow product was retained by the column. MeOH containing 0.5% TFA was used to elute the material. The fractions containing the possible product were combined and concentrated to dryness. The ¹H NMR analysis of this sample indicated the existence of folate, PEG and lauryl-PLGA and the integration of these segments was close to the desired value of 1:1:1 ratio of all three components. High purities were observed from both HPLC and GPC analyses. The Mn based on GPC was 8.7 kDa. The sample in DMSO was recovered by precipitation into MTBE.

### Example 12

### Synthesis and purification of docetaxel-2′-hexanoate-5050 PLGA-O-acetyl

A 500-mL round-bottom flask equipped with a magnetic stirrer was charged with 6-(carbobenzyloxyamino) caproic acid (4.13 g, 15.5 mmol), docetaxel (12.0 g, 14.8 mmol), and dichloromethane (240 mL). The mixture was stirred for 5 min to afford a clear solution, to which 1-ethyl-3-(3-dimethyllaminopropyl)carbodiimide hydrochloride (EDC.HCl) (3.40 g, 17.6 mmol) and 4 dimethylaminopyridine (DMAP) (2.15 g, 17.6 mmol) were added. The mixture was stirred at ambient temperature for 3 h at which time, IPC analysis showed a 57% conversion along with 34% residual docetaxel. An additional 0.2 equivalents of EDC.HCl and DMAP were added and the reaction was stirred for 3 h, at which time IPC analysis showed 63% conversion. An additional 0.1 equivalents of 6-(carbobenzyloxyamino) caproic acid along with 0.2 equivalents of EDC.HCl and DMAP were added. The reaction was stirred for 12 h and IPC analysis indicated 74% conversion and 12% residual docetaxel. To further increase the conversion, an additional 0.1 equivalents of 6-(carbobenzyloxyamino) caproic acid and 0.2 equivalents of EDC.HCl and DMAP were added. The reaction was continued for another 3 h at which time, IPC analysis revealed 82% conversion and the residual docetaxel dropped to 3%. The reaction was diluted with DCM (200 mL) and washed with 0.01% HCl (2×150 mL) and brine (150 mL). The organic layer was separated, dried over sodium sulfate, and filtered. The filtrate was concentrated to a residue and dissolved in ethyl acetate (25 mL). The solution was divided into two portions, each of which was passed through a 120-g silica column (Biotage F40). The flow rate was adjusted to 20 mL/min and 2000 mL of 55:45 ethyl acetate/heptanes was consumed for each of the column purifications. The fractions containing minor impurities were combined, concentrated, and passed through a column a third time. The fractions containing product (shown as a single spot by TLC analysis) from all three column purifications were combined, concentrated to a residue, vacuum-dried at ambient temperature for 16 h to afford the product, H₂N—(CH₂)₅CO—O-2′-docetaxel as a white powder [10 g, yield: 64%]. The ¹H NMR analysis was consistent with the assigned structure of the desired product; however, HPLC analysis (AUC, 227 nm) indicated only a 97% purity along with 3% of bis-adducts. To purify the H₂N—(CH₂)₅CO—O-2′-docetaxel product, ethyl acetate (20 mL) was added to dissolve the batch to produce a clear solution. The solution was divided into two portions, each of which was passed through a 120-g silica column. The fractions containing product were combined, concentrated to a residue, vacuum-dried at ambient temperature for 16 h to afford the desired product (CBZ—NH—(CH₂)₅CO—O-2′-docetaxel) as a white powder [8.6 g, recovery yield: 86%]. HPLC analysis (AUC, 227 nm) indicated >99% purity.

A 1000-mL round-bottom flask equipped with a magnetic stirrer was charged with CBZ—NH—(CH₂)₅CO—O-2′-docetaxel product [5.3 g, 5.02 mmol] and THF (250 mL). To the resultant clear solution, MeOH (2.5 mL) and 5% Pd/C (1.8 g, 10 mol % of Pd) were added. The mixture was cooled to 0° C. and methanesulfonic acid (316 μL, 4.79 mmol) was added. The flask was evacuated for 10 seconds and filled with hydrogen using a balloon. After 3 h, IPC analysis indicated 62% conversion. The ice-bath was removed and the reaction was allowed to warm up to ambient temperature. After an additional 3 h, IPC analysis indicated that the reaction was complete. The solution was filtered through a Celite® pad and the filtrate was black in appearance. To remove the possible residual Pd, charcoal (5 g, Aldrich, Darco®) was added and the mixture was placed in a fridge overnight and filtered through a Celite® pad to produce a clear colorless solution. This was concentrated at <20° C. under reduced pressure to a volume of ˜100 mL, to which methyl tert-butyl ether (MTBE) (100 mL) was added. The resultant solution was added to a solution of cold MTBE (1500 mL) with vigorous stirring over 0.5 h. The suspension was left at ambient temperature for 16 h, the upper clear supernatant was decanted off and the bottom layer was filtered through a 0.45 μm filter membrane. The filter cake was vacuum-dried at ambient temperature for 16 h to afford the desired product (H₂N—(CH₂)₅CO—O-2′-docetaxel) as a white solid [4.2 g, yield: 82%]. HPLC analysis indicated >99% purity and the ¹H NMR analysis indicated the desired product.

A 100-mL round-bottom flask equipped with a magnetic stirrer was charged with 5050 PLGA-O-acetyl (5.0 g, 0.7 mmol), H₂N—(CH₂)₅CO—O-2′-docetaxel [0.85 g, 0.84 mmol, GAO-G-28(3)], DCM (5 mL), and DMF (20 mL). The mixture was stirred for 5 min to produce a clear solution. EDC.HCl (0.2 g, 1.05 mmol) and DMAP (0.21 g, 1.75 mmol) were added and the reaction was stirred for 3 h, at which time IPC analysis indicated 79% conversion along with 18% of H₂N—(CH₂)₅CO—O-2′-docetaxel. Two small impurities were observed at 11.6 min and 11.7 min (2.8%, AUC, 227 nm). An additional portion of EDC.HCl (0.1 g, 0.5 mmol) and DMAP (0.15 g, 1.2 mmol) was added and the reaction was stirred overnight. IPC analysis showed 92% conversion along with 6% of H₂N—(CH₂)₅CO—O-2′-docetaxel; the level of the two impurities did not change. To increase the conversion, an additional amount of 5050 PLGA-O-acetyl (0.5 g) along with EDC.HCl (0.1 g) and DMAP (0.15 g) was added and the reaction was stirred at ambient temperature for 3 h. IPC analysis showed a 95.6% conversion along with 3.0% of H₂N—(CH₂)₅CO—O-2′-docetaxel; the two impurities were about 1.3%. The reaction was combined with a previously prepared product and added to a suspension of Celite® (20 g) in MTBE (600 mL) with mechanical stirring over 30 min. The suspension was stirred at ambient temperature for 0.5 h and filtered. The filter cake was air-dried for 30 min and then vacuum-dried such that the residual MTBE contained no more than 5 wt %. The polymer/Celite® complex was then suspended in acetone (50 mL) and the slurry was stirred for 30 min, filtered through a Celite pad. The filter cake was washed with acetone (3×30 mL). The combined filtrates were concentrated to ˜25 mL and this solution was analyzed by HPLC showing that the level of H₂N—(CH₂)₅CO—O-2′-docetaxel or the impurities was identical to these prior to MTBE precipitation. The solution was added to cold water (500 mL) containing 0.05% acetic acid over 30 min. The suspension was stirred at 0° C. for 1 h and filtered through a PP filter. The filter cake was washed with water (3×50 mL), conditioned for 30 min, vacuum-dried at ambient temperature for 48 h to produce docetaxel-2′-hexanoate-5050 PLGA-O-acetyl as a white powder [6.3 g, 85%]. The ¹H NMR analysis indicated 10.5 wt % of loading. No DMAP or DMF was observed. GPC analysis indicated a Mw of 8.2 kDa and a Mn of 5.7 kDa. HPLC analysis indicated a purity of 98.6% (AUC, 230 nm) and a 0.75% of H₂N—(CH₂)₅CO—O-2′-docetaxel. The two impurities totaled 0.5% (AUC, 230 nm).

### Example 13

### Synthesis, purification and characterization of O-acetyl-5050-PLGA-(2′-β-alanine glycolate)-docetaxel

A 1000 mL round-bottom flask equipped with a magnetic stirrer was charged with carbobenzyloxy-β-alanine (Cbz-β-alanine, 15.0 g, 67.3 mmol), tert-butyl bromoacetate (13.1 g, 67.3 mmol), acetone (300 mL), and potassium carbonate (14 g, 100 mmol). The mixture was heated to reflux at 60° C. for 16 h, cooled to ambient temperature and then the solid was removed by filtration. The filtrate was concentrated to a residue, dissolved in ethyl acetate (EtOAc, 300 mL), and washed with 100 mL of water (three times) and 100 mL of brine. The organic layer was separated, dried over sodium sulfate and filtered. The filtrate was concentrated to clear oil [22.2 g, yield: 99%]. HPLC analysis showed 97.4% purity (AUC, 227 nm) and ¹H NMR analysis confirmed the desired intermediate product, t-butyl (carbobenzyloxy-β-alanine) glycolate.

To prepare the intermediate product, carbobenzyloxy-β-alanine glycolic acid (Cbz-β-alanine glycolic acid), a 100 mL round-bottom flask equipped with a magnetic stirrer was charged with t-butyl (Cbz-β-alanine) glycolate [7.5 g, 22.2 mmol] and formic acid (15 mL, 2 vol). The mixture was stirred at ambient temperature for 3 h to give a red-wine color and HPLC analysis showed 63% conversion. The reaction was continued stirring for an additional 2 h, at which point HPLC analysis indicated 80% conversion. An additional portion of formic acid (20 mL, 5 vol in total) was added and the reaction was stirred overnight, at which time HPLC analysis showed that the reaction was complete. The reaction was concentrated under vacuum to a residue and redissolved in ethyl acetate (7.5 mL, 1 vol.). The solution was added to the solvent heptanes (150 mL, 20 vol.) and this resulted in the slow formation of the product in the form of a white suspension. The mixture was filtered and the filter cake was vacuum-dried at ambient temperature for 24 h to afford the desired product, Cbz-β-alanine glycolic acid as a white powder [5.0 g, yield: 80%]. HPLC analysis showed 98% purity. The ¹H NMR analysis in DMSO-d6 was consistent with the assigned structure of Cbz-β-alanine glycolic acid [δ 10.16 (s, 1H), 7.32 (bs, 5H), 5.57 (bs, 1H), 5.14 (s, 2H), 4.65 (s, 2H), 3.45 (m, 2H), 2.64 (m, 2H)].

To prepare the intermediate, docetaxel-2′-carbobenzyloxy-β-alanine glycolate (docetaxel-2′—Cbz-β-alanine glycolate), a 250-mL round-bottom flask equipped with a magnetic stirrer was charged with docetaxel (5.03 g, 6.25 mmol), Cbz-β-alanine glycolic acid [1.35 g, 4.80 mmol] and dichloromethane (DCM, 100 mL). The mixture was stirred for 5 min to produce a clear solution, to which N-(3-Dimethylaminopropyl)-N′-ethylcarbodiimide hydrochloride (EDC.HCl, 1.00 g, 5.23 mmol) and 4-(dimethylamino)pyridine (DMAP, 0.63 g, 5.23 mmol) were added. The mixture was stirred at ambient temperature for 3 h, at which point HPLC analysis showed 48% conversion along with 46% of residual docetaxel. A second portion of Cbz-β-alanine glycolic acid (0.68 g, 2.39 mmol), EDC.HCl (0.50 g, 1.04 mmol) and DMAP (0.13 g, 1.06 mmol) were added and the reaction was allowed to stirred overnight. At this point, HPLC analysis showed 69% conversion along with 12% of residual docetaxel. The solution was diluted to 200 mL with DCM and then washed with 80 mL of water (twice) and 80 mL of brine. The organic layer was separated, dried over sodium sulfate, and then filtered. The filtrate was concentrated to a residue, re-dissolved in 10 mL of chloroform, and purified using a silica gel column. The fractions containing product (shown as a single spot by TLC analysis) were combined, concentrated to a residue, vacuum-dried at ambient temperature for 16 h to produce docetaxel-2′—Cbz-β-alanine glycolate as a white powder [3.5 g, yield: 52%]. HPLC analysis (AUC, 227 nm) indicated >99.5% purity. The ¹H NMR analysis confirmed the corresponding peaks.

To prepare the intermediate, docetaxel-2′-β-alanine glycolate, a 250 mL round-bottom flask equipped with a magnetic stirrer was charged with docetaxel-2′—Cbz-β-alanine glycolate [3.1 g, 2.9 mmol] and tetrahydrofuran (THF, 100 mL). To the clear solution methanol (MeOH, 4 mL), methanesulfonic acid (172 μL, 2.6 mmol), and 5% palladium on activated carbon (Pd/C, 1.06 g, 10 mol % of Pd) were added. The mixture was evacuated for 15 seconds and filled with hydrogen using a balloon. After 3 h, HPLC analysis indicated that the reaction was complete. Charcoal (3 g, Aldrich, Darco®#175) was then added and the mixture was stirred for 15 min and filtered through a Celite® pad to produce a clear colorless solution. It was concentrated under reduced pressure at <20° C. to ˜5 mL, to which 100 mL of heptanes was added slowly resulting in the formation of a white gummy solid. The supernatant was decanted and the gummy solid was vacuum-dried for 0.5 h to produce a white solid. A volume of 100 mL of heptanes were added and the mixture was triturated for 10 min and filtered. The filter cake was vacuum-dried at ambient temperature for 16 h to produce docetaxel-2′-β-alanine glycolate as a white powder [2.5 g, yield: 83%]. The HPLC analysis indicated >99% purity (AUC, 230 nm). MS analysis revealed the correct molecular mass (m/z: 936.5).

A 100 mL round bottom equipped with a magnetic stirrer was charged with 0-acetyl-5050-PLGA [5.0 g, 0.7 mmol], docetaxel-2′-β-alanine glycolate [0.80 g, 0.78 mmol], dichloromethane (DCM, 5 mL) and dimethylformamide (DMF, 20 mL). The mixture was stirred for 5 min to produce a clear solution. EDC.HCl (0.22 g, 1.15 mmol) and DMAP (0.22 g, 1.80 mmol) were added to the mixture and the reaction was stirred for 3 h, at which time HPLC analysis indicated completion of the reaction. The reaction was concentrated under vacuum to remove DCM and then DCM was twice exchanged with 10 mL of acetone. The residue was diluted with acetone to 30 mL and precipitated in cold water containing 600 mL of 0.1% acetic acid. The resulting suspension was filtered and the filter cake was vacuum-dried for 24 h to afford a crude product as a white powder [yield=5.0 g]. The ¹H NMR analysis indicated the presence of trace amounts of DMF and DMAP. The docetaxel loading was estimated to be approximately 10 wt % and HPLC analysis indicated >99% purity (AUC, 230 nm). To purify the crude product, it was dissolved in 20 mL of acetone and precipitated in 500 mL of cold water. The suspension was filtered through a polypropylene (PP) filter and the filter cake was vacuum-dried for 48 h to produce O-acetyl-5050-PLGA-(2′-β-alanine glycolate)-docetaxel as a white powder [4.8 g, yield: 84%]. GPC analysis showed that Mw=7.4 kDa, Mn=5.0 kDa and PDI=1.48. ¹H NMR analysis indicated a docetaxel loading of 10.7 wt % and HPLC analysis showed >99% purity (AUC, 230 nm).

### Synthetic scheme of O-acetyl-5050-PLGA-(2′-β-alanine glycolate)-docetaxel

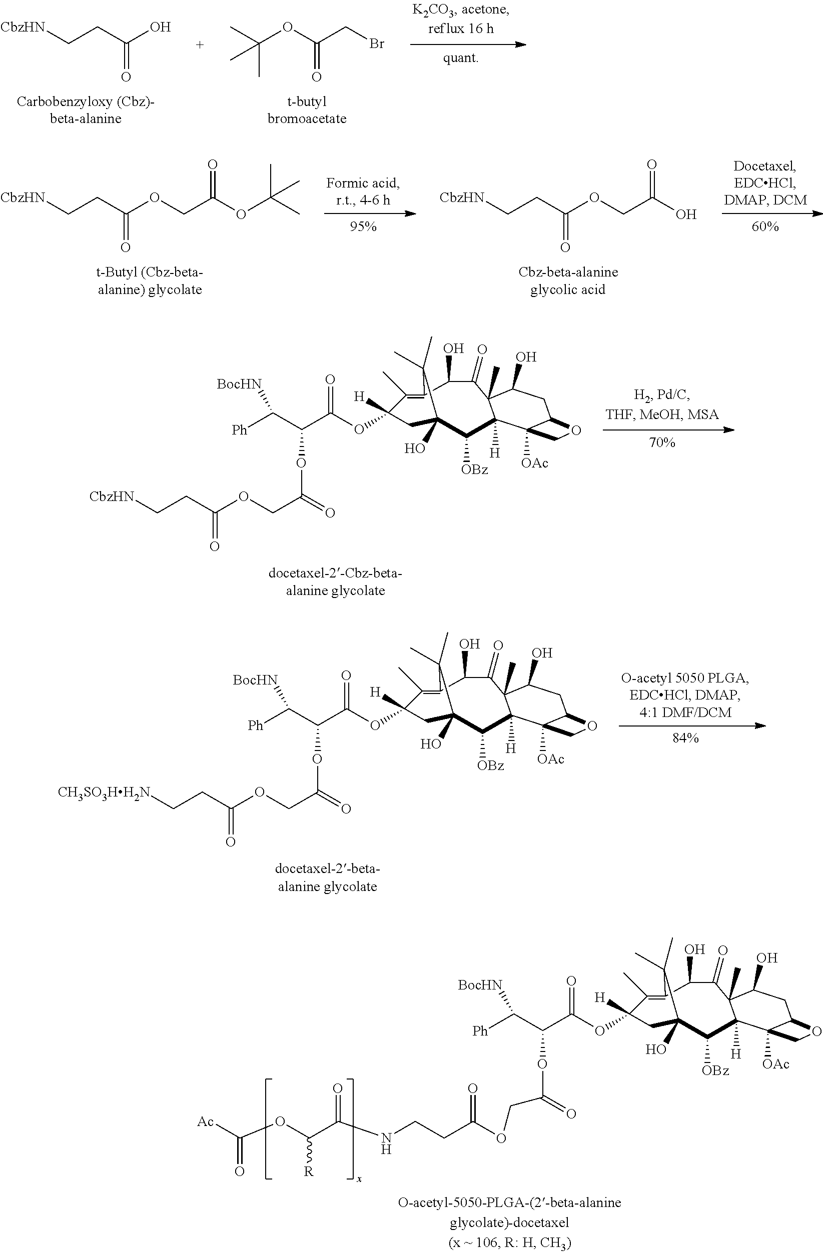

### Example 14

### Synthesis of lauryl-polylactide (PLA)-O—CO—O-docetaxel

To prepare lauryl-PLA-O—CO—O-docetaxel, PLA-lauryl ester (inherent viscosity: 1-2 dL/g) was first purified. A mass of 25 g of PLA lauryl ester was dissolved in a 1:1 MTBE/heptanes mixture (100 vol.) with mechanical stirring at ambient temperature. The entire solution was concentrated to dryness and further dried under vacuum at ambient temperature to afford a white powder (18 g). The ¹H NMR analysis indicated 1.44 equivalents of lauryl segment. GPC analysis indicated a Mn and Mw of 8.5 kDa and 10.7 kDa respectively.

A 250-mL round-bottom flask was charged with purified PLA-lauryl ester (10.0 g, 1.18 mmol] and anhydrous DCM (50 mL) under nitrogen. The mixture was stirred for 10 min to afford a clear solution. p-Nitrophenyl chloroformate (0.5 g, 2.4 mmol) was added to the solution and the mixture was stirred for an additional 10 min. A solution of TEA (0.5 mL) was then added dropwise and the reaction was stirred at ambient temperature for 6 h. An additional one equivalent of p-nitrophenyl chloroformate (0.25 g, 1.2 mmol) and TEA (0.25 mL) were added and the reaction was stirred for 12 h. IPC analysis (¹H NMR) indicated completion of the reaction. The solution was concentrated to a residue and dissolved in acetone (20 mL), resulting in a cloudy mixture. This mixture was filtered to remove TEA.HCl and the filtrate was precipitated into a solution of 2:1 MTBE/heptanes (1000 mL). The resulting gummy solid was dissolved in acetone (20 mL) and concentrated to a residue, which was dried under vacuum at ambient temperature for 24 h to afford 5.6 g of p-NO₂-phenyl-COO-PLA-CO₂-lauryl [yield: ˜50%]. The ¹H NMR analysis confirmed the desired product and GPC analysis showed a Mn and Mw of 9.3 and 11.1 kDa respectively.

A 100-mL round-bottom flask was charged with p-NO₂-phenyl-COO-PLA-CO₂-lauryl [2.5 g, 0.28 mmol], docetaxel (0.20 g, 0.25 mmol) and 1:1 DCM/EtOAc (15 mL). The entire mixture was stirred for 10 min. A catalyst, dialkylaminopyridine (DMAP, 61 mg, 0.5 mmol) was added to the mixture and allowed to stir at ambient temperature under N₂ for 6 h. The reaction was stirred for another 10 h to reach completion as confirmed by IPC analysis (¹H NMR). The reaction was then filtered through a 0.45 μM PTFE membrane and the filtrate was added dropwise into 2:1 MTBE/heptanes (600 mL) with vigorous agitation, resulting in a suspension. The milky supernatant was decanted off and the gummy solid was dissolved in acetone (15 mL). The solution was then added dropwise into an ice-cold solution of 0.1% sodium bicarbonate (300 mL) with agitation. The resulting suspension was filtered and the solid was dried under vacuum at ambient temperature for 24 h to afford 1.34 g of lauryl-PLA-O—CO—O-docetaxel [yield: 51%]. The ¹H NMR analysis indicated 9.3 wt % of docetaxel loading. GPC analysis showed a Mn and Mw of 12.4 and 14.3 kDa respectively.

### Example 15

### Synthesis of PLGA-PEG-PLGA

The triblock copolymer PLGA-PEG-PLGA will be synthesized using a method developed by Zentner et al., Journal of Controlled Release, 72, 2001, 203-215. The molecular weight of PLGA obtained using this method would be ˜3 kDa. A similar method reported by Chen et al., International Journal of Pharmaceutics, 288, 2005, 207-218 will be used to synthesize PLGA molecular weights ranging from 1-7 kDa. The LA/GA ratio would typically be, but not limited to a ratio of 1:1. The minimum PEG molecular weight would be 2 kDa with an upper limit of 30 kDa. The preferred range of PEG would be 3-12 kDa. The PLGA molecular weight would be a minimum value of 4 kDa and a maximum of 30 kDa. The preferred range of PLGA would be 7-20 kDa. Any drug (e.g. docetaxel, paclitaxel, doxorubicin, etc.) could be conjugated to the PLGA through an appropriate linker (i.e. as listed in the previous examples) to form a polymer-drug conjugate. In addition, the same drug or a different drug could be attached to the other PLGA to form a dual drug polymer conjugate with two same drugs or two different drugs. Nanoparticles could be formed from either the PLGA-PEG-PLGA alone or from a single drug or dual polymer conjugate composed of this triblock copolymer.

### Example 16

### Synthesis of polycaprolactone-poly(ethylene glycol)-polycaprolactone (PCL-PEG-PCL)

The triblock PCL-PEG-PCL will be synthesized using a ring open polymerization method in the presence of a catalyst (i.e. stannous octoate) as reported in Hu et al., Journal of Controlled Release, 118, 2007, 7-17. The molecular weights of PCL obtained from this synthesis range from 2 to 22 kDa. A non-catalyst method shown in the article by Ge et al. Journal of Pharmaceutical Sciences, 91, 2002, 1463-1473 will also be used to synthesize PCL-PEG-PCL. The molecular weights of PCL that could be obtained from this particular synthesis range from 9 to 48 kDa. Similarly, another catalyst free method developed by Cerrai et al., Polymer, 30, 1989, 338-343 will be used to synthesize the triblock copolymer with molecular weights of PCL ranging from 1-9 kDa. The minimum PEG molecular weight would be 2 kDa with an upper limit of 30 kDa. The preferred range of PEG would be 3-12 kDa. The PCL molecular weight would be a minimum value of 4 kDa and a maximum of 30 kDa. The preferred range of PCL would be 7-20 kDa. Any drug (e.g. docetaxel, paclitaxel, doxorubicin, etc.) could be conjugated to the PCL through an appropriate linker (i.e. as listed in the previous examples) to form a polymer-drug conjugate. In addition, the same drug or a different drug could be attached to the other PCL to form a dual drug polymer conjugate with two same drugs or two different drugs. Nanoparticles could be formed from either the PCL-PEG-PCL alone or from a single drug or dual polymer conjugate composed of this triblock copolymer.

### Example 17

### Synthesis of polylactide-poly(ethylene glycol)-polylactide (PLA-PEG-PLA)

The triblock PLA-PEG-PLA copolymer will be synthesized using a ring opening polymerization using a catalyst (i.e. stannous octaote) reported in Chen et al., Polymers for Advanced Technologies, 14, 2003, 245-253. The molecular weights of PLA that can be formed range from 6 to 46 kDa. A lower molecular weight range (i.e. 1-8 kDa) could be achieved by using the method shown by Zhu et al., Journal of Applied Polymer Science, 39, 1990, 1-9. The minimum PEG molecular weight would be 2 kDa with an upper limit of 30 kDa. The preferred range of PEG would be 3-12 kDa. The PCL molecular weight would be a minimum value of 4 kDa and a maximum of 30 kDa. The preferred range of PCL would be 7-20 kDa. Any drug (e.g. docetaxel, paclitaxel, doxorubicin, etc.) could be conjugated to the PLA through an appropriate linker (i.e. as listed in the previous examples) to form a polymer-drug conjugate. In addition, the same drug or a different drug could be attached to the other PLA to form a dual drug polymer conjugate with two same drugs or two different drugs. Nanoparticles could be formed from either the PLA-PEG-PLA alone or from a single drug or dual polymer conjugate composed of this triblock copolymer.

### Example 18

### Synthesis of p-dioxanone-co-lactide-poly(ethylene glycol)-p-dioxanone-co-lactide (PDO-PEG-PDO)

The triblock PDO-PEG-PDO will be synthesized in the presence of a catalyst (stannous 2-ethylhexanoate) using a method developed by Bhattari et al., Polymer International, 52, 2003, 6-14. The molecular weight of PDO obtained from this method ranges from 2-19 kDa. The minimum PEG molecular weight would be 2 kDa with an upper limit of 30 kDa. The preferred range of PEG would be 3-12 kDa. The PDO molecular weight would be a minimum value of 4 kDa and a maximum of 30 kDa. The preferred range of PDO would be 7-20 kDa. Any drug (e.g. docetaxel, paclitaxel, doxorubicin, etc.) could be conjugated to the PDO through an appropriate linker (i.e. as listed in the previous examples) to form a polymer-drug conjugate. In addition, the same drug or a different drug could be attached to the other PDO to form a dual drug polymer conjugate with two same drugs or two different drugs. Nanoparticles could be formed from either the PDO-PEG-PDO alone or from a single drug or dual polymer conjugate composed of this triblock copolymer.

### Example 19

### Formulation of Docetaxel-PLGA Particles Via Nanoprecipitation Using PVA as Surfactant

Docetaxel-5050 PLGA-O-acetyl (700 mg, 70 wt % or 600 mg, 60 wt %,) and mPEG-PLGA (300 mg, 30 wt % or 400 mg, 40 wt %, Mw 12.9 kDa) were dissolved to form a total concentration of 1.0% polymer in acetone. In a separate solution, 0.5% w/v PVA (80% hydrolyzed, Mw 9-10 kDa) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The solution was then passed through a 0.22 μm filter, and adjusted to a final concentration of 10% sucrose. The nanoparticles could be lyophilized into powder form. The nanoparticles contain about half the initial amount of mPEG-PLGA, and 15-30% PVA.

Particle properties, evaluated by using the resulting plurality of particles made in the method above: (prior to passing through 0.22 μm filter):

| | Docetaxel-5050 PLGA-O- | Docetaxel-5050 PLGA-O- |
|---|---|---|
| | acetyl/mPEG-PLGA | acetyl/mPEG-PLGA |
| | Starting amt: (70/30 wt %) | Starting amt: (60/40 wt %) |
| Z-average (nm) | 93 | 84 |
| Particle PDI | 0.09 | 0.06 |
| Dv50 (nm) | 76 | 71 |
| Dv90 (nm) | 124 | 109 |

### Example 20

### Formulation of PEGylated Docetaxel-5050 PLGA-O-acetyl Nanoparticles Via Nanoprecipitation Using Polysorbate 80 as the Surfactant

Docetaxel-5050 PLGA-O-acetyl (672 mg, 84 wt %) and mPEG-PLGA (128 mg, 16 wt %, Mw 12.9 kDa,) were dissolved to form a total concentration of 2.0% polymer in acetone. In a separate solution, 0.5% w/v polysorbate 80 in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of 0.5% w/v polysorbate 80 and concentrated using a tangential flow filtration system (300 kDa MW cutoff, membrane area=50 cm²). The solution was then passed through a 0.22 μm Nylon filter, and adjusted to a final concentration of 10% sucrose. The nanoparticles could be lyophilized into powder form. The nanoparticles contain about half the initial amount of mPEG-PLGA, and 5-15% surfactant.

Particle properties, evaluated by using the resulting plurality of particles made in the method above:
Zavg=107 nm
Particle PDI=0.112
Dv50=89 nm
Dv90=150 nm

### Example 21

### Formulation of PEGylated Docetaxel-5050 PLGA-O-acetyl Nanoparticles Via Nanoprecipitation Using Solutol® HS 15 as the Surfactant

Docetaxel-5050 PLGA-O-acetyl (672 mg, 84 wt %) and mPEG-PLGA (128 mg, 16 wt %, Mw 12.9 kDa,) were dissolved to form a total concentration of 2.0% polymer in acetone. In a separate solution, 0.5% w/v Solutol® HS 15 in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of 0.5% w/v Solutol® HS 15 and concentrated using a tangential flow filtration system (300 kDa MW cutoff, membrane area=50 cm²). The solution was then passed through a 0.22 μm Nylon filter, and adjusted to a final concentration of 10% sucrose. The nanoparticles could be lyophilized into powder form. The nanoparticles contain about half the initial amount of mPEG-PLGA, and 5-15% surfactant.

Particle properties, evaluated by using the resulting plurality of particles made in the method above:
Zavg=106 nm
Particle PDI=0.093
Dv50=91 nm
Dv90=147 nm

### Example 22

### Formulation of PEGylated Docetaxel-5050 PLGA-O-acetyl/Doxorubicin 5050 PLGA Amide Nanoparticles Via Nanoprecipitation Using PVA as the Surfactant

Docetaxel-5050 PLGA-O-acetyl (400 mg, 59 wt %), doxorubicin 5050 PLGA amide (200 mg, 8.9 wt %) and mPEG-PLGA (40 mg, 6.25 wt %, Mwt. 8232 Da) were dissolved to form a total concentration of 1.0% polymer in acetone. In a separate solution, 0.5% w/v PVA (viscosity 2.5-3.5 cp) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form.

Particle properties, evaluated by using the resulting plurality of particles made in the method above:
Zavg=146.6 nm
Particle PDI=0.146
Dv50=137 nm
Dv90=211 nm

### Example 23

### Synthesis and Formulation of Rhodamine Labeled PEGylated Docetaxel-5050 PLGA-O-acetyl Via Nanoprecipitation Using PVA as the Surfactant

Para-nitrophenyl protected PEG-PLGA 5050-lauryl ester (150 mg, 1.36×10⁻⁵ moles) was added to rhodamine B ethylene diamine (8 mg, 1.36×10⁻⁵ moles) in N,N dimethylformamide (DMF) in the presence of triethylamine (4 uL, 2.72×10⁻⁵ moles). The reaction mixture was stirred at room temperature overnight. DMF was removed from the reaction mixture under vacuum. Purification of the product was obtained through 3 times precipitation of the crude product dissolved in dichloromethane in methyl tert-butyl ether. The product was then dried under vacuum overnight.

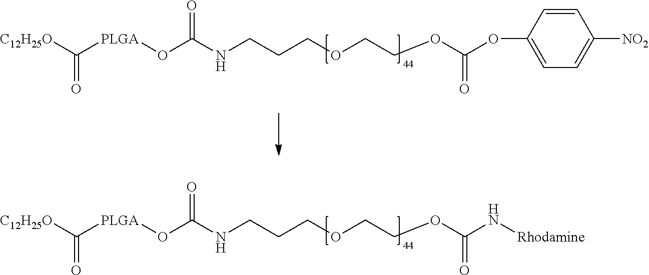

Docetaxel-5050 PLGA-O-acetyl (120 mg, 59 wt %), mPEG-PLGA (18 mg, 8.9 wt %, Mw 12.9 kDa), Rhodamine B-labeled-PEG-PLGA-lauryl ester (4 mg, 1.9 wt %) and purified PLGA (60 mg, 30 wt %) were dissolved to form a total concentration of 1.0% polymer in acetone. In a separate solution, 0.5% w/v PVA (viscosity 2.5-3.5 cp) in water was prepared. The polymer acetone solution was added using a syringe pump at a rate of 1 mL/min to the aqueous solution (v/v ratio of organic to aqueous phase=1:10), with stirring at 500 rpm. Acetone was removed by stirring the solution for 2-3 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form.

### Example 24

### Formulation of Docetaxel-5050 PLGA-O-acetyl Nanoparticles Via Micro-Mixer Using PVA as the Surfactant

5050 purified PLGA (211 mg, 32 μmol), docetaxel-5050 PLGA-O-acetyl (633 mg, 71 μmol) and mPEG-PLGA (Mw 8.3 kDa, 5 wt % total polymer) were combined at a total concentration of 1.0% polymer in acetone.

A separate solution of 0.5% polyvinylalcohol (80% hydrolyzed, Mw 9-10 kDa) in water was prepared. The organic and aqueous solutions were then blended using a Caterpillar MicroMixer (CPMM-v1.2-R300), using flow rates of 5 mL/min and 15 mL/min respectively.

The acetone was removed from the resulting nanoparticle dispersion by rotary evaporation. The aqueous nanoparticle dispersion was washed with 10 volumes of water using a tangential flow filtration system (300 kDa MW cutoff, membrane area=50 cm²). The dispersion was then concentrated using a tangential flow filtration system (300 kDa MW cutoff, membrane area=50 cm²). The solution was then passed through a 0.22 filter, and adjusted to a final concentration of 10% sucrose. The solution was then lyophilized to provide the particles. The nanoparticles contain half the initial amount of mPEG-PLGA, and 15-30% PVA.

Particle Properties:
Zavg=133.9 nm
Particle PDI=0.199
Dv50=110 nm
Dv90=237 nm

### Example 25

### Formulation of Doxorubicin 5050 PLGA Amide Nanoparticles Via Emulsion Using PVA as the Surfactant

Doxorubicin 5050 PLGA amide (100 mg, 100 wt %) was dissolved to form a total concentration of 1.0% polymer in dichloromethane. In a separate solution, 0.5% w/v PVA (viscosity 2.5-3.5 cp) in water was prepared. The dissolved polymer solution in dichloromethane was mixed with the aqueous PVA solution and emulsified through a microfluidizer processor for three cycles at a pressure of 8500 psi. Dichloromethane was removed by stirring the solution for 12 hours. The nanoparticles were then washed with 10 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form and were prepared for purposes of comparison.

Particle Properties:
Zavg=91.19 nm
Particle PDI=0.135
Dv50=70.5 nm
Dv90=120 nm

### Example 26

### Formulation of Embedded Docetaxel/Paclitaxel in Docetaxel-5050 PLGA-O-Acetyl Nanoparticles Via Emulsion Using PVA as the Surfactant

Docetaxel-5050 PLGA-O-acetyl (90 wt %), mPEG-PLGA (10 wt %) and either docetaxel or paclitaxel (30 mg) were dissolved in dichloromethane (DCM, 14 mL). A separate solution of 0.5% polyvinylalcohol (PVA, 80% hydrolyzed, Mw 9-10 kDa) in water was prepared. The dissolved polymer-drug solution was transferred with a syringe into a beaker containing the 0.5% PVA (96 mL, v/v ratio of organic to aqueous phase=˜1:7) and sonicated using a micro-tip horn (tip diameter=½ inch) for 5 minutes to form an emulsion. The emulsion is then transferred to a microfluidizer processor and passed through seven times with processing pressures ranging from 13,000-16,100 psi.

The DCM was removed from the resulting nanoparticle dispersion by rotary evaporation. The aqueous nanoparticle dispersion was washed with 10-20 times volumes of water and concentrated using a tangential flow filtration system (300 kDa MW cutoff, membrane area=50 cm²). The solution was passed through a 0.22 μm filter, and for lyoprotection, 10% sucrose was added. The nanoparticles were lyophilized to form a white powder.

### Particle Properties:

| | Docetaxel | Paclitaxel |
|---|---|---|
| Zavg (nm) | 94 | 102 |
| Particle PDI | 0.107 | 0.103 |
| Dv50 (nm) | 75 | 82 |
| Dv90 (nm) | 128 | 142 |
| Embedded drug (% w/w) | 1.9 | 4.5 |
| Conjugate docetaxel (% w/w) | 4.0 | 4.1 |

### Example 27

### Formulation of docetaxel-2′-hexanoate-5050 PLGA-O-acetyl Nanoparticles

One could prepare nanoparticles by combining docetaxel-2′-hexanoate-5050 PLGA-O-acetyl and mPEG-PLGA at a weight ratio ranging from 84-60/16-40 wt % with a total concentration of 1% polymer in acetone. In a separate solution, 0.5% w/v PVA (viscosity 2.5-3.5 cp) in water could be prepared. The polymer acetone solution could be added using a syringe pump at a rate of 1 mL/min to the aqueous solution (v/v ratio of organic to aqueous phase=1:10), with stirring at 500 rpm. Acetone could be removed by stirring the solution for 2-3 hours. The nanoparticles could be then washed with 10 volumes of water and concentrated using a tangential flow filtration system (300 kDa MW cutoff, membrane area=50 cm²). For lyoprotection, standard lyoprotectants could be used (e.g. sucrose) and the nanoparticles could be lyophilized into powder form.

### Example 28

### Formulation of PEGylated O-acetyl-5050-PLGA-(2′-β-alanine glycolate)-docetaxel Nanoparticles

O-acetyl-5050-PLGA-(2′-β-alanine glycolate)-docetaxel (600 mg, 60 wt %) and mPEG-PLGA (400 mg, 40 wt %) were dissolved to form a total concentration of 1.0% polymer in acetone. In a separate solution, 0.5% w/v PVA (viscosity 2.5-3.5 cp) in water was prepared. The organic and aqueous solutions were then mixed together using a nanoprecipitation method at an organic to aqueous ratio of 1:10. Acetone was removed from the resulting nanoparticle dispersion by passive evaporation. The nanoparticles were then washed with 12 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form. The nanoparticles contain half the initial amount of mPEG-PLGA, and 15-30% PVA.

Particle Properties:
Zavg=74.3 nm
Particle PDI=0.097
Dv50=57.5 nm
Dv90=94.4 nm

### Example 29

### Formulation of PEGylated bis(docetaxel) glutamate-5050 PLGA-O-acetyl Nanoparticles

Bis(docetaxel) glutamate-5050 PLGA-O-acetyl (600 mg, 60 wt %) and mPEG-PLGA (400 mg, 40 wt %) were dissolved to form a total concentration of 1.0% polymer in acetone. In a separate solution, 0.5% w/v PVA (viscosity 2.5-3.5 cp) in water was prepared. The organic and aqueous solutions were then mixed together using a nanoprecipitation method at an organic to aqueous ratio of 1:10. Acetone was removed from the resulting nanoparticle dispersion by passive evaporation. The nanoparticles were then washed with 12 volumes of water and concentrated using a tangential flow filtration system (300 kDa MW cutoff, membrane area=50 cm²). The nanoparticle solution was adjusted to a final concentration of 10% sucrose. The nanoparticles could be lyophilized into powder form. The nanoparticles contain half the initial amount of mPEG-PLGA, and 15-30% PVA.

Particle Properties:
Zavg=68.6 nm
Particle PDI=0.082
Dv50=55.9 nm
Dv90=87.2 nm

### Example 30

### Formulation of PEGylated O-acetyl-5050-PLGA-(2′-β-alanine glycolate)-docetaxel/docetaxel-2′5050 PLGA-o-acetyl Nanoparticles

O-acetyl-5050-PLGA-(2′-β-alanine glycolate)-docetaxel, docetaxel-5050 PLGA-o-acetyl and mPEG-PLGA could be combined at a weight ratio of 84-60/16-40 wt % (polymer drug conjugates/mPEG-PLGA) with a total concentration of 1% polymer in acetone. In a separate solution, 0.5% w/v PVA (viscosity 2.5-3.5 cp) in water could be prepared. The polymer drug conjugates could vary from a ratio of 10:1 to 1:10. The organic and aqueous solutions could then be mixed together using a nanoprecipitation method at an organic to aqueous ratio of 1:10. The acetone could be removed from the resulting nanoparticle dispersion by passive evaporation. The nanoparticles could be washed with 15 volumes of water and concentrated using a tangential flow filtration system (300 kDa MW cutoff, membrane area=50 cm²). The nanoparticle solution could be adjusted to a final concentration of 10% sucrose. The nanoparticles could be lyophilized into powder form. This particular nanoparticle configuration could allow for different release rates of docetaxel.

### Example 31

### Preparation of Docetaxel-PLGA Nanoparticles Samples for Imaging Using Cryo Scanning Electron Microscopy (Cryo-SEM)

Lyophilized samples of docetaxel-PLGA nanoparticles containing PVA were reconstituted and fixed in 0.5% osmium tetroxide (OsO₄) in water for ca. 15 min prior to centrifugation and washing with water. Sample droplets were placed into a rivet holder, which was fast frozen in liquid nitrogen slush (ca. −210° C.) A vacuum was pulled and the sample was transferred to a Gatan Alto 2500-pre chamber (cooled to ca. −160° C.). The sample was fractured, sublimated at −90° C. for 7-10 minutes and coated with platinum using a sputter coating for 120 sec. Finally the samples were transferred to the microscope cryostage which is maintained at −130° C. The samples were imaged with an FEI NOVA nanoSEM field emission scanning electron microscope operating at an accelerating velocity of 5 kV.

The cryo-SEM images showed that the docetaxel-PLGA nanoparticles containing PVA were spherical and no apparent surface structure was evident. The particle sizes ranged from 50-75 nm.

### Example 32

### Preparation of Docetaxel-PLGA Nanoparticles Samples for Imaging Using Transmission Electron Microscopy (TEM)

Carbon coated formvar grids (400 mesh) were glow-discharged prior to use. A droplet sample of docetaxel-PLGA nanoparticles containing PVA was added to the carbon grids and allowed to sit for ca. 2 min. The grids were then quickly touched to droplets for 2% uranyl acetate. The excess stain was removed with filter paper and allowed to dry. The samples were imaged with a Phillips CM-100 transmission electron microscope operating at an accelerating velocity of 80 kV.

The TEM images showed that the docetaxel-PLGA nanoparticles containing PVA were spherical and relatively uniform in size. The particle size from the TEM micrograph were typically less than 150 nm.

### Example 33

### Synthesis, Purification and Characterization of Doxorubicin Tosylate

In a 250-mL round-bottom flask equipped with a magnetic bar and a thermocouple, doxorubicin.HCl (NetQem, 1.43 g, 2.46 mmol) was suspended in anhydrous THF (143 mL, 100 vol). The mixture was evacuated for 15 seconds while being stirred and filled up with nitrogen (1 atm). 1 M potassium tert-butoxide (KOtBu)/THF solution (2.7 mL, 2.70 mmol) was added dropwise with stirring within 10 min. The solution turned a purple color and a slight exotherm was observed. The reaction temperature rose from 19° C. to 21.7° C. within 15 min and then slightly climbed up to a maximum of 22.4° C. in half hour. The mixture was stirred for another hour at 22.4° C. and then p-Toluenesulfonic acid (p-TSA, 0.70 g, 3.96 mmol) was added in one portion. The solution immediately turned a red color along with the precipitation of fine particles. The mixture was stirred for an additional half hour at ambient temperature and then cooled to 5° C. and stirred for 1 h. The resulting red suspension was filtered under nitrogen. The filter cake was washed with THF (3×10 mL) and dried under vacuum at 25° C. for 16 h to produce doxorubicin tosylate [1.73 g, 97% yield)]. HPLC analysis indicated a 97% purity (AUC, 480 nm).

To remove the excess p-TSA, the product was slurried in 5:1 MTBE/MeOH (60 mL) at ambient temperature for 3 h. The filtered solid was dried under vacuum at 25° C. for 16 h to afford 1.32 g of product. HPLC analysis indicated 99% purity (AUC, 480 nm); however, the ¹H NMR analysis showed that the equivalents of p-TSA were still ˜1.2. DSC analysis of doxorubicin tosylate showed a sharp peak with a melting range of 188.5-196.5° C.

### Example 34

### Synthesis and Characterization of Doxorubicin Octanesulfonate

In a 250 mL round-bottom flask equipped with a magnetic stirrer, 1-octanesulfonic acid sodium salt monohydrate (0.44 g, 1.86 mmol) was dissolved in water (50 mL). The mixture was stirred for 10 min to afford a clear solution, to which doxorubicin.HCl (1.08 g, 1.86 mmol) was added in one portion. The solution became a dark red color after being stirred for a few minutes. After about 30 min, an orange powder formed. The mixture was stirred at ambient temperature for 2 h. The suspension was stored in fridge for 16 h and filtered through a Sharkskin® filter paper. The filtrate had a slightly red color and contained trace amounts of doxorubicin as evidenced by HPLC analysis. The presence of chloride in the filtrate was confirmed by the silver nitrate test. The filter cake was dried under vacuum at 28° C. for 16 h to afford doxorubicin octanesulfonate [1.16 g, yield: 85%] as an orange powder. The ¹H NMR analysis indicated the desired product and HPLC analysis indicated >99.5% purity. DSC analysis of doxorubicin octanesulfonate showed a sharp peak with a melting range of 198.7-202.0° C.

### Example 35

### Synthesis, Purification and Characterization of Doxorubicin Naphthalene-2-Sulfonate

A 250-mL round-bottom flask equipped with a magnetic bar and a thermocouple was charged with doxorubicin.HCl (NetQem, 1.47 g, 2.53 mmol) and anhydrous THF (150 mL, 100 vol). The suspension was evacuated for 15 seconds with stirring and filled up with nitrogen (1 atm). 1 M (KOtBu)/THF solution (2.7 mL, 2.70 mmol) was added dropwise with stirring over 10 min. The mixture turned a purple color and a slight exotherm was observed, causing the reaction temperature to rise from 20.2° C. to 21.4° C. within 15 min. The solution was stirred at 21.1° C. for one hour and 2-naphthalenesulfonic acid (0.63 g, 3.04 mmol) was added in one portion. The mixture immediately turned to a red color and the precipitation of fine particles was observed. The solution was stirred for an hour at ambient temperature and then filtered under nitrogen. The filtration was slow and took about 1 h. The filter cake was washed with THF (3×10 mL) and dried under vacuum at 25° C. for 16 h to afford 2.1 g of doxorubicin naphthalene-2-sulfonate as a dark red solid [yield: >100%]. HPLC analysis indicated a 98% purity (AUC, 480 nm). The ¹H NMR analysis showed that the ratio of 2-naphthalenesulfonic acid to doxorubicin was ˜1.08.

To remove residual 2-naphthalenesulfonic acid, the doxorubicin naphthalene-2-sulfonate was slurried in 5:1 MTBE/MeOH (60 mL) for 3 h. The suspension was filtered and the filter cake was dried under vacuum at 25° C. for 24 h to afford 1.90 g of the product as a fine red powder [yield: 100%]. The ¹H NMR analysis indicated a clean product with a 1:1 ratio of doxorubicin to 2-naphthalenesulfonic acid. HPLC analysis showed >98% purity (AUC, 480 nm). The physical appearance of the product was similar to doxorubicin.HCl. DSC analysis of doxorubicin naphthalene-2-sulfonate showed a sharp peak with a melting range of 203.1-207.4° C.

### Example 36

### Cytotoxicity of Nanoparticles Formed from Polymer Drug Conjugates

To measure the cytotoxic effect of nanoparticles formed from doxorubicin 5050 PLGA amide, paclitaxel-5050 PLGA-O-acetyl, docetaxel-5050 PLGA-O-acetyl or bis(docetaxel) glutamate-5050 PLGA-O-acetyl, the CellTiter-Glo® Luminescent Cell Viability Assay (CTG) (Promega) was used. Briefly, ATP and oxygen in viable cells reduce luciferin to oxyluciferin in the presence of luciferase to produce energy in the form of light. B16.F10 cells, grown to 85-90% confluency in 150 cm² flasks (passage <30), were resuspended in media (MEM-alpha, 10% HI-FBS, 1× antibiotic-antimycotic) and added to 96-well opaque-clear bottom plates at a concentration of 1500 cells/well in 200 μL/well. The cells were incubated at 37° C. with 5% CO₂ for 24 hours. The following day, serial dilutions of 2× concentrated particles and 2× concentrated free drug were made in 12-well reservoirs with media to specified concentrations. The media in the plates was replaced with 100 μL of fresh media and 100 μL of the corresponding serially diluted drug. Three sets of plates were prepared with duplicate treatments. Following 24, 48 and 72 hours of incubation at 37° C. with 5% CO₂, the media in the plates was replaced with 100 μL of fresh media and 100 μL of CTG solution, and then incubated for 5 minutes on a plate shaker at room temperature set to 450 rpm and allowed to rest for 15 minutes. Viable cells were measured by luminescence using a microtiter plate reader. The data was plotted as viability vs. concentration and standardized to untreated cells. The doxorubicin 5050 PLGA amide, paclitaxel-5050 PLGA-O-acetyl, docetaxel-5050 PLGA-O-acetyl and bis(docetaxel) glutamate-5050 PLGA-O-acetyl polymer drug conjugates inhibited the growth of B16.F10 cells in a dose and time dependent manner. Also, in comparison to the corresponding free drug, the polymer drug conjugates exhibited a slower release profile.

### IC₅₀ on Day 3:

| Group | IC₅₀ (μM) |
|---|---|
| Free doxorubicin | 14 |
| Doxorubicin 5050 PLGA amide nanoparticles | 2.9 |
| Free paclitaxel | 7 |
| Paclitaxel-5050 PLGA-O-acetyl nanoparticles | 480 |
| Free docetaxel | 0.13 |
| Docetaxel-5050 PLGA-O-acetyl nanoparticles | 20 |
| bis(docetaxel) glutamate-5050 PLGA-O-acetyl nanoparticles | 25 |

### Example 37

### Bioburden Test for Contamination of Nanoparticles Formed from Polymer Drug Conjugate

To measure the formulation sterility for PEGylated docetaxel-5050 PLGA-O-acetyl nanoparticles, the spot colony forming units per gram (CFU) assay, a modified plate count method, was used. A positive control was prepared by inoculating 10 mL of trypticase soy broth (TSB) with an isolated colony from an in house bacterial stock and grown at 37° C. in a shaking incubator at 350 rpm for 24 hours. A subculture (1:100) was then prepared and grown at 37° C. in a shaking incubator (350 rpm for 3 hours). The bacteria were then pelleted, washed with PBS and resuspended with fresh TSB. A 0.5 McFarland standard bacterial solution (equal to 1.5×10⁶ CFU/mL based on turbidity measurement) was then prepared. An aliquot of 100 μL was sampled from each of the following solutions: a ca. 1.5 mg/ml nanoparticle solution (4-5 mL batch size), a positive control and TSB, as well as a negative control. These were each mixed with 400 μL of TSB in a 1.5 mL microcentrifuge tube and cultured in a shaking incubator at 37° C. (450 rpm for 3 days). On days 0 and 3, 50 μL of each sample were removed from the sample mix and serially diluted at a ratio of 1:10 with TSB in a 96-well plate. The diluted samples (6 μL) were spotted onto pre-dried trypticase soy agar (TSA) plates using a multichannel pipet. The spots were allowed to dry and the plates were incubated at 37° C. for 24 hours. After 24 hours, the isolated colonies were counted and the CFU/mL calculated. To detect very low concentrations of contaminants, 200 μL of each sample mix were spread onto agar plates on day 3 and incubated at 37° C. for 24 hours. The tests were carried out over an open flame.

### Colony Forming Units Per Gram

| | T₀ Spot | T₇₂ Spot | T₇₂ Plate |
|---|---|---|---|
| | CFU | CFU | CFU |
| Description | CFU/mL | CFU/mL | CFU/mL |
| PEGylated docetaxel-5050 | 0 | 0 | 0 |
| PLGA-O-acetyl nanoparticles, | | | |
| Filtered with 0.22 μm Steriflip | | | |
| PEGylated docetaxel-5050 | 0 | 0 | 0 |
| PLGA-O-acetyl nanoparticles, | | | |
| Filtered with 0.45 μm Steriflip | | | |
| Positive control, 1.5 × 10⁶ | 6.67 × 10⁵ | 3.80 × 10¹¹ | Lawn |
| CFU/mL standardized stock | | | |
| solution in TSB | | | |
| Negative control, TSB | 0 | 0 | 0 |

### Example 38

### In Vivo Efficacy of PEGylated Doxorubicin 5050 PLGA Amide Nanoparticles in a B16.F10 Mouse Model of Melanoma

B16.F10 cells were grown in culture to 85-90% confluency in MEM-α medium supplemented with 10% FBS and 1% penicillin/streptomycin (passage=4) and then resuspended in PBS. B16.F10 cells (density=5×10⁶ cells/mL) were implanted subcutaneously (SC) into the right flank of male C57BL/6 mice (20-22 g on day 1.

The five treatment groups that were administered to the mice were: 1) 0.9% NaCl solution; 2) Doxil (liposomal formulation of doxorubicin HCl containing 2 mg/mL doxorubicin HCl, Ortho Biotech) at 1 mg/kg dose; 3) three PEGylated doxorubicin 5050 PLGA amide nanoparticles with 1, 2 and 3 mg/kg doxorubicin equivalent doses.

The treatments were administered IV into the tail vein of the mouse at a dose volume of 6 mL/kg, beginning on day 5 post-implantation, when the mean tumor volume was 50 mm³. The treatments were administered on days 5, 8, and 12 (biweekly×3 injections) post tumor implantation. Health status of the animals was monitored and the tumor was measured three times a week. On day-17 post-tumor implantation, mice were euthanized by CO₂ inhalation according to the IUCAC procedure guideline. Tumor from each animal was dissected and tumor volume as well as tumor growth inhibition (TGI) was measured. Tumor volume was calculated using the formula: (width×width×length)/2 mm³. TGI represented as % was calculated using the formula: (1−(treated tumor volume/control tumor volume))×100.

### Tumor Growth Inhibition (TGI)

The treatment groups of Doxil and all the PEGylated doxorubicin 5050 PLGA amide nanoparticles showed inhibition of tumor growth on day-17. A dose-dependent tumor growth inhibition was seen with PEGylated doxorubicin 5050 PLGA amide nanoparticles; 37% TGI at 1 mg/kg, 48% TGI at 2 mg/kg and 57% TGI at 3 mg/kg. Doxil at 1 mg/kg exhibited 60% TGI on day 17.

Tumor Growth Inhibition (n=4)

| | Dose | Day-17 |
|---|---|---|
| Group | mg/kg | TGI, % |
| 0.9% NaCl control | — | — |
| Doxil | 1 | 60% |
| PEGylated doxorubicin 5050 | 1 | 37% |
| PLGA amide nanoparticles | | |
| PEGylated doxorubicin 5050 | 2 | 48% |
| PLGA amide nanoparticles | | |
| PEGylated doxorubicin 5050 | 3 | 58% |
| PLGA amide nanoparticles | | |

### Example 39

### In Vivo Efficacy of PEGylated Paclitaxel-5050 PLGA-O-Acetyl Nanoparticles in a B16.F10 Mouse Model of Melanoma

B16.F10 cells were grown in culture to 85-90% confluency in MEM-α medium supplemented with 10% FBS and 1% penicillin/streptomycin (passage=4) and then resuspended in PBS. B16.F10 cells (density=5×10⁶ cells/mL) were implanted subcutaneously (SC) into the right flank of male C57BL/6 mice (20-22 g on day 1.

The four treatment groups that were administered to the mice were: 1) 0.9% NaCl solution; 2) Abraxane® (Abraxis) at 1.5, 6 and 15 mg/kg dose; 3) free paclitaxel at doses of 1.5, 6 and 15 mg/kg and 4) PEGylated paclitaxel-5050 PLGA-O-acetyl nanoparticles at doses of 1.5, 3, 6, 9, and 15 mg/kg paclitaxel equivalent.

The treatments were administered IV into the tail vein at a dose volume of 6 mL/kg, beginning on day-5 post-implantation, when the mean tumor volume was 55 mm³. The treatments were administered on days 5, 8, and 12 (biweekly×3 injections) post tumor implantation. Health status of the animals was monitored and tumor size was measured three times a week. On day 17, post-tumor implantation, mice were euthanized by CO₂ inhalation according to the IUCAC procedure guideline. Tumors from each animal were dissected and tumor size was measured. Tumor volume was calculated using the formula: (width×width×length)/2 mm³. TGI represented as % was calculated using the formula: (1−(treated tumor volume/control tumor volume))×100.

### Tumor Growth Inhibition

Abraxane®, free paclitaxel and all PEGylated paclitaxel-5050 PLGA-O-acetyl nanoparticles groups showed inhibition of tumor growth on day 17. A dose-dependent TGI was seen with the free paclitaxel treated groups; 37% TGI at 1.5 mg/kg, 57% % TGI at 6 mg/kg and 83% TGI at 15 mg/kg doses. Abraxane® showed a 36% TGI at 1.5 mg/kg, 13% % TGI at 6 mg/kg and 70% TGI at 15 mg/kg doses. At the lowest dose of 1.5 mg/kg, PEGylated paclitaxel-5050 PLGA-O-acetyl nanoparticles exhibited a 42% TGI, which is similar to free paclitaxel and Abraxane® treated groups at the same dose. However, PEGylated paclitaxel-5050 PLGA-O-acetyl nanoparticles showed a 42% TGI at 1.5 mg/kg, 40% TGI at 3 mg/kg, 46% TGI at 6 mg/kg, 61% TGI at 9 mg/kg and 58% TGI at 15 mg/kg doses.

Tumor Growth Inhibition (n=4)

| | Dose | Day-17 |
|---|---|---|
| Group | mg/kg | TGI, % |
| 0.9% NaCl control | — | — |
| Abraxane ® | 1.5 | 36% |
| Abraxane ® | 6 | 13% |
| Abraxane ® | 15 | 70% |
| Free paclitaxel | 1.5 | 37% |
| Free paclitaxel | 6 | 57% |
| Free paclitaxel | 15 | 83% |
| PEGylated paclitaxel-5050 | 1.5 | 42% |
| PLGA-O-acetyl nanoparticles | | |
| PEGylated paclitaxel-5050 | 3 | 40% |
| PLGA-O-acetyl nanoparticles | | |
| PEGylated paclitaxel-5050 | 6 | 46% |
| PLGA-O-acetyl nanoparticles | | |
| PEGylated paclitaxel-5050 | 9 | 61% |
| PLGA-O-acetyl nanoparticles | | |
| PEGylated paclitaxel-5050 | 15 | 58% |
| PLGA-O-acetyl nanoparticles | | |

### Example 40

### Tolerability and In Vivo Efficacy of PEGylated Docetaxel-5050 PLGA-O-Acetyl Nanoparticles in a B16.F10 Mouse Model of Melanoma

B16F10 cells were grown in culture to 85% confluency in MEM-α medium containing 10% FBS and 1% penicillin/streptomycin (passage=4) and then resuspended in PBS. B1610 cells (density=10×10⁶ cells) were implanted subcutaneously (SC) into the right flank of male C57BL/6 mice on Day 1. On Day 5 following tumor inoculations, animals were assigned to different treatment groups according to the tumor size.

The three treatment groups that were administered to the mice included: 1) a docetaxel vehicle formulation consisting of a 10 mg/mL stock solution (prepared with 20 mg of docetaxel, 0.2 mL ethanol, 0.5 mL polysorbate 80 and 1.3 mL water, added in that specific order and vortexed to ensure proper mixing). The stock solution was diluted further with PBS to 0.6 and 1.5 mg/mL (for a corresponding dose of 6 and 15 mg/kg) so that all the groups received the same amount of ethanol, polysorbate 80, water and PBS. 2) PEGylated (10 mol %) docetaxel-5050 PLGA-O-acetyl nanoparticles at doses of 6, 15 and 30 mg/kg). 3) Docetaxel vehicle.

Animals were treated with different concentrations of docetaxel and PEGylated docetaxel-5050 PLGA-O-acetyl nanoparticles as per the schedule (on Days 5, 8 and 12 following inoculation). The schedule consisted of 3 injections biweekly. The animals were monitored three times a week for health status and adverse effects from tumor cell inoculation to the end of the study. The body weight and tumor volume were also measured three times a week to evaluate the effect of the treatment.

### Tumor Growth Inhibition

On Day 17, the PEGylated (10 mol %) docetaxel-5050 PLGA-O-acetyl nanoparticles showed dose-dependent TGI. At 6, 15 and 30 mg/kg, the TGI was 53%, 88% and 93% after biweekly×3 injections.

### Example 41

### Tolerability and Maximum Tolerated Dose of PEGylated bis(docetaxel) Glutamate-5050 PLGA-O-Acetyl Nanoparticles in a B16.F10 Mouse Model of Melanoma

B16F10 cells were grown in culture to confluency in MEM-α medium containing 10% FBS and 1% penicillin/streptomycin (passage=4) and then resuspended in PBS. B1610 cells (density=1×10⁶ cells/mL in a 0.1 mL volume) were subcutaneously (SC) implanted into the right flank of male C57BL/6 mice on Day 1.

The five treatment groups that were administered to the mice included: 1) a docetaxel vehicle formulation consisting of a 10 mg/mL stock solution (prepared with 20 mg of docetaxel, 0.2 mL ethanol, 0.5 mL polysorbate 80 and 1.3 mL water, added in that specific order and vortexed to ensure proper mixing). The stock solution was diluted further with PBS to 0.6, 1.5, 3, 4.5 and 6 mg/mL (for a corresponding dose of 6, 15, 30, 45 and 60 mg/kg) so that all the groups received the same amount of ethanol, polysorbate 80, water and PBS. 2) PEGylated bis(docetaxel) glutamate-5050 PLGA-O-acetyl nanoparticles at doses of 6, 15, 30, 45 and 60 mg/kg. 3) Docetaxel vehicle at the highest concentration of 6 mg/mL consisting of 6% ethanol/15% polysorbate 80/39% water and 40% PBS. 4) Sucrose vehicle (100 mg/kg). 5) PEGylated O-acetyl-5050-PLGA nanoparticle vehicle at the highest concentration of 6 mg/mL.

The treatments were administered IV into the tail vein at a dose volume of 10 mL/kg, beginning on post-implantation Day 5, when the mean tumor volume was 55 mm³. The treatments were administered 4 times, on Days 5, 8, 12 and 15 (biweekly×4 injections). On Day 17 post-tumor implantation, mice were euthanized by CO₂ inhalation according to the procedure guideline. Blood was collected by cardiac puncture and put into ethylenediaminetetraacetic acid (EDTA) or serum separation blood collection tubes. Whole blood was analyzed on the day of collection for CBC analyses. After the blood clotted and was centrifuged, serum was frozen immediately on dry ice for serum chemistry analyses. The tumors were removed by dissection, frozen immediately on dry ice and stored at −80° C., in which they were later analyzed for bis(docetaxel) glutamate-5050 PLGA-O-acetyl and free docetaxel levels.

Tolerability was determined by changes in body weight, expressed as a percent of the initial body weight on post-implantation Day 5. The criterion at which a group was removed from the study was a mean of 20% body weight loss. Health monitoring was conducted daily, but no mice warranted removal due to indications of lethargy, tremors, hypothermia, etc. The maximum tolerated dose (MTD) was determined as the highest dose that did not cause a 20% body weight loss. Other indices of toxicity, complete blood count (CBC) and serum chemistry were determined from blood collected from animals that were euthanized on Day 17 by CO₂ inhalation, according to the procedure guideline.

### Body Weight Changes

The groups administered 6, 15, 30 and 45 mg/kg of PEGylated bis(docetaxel) glutamate-5050 PLGA-O-acetyl nanoparticles all gained weight on Day 17, a mean of 111%, 112%, 106% and 106%, 112% of the initial body weight was observed respectively. For the 60 mg/kg, at Day 17, a mean of 91% of the initial body weight was observed. In comparison, the three vehicle-treated groups all gained weight similarly, i.e. the docetaxel vehicle treatment gained 14.8%, the sucrose vehicle gained 13.8% and the PEGylated acetyl-5050-PLGA vehicle gained 16.2%. In contrast, there was a dose-related decline in body weights of mice administered docetaxel, i.e., the higher doses (e.g. 45 and 60 mg/kg) caused a mean 20% of body weight loss earlier (Day 15) compared to the lower doses (e.g. 30 mg/kg occurred at Day 17). The 6 and 15 mg/kg of docetaxel groups caused a mean of 4 and 8% body weight respectively by Day 17.

### Tumor Growth and Tumor Growth Inhibition

On Day 17, all PEGylated bis(docetaxel) glutamate-5050 PLGA-O-acetyl nanoparticles groups showed inhibition of tumor growth. The lower 2 doses, 6 and 15 mg/kg caused similar inhibition of tumor growth, 49% and 48% TGI, respectively. For 30, 45 and 60 mg/kg, a 73%, 83% and 93% TGI was shown. The TGI was directly related to the tumor docetaxel content, r>0.9. In comparison, for the docetaxel control, at 6 and 15 mg/kg, a 78% and 94% TGI, respectively was observed. In contrast, there was no effect by any vehicle on tumor growth, compared to the other vehicle-treated groups.

### Complete Blood Count

PEGylated bis(docetaxel) glutamate-5050 PLGA-O-acetyl nanoparticles showed a trend for a decline in the white blood cell (WBC) number, lymphocyte number and neutrophil number. However, there was no significant effect on either the WBC number (ranged from 10.8-6.2×1000 cells/μL for 6-60 mg/kg doses), lymphocyte number (ranged from 6221-4317 cells/μL for 6-60 mg/kg doses) or neutrophil number (ranged from 4404-1889 cells/μL for 6-60 mg/kg doses). In addition, other CBC parameters were not affected by PEGylated bis(docetaxel) glutamate-5050 PLGA-O-acetyl nanoparticles at doses up to 60 mg/kg. In comparison, for the 3 vehicle treated groups (sucrose, docetaxel, O-acetyl-5050-PLGA PEGylated nanoparticle), the WBC (ranged from 11.4-14.1×1000 cells/μL), lymphocyte number (7592-10222 cells/μL) and neutrophil number (3524-4557 cells/μL) all were within the normal range for mice.

### Serum Chemistry

The PEGylated bis(docetaxel) glutamate-5050 PLGA-O-acetyl nanoparticles did not affect any serum chemistry parameter at doses up to 15 mg/kg and 60 mg/kg respectively. In comparison, docetaxel did not affect any serum chemistry parameter at doses up to 30 mg/kg. The vehicle formulations did not affect any serum chemistry parameter. (Serum chemistry parameters determined were alkaline phosphatase, ALT, AST, CPK, albumin, total protein, total bilirubin, direct bilirubin, BUN, creatinine, cholesterol, glucose, calcium, bicarbonate and A/G ratio.)

### Maximum Tolerated Dose

The maximum tolerated dose (MTD) of PEGylated bis(docetaxel) glutamate-5050 PLGA-O-acetyl nanoparticles was 60 mg/kg at the 4-dose treatment schedule administered, 4-fold greater than free docetaxel (MTD=15 mg/kg when administered IV biweekly for 2 weeks).

### Tumor Growth Inhibition of B16F10 Tumor-Bearing Mice Administered Treatments.

| | Dose | Day 17 |
|---|---|---|
| Group | mg/kg | Tumor Growth Inhibition, % |
| Sucrose Vehicle control | 0 | — |
| PNP Vehicle | 0 | 107% |
| Free docetaxel | 6 | 78% |
| Free docetaxel | 15 | 96% |
| Free docetaxel | 30 | 95% |
| bis(docetaxel) glutamate-5050 | 6 | 49% |
| PLGA-O-acetyl nanoparticles | | |
| bis(docetaxel) glutamate-5050 | 15 | 48% |
| PLGA-O-acetyl nanoparticles | | |
| bis(docetaxel) glutamate-5050 | 30 | 73% |
| PLGA-O-acetyl nanoparticles | | |
| bis(docetaxel) glutamate-5050 | 45 | 83% |
| PLGA-O-acetyl nanoparticles | | |
| bis(docetaxel) glutamate-5050 | 60 | 93% |
| PLGA-O-acetyl nanoparticles | | |

### Example 42

### In Vivo Efficacy of PEGylated Docetaxel-5050 PLGA-O-Acetyl Nanoparticles in a A2780 Ovarian Human Xenograft Model

A2780 cells were grown in culture in RPMI-1640 containing 10% FBS and 1% penicillin/streptomycin (passage=2). When confluent, the cells were removed using 0.05% trypsin and suspended in 1:1 mixture of RPMI-1640/Matrigel at a density of 50×10⁶ cells/mL. The tumors were implanted SC by injecting 5×10⁶ A2780 cells in a 0.1 mL volume into the mammary fat pad of female CD-1 nude mice that were 6-8 weeks old.

The three treatment groups that were administered to the mice consisted of: 1) a docetaxel vehicle formulation consisting of a 10 mg/mL stock solution (prepared with 20 mg of docetaxel, 0.2 mL ethanol, 0.5 mL polysorbate 80 and 1.3 mL water, added in that specific order and vortexed to ensure proper mixing). The stock solution was diluted further with PBS to 1.5 mg/mL (for a dose of 15 mg/kg at 10 mL/kg and 30 mg/kg at 20 mL/kg). This formulation was made within 30 minutes of administration to mice. 2) Filtered PEGylated O-acetyl-5050-PLGA nanoparticles at a dose of 30 mg/kg, 3) docetaxel vehicle at the highest concentration of 1.5 mg/mL consisting of 1.5% ethanol, 3.8% polysorbate 80, 9.8% water and 85% PBS.

The treatments were administered IV into the tail vein at a dose volume of 10 mL/kg for the 15 mg/kg group and 20 mL/kg for the other groups, beginning on post-implantation Day 8, when the mean tumor volume was 128 mm³. The treatments were administered 2 times, on Day 8 and Day 15 (weekly×2 injections) for n=8 mice per group. The study endpoint for the vehicle-treated and the docetaxel 15 mg/kg groups was a group mean tumor size of 1000 mm³. The study endpoint for the docetaxel 30 mg/kg and the nanoparticles groups was an individual mouse tumor size of 1000 mm³. On Day 50, the study was ended for all remaining mice. When removed from the study, mice were euthanized by CO₂ inhalation.

### Body Weight Changes

On Day 8, the PEGylated O-acetyl-5050-PLGA nanoparticles (dose=30 mg/kg) treatment group had a mean body weight of 27.6±1.0 g. On Day 29, this group had a mean body weight of 26.1±1.1 g, representing a maximum body weight loss of 5±3%. On the last day in the study (i.e. Day 50), the mean body weight was 27.2±1.7 g. The mice were regaining weight, to 97±3% of this group's initial body weight. The formulation administered as a treatment to the mice was shown to be sterile using a bioburden assay.

The initial mean body weight of the docetaxel vehicle treated group was 26.3±1.9 g on Day 8. When this group was removed from the study on Day 25, the mean body weight was 27.8±2.3 g. This represented a 106±2% of the initial mean body weight. In comparison for the mice administered with docetaxel, on Day 8, the mean body weight of the docetaxel administered 15 mg/kg group was 27.3±2.3 g. On Day 22, this group decreased in body weight to 25.3±1.7 g, representing a maximum of 7% body weight loss. On Day 36, when the docetaxel administered 15 mg/kg group was removed from the study, the mean body weight was 30.7±2.5 g, representing a 113±11% of the initial body weight. Similarly, on Day 8, the mean body weight of the docetaxel administered 30 mg/kg group was 26.3±1.3 g. On Day 22, the mean body weight decreased to 23.7±1.9 g, representing a maximum of 10% body weight loss. On Day 36, this group weighed 30.7±2.5 g, representing a 105±9% of the initial body weight. Overall, there was a dose-related decline in body weights of mice administered with docetaxel.

### Tumor Growth Inhibition and Tumor Growth Delay (TGD)

Tumor growth delay (TGD) is calculated by the difference between the day when the treatment group tumor size reached the maximum tumor volume of 3000 mm³ and the day when the vehicle treated group reached a tumor volume of 3000 mm³.

For the PEGylated O-acetyl-5050-PLGA nanoparticles administered at a dose of 30 mg/kg, on Day 25, the tumor volume was 110±135 mm³ (range 30-408 mm³), with a TGI of 91%. The group mean tumor volume did not reach the endpoint during the duration of the study. One individual mouse reached 1000 mm³ on Day 29, however 6 mice remained in the study on Day 50. The TGD could not be calculated, but is estimated to be greater than 25 days.

For the docetaxel treatment group, on Day 25, the tumor volume of the 15 mg/kg group was 349±470 mm³ (range 68-1481 mm³), with a TGI of 71%. This group surpassed the endpoint on Day 32 with a tumor volume of 1477±1730 mm³ (range 165-5692 mm³). No difference in the slope of the growth curve was apparent. The TGD was determined to be 5 days for the docetaxel treatment group (15 mg/kg) by extrapolating to when the tumor growth curve crossed 1000 mm³. On Day 25, the tumor volume of the 30 mg/kg group was 63±68 mm³ (range 7-218 mm³), with a TGI of 95%. This group reached the endpoint on Day 39 with a tumor volume of 950±1239 (0-3803 mm³). Individual mice reached 1000 mm³ on Day 32 (1 mouse), Day 39 (1 mouse), Day 42 (3 mice) and Day 46 (1 mouse). On Day 50, 2 mice still remained in the study. No difference in the slope of the growth curve was apparent. The TGD was calculated to be 14 days. There was a dose-related inhibition of tumor growth of mice administered with the docetaxel treatment groups.

In contrast, on Day 25, the mean tumor volume was 1000 mm³ for the docetaxel vehicle treatment group and the tumor doubling time was 4 days. There was no effect by the docetaxel vehicle on tumor growth, compared to the other treatment groups. The PEGylated O-acetyl-5050-PLGA nanoparticles administered at a dose of 30 mg/kg showed improved efficacy and a greater TGD, compared to docetaxel, at the same dose and schedule.

### Tumor Growth Inhibition and Tumor Growth Delay of A2780 Tumor-Bearing Mice Administered Treatments.

| | | Day 25 | |
|---|---|---|---|
| | | Tumor Growth | Tumor Growth |
| | Dose | Inhibition | Delay |
| Group | (mg/kg) | (%) | (day) |
| Docetaxel Vehicle control | 0 | — | |
| Free docetaxel | 15 | 71 | 5 |
| Free docetaxel | 30 | 95 | 14 |
| PEGylated O-acetyl-5050- | 30 | 91 | >25 |
| PLGA nanoparticles | | | |

In the following examples when reference is made to “mPEG(Xk)-PLGA Y wt %”, Xk indicates the weight average molecular weight of the mPEG portion of the mPEG-PLGA polymer (e.g., mPEG(2k) indicates that 2 kDa mPEG is conjugated to PLGA), and Y indicates the weight percentage of mPEG-PLGA as compared to the PLGA-drug conjugate in the initial mixture used to make the nanoparticles. For example, 16 wt % indicates that an 84:16 weight ratio of PLGA-drug conjugate to mPEG-PLGA was prepared and added to surfactant in order to prepare the nanoparticles. Typically, approximately half of the mPEG-PLGA used in the reaction is incorporated in to the product nanoparticles. Thus the approximate components of the nanoparticles in the following examples are as follows:

mPEG(2k)-PLGA 16 wt %=In the particle: mPEG(2k)-PLGA˜8 wt %, PVA˜23 wt %, Docetaxel-5050 PLGA-O-acetyl˜69 wt %
mPEG(2k)-PLGA 30 wt %=In the particle: mPEG(2k)-PLGA˜17 wt %, PVA˜23 wt %, Docetaxel-5050 PLGA-O-acetyl˜60 wt %
mPEG(2k)-PLGA 40 wt %=In the particle: mPEG(2k)-PLGA˜23 wt %, PVA˜26 wt %, Docetaxel-5050 PLGA-O-acetyl˜51 wt %
mPEG(5k)-PLGA 16 wt %=In the particle: mPEG(5k)-PLGA˜8 wt %, PVA˜22%, Docetaxel-5050 PLGA-O-acetyl˜70%
mPEG(5k)-PLGA 30 wt %=In the particle: mPEG(5k)-PLGA˜16 wt %, PVA˜24%, Docetaxel-5050 PLGA-O-acetyl˜60%
mPEG(5k)-PLGA 40 wt %=In the particle: mPEG(5k)-PLGA˜18 wt %, PVA˜24%, Docetaxel-5050 PLGA-O-acetyl˜58%

### Example 43

### Efficacy and Tolerability of PEGylated Docetaxel-5050 PLGA-O-Acetyl Nanoparticles in a B16.F10 Murine Melanoma Model

B16.F10 cells were grown in culture to confluency in MEM-α medium supplemented with 10% fetal bovine serum (FBS, passage 4) and 1% penicillin/streptomycin and then resuspended in PBS. A volume of 0.1 mL containing 1×10⁶ cells was subcutaneously implanted into the right flank of male C57BL/6 mice on day-1.

The seven treatment groups that were administered to the mice included: 1) A docetaxel formulation prepared at 10 mg/mL stock solution (with 20 mg of docetaxel, 0.2 mL ethanol, 0.5 mL polysorbate 80 and 1.3 mL water, added in that specific order and vortexed to ensure proper mixing) diluted further with PBS to 1.5 and 3 mg/mL for a corresponding dose of 15 and 30 mg/kg. For a 60 mg/kg dose, a 20 mL/kg injection volume of a concentration of 3 mg/mL docetaxel formulation was administered. 2) PEGylated docetaxel-5050 PLGA-O-acetyl nanoparticles (mPEG(2k)-PLGA at 16 wt %) administered at doses of 15 and 30 mg/kg. 3) PEGylated docetaxel-5050 PLGA-O-acetyl nanoparticles (mPEG(2k)-PLGA at 30 wt %) administered at doses of 15, 30 and 60 mg/kg. 4) PEGylated docetaxel-5050 PLGA-O-acetyl nanoparticles (mPEG(2k)-PLGA at 40 wt %)) administered at doses of 15 and 30 mg/kg. 5) PEGylated docetaxel-5050 PLGA-O-acetyl nanoparticles (mPEG(5k)-PLGA at 16 wt %) administered at a dose of 15 mg/kg. 6) PEGylated docetaxel-5050 PLGA-O-acetyl nanoparticles (mPEG(5k)-PLGA at 30 wt %) administered at doses of 15 and 30 mg/kg. 7) PEGylated docetaxel-5050 PLGA-O-acetyl nanoparticles (mPEG(5k)-PLGA at 40 wt %) administered at a dose of 15 mg/kg. Refer to table for detailed description of formulations.

The treatments were administered IV into the tail vein at a dose volume of 10 or 20 mL/kg depending on the treatment group, beginning on post-implantation day 5, when the mean tumor volume was approximately 55 mm³. Animals were monitored for any morbidity and adverse effect three times a week. Body weight and tumor volume were also measured three times a week.

Tumor volume was calculated with the following equation: (width×width×length)/2 mm³. Efficacy was determined by tumor growth inhibition (TGI), tumor growth delay (TGD) and survival. TGI was represented as % and calculated as follows: (1−(treated tumor volume/control tumor volume))×100 when the control group mean tumor volume reached >3000 mm³. Tolerability was determined by changes in body weight, expressed as a percent of the initial body weight on post-implantation day-5. Health monitoring was conducted three times a week to evaluate lethargy, tremors, hypothermia, ataxia, hind limb paralysis etc. The criteria at which a mouse was removed from the study were >20% body weight loss or severe morbidity or hind limb paralysis.

PEGylated Nanoparticles (mPEG(2k)-PLGA at 16 wt %)-q3dq4d

The docetaxel control group and the PEGylated nanoparticles were administered three times over a two week schedule at a dose of 15 mg/kg and 30 mg/kg respectively. The docetaxel group showed a TGI of 90% in comparison to the PEGylated nanoparticles, which had a TGI of 84%. The docetaxel group exhibited a similar TGD of 12 days compared to 13 days for the PEGylated nanoparticles. The PEGylated nanoparticles did not cause any body weight loss and was better tolerated than the docetaxel group which caused a 12% maximum body weight loss.

PEGylated Nanoparticles (mPEG(2k)-PLGA at 30 wt %)-q3dq4d

The docetaxel control group and the PEGylated nanoparticles were administered three times over a two week schedule at a dose of 15 mg/kg. Both the PEGylated nanoparticles and the docetaxel groups were equally efficacious. The TGI of the docetaxel and PEGylated groups were 90% and 86% respectively. Similarly both groups exhibited the same TGD of 11 days. The PEGylated nanoparticles did not show any body weight loss and was better tolerated than docetaxel, which caused a 11% maximum body weight loss.

PEGylated Nanoparticles (mPEG(2k)-PLGA at 30 wt %)-q7d

Both the docetaxel control group and the PEGylated nanoparticles were administered three times, once every week at a dose of 30 mg/kg. The TGI for the docetaxel and PEGylated nanoparticles group was 90% and 96% respectively. The PEGylated nanoparticles showed a greater TGD (25 days) and survival compared to the docetaxel group (17 days). In addition, the PEGylated nanoparticles were better tolerated and caused no body weight loss, whereas the docetaxel group had a maximum body weight loss of 11%.

PEGylated Nanoparticles (mPEG(2k)-PLGA at 30 wt %)-q14d

Both the docetaxel control group and the PEGylated nanoparticles were administered two times, once every two weeks at a dose of 60 mg/kg. The TGI for the PEGylated nanoparticles group was greater (i.e. 97%) than that of the docetaxel group (i.e. 71%). The PNP also exhibited an increased TGD and survival compared to docetaxel. The docetaxel group reached the tumor volume end point on day 29 and showed a TGD of 11 days. In the case of the PEGylated nanoparticles group, the average tumor volume was 118 mm³ on day 42. A TGD for the PEGylated nanoparticles could not be determined because at the time of measurement, the group still had not reached the tumor volume end point (i.e. on day 56, the average tumor volume was 840 mm³). In addition, the PEGylated nanoparticles were well tolerated and caused only 8% maximum body weight loss. The control group docetaxel did not show any body weight loss.

PEGylated Nanoparticles (mPEG(2k)-PLGA at 40 wt %)-q7d

Both the docetaxel control group and the PEGylated nanoparticles were administered three times, once every week at a dose of 15 mg/kg. The TGI of the docetaxel group and the PEGylated nanoparticles was shown to be similar (approximately 90%). The TGD of the free docetaxel and the PEGylated nanoparticles was 11 and 13 days respectively. There was no body weight loss associated with the PEGylated nanoparticles; in contrast, the docetaxel group showed a maximum body weight loss of 11%.

PEGylated Nanoparticles (mPEG(5k)-PLGA at 16 wt %)-q3dq4d

The docetaxel and the PEGylated nanoparticles groups were administered three times over a two week schedule at a dose of 15 mg/kg. The docetaxel group had a TGI of 90% compared to the PEGylated nanoparticles group which had a TGI of 71%. The TGD of the docetaxel and PEGylated nanoparticles groups were 11 and 7 days respectively. The PEGylated nanoparticles were better tolerated and showed no body weight loss compared to the docetaxel group, which exhibited an 11% maximum body weight loss.

PEGylated Nanoparticles (mPEG(5k)-PLGA at 30 wt %)-q3dq4d

The docetaxel and the PEGylated nanoparticles groups were administered three times over a two week schedule at a dose of 15 mg/kg. The docetaxel and PEGylated nanoparticles groups showed a similar TGI (i.e. 90%). In terms of the TGD, the docetaxel group showed 11 days compared to the PEGylated nanoparticles (i.e. 13 days). The PEGylated nanoparticles were better tolerated than the docetaxel control group. Also, the docetaxel group exhibited a maximum body weight loss of 11% compared to no body weight loss shown by the PEGylated nanoparticles group.

PEGylated Nanoparticles (mPEG(5k)-PLGA at 30 wt %)-q7d

Both the docetaxel and PEGylated nanoparticles groups were administered three times, once a week at a dose of 30 mg/kg. The TGI of the docetaxel and PEGylated nanoparticles groups were 90% and 97% respectively. The TGD of the docetaxel group was determined to be 17 days as the average tumor volume reached the end point of 3000 mm³ at day 37. A TGD for the PEGylated nanoparticles could not be determined because at the time of measurement, the group still had not reached the tumor volume end point (i.e. on day 47, the average tumor volume was 2100 mm³). The PEGylated nanoparticles did not cause any body weight loss and was better tolerated than free docetaxel which caused a 11% body weight loss.

PEGylated Nanoparticles (mPEG(5k)-PLGA at 40 wt %)-q4dq3d

The docetaxel and PEGylated nanoparticles groups were administered three times over a two week schedule at a dose of 15 mg/kg. The TGI for both groups was similar (approximately 90-92%). The TGD for the PEGylated nanoparticles (i.e. 15 days) was greater than that for the docetaxel group (i.e. 11 days). The PEGylated nanoparticles did not cause any body weight loss to the mice and were better tolerated compared to the docetaxel group which resulted in a 11% maximum body weight loss.

### Comparison of Efficacy and Tolerability of Different PEGylated Nanoparticles (2k) Formulation and the Control Docetaxel Treatment Group

| | | | Tumor | Tumor | Maximum |
|---|---|---|---|---|---|
| | | | growth | growth | body |
| | | Dose | inhibition | delay | weight |
| | | (mg/ | (TGI) | (TGD) | loss |
| Formulation | Schedule | kg) | (%) | (days) | (%) |
| Docetaxel | q3dq4dx3 | 15 | 90 | 12 | 12 |
| PEGylated nps | q3dq4dx3 | 30 | 84 | 13 | 0 |
| (mPEG(2k)-PLGA | | | | | |
| 16 wt %) | | | | | |
| Docetaxel | q3dq4dx3 | 15 | 90 | 11 | 11 |
| PEGylated nps | q3dq4dx3 | 15 | 86 | 11 | 0 |
| (mPEG(2k)-PLGA | | | | | |
| 30 wt %) | | | | | |
| Docetaxel | q7dx3 | 30 | 90 | 17 | 11 |
| PEGylated nps | q7dx3 | 30 | 96 | 25 | 0 |
| (mPEG(2k)-PLGA | | | | | |
| 30 wt %) | | | | | |
| Docetaxel | q14dx2 | 60 | 71 | 11 | 0 |
| PEGylated nps | q14dx2 | 60 | 97 | >38 | 8 |
| (mPEG(2k)-PLGA | | | | | |
| 30 wt %) | | | | | |
| Docetaxel | q3dq4dx3 | 15 | 90 | 11 | 11 |
| PEGylated nps | q3dq4dx3 | 15 | 89 | 13 | 0 |
| (mPEG(2k)-PLGA | | | | | |
| 40 wt %) | | | | | |

* q3dq4dx3—three injections administered over 2 weeks (3 days in between 1^{st} and 2^{nd} injection, 4 days in between 2^{nd} and 3^{rd} injection).
* q7dx3—three injections seven days apart.
* q14dx2—two injections 14 days apart.

### Comparison of Efficacy and Tolerability of Different PEGylated Nanoparticles (5k) Formulation and the Control Docetaxel Treatment Group

| | | | Tumor | Tumor | Maximum |
|---|---|---|---|---|---|
| | | | growth | growth | body |
| | | Dose | inhibition | delay | weight |
| | | (mg/ | (TGI) | (TGD) | loss |
| Formulation | Schedule | kg) | (%) | (days) | (%) |
| Docetaxel | q3dq4dx3 | 15 | 90 | 11 | 11 |
| PEGylated nps | q3dq4dx3 | 15 | 71 | 7 | 0 |
| (PEG(5k)-PLGA | | | | | |
| 16 wt %) | | | | | |
| Docetaxel | q3dq4dx3 | 15 | 90 | 11 | 11 |
| PEGylated nps | q3dq4dx3 | 15 | 90 | 13 | 0 |
| (PEG(5k)-PLGA | | | | | |
| 30 wt %) | | | | | |
| Docetaxel | q7dx3 | 30 | 90 | 17 | 11 |
| PEGylated nps | q7dx3 | 30 | 97 | >38 | 0 |
| (PEG(5k)-PLGA | | | | | |
| 30 wt %) | | | | | |
| Docetaxel | q4dq3dx3 | 15 | 90 | 11 | 11 |
| PEGylated nps | q4dq3dx3 | 15 | 92 | 15 | 0 |
| (PEG(5k)-PLGA | | | | | |
| 40 wt %) | | | | | |

* q3dq4dx3—three injections administered over 2 weeks (3 days in between 1^{st} and 2^{nd} injection, 4 days in between 2^{nd} and 3^{rd} injection).
* q4dq3dx3—three injections administered over 2 weeks (4 days in between 1^{st} and 2^{nd} injection, 3 days in between 2^{nd} and 3^{rd} injection).
* q7dx3—three injections seven days apart.

### Example 44

### In Vivo Efficacy of PEGylated Docetaxel-5050 PLGA-O-Acetyl Nanoparticles in a HCT-116 Colon Xenograft Model

HCT-116 cells were grown in culture to confluency in McCoy's 5a medium containing 10% FBS and 1% penicillin/streptomycin and then resuspended in McCoy's 5a (passage 4). This suspension of HCT-116 cells (density=3.7×10⁶ cells/mL) was implanted subcutaneously above the right hind leg of male CD-1 nude mice on day 1.

The three treatment groups that were administered to HCT-116 tumor bearing mice (n=6-7 per group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to 1.5 mg/mL for a corresponding dose of 30 mg/kg at an injection volume of 20 mL/kg respectively; 3) PEGylated docetaxel-5050 PLGA-O-acetyl nanoparticle formulation (mPEG(2k)-PLGA with initial amount of 16 wt %) at a docetaxel equivalent concentration of 1.5 mg/mL for a corresponding dose of 30 mg/kg at an injection volume of 20 mL/kg

The treatments were administered IV into the tail vein at the respective dose volumes (refer to previous paragraph), beginning on post-implantation Day 13, when the mean tumor volume was 131 mm³. The vehicle and docetaxel treatments were administered two times, on Days 13 and 20 (weekly×two injections).

The mice that were administered docetaxel at a dose of 30 mg/kg lost a maximum body weight of 14%. In comparison, the PEGylated formulation administered at a dose of 30 mg/kg, did not lose any weight during the study.

### Tumor Growth Inhibition

The tumor growth inhibition (TGI) of the mice treated with docetaxel at a dose of 30 mg/kg was 88%. Extrapolating to where the tumor growth curve reached the end point at a tumor volume of 1000 mm³, the TGD was calculated to be 22 days. For the PEGylated nanoparticles at a dose of 30 mg/kg, the TGI was 77%. The TGD was determined to be 21 days.

### Example 45

### In Vivo Efficacy of PEGylated Docetaxel-5050 PLGA-O-Acetyl Nanoparticles in a SK-OV-3 Ovarian Human Xenograft Model

SK-OV-3 cells were grown in culture to confluency in RPMI medium containing 10% FBS and 1% penicillin/streptomycin and then resuspended in RPMI (passage 4) for implantation into mice. This suspension of SK-OV-3 cells (density=30×10⁶ cells/mL) was implanted into the mammary gland of female CD-1 nude mice on Day 1.

Treatment groups that were administered to SK-OV-3 tumor-bearing mice (n=4-5 per group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to A) 1.5 mg/mL for a corresponding dose of 15 mg/kg and 30 mg/kg at an injection volume of 10 mL/kg and 20 mL/kg respectively, and B) 3 mg/mL for a dose of 60 mg/kg at an injection volume of 20 mL/kg; 3) PEGylated docetaxel-5050 PLGA-O-acetyl nanoparticle formulation (mPEG(2k)-PLGA with initial amount of 16 wt %) at a docetaxel equivalent concentration of 2.9 mg/mL for a corresponding dose of 60 mg/kg at an injection volume of 21 mL/kg.

The treatments were administered IV into the tail vein at the dose volumes stated above, beginning on post-implantation Day 51, when the mean tumor volume was 232 mm³. The vehicle and docetaxel treatments were administered two times, on Days 51 and 58 (weekly×two injections). The PEGylated nanoparticles treatment was administered once, on Day 51.

The high dose of docetaxel, 60 mg/kg, caused greater than 20% body weight loss. Ataxia, which is defined as the inability to coordinate voluntary muscular movements that is symptomatic of some CNS disorders and injuries and not due to muscle weakness, was observed in all the mice four days after the second treatment of docetaxel. This group was removed 18 days after the second treatment, despite supportive measures (fluid replacement, easier access to food), due to the ataxia becoming more severe and affecting the forelimbs. The lower dose of docetaxel, 30 mg/kg, did not cause ataxia. Maximum body weight loss in the group administered docetaxel 30 mg/kg was 13%. The group administered the PEGylated nanoparticles at a dose of 60 mg/kg was only administered that treatment one time. No ataxia developed in this group, but this could not be compared to the high dose of docetaxel because of the different numbers of treatments. Maximum body weight loss in the group administered the PEGylated nanoparticles at 60 mg/kg was 11%, equivalent to the free drug (i.e. docetaxel) at 30 mg/kg.

### Tumor Growth Inhibition

All treatments inhibited tumor growth. The tumor growth delay (TGD) for docetaxel at a dose of 15 mg/kg was 18 days. The TGD for docetaxel at a dose of 30 mg/kg was 42 days. At this time, this group had a large variation, with two mice>1000 mm³ and three mice<50 mm³. The TGD for PEGylated nanoparticles at 60 mg/kg was 94 days, with a large intragroup variation with two mice>1000 mm³ and three mice<325 mm³, a similar pattern to free drug at a dose of 30 mg/kg, but delayed approximately 54 days relative to free drug.

### Example 46

### In Vivo Efficacy of PEGylated Docetaxel-5050 PLGA-O-acetyl Nanoparticles in a MDA-MB-435 Melanoma Human Xenograft Model

MDA-MB-435 cells were grown in culture to confluency in RPMI medium containing 10% FBS and 1% penicillin/streptomycin and then resuspended in RPMI (passage 4) for implantation into mice. A volume of 0.1 mL containing 4.0×10⁶ cells MDA-MB-435 cells were implanted into the mammary gland of female CD-1 nude mice on Day 1.

Treatments that were administered to the mice (n=6-7/group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to A) 1.5 mg/mL for a corresponding dose of 15 and 30 mg/kg at an injection volume of 10 mL/kg and 20 mL/kg, respectively, B) 3.0 mg/mL for a dose of 60 mg/kg at an injection volume of 20 mL/kg; 3) PEGylated docetaxel-5050 PLGA-O-acetyl nanoparticle formulation (mPEG(2k)-PLGA with initial amount of 16 wt %) made at a docetaxel equivalent concentration of 1.1 mg/mL for a corresponding dose of 30 mg/kg at an injection volume of 26 mL/kg; 4) PEGylated docetaxel-5050 PLGA-O-acetyl nanoparticle formulation (mPEG(2k)-PLGA with initial amount of 30 wt %) made at a docetaxel equivalent of 1.5 and 2.85 mg/mL for corresponding doses of A) 15 mg/kg at an injection volume of 10 mL/kg, B) 30 and 60 mg/kg at an injection volume of 11 mL/kg and 21 mL/kg, respectively.

The treatments were administered IV into the tail vein at the dose volumes stated above, beginning on post-implantation Day 21, when the mean tumor volume was 150 mm³ or, for one group, on Day 37, when the mean tumor volume for that group was 433 mm³. The treatments were administered two times, on Days 21 and 28 (weekly×two injections) for the vehicle, docetaxel and PEGylated nanoparticles groups and on Days 37 and 44 for a group that was administered PEGylated nanoparticles when the tumors were at a larger tumor volume (i.e. 433 mm³).

For groups administered the free docetaxel, the high dose, 60 mg/kg, caused greater than 20% body weight loss. Ataxia was observed four days after the second treatment. This group was removed nine days after the second treatment, despite supportive measures (fluid replacement, easier access to food), due to severe ataxia. The docetaxel group administered at a dose of 30 mg/kg did not cause ataxia. Maximum body weight loss in the docetaxel dosed at 30 mg/kg group was 14% and in the case of the 15 mg/kg group, it was 10% of initial body weight.

Groups administered PEGylated nanoparticles had different responses depending on the wt % and dose. The PEGylated nanoparticles (PEG at initial amount of 16 wt %) administered at a dose of 30 mg/kg did not show any weight loss. The PEGylated nanoparticles (PEG at initial amount of 30 wt %) administered at a dose of 15 mg/kg also did not show any weight loss. At a higher dose (30 mg/kg), the PEGylated nanoparticles treatment group lost 6% of its initial body weight. At an even higher dosage (60 mg/kg), the treatment group receiving PEGylated nanoparticles administered starting on Day 21 (i.e. when the mean tumor size was 150 mm³) lost 11% body weight, which was equivalent to the free drug at a dose of 30 mg/kg. The treatment group receiving same PEGylated nanoparticles at a dose of 60 mg/kg were also administered on Day 37 (i.e. when the mean tumor size was 433 mm³) lost 19% body weight. This exaggerated weight loss was likely due to undetermined necrotic factors released from a relatively large amount of dead tumor tissue. One mouse in this latter group was found dead on Day 64 despite supportive measures (fluid replacement, easier access to food). The other mice in that group almost fully recovered their lost body weight and do not appear to be at any health risk at this time (Day 76).

### Ataxia

Mice administered docetaxel at a dose of 60 mg/kg developed ataxia. The entire group showed abnormal gait and lack of coordination of the front limbs nine days after the second treatment. No other doses of docetaxel were observed to cause ataxia. In contrast to docetaxel, none of the mice administered PEGylated nanoparticles at any dose developed ataxia.

### Tumor Growth Inhibition

All treatments groups resulted in tumor growth inhibition. The mean tumor volume of vehicle-treated group reached the endpoint of 1000 mm³ on Day 58 post-tumor implantation. As of Day 76, it appears that the treatment at a dose of 15 mg/kg resulted in the same TGI for free docetaxel and PEGylated nanoparticles. At a dose of 30 mg/kg, the TGI for free docetaxel was greater than that for PEGylated nanoparticles (mPEG-PLGA initial amount of 30 wt %>mPEG-PLGA initial amount of 16 wt %). At a dose of 60 mg/kg, free docetaxel was equivalent to PEGylated nanoparticles until the free drug group was removed from the study. As the study continues, docetaxel at a dose of 30 mg/kg is equivalent to PEGylated nanoparticles at a dose of 60 mg/kg.

### Example 47

### Tolerability of the Free Drug Docetaxel and PEGylated Docetaxel-5050 PLGA-O-Acetyl Nanoparticles in Normal Male C57BL/6 Non-Tumor-Bearing Mice

Treatments that were administered to the male C57BL/6 mice (n=5/group) included: 1) a docetaxel vehicle formulation consisting of 1.5% ethanol/3.75% polysorbate 80/9.75% water/85% PBS at 20 mL/kg; 2) 10 mg/mL docetaxel stock solution (prepared with 20 mg of docetaxel, 0.2 mL ethanol, 0.5 mL polysorbate 80 and 1.3 mL water, added in that specific order and vortexed to ensure proper mixing) diluted in PBS to 1.5, 2.25 and 3 mg/mL for a corresponding dose of 30, 45 and 60 mg/kg at an injection volume of 20 mL/kg; 3) PEGylated docetaxel-5050 PLGA-O-acetyl nanoparticles formulation (mPEG(2k)-PLGA initial amount of 30 wt %) at a docetaxel equivalent of 2.85 mg/mL for a dose of 60 mg/kg at an injection volume of 21 mL/kg.

Treatments were administered intravenously on a q7dx2 schedule, i.e., two treatments seven days apart (the first treatment was on Day one). The study ended on Day 14, six days after the 2^{nd} treatment. Blood was collected for complete blood count (CBC) and serum chemistry. Leg muscles were collected so that nerve degeneration could be assessed from the sciatic nerve.

The vehicle-treated group gained 23% of its initial body weight by the end of the study. Docetaxel administered at doses of 30 and 45 mg/kg gained weight, up to 7% at the second treatment, weighing 3% and 2% respectively more than the initial on Day 14. The group administered docetaxel at a dose of 60 mg/kg did not gain weight after the first treatment and lost weight (19%) after the second treatment, by the end of the study. The group administered PEGylated nanoparticles at a dose of 60 mg/kg did not gain weight after the first treatment and lost weight (16%) after the second treatment, by the end of the study.

### Complete Blood Count

From the table below, the CBC analyses showed that the white blood cell number, neutrophil number and lymphocyte number were lower in the groups administered docetaxel and PNP at a dose of 60 mg/kg. The white blood cells are expressed in units of ×1000 cells/μL, the neutrophils and lymphocytes are expressed in units of cells/μL.

| | WBC # | | Neutrophil | | Lymphocyte | |
|---|---|---|---|---|---|---|
| Treatment | mean | SD | mean | SD | mean | SD |
| Docetaxel vehicle group | 8.3 | 1.0 | 1474 | 390 | 6563 | 757 |
| Docetaxel, 30 mg/kg | 5.1 | 1.7 | 556 | 254 | 4350 | 1394 |
| Docetaxel, 45 mg/kg | 7.8 | 1.7 | 752 | 266 | 6780 | 1855 |
| Docetaxel, 60 mg/kg | 6.2 | 1.0 | 470 | 159 | 5590 | 938 |
| PEGylated docetaxel-5050 | 4.6 | 0.9 | 488 | 162 | 3958 | 1001 |
| PLGA-O-acetyl nanoparticles | | | | | | |
| (mPEG(2k)-PLGA initial | | | | | | |
| amount of 30 wt %) | | | | | | |

### Serum Chemistry

Both the free docetaxel group and the PEGylated docetaxel-5050 PLGA-O-acetyl nanoparticles formulation (mPEG(2k)-PLGA initial amount of 30 wt %) did not affect any serum chemistry parameter at doses up to 60 mg/kg.

### Sciatic Nerve Histopathology Assessment

Mice administered the free docetaxel was observed to develop ataxia during the study with a dose-related effect. Specifically, no mice in the 30 mg/kg group were seen to develop ataxia or any overt signs of nerve damage. One mouse in the 45 mg/kg group was observed to develop ataxia on Day 14, while the others in that group had a normal gait. Five out of five mice in the 60 mg/kg group was observed to develop ataxia—one on Day 12, all on Day 14. None of the mice in the group administered PEGylated nanoparticles at a dose of 60 mg/kg was shown to develop ataxia. Refer to the table below for results.

| | Dose | Ataxia |
|---|---|---|
| Group | (mg/kg) | (%) |
| Docetaxel vehicle control | 0 | — |
| Free docetaxel | 30 | 0 |
| Free docetaxel | 45 | 20 |
| Free docetaxel | 60 | 100 |
| PEGylated docetaxel-5050 PLGA-O-acetyl | 60 | 0 |
| nanoparticles (mPEG(2k)-PLGA initial | | |
| amount of 30 wt %) | | |

These data showed that, contrary to the MDA-MB-435 study described above and historical data, free docetaxel and PEGylated docetaxel-5050 PLGA-O-acetyl nanoparticles (mPEG(2k)-PLGA initial amount of 30 wt %) at a dose of 60 mg/kg q7dx2 (i.e. two treatments seven days apart) are equivalent regarding body weight loss. Further, and also contrary to historical data, these treatments were similar regarding effects on the CBC.

A pathologist's assessment of the sciatic nerve histology found no treatment effects in any animals. Since ataxia was observed to be severe in the docetaxel group at a dose of 60 mg/kg, and damage by taxanes of the sciatic nerve at the level of the muscle was shown previously in published studies, it was suggested by the pathologist that the section of sciatic nerve that was examined was too far from the spinal chord, and damage did not yet develop in that part of the sciatic nerve at the time of tissue collection.

### Example 48

### Synthesis of Polyfunctionalized PLGA/PLA Based Polymers

One could synthesize a PLGA/PLA related polymer with functional groups that are dispersed throughout the polymer chain that is readily biodegradable and whose components are all bioacceptable components (i.e. known to be safe in humans). Specifically, PLGA/PLA related polymers derived from 3-S-[benxyloxycarbonyl)methyl]-1,4-dioxane-2,5-dione (BMD) could be synthesized (see structures below). (The structures below are intended to represent random copolymers of the monomeric units shown in brackets.)

1. PLGA/PLA Related Polymer Derived from BMD

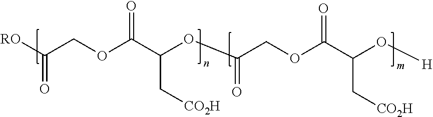

2. PLGA/PLA Related Polymer with BMD and 3,5-dimethyl-1,4-dioxane-2,5-dione (bis-DL-lactic Acid Cyclic Diester)

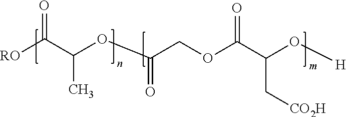

3. PLGA/PLA Related Polymer with BMD and 1,4-dioxane-2,5-dione (bis-glycolic Acid Cyclic Diester

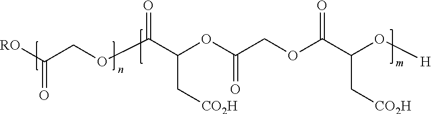

In a preferred embodiment, PLGA/PLA polymers derived from BMD and bis-DL-lactic acid cyclic diester will be prepared with a number of different pendent functional groups by varying the ratio of BMD and lactide. For reference, if it is assumed that each polymer has a number average molecular weight (Mn) of 8 kDa, then a polymer that is 100 wt % derived from BMD has approximately 46 pendant carboxylic acid groups (1 acid group per 0.174 kDa). Similarly, a polymer that is 25 wt % derived from BMD and 75 wt % derived from 3,5-dimethyl-1,4-dioxane-2,5-dione (bis-DL-lactic acid cyclic diester) has approximately 11 pendant carboxylic acid groups (1 acid group per 0.35 kDa). This compares to just 1 acid group for an 8 kDa PLGA polymer that is not functionalized and 1 acid group/2 kDa if there are 4 sites added during functionalization of the terminal groups of a linear PLGA/PLA polymer or 1 acid group/1 kDa if a 4 kDa molecule has four functional groups attached.

Specifically, the PLGA/PLA related polymers derived from BMD will be developed using a method by Kimura et al., Macromolecules, 21, 1988, 3338-3340. This polymer would have repeating units of glycolic and malic acid with a pendant carboxylic acid group on each unit [RO(COCH₂OCOCHR₁O)ₙH where R is H, or alkyl or PEG unit etc. and R₁ is CO₂H]. There is one pendant carboxylic acid group for each 174 mass units. The molecular weight of the polymer and the polymer polydispersity can vary with different reaction conditions (i.e. type of initiator, temperature, processing condition). The Mn could range from 2 to 21 kDa. Also, there will be a pendant carboxylic acid group for every two monomer components in the polymer. Based on the reference previously sited, NMR analysis showed no detectable amount of the β-malate polymer was produced by ester exchange or other mechanisms.

Another type of PLGA/PLA related polymer derived from BMD and 3,5-dimethyl-1,4-dioxane-2,5-dione (bis-DL-lactic acid cyclic diester) will be synthesized using a method developed by Kimura et al., Polymer, 1993, 34, 1741-1748. They showed that the highest BMD ratio utilized was 15 mol % and this translated into a polymer containing 14 mol % (16.7 wt %) of BMD-derived units. This level of BMD incorporation represents approximately 8 carboxylic acid residues per 8 kDa polymer (1 carboxylic acid residue/kDa of polymer). Similarly to the use of BMD alone, no β-malate derived polymer was detected. Also, Kimura et al. reported that the glass transition temperatures (T_{g}) were in the low 20° C.'s despite the use of high polymer molecular weights (36-67 kDa). The T_{g}'s were in the 20-23° C. for these polymers whether the carboxylic acid was free or still a benzyl group. The inclusion of more rigidifying elements (i.e. carboxylic acids which can form strong hydrogen bonds) should increase the T_{g}. Possible prevention of aggregation of any nanoparticles formed from a polymer drug conjugate derived from this specific polymer will have to be evaluated due to possible lower T_{g} values.

Another method for synthesizing a PLA-PEG polymer that contains varying amounts of glycolic acid malic acid benzyl ester involves the polymerization of BMD in the presence of 3,5-dimethyl-1,4-dioxane-2,5-dione (bis-DL-lactic acid cyclic diester), reported by Lee et al., Journal of Controlled Release, 94, 2004, 323-335. They reported that the synthesized polymers contained 1.3-3.7 carboxylic acid units in a PLA chain of approximately 5-8 kDa (total polymer weight was approximately 11-13 kDa with PEG being 5 kDa) depending on the quantity of BMD used in the polymerization. In one polymer there were 3.7 carboxylic acid units/hydrophobic block in which the BMD represents approximately 19 wt % of the weight of the hydrophobic block. The ratio of BMD to lactide was similar to that observed by Kimura et al., Polymer, 1993, 34, 1741-1748 and the acid residues were similar in the resulting polymers (approximately 1 acid unit/kDa of hydrophobic polymer).

Polymers functionalized with BMD that are more readily hydrolysable will be prepared using the method developed by Kimura et al., International Journal of Biological Macromolecules, 25, 1999, 265-271. They reported that the rate of hydrolysis was related to the number of free acid groups present (with polymers with more acid groups hydrolyzing faster). The polymers had approximately 5 or 10 mol % BMD content. Also, in the reference by Lee et al., Journal of Controlled Release, 94, 2004, 323-335, the rate of hydrolysis of the polymer was fastest with the highest concentration of pendent acid groups (6 days for polymer containing 19.5 wt % of BMD and 20 days for polymer containing 0 wt % of BMD.

A drug (e.g. docetaxel, paclitaxel, doxorubicin, etc.) could be conjugated to a PLGA/PLA related polymer with BMD (refer to previous examples above). Similarly, a nanoparticle could be prepared from such a polymer drug conjugate.

### Example 49

### Synthesis of Polymers Prepared Using β-Lactone of Malic Acid Benzyl Esters

One could prepare a polymer by polymerizing MePEGOH with RS-β-benzyl malolactonate (a β-lactone) with DL-lactide (cyclic diester of lactic acid) to afford a polymer containing MePEG (lactic acid) (malic acid) Me(OCH2CH2O)[OCCCH(CH3)O]m[COCH2CH(CO2H)O]. as developed by Wang et al., Colloid Polymer Sci., 2006, 285, 273-281. These polymers would potentially degrade faster because they contain higher levels of acidic groups. It should be noted that the use of β-lactones generate a different polymer from that obtained using 3-[(benzyloxycarbonyl)methyl]-1,4-dioxane-2,5-dione. In these polymers, the carboylic acid group is directly attached to the polymer chain without a methylene spacer.

Another polymer that could be prepared directly from a β-lactone was reported by Ouhib et al., Ch. Des. Monoeres. Polym, 2005, 1, 25. The resulting polymer (i.e. poly-3,3-dimethylmalic acid) is water soluble as the free acid, has pendant carboxylic acid groups on each unit of the polymer chain and as well it has been reported that 3,3-dimethylmalic acid is a nontoxic molecule.

One could polymerize 4-benzyloxycarbonyl-,3,3-dimethyl-2-oxetanone in the presence of 3,5-dimethyl-1,4-dioxane-2,5-dione (DDD) and β-butyrolactone to generate a block copolymer with pendant carboxlylic acid groups as shown by Coulembier et al., Macromolecules, 2006, 39, 4001-4008. This polymerization reaction was carried out with a carbene catalyst in the presence of ethylene glycol. The catalyst used was a triazole carbene catalyst which leads to polymers with narrow polydispersities.

### Example 50

### Regioselective Synthesis of Docetaxel-2′-5050 PLGA-O-Acetyl

Docetaxel-2′-5050 PLGA-O-acetyl could be regioselectively prepared as illustrated in the following scheme. The 2′ hydroxyl group of docetaxel is first protected using benzylchloroformate. Following purification of the 2′ Cbz-protected docetaxel, the product may be orthogonally protected on the 7 and 10 hydroxyl groups using a silyl chloride (e.g., tert-butyldimethylsilyl chloride). The Cbz group may then be removed using hydrogenation over Pd/C, followed by coupling of PLGA-O-acetyl using EDC and DMAP. Final deprotection of the silyl protecting groups using TBAF would produce the docetaxel-2′-5050 PLGA-O-acetyl selectively coupled via the 2′ hydroxyl group.

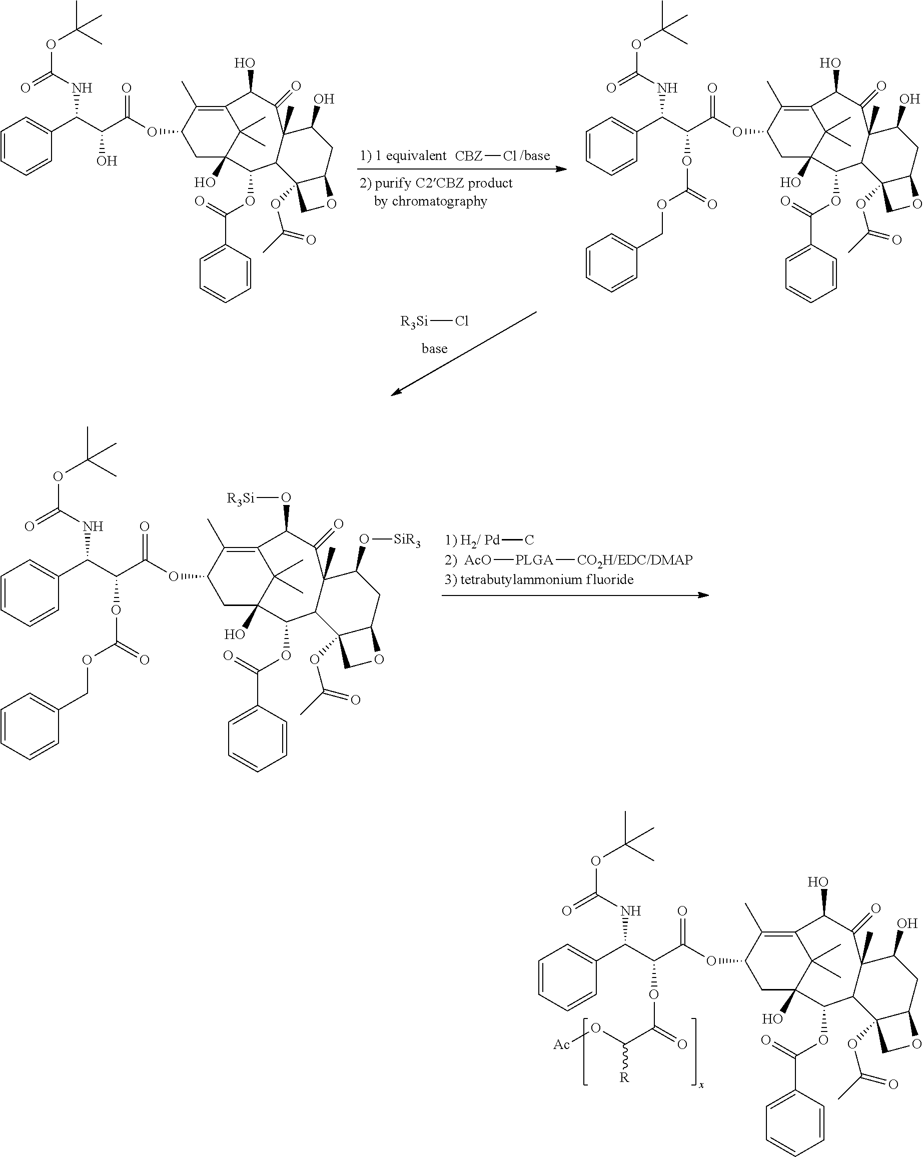

Alternatively, docetaxel-2′-5050 PLGA-O-acetyl could be regioselectively prepared as illustrated in the scheme below. The 2′ hydroxyl group of docetaxel is first protected using tert-butyldimethylsilyl chloride. Following purification of the 2′ TBDMS-protected docetaxel, the product may be orthogonally protected on the 7 and 10 hydroxyl groups using a benzylchloroformate. The TBDMS group may then be removed using TBAF, followed by coupling of PLGA-O-acetyl using EDC and DMAP. Final deprotection of the Cbz protecting groups via hydrogenation over Pd/C would produce the docetaxel-2′-5050 PLGA-O-acetyl selectively coupled via the 2′ hydroxyl group.

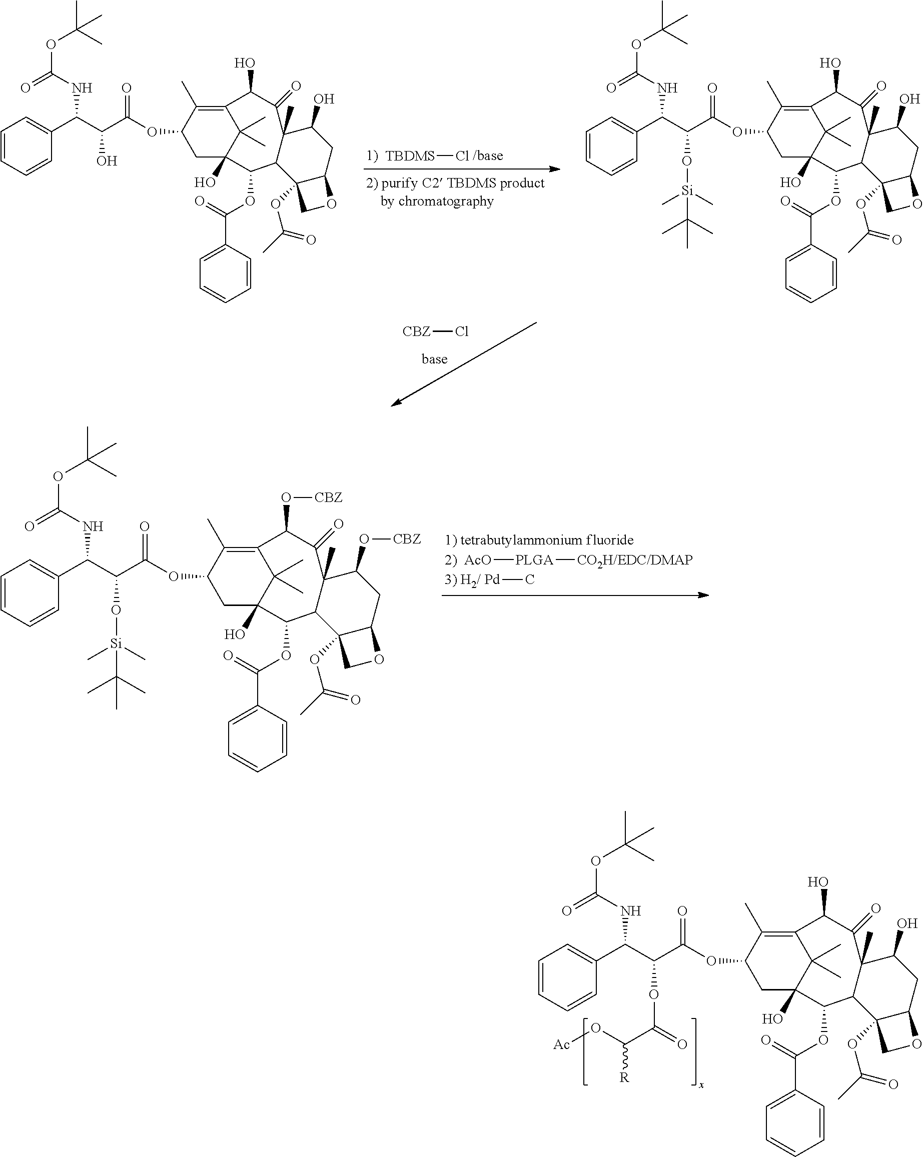

### Example 51

### Regioselective Synthesis of Docetaxel-7-5050 PLGA-O-acetyl and Docetaxel-10-5050 PLGA-O-acetyl

Docetaxel-7-5050 PLGA-O-acetyl and docetaxel-10-5050 PLGA-O-acetyl could be regioselectively prepared as illustrated in the following scheme. Docetaxel is first protected using two equivalents of benzylchloroformate, yielding a mixture of products. Two products, C2′/C7-bis-Cbz-docetaxel, and C2′/C10-bis-Cbz-docetaxel, can each be selectively purified.

C2′/C7-bis-Cbz-docetaxel can then be coupled to PLGA-O-acetyl using EDC and DMAP, which would result in attachment of PLGA-O-acetyl to the hydroxyl group at the 10-position of docetaxel. Deprotection of the Cbz protecting groups via hydrogenation over Pd/C would produce the docetaxel-10-5050 PLGA-O-acetyl selectively coupled via the 10 hydroxyl group.

C2′/C10-bis-Cbz-docetaxel can then be coupled to PLGA-O-acetyl using EDC and DMAP, which would result in attachment of PLGA-O-acetyl to the hydroxyl group at the 7-position of docetaxel. Deprotection of the Cbz protecting groups via hydrogenation over Pd/C would produce the docetaxel-7-5050 PLGA-O-acetyl selectively coupled via the 7 hydroxyl group.

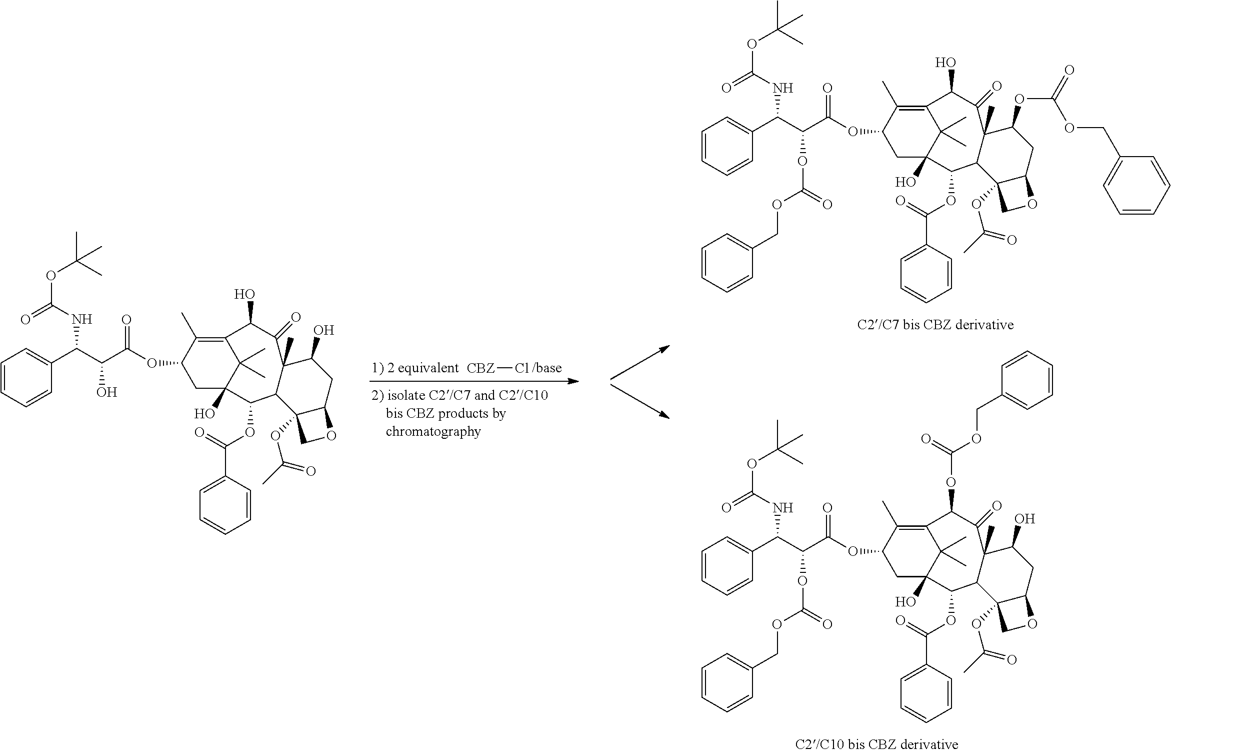

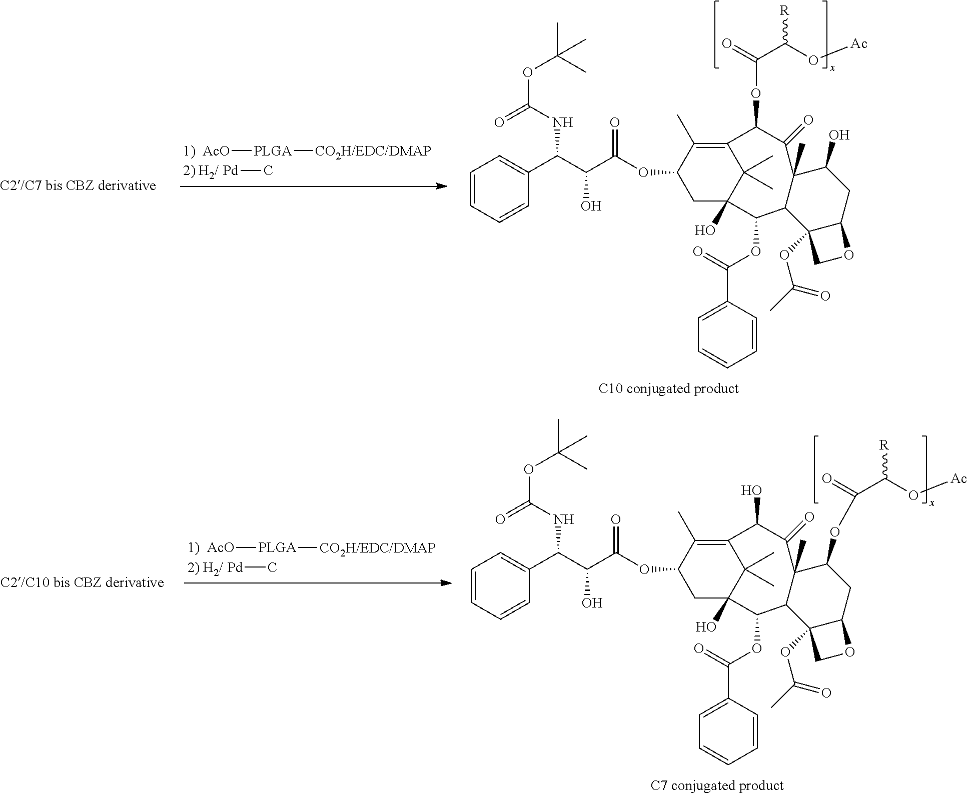

### Example 52

### 1,2-Diol Based Boronic Acid—Conjugate of Bortezomib with [(6-(acetoxy-PLGA-carboxamido)-2,3-dihydroxy-2,3-dimethylnonane

Method A:

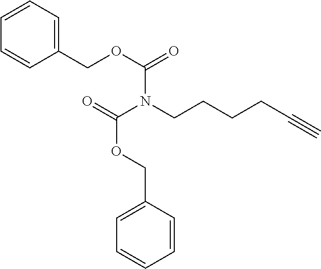 [SMILES: C#CCCCCN(C(=O)OCc1ccccc1)C(=O)OCc1ccccc1]

### Step 1: 6-Bis-(benzyloxycarbonyl)amino-1-hexyne

6-Chloro-1-hexyne (1.0 mmol) in THF will be treated with bis(benzyloxycarbonyl)amine (1.0 mmol) and potassium carbonate (1.2 mmol) in DMF (10 mL). After 16 h the reaction will be diluted with diethyl ether and washed successively with water, 1N hydrochloric acid and saturated sodium bicarbonate. After drying with sodium sulfate, the extract will be filtered and concentrated to give the crude product. This will be purified by chromatography. The structure will be confirmed with 1H-NMR and LC/MS.

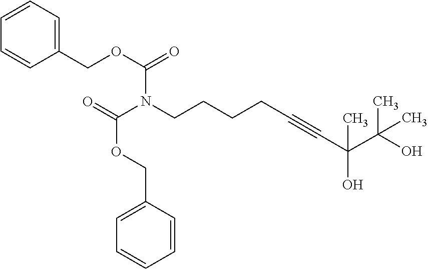 [SMILES: CC(C)(O)C(C)(O)C#CCCCCN(C(=O)OCc1ccccc1)C(=O)OCc1ccccc1]

### Step 2: 9-Bis-(benzyloxycarbonyl)amino-2,3-dihydroxy-2,3-dimethyl-4-nonyne

6-Bis-(benzyloxycarbonyl)amino-1-hexyne (1.0 mmol) will be treated with lithium diisopropylamide in THF at −78° C. After 15 minutes, 3-hydroxy-3-methyl-2-butanone in THF will be added. After 1 hour at −78° C. the reaction will be quenched with saturated ammonium chloride solution and allowed to warm to room temperature. The reaction mixture will then be diluted with diethyl ether and successively washed with water, 1N hydrochloric acid, and saturated sodium bicarbonate. After drying with sodium sulfate, the extract will be filtered and the solvent evaporated to give the crude product. This will be purified by chromatography. The structure will be verified by 1H-NMR and LC/MS.

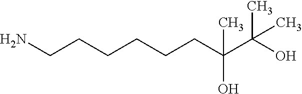

### Step 3: 9-amino-2,3-dihydroxy-2,3-dimethylnonane

To a suspension of 10% Pd/C in methanol (˜1 g of catalyst per 1 g of substrate) in an appropriately sized flask will be added a solution of 9-bis-(benzyloxycarbonyl)amino-2,3-dihydroxy-2,3-dimethyl-4-nonyne in methanol. The flask will be evacuated and after 1 minute filled with hydrogen gas. After the reaction is complete the mixture will be filtered to remove the catalyst and the solvent evaporated to yield the title product. The structure will be verified by 1H-NMR and LC/MS.

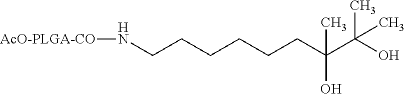

### Step 4: 9-(acetoxy-PLGA-carboxamido)-2,3-dihydroxy-2,3-dimethylnonane

A 100-mL round-bottom flask will be charged with 9-amino-2,3-dihydroxy-2,3-dimethylnonane (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

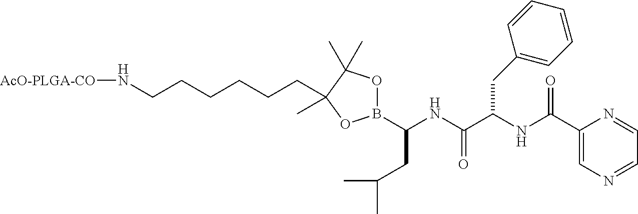

### Step 5: Conjugate of Bortezomib with 9-(acetoxy-PLGA-carboxamido)-2,3-dihydroxy-2,3-dimethylnonane

In a manner similar to that described by Hebel et al. (J. Org. Chem. 2002, 67, 9452) bortezomib (1.0 mmol) will be dissolved in DMF and treated with a solution of 9-(acetoxy-PLGA-carboxamido)-2,3-dihydroxy-2,3-dimethylnonane (1.0 mmol) in DMF and 4 Å MS. After 6 h at room temperature, the reaction mixture will be added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

Method B:

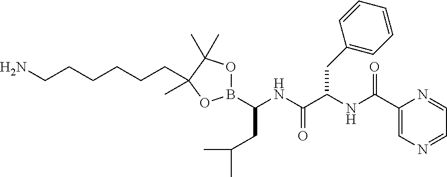 [SMILES: CC(C)C[C@H](NC(=O)[C@H](Cc1ccccc1)NC(=O)c1cnccn1)B1OC(C)(C)C(C)(CCCCCCN)O1]

### Step 1: Conjugate of Bortezomib with 9-amino-2,3-dihydroxy-2,3-dimethylnonane

In a manner similar to that described by Hebel et al. (J. Org. Chem. 2002, 67, 9452) bortezomib (1.0 mmol) will be dissolved in DMF and treated with a solution of 9-amino-2,3-dihydroxy-2,3-dimethylnonane (from Method A, Step 3) (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

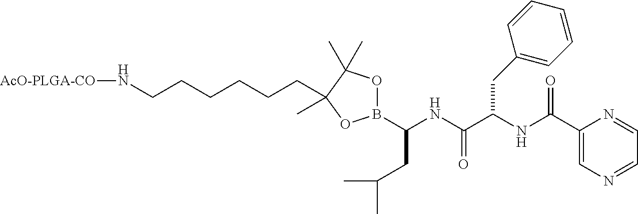

### Step 2: Conjugate of Bortezomib with 9-(acetoxy-PLGA-carboxamido)-2,3-dihydroxy-2,3-dimethylnonane

A 100-mL round-bottom flask will be charged with the conjugate of bortezomib with 9-amino-2,3-dihydroxy-2,3-dimethylnonane (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

### Example 53

### Diethanolamine Based Boronic Acid—Conjugate of Bortezomib with [(6-(acetoxy-PLGA-carboxamidohexyl)-bis-(2-hydroxyethyl]amine

Method A:

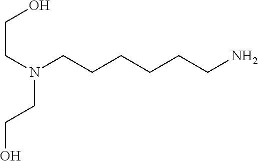 [SMILES: NCCCCCCN(CCO)CCO]

### Step 1: Bis-(2-hydroxyethyl)hexylamine

In the manner described by R. M. Peck et al. (J. Am. Chem. Soc. 1959, 81, 3984) the title compound will be prepared.

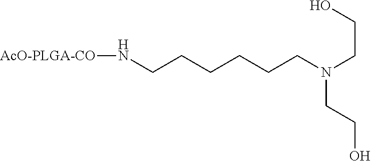

### Step 2: Bis-(2-hydroxyethyl)-[(6-(acetoxy-PLGA-carboxamidohexyl)amine

A 100-mL round-bottom flask will be charged with bis-(2-hydroxyethyl)hexylamine (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

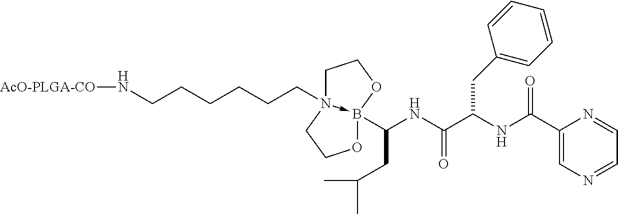

### Step 3: Conjugate of Bortezomib with bis-(2-hydroxyethyl)-[(6-(acetoxy-PLGA-carboxamidohexyl)amine

In a manner similar to that described by Hebel et al. (J. Org. Chem. 2002, 67, 9452) bortezomib (1.0 mmol) will be dissolved in DMF and treated with a solution of bis-(2-hydroxyethyl)-[(6-(acetoxy-PLGA-carboxamidohexyl)amine (1.0 mmol) in DMF and 4 Å MS. After 6 h at room temperature, the reaction mixture will be added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

Method B:

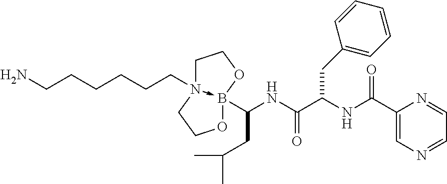 [SMILES: CC(C)C[C@H](NC(=O)[C@H](Cc1ccccc1)NC(=O)c1cnccn1)[B-]12OCC[N+]1(CCCCCCN)CCO2]

### Step 1: Conjugate of Bortezomib with bis-(2-hydroxyethyl)hexylamine

In a manner similar to that described by Hebel et al. (J. Org. Chem. 2002, 67, 9452) bortezomib (1.0 mmol) will be dissolved in DMF and treated with a solution of bis-(2-hydroxyethyl)hexylamine (from Method A, Step 1) (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

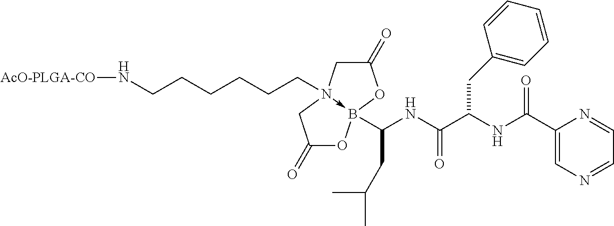

### Step 2: Conjugate of Bortezomib with bis-(2-hydroxyethyl)-[(6-(acetoxy-PLGA-carboxamidohexyl)amine

A 100-mL round-bottom flask will be charged with the conjugate of bortezomib with bis-(2-hydroxyethyl)hexylamine (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

### Example 54

### Iminodiacetic Acid Based Boronic Acid—Conjugate of Bortezomib with [(6-(acetoxy-PLGA-carboxamidohexyl)-carboxymethylamino]-acetate

Method A:

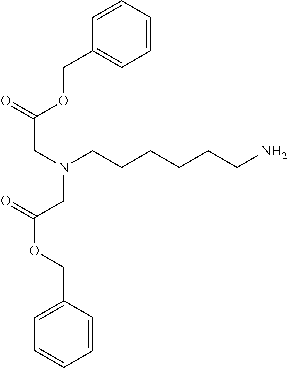 [SMILES: NCCCCCCN(CC(=O)OCc1ccccc1)CC(=O)OCc1ccccc1]

Step 1: Benzyl-[(6-aminohexyl)-benzyloxycarbonylmethylamino]-acetate hydrochloride:

In a manner similar to that described by M. Kruppa et al. (J. Am. Chem. Soc. 2005, 127, 3362) N-t-BOC-1,6-diamino-hexane (4.9 mmol) will be dissolved in MeCN (20 ml) and mixed with benzyl bromoacetate (10.6 mmol), potassium carbonate (2.92 g, 21.1 mmol) and a spatula tip of potassium iodide. The suspension will be stirred 2 days at 60° C. and monitored by TLC (ethyl acetate). The mixture will be filtrated, diluted with water and extracted with ethyl acetate. After drying over sodium sulfate the organic solvents will be evaporated to yield the crude product. Purification using column chromatography will give the t-BOC-protected iminodiacetic acid-intermediate.

To deprotect the t-BOC-group the purified product will be dissolved 4N HCl in dioxane. After approximately 1 h, the solvents will be evaporated to dryness to give the product as its hydrochloride salt. The structure will be confirmed with LC/MS and 1H-NMR.

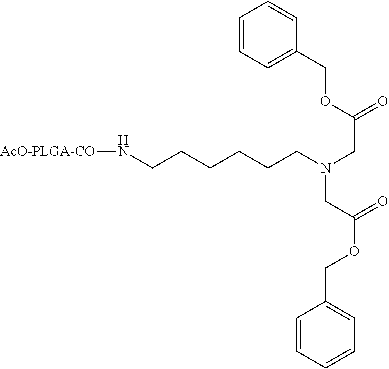

Step 2: Benzyl-[(6-(acetoxy-PLGA-carboxamidohexyl)-benzyloxycarbonylmethylamino]-acetate:

A 100-mL round-bottom flask will be charged with benzyl-[(6-aminohexyl)-benzyloxycarbonylmethylamino]-acetate hydrochloride (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

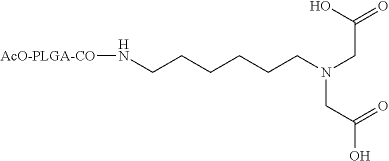

### Step 3: [(6-(acetoxy-PLGA-carboxamidohexyl)-carboxymethylamino]-acetate

A 100-mL, round-bottom flask equipped with a magnetic stirrer will be charged with benzyl-[(6-(acetoxy-PLGA-carboxamidohexyl)-benzyloxycarbonylmethylamino]-acetate [1.06 mmol], EtOAc (36 mL), and MeOH (0.5 mL). The mixture will stirred for 5 min to afford a clear solution. 5% Pd/C (200 mg, 50% moisture) will be charged. The mixture will be evacuated for 1 min and then filled with H2 with a balloon. The reaction will be stirred at ambient temperature for 3 h or until the reaction is complete. The mixture will be filtered through a Celite® pad to remove the catalyst; the combined filtrate concentrated and added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

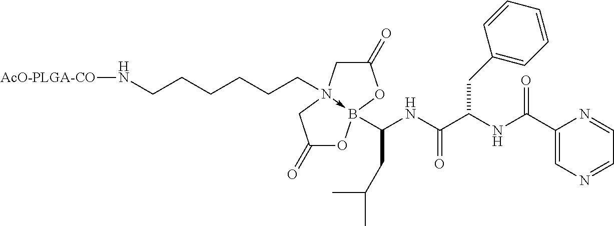

### Step 4: Conjugate of Bortezomib with [(6-(acetoxy-PLGA-carboxamidohexyl)-carboxymethylamino]-acetate

In a manner similar to that described by Hebel et al. (J. Org. Chem. 2002, 67, 9452) bortezomib (1.0 mmol) will be dissolved in DMF and treated with a solution of [(6-(acetoxy-PLGA-carboxamidohexyl)-carboxymethylamino]-acetate (1.0 mmol) in DMF and 4 Å MS. After 6 h at room temperature, the reaction mixture will be added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

Method B:

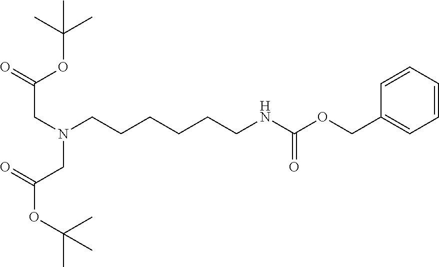 [SMILES: CC(C)(C)OC(=O)CN(CCCCCCNC(=O)OCc1ccccc1)CC(=O)OC(C)(C)C]

### Step 1: tert-Butyl-[(6-benzyloxycarbonylaminohexyl)-tert-butoxycarbonylmethylamino]-acetate

In the manner described by M. Kruppa et al. (J. Am. Chem. Soc. 2005, 127, 3362) the title compound will be produced.

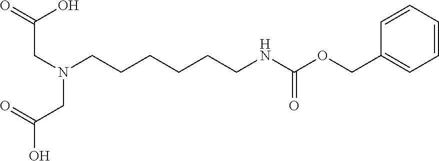 [SMILES: O=C(O)CN(CCCCCCNC(=O)OCc1ccccc1)CC(=O)O]

### Step 2: [(6-Benzyloxycarbonylaminohexyl)-carboxymethylamino]-acetate

To a solution of tert-butyl-[(6-benzyloxycarbonylaminohexyl)-tert-butoxycarbonylmethylamino]-acetate in dichloromethane will be added at 0° C. trifluoroacetic acid. After 1 hour the solvent will be evaporated to yield the title product. The structure will be confirmed with 1H-NMR and LC/MS.

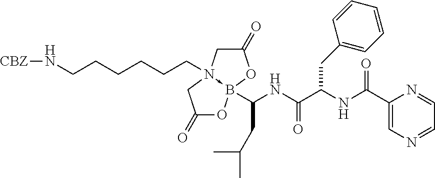

### Step 3: Conjugate of Bortezomib with [(6-(benzyloxycarbonylaminohexyl)-carboxymethylamino]-acetate

In a manner similar to that described by Hebel et al. (J. Org. Chem. 2002, 67, 9452) bortezomib (1.0 mmol) will be dissolved in DMF and treated with a solution of [(6-benzyloxycarbonylaminohexyl)-carboxymethylamino]-acetate (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

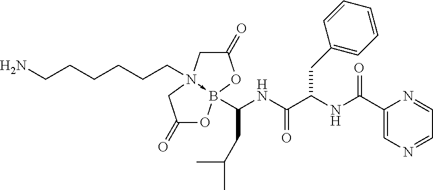 [SMILES: CC(C)C[C@H](NC(=O)[C@H](Cc1ccccc1)NC(=O)c1cnccn1)[B-]12OC(=O)C[N+]1(CCCCCCN)CC(=O)O2]

Step 4: Conjugate of Bortezomib with [(6-(aminohexyl)-carboxymethylamino]-acetate:

A 100-mL, round-bottom flask equipped with a magnetic stirrer will be charged with the conjugate of bortezomib with [(6-(benzyloxycarbonylaminohexyl)-carboxymethylamino]-acetate[1.06 mmol], EtOAc (36 mL), and MeOH (0.5 mL). The mixture will stirred for 5 min to afford a clear solution. 5% Pd/C (200 mg, 50% moisture) will be charged. The mixture will be evacuated for 1 min and then filled with H2 with a balloon. The reaction will be stirred at ambient temperature for 3 h or until the reaction is complete. The mixture will be added to MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

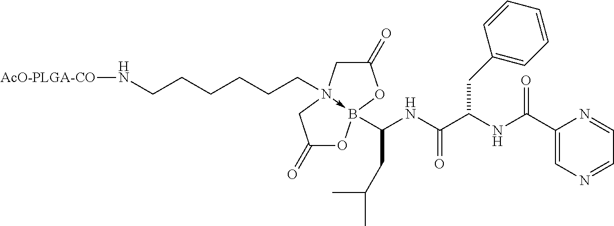

Step 5: Conjugate of Bortezomib with [(6-(acetoxy-PLGA-carboxamidohexyl)-carboxymethylamino]-acetate:

A 100-mL round-bottom flask will be charged with the conjugate of bortezomib with [(6-(aminohexyl)-carboxymethylamino]-acetate (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

### Example 55

### (Aminoethyl)(hydroxyethyl)amine Based Boronic Acid—Conjugate of Bortezomib with [(6-(acetoxy-PLGA-carboxamidohexyl)-(2-methylaminoethyl)-(2-hydroxyethyl)]amine

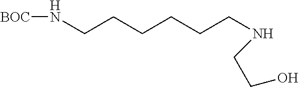 [SMILES: CC(C)(C)OC(=O)NCCCCCCNCCO]

### Step 1: (6-t-Butoxycarbonylaminohexyl)(2-hydroxyethyl)amine

In a manner similar to that described by Pellacini et al. (U.S. Pat. No. 6,455,576) the title compound will be prepared from 6-t-butoxycarbonylaminohexanol.

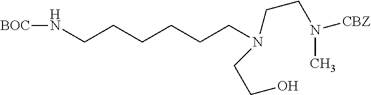

### Step 2: (6-t-Butoxycarbonylaminohexyl)-((2-benzyloxycarbonyl)methylaminoethyl)-(2-hydroxyethyl)amine

In a manner similar to that described by Ackerman et al. (US Patent Appl. 2005065210) the title compound will be prepared from ((2-benzyloxycarbonyl)methylaminoethanol and (6-t-butoxycarbonylaminohexyl)(2-hydroxyethyl)amine (from Step 1).

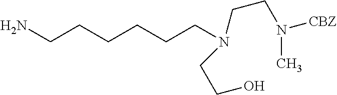

### Step 3: (6-Aminohexyl)-((2-benzyloxycarbonyl)methylaminoethyl)-(2-hydroxyethyl)amine bis-hydrochloride

(6-t-Butoxycarbonylaminohexyl)-((2-benzyloxycarbonyl)methylaminoethyl)-(2-hydroxyethyl)amine will be dissolved 4N HCl in dioxane. After approximately 1 h, the solvents will be evaporated to dryness to give the product as its bis-hydrochloride salt. The structure will be confirmed with LC/MS and 1H-NMR.

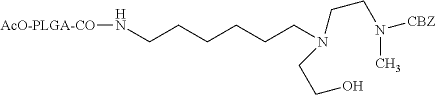

### Step 4: (6-(Acetoxy-PLGA-carboxamidohexyl)-((2-benzyloxycarbonyl)methylaminoethyl)-(2-hydroxyethyl)amine

A 100-mL round-bottom flask will be charged with (6-aminohexyl)-((2-benzyloxycarbonyl)methylaminoethyl)-(2-hydroxyethyl)amine bis-hydrochloride (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (4.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

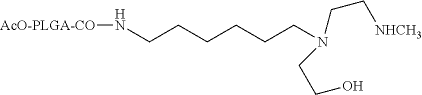

### Step 5: (6-(Acetoxy-PLGA-carboxamidohexyl)-(methylaminoethyl)-(2-hydroxyethyl)amine

A 100-mL, round-bottom flask equipped with a magnetic stirrer will be charged with (6-(acetoxy-PLGA-carboxamidohexyl)-((2-benzyloxycarbonyl)methylaminoethyl)-(2-hydroxyethyl)amine [1 mmol], EtOAc (36 mL), and MeOH (0.5 mL). The mixture will stirred for 5 min to afford a clear solution. 5% Pd/C (200 mg, 50% moisture) will be charged. The mixture will be evacuated for 1 min and then filled with H2 with a balloon. The reaction will be stirred at ambient temperature for 3 h or until the reaction is complete. The mixture will be filtered through a Celite® pad to remove the catalyst; the combined filtrate concentrated and added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

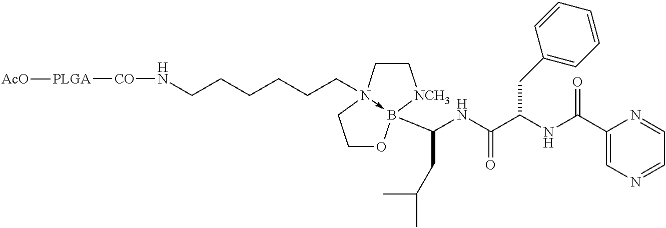

### Step 6: Conjugate of Bortezomib with (6-(acetoxy-PLGA-carboxamidohexyl)-(methylaminoethyl)-(2-hydroxyethyl)amine

In a manner similar to that described by Hebel et al. (J. Org. Chem. 2002, 67, 9452) bortezomib (1.0 mmol) will be dissolved in DMF and treated with a solution of (6-(acetoxy-PLGA-carboxamidohexyl)-(methylaminoethyl)-(2-hydroxyethyl)amine (1.0 mmol) in DMF and 4 Å MS. After 6 h at room temperature, the reaction mixture will be added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

### Example 56

### 1,2-amino Alcohol Based Boronic Acid—Conjugate of Bortezomib with (8-(acetoxy-PLGA-carboxamido)-2-hydroxy-2-methyl-1-methylaminooctane

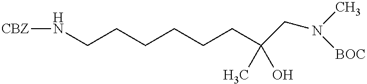

### Step 1: (8-(benzyloxycarbonylamino)-2-hydroxy-2-methyl-1-((t-butoxycarbonyl)methylamino)octane

In the manner described by Ortiz et al. (Tetrahedron 1999, 55, 4831) the title compound will be prepared from 8-benzyloxycarbonylamino-2-octanone. The structure will be confirmed with 1H-NMR and LC/MS.

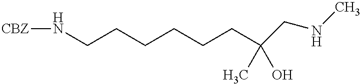

### Step 2: (8-(Benzyloxycarbonylamino)-2-hydroxy-2-methyl-1-(methylamino)octane

(8-(benzyloxycarbonylamino)-2-hydroxy-2-methyl-1-((t-butoxycarbonyl)methylamino)octane will be dissolved 4N HCl in dioxane. After approximately 1 h, the solvents will be evaporated to dryness to give the product as its hydrochloride salt. The structure will be confirmed with LC/MS and 1H-NMR.

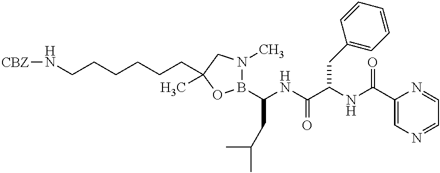

### Step 3: Conjugate of Bortezomib (8-(benzyloxycarbonylamino)-2-hydroxy-2-methyl-1-(methylamino) octane

In a manner similar to that described by Hebel et al. (J. Org. Chem. 2002, 67, 9452) bortezomib (1.0 mmol) will be dissolved in DMF and treated with a solution of (8-(benzyloxycarbonylamino)-2-hydroxy-2-methyl-1-(methylamino)octane (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

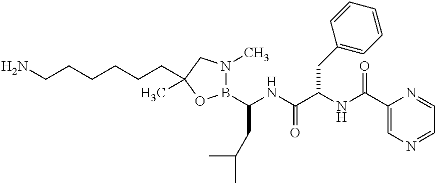 [SMILES: CC(C)C[C@H](NC(=O)[C@H](Cc1ccccc1)NC(=O)c1cnccn1)B1OC(C)(CCCCCCN)CN1C]

### Step 4: Conjugate of Bortezomib with (8-amino-2-hydroxy-2-methyl-1-(methylamino)octane

A 100-mL, round-bottom flask equipped with a magnetic stirrer will be charged with the conjugate of bortezomib (8-(benzyloxycarbonylamino)-2-hydroxy-2-methyl-1-(methylamino)octane [1 mmol], EtOAc (36 mL), and MeOH (0.5 mL). The mixture will be stirred for 5 min to afford a clear solution. 5% Pd/C (200 mg, 50% moisture) will be charged. The mixture will be evacuated for 1 min and then filled with H2 with a balloon. The reaction will be stirred at ambient temperature for 3 h or until the reaction is complete. The mixture will be filtered through a Celite® pad to remove the catalyst; the combined filtrate concentrated and added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

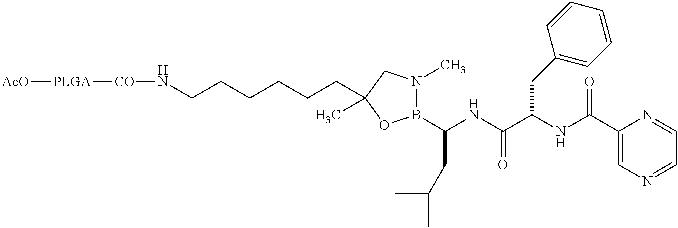

### Step 5: Conjugate of Bortezomib with (8-(acetoxy-PLGA-carboxamido)-2-hydroxy-2-methyl-1-(methylamino)octane

A 100-mL round-bottom flask will be charged with the conjugate of bortezomib with (8-amino-2-hydroxy-2-methyl-1-(methylamino)octane (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

### Example 57

### 1,3-Diol Based Boronic Acid—Conjugate of Bortezomib with (6-(acetoxy-PLGA-carboxamido)-1-hydroxy-2-(hydroxymethyl)hexane

Method A:

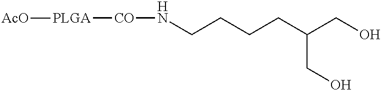

### Step 1:6-(Acetoxy-PLGA-carboxamido)-1-hydroxy-2-(hydroxymethyl)hexane

A 100-mL round-bottom flask will be charged with 6-amino-1-hydroxy-2-(hydroxymethyl)hexane (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

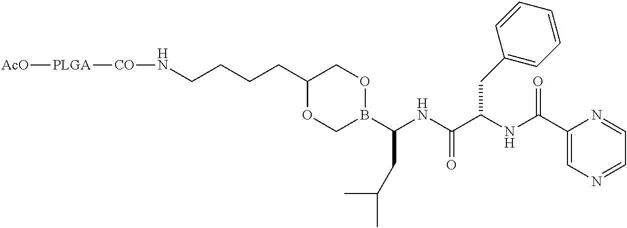

### Step 2: Conjugate of Bortezomib with (6-(acetoxy-PLGA-carboxamido)-1-hydroxy-2-(hydroxymethyl)hexane

In a manner similar to that described by Hebel et al. (J. Org. Chem. 2002, 67, 9452) bortezomib (1.0 mmol) will be dissolved in DMF and treated with a solution of 6-(acetoxy-PLGA-carboxamido)-1-hydroxy-2-(hydroxymethyl)hexane (1.0 mmol) in DMF and 4 Å MS. After 6 h at room temperature, the reaction mixture will be added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

Method B:

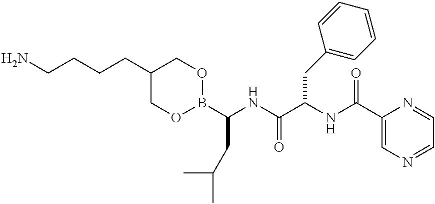 [SMILES: CC(C)C[C@H](NC(=O)[C@H](Cc1ccccc1)NC(=O)c1cnccn1)B1OCC(CCCCN)CO1]

### Step 1: Conjugate of Bortezomib with 6-amino-1-hydroxy-2-(hydroxymethyl)hexane

In a manner similar to that described by Hebel et al. (J. Org. Chem. 2002, 67, 9452) bortezomib (1.0 mmol) will be dissolved in DMF and treated with a solution of 6-amino-1-hydroxy-2-(hydroxymethyl)hexane (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

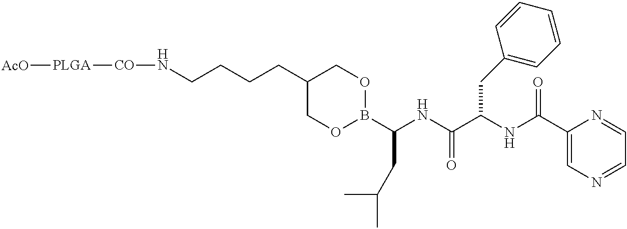

Step 2: Conjugate of Bortezomib with 6-(acetoxy-PLGA-carboxamido)-1-hydroxy-2-(hydroxymethyl)hexane:

A 100-mL round-bottom flask will be charged with the conjugate of bortezomib with 6-amino-1-hydroxy-2-(hydroxymethyl)hexane (1 mmol) and DMF (5 mL). The mixture will be stirred for 15 min to afford a clear solution. AcO-PLGA-CO2H (1.0 mmol) and DCM (20 mL) will be added and the mixture stirred for 10 min. EDC.HCl (1.3 mmol), DMAP (0.5 mmol), and TEA (2.5 mmol) will be added and the reaction stirred at ambient temperature for 6 h or until completion of the reaction. The reaction will be concentrated and added into a suspension of Celite® (13 g) in MTBE (300 mL) over 1 h with overhead stirring. The suspension will be stirred for another hour and filtered through a PP filter. The product/Celite® complex will be suspended in acetone (35 mL) after having been dried at ambient temperature for 16 h, stirred for 0.5 h, and filtered through a PP filter. The filter cake will be washed with acetone (3×10 mL). The filtrate will be concentrated and added dropwise into cold water (300 mL) over 1 h with overhead stirring. The suspension will be filtered through a PP filter; the filter cake washed with water (3×30 mL) and dried under vacuum at 28° C. for 2 days to afford the title product. The structure will be confirmed with 1H-NMR, HPLC and GPC.

### Example 58

### Formulation of 6-Aminohexyl-Carboxymethylamino Acetate Bortezomib PLGA Particles Via Nanoprecipitation Using PVA as the Surfactant

6-aminohexyl-carboxymethylamino acetate Bortezomib-5050 PLGA-O-acetyl (700 mg, 70 wt % or 600 mg, 60 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

### Example 59

### Formulation of 6-Aminohexyl-Carboxymethylamino Acetate Bortezomib PLGA Particles Via Nanoprecipitation Using Tween 80 as the Surfactant

6-aminohexyl-carboxymethylamino acetate Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Tween (Fisher Scientific) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Tween 80 and concentrated using a tangential flow filtration system (300 kDa MW cutoff, membrane area=50 cm²). The solution will be then passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 60

### Formulation of 6-Aminohexyl-Carboxymethylamino Acetate Bortezomib PLGA Particles Via Nanoprecipitation Using Solutol as the Surfactant

6-aminohexyl-carboxymethylamino acetate Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Solutol HS 15 (BASF) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Solutol HS 15 and concentrated using a tangential flow filtration system (300 kDa MW cutoff, membrane area=50 cm²). The solution will then be passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 61

### Formulation of 6-Aminohexyl-Carboxymethylamino Acetate Bortezomib PLGA Particles Via Micro-Mixer Using PVA as the Surfactant

5050 purified PLGA (211 mg, 24 wt %), 6-aminohexyl-carboxymethylamino acetate Bortezomib-5050 PLGA-O-acetyl (633 mg, 71 wt %) and mPEG-PLGA (Mw 8.3 kDa, 5 wt % total polymer, Akina) will be combined at a total concentration of 1.0% polymer in acetone. A separate solution of 0.5% PVA (80% hydrolyzed, Mw 9-10 kDa, Sigma) in water will be prepared. The organic and aqueous solutions will then be blended using a Caterpillar MicroMixer, using flow rates of 5 mL/min and 15 mL/min respectively. The acetone will be removed from the resulting nanoparticle dispersion by rotary evaporation. The aqueous nanoparticle dispersion will be washed with 10 volumes of water using a tangential flow filtration system (300 kDa MW cutoff, membrane area=50 cm²). The dispersion will then be concentrated using a tangential flow filtration system (300 kDa MW cutoff, membrane area=50 cm²). The solution will then be passed through a 0.22 μm filter, and adjusted to a final concentration of 10% sucrose. The solution was then lyophilized to provide the particles. Particle solution properties will be characterized by dynamic light scattering (DLS) spectrometer.

### Example 62

### Formulation of 6-Aminohexyl-Carboxymethylamino Acetate Bortezomib PLGA Particles Via Emulsion Using PVA as the Surfactant

6-aminohexyl-carboxymethylamino acetate Bortezomib-5050 PLGA-O-acetyl (100 mg, 100 wt %) will be dissolved to form a total concentration of 1.0% polymer in dichloromethane. In a separate solution, 0.5% w/v PVA (viscosity 2.5-3.5 cp, Sigma-Aldrich) in water will be prepared. The dissolved polymer solution in dichloromethane will be mixed with the aqueous PVA solution and emulsified through a microfluidizer processor (M-110 EH Microfluidics) for three cycles at a pressure of 8500 psi. Dichloromethane will be removed by stirring the solution for 12 hours. The nanoparticles will then be washed with 10 volumes of water and concentrated using a tangential flow filtration system (300 kDa MW cutoff, membrane area=50 cm²). The nanoparticle solution will be adjusted to a final concentration of 10% sucrose. The nanoparticles could be lyophilized into powder form. Particle solution properties will be characterized by dynamic light scattering (DLS) spectrometer.

### Example 63

### Formulation of 6-aminohexyl-carboxymethylamino Acetate Bortezomib PLGA and 2,3-dihydroxy-2,3-dimethylnonane Bortezomib PLGA Particles Via Nanoprecipitation Using PVA as the Surfactant

6-aminohexyl-carboxymethylamino acetate Bortezomib-5050 PLGA-O-acetyl (350 mg, 35 wt % or 300 mg, 30 wt %,), 2,3-dihydroxy-2,3-dimethylnonane Bortezomib PLGA (350 mg, 35 wt % or 300 mg, 30 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

### Example 64

### Formulation of bis-(2-hydroxyethyl)hexyamine Bortezomib PLGA Particles Via Nanoprecipitation Using PVA as the Surfactant

Bis-(2-Hydroxyethyl)hexyamine Bortezomib-5050 PLGA-O-acetyl (700 mg, 70 wt % or 600 mg, 60 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

### Example 65

### Formulation of bis-(2-hydroxyethyl)hexyamine Bortezomib PLGA Particles Via Nanoprecipitation Using Tween 80 as the Surfactant

Bis-(2-Hydroxyethyl)hexyamine Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Tween (Fisher Scientific) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Tween 80 and concentrated using a tangential flow filtration system (300 kDa MW cutoff, membrane area=50 cm²). The solution will be then passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 66

### Formulation of bis-(2-hydroxyethyl)hexyamine Bortezomib PLGA Particles Via Nanoprecipitation Using Solutol as the Surfactant

bis-(2-Hydroxyethyl)hexyamine Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Solutol HS 15 (BASF) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Solutol HS 15 and concentrated using a tangential flow filtration system (300 kDa MW cutoff, membrane area=50 cm²). The solution will then be passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 67

### Formulation of bis-(2-hydroxyethyl)hexyamine Bortezomib PLGA Particles Via Micro-Mixer Using PVA as the Surfactant

5050 purified PLGA (211 mg, 24 wt %), bis-(2-Hydroxyethyl)hexyamine Bortezomib-5050 PLGA-O-acetyl (633 mg, 71 wt %) and mPEG-PLGA (Mw 8.3 kDa, 5 wt % total polymer, Akina) will be combined at a total concentration of 1.0% polymer in acetone. A separate solution of 0.5% PVA (80% hydrolyzed, Mw 9-10 kDa, Sigma) in water will be prepared. The organic and aqueous solutions will then be blended using a Caterpillar MicroMixer, using flow rates of 5 mL/min and 15 mL/min respectively. The acetone will be removed from the resulting nanoparticle dispersion by rotary evaporation. The aqueous nanoparticle dispersion will be washed with 10 volumes of water using a tangential flow filtration system (300 kDa MW cutoff, membrane area=50 cm²). The dispersion will then be concentrated using a tangential flow filtration system (300 kDa MW cutoff, membrane area=50 cm²). The solution will then be passed through a 0.22 μm filter, and adjusted to a final concentration of 10% sucrose. The solution was then lyophilized to provide the particles. Particle solution properties will be characterized by dynamic light scattering (DLS) spectrometer.

### Example 68

### Formulation of bis-(2-hydroxyethyl)hexyamine Bortezomib PLGA Particles Via Emulsion Using PVA as the Surfactant

6 bis-(2-Hydroxyethyl)hexyamine Bortezomib-5050 PLGA-O-acetyl (100 mg, 100 wt %) will be dissolved to form a total concentration of 1.0% polymer in dichloromethane. In a separate solution, 0.5% w/v PVA (viscosity 2.5-3.5 cp, Sigma-Aldrich) in water will be prepared. The dissolved polymer solution in dichloromethane will be mixed with the aqueous PVA solution and emulsified through a microfluidizer processor (M-110 EH Microfluidics) for three cycles at a pressure of 8500 psi. Dichloromethane will be removed by stirring the solution for 12 hours. The nanoparticles will then be washed with 10 volumes of water and concentrated using a tangential flow filtration system (300 kDa MW cutoff, membrane area=50 cm²). The nanoparticle solution will be adjusted to a final concentration of 10% sucrose. The nanoparticles could be lyophilized into powder form. Particle solution properties will be characterized by dynamic light scattering (DLS) spectrometer.

### Example 69

### Formulation of bis-(2-hydroxyethyl)hexyamine Bortezomib PLGA and 6-aminohexyl-carboxymethylamino Acetate Bortezomib PLGA Particles Via Nanoprecipitation Using PVA as the Surfactant

bis-(2-Hydroxyethyl)hexyamine Bortezomib-5050 PLGA-O-acetyl (350 mg, 35 wt % or 300 mg, 30 wt %,), 6-aminohexyl-carboxymethylamino acetate Bortezomib PLGA (350 mg, 35 wt % or 300 mg, 30 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

### Example 70

### Formulation of 2,3-dihydroxy-2,3-dimethylnonane Bortezomib PLGA Particles Via Nanoprecipitation Using PVA as the Surfactant

2,3-dihydroxy-2,3-dimethylnonane Bortezomib-5050 PLGA-O-acetyl (700 mg, 70 wt % or 600 mg, 60 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

### Example 71

### Formulation of 2,3-dihydroxy-2,3-dimethylnonane Bortezomib PLGA Particles Via Nanoprecipitation Using Tween 80 as the Surfactant

2,3-dihydroxy-2,3-dimethylnonane Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Tween (Fisher Scientific) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Tween 80 and concentrated using a tangential flow filtration system (300 kDa MW cutoff, membrane area=50 cm²). The solution will be then passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 72

### Formulation of 2,3-dihydroxy-2,3-dimethylnonane Bortezomib PLGA Particles Via Nanoprecipitation Using Solutol as the Surfactant

2,3-dihydroxy-2,3-dimethylnonane Bortezomib-5050 PLGA-O-acetyl (672 mg, 84 wt %) and mPEG-PLGA (128 mg, 16 wt %, Mw 12.9 kDa, Lakeshore) will be dissolved to form a total concentration of 2.0% polymer in acetone. In a separate solution, 0.5% w/v Solutol HS 15 (BASF) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of 0.5% w/v Solutol HS 15 and concentrated using a tangential flow filtration system (300 kDa MW cutoff, membrane area=50 cm²). The solution will then be passed through a 0.22 μm Nylon filter, and adjusted to a final concentration of 10% sucrose. The nanoparticles could be lyophilized into powder form. Particle solution properties will be characterized by dynamic light scattering (DLS) spectrometer.

### Example 73

### Formulation of 2,3-dihydroxy-2,3-dimethylnonane Bortezomib PLGA Particles Via Micro-Mixer Using PVA as the Surfactant

5050 purified PLGA (211 mg, 24 wt %), 2,3-dihydroxy-2,3-dimethylnonane Bortezomib-5050 PLGA-O-acetyl (633 mg, 71 wt %) and mPEG-PLGA (Mw 8.3 kDa, 5 wt % total polymer, Akina) will be combined at a total concentration of 1.0% polymer in acetone. A separate solution of 0.5% PVA (80% hydrolyzed, Mw 9-10 kDa, Sigma) in water will be prepared. The organic and aqueous solutions will then be blended using a Caterpillar MicroMixer, using flow rates of 5 mL/min and 15 mL/min respectively. The acetone will be removed from the resulting nanoparticle dispersion by rotary evaporation. The aqueous nanoparticle dispersion will be washed with 10 volumes of water using a tangential flow filtration system (300 kDa MW cutoff, membrane area=50 cm²). The dispersion will then be concentrated using a tangential flow filtration system (300 kDa MW cutoff, membrane area=50 cm²). The solution will then be passed through a 0.22 μm filter, and adjusted to a final concentration of 10% sucrose. The solution was then lyophilized to provide the particles. Particle solution properties will be characterized by dynamic light scattering (DLS) spectrometer.

### Example 74

### Formulation of 2,3-dihydroxy-2,3-dimethylnonane Bortezomib PLGA Particles Via Emulsion Using PVA as the Surfactant

2,3-dihydroxy-2,3-dimethylnonane Bortezomib-5050 PLGA-O-acetyl (100 mg, 100 wt %) will be dissolved to form a total concentration of 1.0% polymer in dichloromethane. In a separate solution, 0.5% w/v PVA (viscosity 2.5-3.5 cp, Sigma-Aldrich) in water will be prepared. The dissolved polymer solution in dichloromethane will be mixed with the aqueous PVA solution and emulsified through a microfluidizer processor (M-110 EH Microfluidics) for three cycles at a pressure of 8500 psi. Dichloromethane will be removed by stirring the solution for 12 hours. The nanoparticles will then be washed with 10 volumes of water and concentrated using a tangential flow filtration system (300 kDa MW cutoff, membrane area=50 cm²). The nanoparticle solution will be adjusted to a final concentration of 10% sucrose. The nanoparticles could be lyophilized into powder form. Particle solution properties will be characterized by dynamic light scattering (DLS) spectrometer.

### Example 75

### Formulation of 2,3-dihydroxy-2,3-dimethylnonane Bortezomib PLGA and 6-aminohexyl-carboxymethylamino Acetate Bortezomib PLGA Particles Via Nanoprecipitation Using PVA as the Surfactant

2,3-dihydroxy-2,3-dimethylnonane Bortezomib-5050 PLGA-O-acetyl (350 mg, 35 wt % or 300 mg, 30 wt %,), 6-aminohexyl-carboxymethylamino acetate Bortezomib PLGA (350 mg, 35 wt % or 300 mg, 30 wt %,) and mPEG-PLGA (300 mg, 30 wt % or 400 mg, 40 wt %, Mw 12.9 kDa, Lakeshore) will be dissolved to form a total concentration of 1.0% polymer in acetone. In a separate solution, 0.5% w/v polyvinylalcohol (PVA) (80% hydrolyzed, Mw 9-10 kDa, Sigma) in water will be prepared. The polymer acetone solution will be added using a syringe pump at a rate of 1 mL/min to the aqueous solution (v/v ratio of organic to aqueous phase=1:10), with stirring at 500 rpm. Acetone will be removed by stirring the solution for 2-3 hours. The nanoparticles will then be washed with 10 volumes of water and concentrated using a tangential flow filtration system (300 kDa MW cutoff, membrane area=50 cm²). The solution will be then passed through a 0.22 μm filter, and adjusted to a final concentration of 10% sucrose. The nanoparticles could be lyophilized into powder form. Particle solution properties will be characterized by dynamic light scattering (DLS) spectrometer.

1.-121. (canceled)

122. A particle comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) said hydrophobic polymer covalently bonded to said therapeutic agent is a homopolymer or a polymer made up of more than one kind of monomeric subunit,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 25-80 weight % of said particle;
b) a plurality of hydrophilic-hydrophobic polymers, wherein
i) each of said hydrophilic-hydrophobic polymers of said plurality comprises a hydrophilic portion covalently bonded to a hydrophobic portion,
ii) said hydrophilic portion has a weight average molecular weight of about 1-6 kD, and
iii) said plurality of hydrophilic-hydrophobic polymers is about 5-30 weight % of said particle; and
c) a surfactant, wherein said surfactant is about 15-35 weight % of said particle.

123. The particle of claim 122, comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) said hydrophobic polymer covalently bonded to the therapeutic agent is a homopolymer or a polymer made up of more than one kind of monomeric subunit,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 25-80 weight % of said particle;
b) a plurality of hydrophilic-hydrophobic polymers, wherein
i) each of said hydrophilic-hydrophobic polymers of said plurality comprises a hydrophilic portion covalently bonded to a hydrophobic portion,
ii) said hydrophilic portion has a weight average molecular weight of about 1-6 kD, wherein
if the weight average molecular weight of said hydrophilic portion is about 1-3 kD, the ratio of the weight average molecular weight of said hydrophilic portion to the weight average molecular weight of said hydrophobic portion is between 1:3-1:7, and if the weight average molecular weight of said hydrophilic portion is about 4-6 kD, the ratio of the weight average molecular weight of said hydrophilic portion to the weight average molecular weight of said hydrophobic portion is between 1:1-1:4, and
iii) said plurality of hydrophilic-hydrophobic polymers is about 5-30 weight % of said particle; and
c) a surfactant, wherein said surfactant is about 15-35 weight % of said particle.

124. The particle of claim 122, comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) said hydrophobic polymer covalently bonded to said therapeutic agent is a homopolymer or a polymer made up of more than one kind of monomeric subunit,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 35-80 weight % of said particle;
b) a plurality of hydrophilic-hydrophobic polymers, wherein
i) each of said hydrophilic-hydrophobic polymers of said plurality comprises a hydrophilic portion covalently bonded to a hydrophobic portion,
ii) said hydrophilic portion has a weight average molecular weight of about 2-6 kD and said hydrophobic portion has a weight average molecular weight of between about 8-13 kD,
iii) said plurality of hydrophilic-hydrophobic polymers is about 10-25 weight % of said particle, and
iv) said hydrophilic portion of said hydrophilic-hydrophobic polymer terminates in an OMe; and
c) a surfactant, wherein said surfactant is about 15-35 weight % of said particle; wherein
said particle further comprises a hydrophobic polymer having a terminal acyl moiety.

125. A method of making the particle of claim 122, comprising:
providing an organic solution comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) said hydrophobic polymer covalently bonded to said therapeutic agent is a homopolymer or a polymer made up of more than one kind of monomeric subunit,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 25-80 weight % of said particle;
b) a plurality of hydrophilic-hydrophobic polymers, wherein
i) each of said hydrophilic-hydrophobic polymers of said plurality comprises a hydrophilic portion covalently bonded to a hydrophobic portion,
ii) said hydrophilic portion has a weight average molecular weight of about 1-6 kD, and
iii) said plurality of hydrophilic-hydrophobic polymers is about 5-30 weight % of said particle; and
combining said organic solution with an aqueous solution comprising a solvent to provide said particles.

126. A pharmaceutically acceptable composition comprising a plurality of particles of claim 122.

127. A kit comprising a plurality of particles of claim 122.

128. A dosage unit comprising a plurality of particles of claim 122.

129. The particle of claim 122, comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) said hydrophobic polymer covalently bonded to said therapeutic agent is a homopolymer or a polymer made up of more than one kind of monomeric subunit,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 25-80 weight % of said particle;
b) a plurality of polyethylene glycol (“PEG”)-hydrophobic polymers, wherein
i) each of said PEG-hydrophobic polymers of said plurality comprises a PEG portion covalently bonded to a hydrophobic portion,
ii) said PEG portion has a weight average molecular weight of about 1-6 kD, and
iii) said plurality of PEG-hydrophobic polymers is about 5-30 weight % of said particle; and
c) a surfactant, wherein said surfactant is about 15-35 weight % of said particle.

130. The particle of claim 129, comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) the hydrophobic polymer is made up of a first and a second type of monomeric subunit, and the ratio of the first to second type of monomeric subunit is said hydrophobic polymer covalently bonded to said therapeutic agent is from about 25:75 to about 75:25,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 25-80 weight % of said particle;
b) a plurality of PEG-hydrophobic polymers, wherein
i) each of said PEG-hydrophobic polymers of said plurality comprises a PEG portion covalently bonded to a hydrophobic portion,
ii) said PEG portion has a weight average molecular weight of about 1-6 kD, wherein
if the weight average molecular weight of said PEG portion is about 1-3 kD, the ratio of the weight average molecular weight of said PEG portion to the weight average molecular weight of said hydrophobic portion is between 1:3-1:7, and if the weight average molecular weight of said PEG portion is about 4-6 kD, the ratio of the weight average molecular weight of said PEG portion to the weight average molecular weight of said hydrophobic portion is between 1:1-1:4, and
iii) said plurality of PEG-hydrophobic polymers is about 5-30 weight % of said particle; and
c) a surfactant, wherein said surfactant is about 15-35 weight % of said particle.

131. The particle of claim 129, comprising:
a) a plurality of hydrophobic polymer-therapeutic agent conjugates, wherein
i) each hydrophobic polymer-therapeutic agent conjugate of said plurality comprises a hydrophobic polymer covalently bonded to a therapeutic agent,
ii) the hydrophobic polymer is made up of a first and a second type of monomeric subunit, and the ratio of the first to the second type of monomeric subunit in said hydrophobic polymer covalently bonded to said therapeutic agent is from about 25:75 to about 75:25,
iii) said hydrophobic polymer covalently bonded to said therapeutic agent has a weight average molecular weight of about 4-15 kD,
iv) said therapeutic agent is about 1-30 weight % of said particle, and
v) said plurality of hydrophobic polymer-therapeutic agent conjugates is about 35-80 weight % of said particle;
b) a plurality of PEG-hydrophobic polymers, wherein
i) each of said PEG-hydrophobic polymers of said plurality comprises a PEG portion covalently bonded to a hydrophobic portion,
ii) said PEG portion has a weight average molecular weight of about 2-6 kD and said hydrophobic portion has a weight average molecular weight of between about 8-13 kD,
iii) said plurality of PEG-hydrophobic polymers is about 10-25 weight % of said particle, and
iv) said PEG portion of said PEG-hydrophilic polymer terminates in an OMe; and
c) a surfactant, wherein said surfactant is about 15-35 weight % of said particle.

132. The particle of claim 122 comprising:
a) a plurality of poly(lactic-co-glycolic acid) (“PLGA”)-therapeutic agent conjugates, wherein
i) each PLGA-therapeutic agent conjugate of said plurality comprises a PLGA polymer covalently bonded to a therapeutic agent,
ii) the ratio of lactic acid to glycolic acid in said PLGA polymer covalently bonded to said therapeutic agent is from about 25:75 to about 75:25,
iii) said PLGA polymer covalently bonded to said therapeutic agent has a weight average molecular weight of about 4-15 kD,
iv) said therapeutic agent is about 1-30 weight % of said particle, and
v) said plurality of PLGA-therapeutic agent conjugates is about 25-80 weight % of said particle;
b) a plurality of PEG-PLGA polymers, wherein
i) each of said PEG-PLGA polymers of said plurality comprises a PEG portion covalently bonded to a PLGA portion,
ii) said PEG portion has a weight average molecular weight of about 1-6 kD, and
iii) said plurality of PEG-PLGA polymers is about 5-30 weight % of said particle; and
c) a surfactant, wherein said surfactant is about 15-35 weight % of said particle.

133. The particle of claim 132, comprising:
a) a plurality of PLGA-therapeutic agent conjugates, wherein
i) each PLGA-therapeutic agent conjugate of said plurality comprises a PLGA polymer covalently bonded to a therapeutic agent,
ii) the ratio of lactic acid to glycolic acid in said PLGA polymer covalently bonded to said therapeutic agent is from about 25:75 to about 75:25,
iii) said PLGA polymer covalently bonded to said therapeutic agent has a weight average molecular weight of about 4-15 kD,
iv) said therapeutic agent is about 1-30 weight % of said particle, and
v) said plurality of PLGA-therapeutic agent conjugates is about 25-80 weight % of said particle;
b) a plurality of PEG-PLGA polymers, wherein
i) each of said PEG-PLGA polymers of said plurality comprises a PEG portion covalently bonded to a PLGA portion,
ii) said PEG portion has a weight average molecular weight of about 1-6 kD, wherein
if the weight average molecular weight of said PEG portion is about 1-3 kD, the ratio of the weight average molecular weight of said PEG portion to the weight average molecular weight of said PLGA portion is between 1:3-1:7, and if the weight average molecular weight of said PEG portion is about 4-6 kD, the ratio of the weight average molecular weight of said PEG portion to the weight average molecular weight of said PLGA portion is between 1:1-1:4, and
iii) said plurality of PEG-PLGA polymers is about 5-30 weight % of said particle; and
c) a surfactant, wherein said surfactant is about 15-35 weight % of said particle.

134. The particle of claim 132, comprising:
a) a plurality of PLGA-therapeutic agent conjugates, wherein
i) each PLGA-therapeutic agent conjugate of said plurality comprises a PLGA polymer covalently bonded to a therapeutic agent,
ii) the ratio of lactic acid to glycolic acid in said PLGA polymer covalently bonded to said therapeutic agent is from about 25:75 to about 75:25,
iii) said PLGA polymer covalently bonded to said therapeutic agent has a weight average molecular weight of about 4-15 kD,
iv) said therapeutic agent is about 1-30 weight % of said particle, and
v) said plurality of PLGA-therapeutic agent conjugates is about 35-80 weight % of said particle;
b) a plurality of PEG-PLGA polymers, wherein
i) each of said PEG-PLGA polymers of said plurality comprises a PEG portion covalently bonded to a PLGA portion,
ii) said PEG portion has a weight average molecular weight of about 2-6 kD and said PLGA portion has a weight average molecular weight of between about 8-13 kD,
iii) said plurality of PEG-PLGA polymers is about 10-25 weight % of said particle, and
iv) said PEG portion of said PEG-PLGA polymer terminates in an OMe; and
c) a surfactant, wherein said surfactant is about 15-35 weight % of said particle; wherein
said particle further comprises PLGA having a terminal acyl moiety.

135. The particle of any one of claim 122, 123, 124, 129, 130, 131, 132, 133, or 134, wherein the therapeutic agent is a compound of Formula (A): 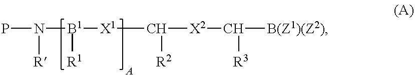
or a pharmaceutically acceptable salt thereof, wherein:
P is hydrogen or an amino-group-protecting moiety;
B¹, at each occurrence, is independently one of N or CH;
X¹, at each occurrence, is independently one of —C(O)—NH—, —CH₂—NH—, —CH(OH)—CH₂—, —CH(OH)—CH(OH)—, —CH(OH)—CH₂—NH—, —CH═CH—, —C(O)CH₂—, —SO₂—NH—, —SO₂—CH₂— or —CH(OH)—CH₂—C(O)—NH—, provided that when B¹ is N, then the X¹ is —C(O)—NH—;
X² is one of —C(O)—NH—, —CH(OH)—CH₂—, —CH(OH)—CH(OH)—, —C(O)—CH₂—, —SO₂—NH—, —SO₂—CH₂— or —CH(OH)—CH₂—C(O)—NH—;
R′ is hydrogen or alkyl, or R′ forms together with the adjacent R¹, or when A is zero, forms together with the adjacent R², a nitrogen-containing mono-, bi- or tri-cyclic, saturated or partially saturated ring system having 4-14 ring members, that is optionally substituted by one or two of keto, hydroxy, alkyl, aryl, aralkyl, alkoxy or aryloxy;
R¹, at each occurrence, is independently one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle is optionally substituted;
R² is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle is optionally substituted;
R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle is optionally substituted;
R⁵, in each instance, is one of aryl, aralkyl, alkaryl, cycloalkyl, a 5-10 membered saturated, partially unsaturated or aromatic heterocycle or —W—R⁶, where W is a chalcogen and R⁶ is alkyl, where the ring portion of any of said aryl, aralkyl, alkaryl or heterocycle is optionally substituted;
Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, one or more heteroatoms independently selected from N, S, and O; and
A is 0, 1, or 2.

136. The particle of claim 135, wherein:
P is R⁷—C(O)—, where R⁷ is heteroaryl or heteroarylalkyl;
X² is —C(O)—NH—;
R′ is hydrogen or alkyl, or R′ forms together with the adjacent R¹, or when A is zero, forms together with the adjacent R², a nitrogen-containing mono-, bi- or tri-cyclic, saturated or partially saturated ring system having 4-14 ring members, that is optionally substituted by one or two of keto, hydroxy, alkyl, aryl, aralkyl, alkoxy or aryloxy;
R² is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle is optionally substituted;
R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle is optionally substituted;
R⁵, in each instance, is one of aryl, aralkyl, alkaryl, cycloalkyl, a 5-10 membered saturated, partially unsaturated or aromatic heterocycle or —W—R⁶, where W is a chalcogen;
R⁶ is alkyl, where the ring portion of any of said aryl, aralkyl, alkaryl or heterocycle is optionally substituted;
Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, one or more heteroatoms independently selected from N, S, and O; and
A is zero.

137. The particle of claim 135, wherein:
P is hydrogen or an amino group protecting moiety;
A is zero;
X² is —C(O)—NH—;
R′ is hydrogen or C₁₋₈ alkyl;
R² is —CH₂—R⁵;
R³ is C₄ alkyl;
R⁵ is aryl, wherein R⁵ is optionally substituted by one or two substituents independently selected from the group consisting of C₁₋₆ alkyl, C₃₋₈ cycloalkyl, C₁₋₆ alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₁₋₆ alkyl(C₆₋₁₀)aryl, and halo(C₆₋₁₀)aryl;
Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, one or more heteroatoms independently selected from N, S, and O; and
A is zero.

138. The particle of claim 135, wherein:
P is hydrogen or an amino-group protecting moiety;
R′ is hydrogen or alkyl;
A is 0, 1, or 2;
R¹, R², and R³ are each independently hydrogen, alkyl, cycloalkyl, aryl, or —CH₂—R⁵;
R⁵, in each instance, is aryl, aralkyl, alkaryl, cycloalkyl, 5 to 10 membered saturated, partially unsaturated or aromatic heterocycle, or —W—R⁶, where W is a chalcogen and R⁶ is alkyl;
wherein the ring portion of any of said aryl, aralkyl, alkaryl, or heterocycle in R¹, R², R³, or R⁵ is optionally substituted; and
Z¹ and Z² together form a moiety derived from a sugar, wherein the atom covalently bonded to boron in each case is an oxygen atom.

139. The particle of claim 135, wherein:
P is R′ or R⁷—C(O)— or R⁷—SO₂—, wherein R⁷ selected from the group consisting of 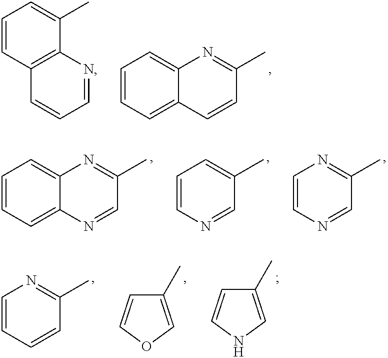
or P is 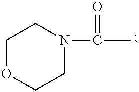
X₂ is selected from the group consisting of 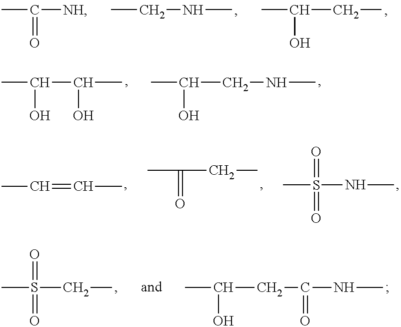
R′ is hydrogen or alkyl;
R₂ and R₃ are independently selected from the group consisting of hydrogen, alkyl, cycloalkyl, aryl, heterocycle and —CH₂—R₅, where R₅ is aryl, aralkyl, alkaryl, cycloalkyl, heterocycle or —Y—R₆,
where Y is a chalcogen, and R₆ is alkyl; and
Z₁ and Z₂ are independently one of hydroxy, alkoxy, or aryloxy, or together Z₁ and Z₂ form a moiety derived from a dihydroxy compound having at least two hydroxy groups separated by at least two connecting atoms in a chain or ring, said chain or ring comprising carbon atoms, and optionally, one or more heteroatoms independently selected from N, S, and O.

140. The particle of claim 135, wherein the compound of formula (A) is represented by the following structural formula: 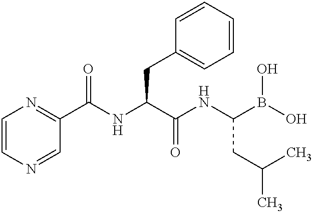 [SMILES: CC(C)C[C@H](NC(=O)[C@H](Cc1ccccc1)NC(=O)c1cnccn1)B(O)O]
or a pharmaceutically acceptable salt thereof.

141. The particle of any one of claim 122, 123, 124, 129, 130, 131, 132, 133, or 134, wherein the diameter of said particle is less than about 200 nm.

142. The pharmaceutically acceptable composition of claim 126, wherein the pharmaceutical composition further comprises a lyoprotectant.

143. The particle of any one of claim 122, 123, 124, 129, 130, 131, 132, 133, or 134, wherein the surfactant in c) is poly(vinyl alcohol) (“PVA”), wherein the PVA has a weight average molecular weight of about 5-45 kD.

144. The particle of either of claim 123 or 124, wherein the PEG-hydrophobic polymers in b) are PEG-PLGA polymers.

145. The particle of either of claim 130 or 131, wherein the PEG-hydrophobic polymers in b) are PEG-PLGA polymers.

146. The particle of any of claim 122, 123, 124, 130 or 131, wherein the hydrophobic polymer of the hydrophobic polymer-therapeutic agent conjugates in a) is PLGA.

147. The particle of claim 144, wherein the hydrophobic polymer of the hydrophobic polymer-therapeutic agent conjugates in a) is PLGA.

148. The particle of either of claim 130 or 131, wherein the hydrophobic polymer of the hydrophobic polymer-therapeutic agent conjugates in a) is PLGA.

149. The particle of any one of claim 122, 123, 124, 129, 130, 131, 132, 133 or 134, wherein the therapeutic agent is an anti-cancer agent.

150. The particle of any one of claim 122, 123, 124, 129, 130, 131, 132, 133, or 134, wherein the therapeutic agent is an anti-inflammatory agent.

151. The particle of any one of claim 122, 123, 124, 129, 130, 131, 132, 133, or 134, wherein the therapeutic agent is an agent for the treatment of a cardiovascular disease.

152. The particle of claim 151, wherein the agent for the treatment of a cardiovascular disease is efonidipine.

153. The particle of claim 143, wherein the therapeutic agent is an anti-cancer agent.

154. The particle of claim 143, wherein the therapeutic agent is an anti-inflammatory agent.

155. The particle of claim 143, wherein the therapeutic agent is an agent for the treatment of a cardiovascular disease.

156. The particle of claim 144, wherein the therapeutic agent is an anti-cancer agent.

157. The particle of claim 144, wherein the therapeutic agent is an anti-inflammatory agent.

158. The particle of claim 144, wherein the therapeutic agent is an agent for the treatment of a cardiovascular disease.

159. The particle of claim 145, wherein the therapeutic agent is an anti-cancer agent.

160. The particle of claim 145, wherein the therapeutic agent is an anti-inflammatory agent.

161. The particle of claim 145, wherein the therapeutic agent is an agent for the treatment of a cardiovascular disease.

162. The particle of claim 146, wherein the therapeutic agent is an anti-cancer agent.

163. The particle of claim 146, wherein the therapeutic agent is an anti-inflammatory agent.

164. The particle of claim 146, wherein the therapeutic agent is an agent for the treatment of a cardiovascular disease.

165. The particle of claim 147, wherein the therapeutic agent is an anti-cancer agent.

166. The particle of claim 147, wherein the therapeutic agent is an anti-inflammatory agent.

167. The particle of claim 147, wherein the therapeutic agent is an agent for the treatment of a cardiovascular disease.
